(19) United States
(12) Patent Application Publication
(10) Pub. No.: US 2020/0255523 A1
(43) Pub. Date: Aug. 13, 2020

(54) FC SILENCED ANTIBODY DRUG CONJUGATES (ADCS) AND USES THEREOF

(71) Applicant: Magenta Therapeutics, Inc., Cambridge, MA (US)

(72) Inventors: Rahul Palchaudhuri, Somerville, MA (US); Anthony Boitano, Newton, MA (US); Charlotte Fenton McDonagh, Winchester, MA (US); Bradley R. Pearse, Watertown, MA (US); Ganapathy N. Sarma, Belmont, MA (US)

(21) Appl. No.: 16/863,948

(22) Filed: Apr. 30, 2020

Related U.S. Application Data
(63) Continuation of application No. PCT/US2019/057741, filed on Oct. 23, 2019.
(60) Provisional application No. 62/807,363, filed on Feb. 19, 2019.
(60) Provisional application No. 62/773,839, filed on Nov. 30, 2018.
(60) Provisional application No. 62/749,662, filed on Oct. 23, 2018.

(51) Int. Cl. C07K 16/28; A61K 47/68

(52) U.S. Cl. CPC C07K 16/289; C07K 16/2806; C07K 16/2896; C07K 16/2878; C07K 2317/24; A61K 47/6831; C07K 2317/52; C07K 2319/55; C07K 2317/21; C07K 16/2875

(57) ABSTRACT

Disclosed are antibodies and antibody drug conjugates having an Fc region with substitutions resulting in essentially a silent Fc region. The antibodies and antibody drug conjugates described herein are useful for the depletion of cells and for the treatment of various hematopoietic diseases, metabolic disorders, cancers, e.g., acute myeloid leukemia (AML) and autoimmune diseases, among others. The compositions and methods described herein can be used to treat a disorder directly, for instance, by depleting, e.g., a population of CD45+ or CD117+ cancer cells or CD45+ autoimmune cells. The compositions and methods described herein can also be used to prepare a patient for hematopoietic stem cell transplant therapy and to improve the engraftment of hematopoietic stem cell transplants by selectively depleting endogenous hematopoietic stem cells prior to the transplant procedure.

### RELATED APPLICATIONS

This application is a continuation of International Application No. PCT/US2019/057741, filed on Oct. 23, 2019. International Application No. PCT/US2019/057741 claims priority to U.S. Provisional Application No. 62/749,662, filed on Oct. 23, 2018; U.S. Provisional Application No. 62/773,839, filed on Nov. 30, 2018; and U.S. Provisional Application No. 62/807,363, filed on Feb. 19, 2019. The contents of each of the priority applications is incorporated by reference herein.

### FIELD

The present disclosure relates to the field of antibodies or antibody drug conjugates thereof comprising an Fc region that has altered effector functions as a result of one or more amino acid substitutions in the Fc region. The present disclosure further relates to the treatment of patients suffering from various pathologies, such as blood diseases, metabolic disorders, cancers, and autoimmune diseases, among others, by administration of an antibody or antibody drug conjugate (ADC) having a modified Fc region, wherein the antibody or ADC is capable of binding an antigen expressed by a hematopoietic cell, such as a hematopoietic stem cell or cells of the host immune system.

### SEQUENCE LISTING

This application contains a Sequence Listing which is submitted herewith in electronically readable format. The Sequence Listing file was created on Apr. 30, 2020, is named “M103034_1700USC1_SL.txt” and its size is 197 KB. The entire contents of the Sequence Listing in the sequencelisting.txt file are incorporated by reference herein.

### BACKGROUND

The Fc region of an antibody controls antibody cytotoxic activities and can impact serum half-life of the antibody. In a therapeutic context, however, the cytotoxic effector function of an antibody is often not desirable and can create safety concerns and unwanted side effects by activating host immune defenses. Several amino acid changes in the Fc region have been reported to silence or reduce the effector function of antibodies. In fact, previous studies have identified amino acid positions within the Fc region of antibodies that impact the ability of the antibody to bind to an Fc receptor (see, for example, Wang et al. (2018) Protein Cell. 2018 January; 9(1): 63-73). For example, Fc mutations S239D and I332E have been described in the literature as enhancing ADCC function (see, e.g., Lazar et al. (2006) Engineered antibody Fc variants with enhanced effector function. Proc Natl Acad Sci USA. 103:4005-4010). Other mutations are associated with reduced FcγR and C1q binding, e.g., in an IgG1, amino acid changes L234A/L235A, or in an IgG4, amino acid changes F234A/L235A (Xu et al. In vitro characterization of five humanized OKT3 effector function variant antibodies. Cell Immunol. 2000; 200:16-26). What is less known, however, is how Fc mutations may impact antibody drug conjugates (ADCs), especially when the toxin is conjugated to the antibody or Fc containing fragment within the Fc region.

Despite advances in the medicinal arts, there remains a demand for treating pathologies of the hematopoietic system, such as diseases of a particular blood cell, metabolic disorders, cancers, and autoimmune conditions, among others. While hematopoietic stem cells have significant therapeutic potential, a limitation that has hindered their use in the clinic has been the difficulty associated with ensuring engraftment of hematopoietic stem cell (HSC) transplants in a host. In particular, hematopoietic stem cell therapies involving antibodies that target cell surface antigens on endogenous HSCs can trigger unwanted immunostimulatory and effector functions that impede engraftment of an exogenous HSC transplant. There is currently a need for compositions and methods for promoting the engraftment of exogenous hematopoietic stem cell grafts such that the multi-potency and hematopoietic functionality of these cells is preserved following transplantation. There is also a need for improved ADCs, for example, that can be used for conditioning having reduced effector function to reduce potential cytokine secretion and possible side effects.

### SUMMARY

Described herein are antibodies, and antigen binding portions thereof, comprising an Fc region that has altered effector functions as a result of one or more amino acid substitutions in the Fc region, as well as antibody drug conjugates, compositions, and methods of using said antibodies. In particular, provided herein are antibodies or antibody drug conjugates (ADC) that have modified Fc regions, wherein the antibodies or ADCs are capable of binding an antigen expressed by a hematopoietic cell, such as a hematopoietic stem cell or a mature immune cell (e.g., T cells). Further, provided herein are ADCs containing Fc mutations that provide a conjugation site for the toxin and reduce effector function, as well as provide stability. Thus, the disclosure provides unique combinations of Fc mutations for ADCs.

In one aspect, provided herein is an antibody comprising an Fc region, wherein the Fc region comprises an amino acid substitution at positions L234 and L235 (EU index), and amino acid substitution D265C (EU index), and wherein the antibody is an intact IgG antibody. In one embodiment, the Fc region comprises an amino acid substitution at positions L234 and L235 (EU index), and amino acid substitution D265A (EU index), and wherein the antibody is an intact IgG antibody. In one embodiment, the L234 amino acid substitution is L234A. In one embodiment, the L235 amino acid substitution is L235A.

In some embodiments, the Fc region further comprises an amino acid substitution at position H435 (EU index). In one embodiment, the H435 amino acid substitution is H435A. In one embodiment, the antibody comprising amino acid substitution H435A has a decreased half-life relative to an identical intact IgG antibody comprising an unmodified Fc region.

In another aspect, provided herein is an antibody comprising an Fc region having amino acid substitutions consisting essentially of amino acid substitutions L234A, L235A, and D265C (EU index), and wherein the antibody is an intact IgG antibody. In one embodiment, the antibody comprising an Fc region having amino acid substitutions consisting essentially of amino acid substitutions L234A, L235A, and D265A (EU index), and wherein the antibody is an intact IgG antibody.

In another aspect, provided herein is an antibody, or an antigen-binding portion thereof, comprising an Fc region, wherein the Fc regions comprises amino acid substitutions at positions L234, L235 (EU index), and D265 (EU index). In one embodiment, the D265 amino acid substitution is D265C or D265A (EU index). In another embodiment, the L234 amino acid substitution is L234A or L234V. In another embodiment, the L235 amino acid substitution is L235A. In another embodiment, the Fc region further comprises an amino acid substitution at position N297 (EU index). In another embodiment, the N297 amino acid substitution is selected from the group consisting of N297A, N297G and N297Q (EU index). In another embodiment, the Fc region further comprises an amino acid substitution at position E233 (EU index). In another embodiment, the E233 amino acid substitution is E233P (EU index). In another embodiment, the Fc region further comprises a deletion of G236 (EU index). In another embodiment, the Fc region further comprises an amino acid substitution at position P331 (EU index). In another embodiment, the P331 amino acid substitution is P331G. In another embodiment, the Fc region does not include a substitution at position P331 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position I253 (EU index). In another embodiment, the I253 amino acid substitution is I253A. In another embodiment, the Fc region further comprises an amino acid substitution at position H310 (EU index). In another embodiment, the H310 amino acid substitution is H310A.

In another aspect, provided herein is an antibody, or antigen-binding portion thereof, comprising an Fc region, wherein the Fc region comprises an amino acid substitution at position N297 and D265 (EU index). In one embodiment, the amino acid substitution at position D265 is D265C or D265A (EU index). In another embodiment, the N297 amino acid substitution is selected from the group consisting of N297A, N297G and N297Q (EU index). In another embodiment, the Fc region further comprises an amino acid substitution at positions L234 and L235 (EU index). In another embodiment, the L234 amino acid substitution is L234A or L234V. In another embodiment, the L235 amino acid substitution is L235A. In another embodiment, the Fc region further comprises an amino acid substitution at position E233 (EU index). In another embodiment, the E233 amino acid substitution is E233P (EU index). In another embodiment, the Fc region further comprises a deletion of G236 (EU index). In another embodiment, the Fc region further comprises an amino acid substitution at position P331 (EU index). In another embodiment, the P331 amino acid substitution is P331G. In another embodiment, the Fc region does not include a substitution at position P331 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position I253 (EU index). In another embodiment, the I253 amino acid substitution is I253A. In another embodiment, the Fc region further comprises an amino acid substitution at position H310 (EU index). In another embodiment, the H310 amino acid substitution is H310A.

In another aspect, provided herein is an antibody, or antigen-binding portion thereof, comprising an Fc region, wherein the Fc region comprises an amino acid substitution at positions E233, L234, L235, and D265 (EU index) and a deletion of G236 (EU index), and an amino acid substitution at D265 (EU index). In one embodiment, the amino acid substitution at D265 is D265C or D265A (EU index). In another embodiment, the L234 amino acid substitution is L234A or L234V. In another embodiment, the L235 amino acid substitution is L235A. In another embodiment, the E233 amino acid substitution is E233P (EU index). In another embodiment, the Fc region further comprises an amino acid substitution at position N297 (EU index). In another embodiment, the N297 amino acid substitution is selected from the group consisting of N297A, N297G and N297Q (EU index). In another embodiment, the Fc region further comprises an amino acid substitution at position P331 (EU index). In another embodiment, the P331 amino acid substitution is P331G. In another embodiment, the Fc region does not include a substitution at position P331 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position I253 (EU index). In another embodiment, the I253 amino acid substitution is I253A. In another embodiment, the Fc region further comprises an amino acid substitution at position H310 (EU index). In another embodiment, the H310 amino acid substitution is H310A.

In another aspect, provided herein is an antibody, or antigen-binding portion thereof, comprising an Fc region, wherein the Fc region comprises an amino acid substitution at position H435 and D265 (EU index). In one embodiment, the amino acid substitution at position D265 is D265C or D265A (EU index). In another embodiment, the H435 amino acid substitution is H435A. In another embodiment, the Fc region further comprises an amino acid substitution at position N297 (EU index). In another embodiment, the N297 amino acid substitution is selected from the group consisting of N297A, N297G and N297Q (EU index). In another embodiment, the Fc region further comprises an amino acid substitution at positions L234 and L235 (EU index). In another embodiment, the L234 amino acid substitution is L234A or L234V. In another embodiment, the L235 amino acid substitution is L235A. In another embodiment, the Fc region further comprises an amino acid substitution at position E233 (EU index). In another embodiment, the E233 amino acid substitution is E233P (EU index). In another embodiment, the Fc region further comprises a deletion of G236 (EU index). In another embodiment, the Fc region further comprises an amino acid substitution at position P331 (EU index). In another embodiment, the P331 amino acid substitution is P331G. In another embodiment, the Fc region does not include a substitution at position P331 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position I253 (EU index). In another embodiment, the I253 amino acid substitution is I253A. In another embodiment, the Fc region further comprises an amino acid substitution at position H310 (EU index). In another embodiment, the H310 amino acid substitution is H310A.

In another aspect, provided herein is an antibody, or antigen-binding portion thereof, comprising an Fc region, wherein the Fc region comprises an amino acid substitution at positions L234 and L235 (EU index), and amino acid substitution P329 (EU index). In one embodiment, the L234 amino acid substitution is L234A or L234V. In another embodiment, the L235 amino acid substitution is L235A. In one embodiment, the Fc region further comprises an amino acid substitution at position D265 (EU index). In one embodiment, the D265 amino acid substitution is D265C or D265A (EU index). In one embodiment, the Fc region further comprises an amino acid substitution at position N297 (EU index). In one embodiment, the N297 amino acid substitution is selected from the group consisting of N297A, N297G and N297Q (EU index). In one embodiment, the Fc region further comprises an amino acid substitution at position E233 (EU index). In one embodiment, the E233 amino acid substitution is E233P (EU index). In one embodiment, the Fc region further comprises a deletion of G236 (EU index). In one embodiment, the Fc region further comprises an amino acid substitution at position P331 (EU index). In one embodiment, the P331 amino acid substitution is P331G. In one embodiment, the Fc region does not include a substitution at position P331 (EU index). In one embodiment, the Fc region further comprises an amino acid substitution at position I253 (EU index). In one embodiment, the I253 amino acid substitution is I253A. In one embodiment, the Fc region further comprises an amino acid substitution at position H310 (EU index). In one embodiment, the H310 amino acid substitution is H310A.

In another aspect, provided herein is an antibody, or antigen-binding portion thereof, comprising an Fc region, wherein the Fc region comprises an amino acid substitution at positions L234 and L235 (EU index), and amino acid substitution P331 (EU index). In one embodiment, the L234 amino acid substitution is L234A or L234V. In one embodiment, the L235 amino acid substitution is L235A. In one embodiment, the Fc region further comprises an amino acid substitution at position D265 (EU index). In one embodiment, the D265 amino acid substitution is D265C or D265A (EU index). In one embodiment, the Fc region further comprises an amino acid substitution at position N297 (EU index). In one embodiment, the N297 amino acid substitution is selected from the group consisting of N297A, N297G and N297Q (EU index). In one embodiment, the Fc region further comprises an amino acid substitution at position E233 (EU index). In one embodiment, the E233 amino acid substitution is E233P (EU index). In one embodiment, the Fc region further comprises a deletion of G236 (EU index). In one embodiment, the Fc region further comprises an amino acid substitution at position P329 (EU index). In one embodiment, the P329 amino acid substitution is P329G. In one embodiment, the Fc region does not include a substitution at position P329 (EU index). In one embodiment, the Fc region further comprises an amino acid substitution at position I253 (EU index). In one embodiment, the I253 amino acid substitution is I253A. In one embodiment, the Fc region further comprises an amino acid substitution at position H310 (EU index). In one embodiment, the H310 amino acid substitution is H310A.

In another aspect, provided herein is an antibody, or antigen-binding portion thereof, comprising an Fc region, wherein the Fc region comprises an amino acid substitution at positions E233 and L234 and L235 (EU index), a deletion of G236 (EU index). In one embodiment, the L234 amino acid substitution is L234A or L234V. In one embodiment, the L235 amino acid substitution is L235A. In one embodiment, the E233 amino acid substitution is E233P (EU index). In one embodiment, the Fc region further comprises an amino acid substitution at position H435 (EU index). In one embodiment, the H435 amino acid substitution is H435A. In one embodiment, the Fc region further comprises an amino acid substitution at position N297 (EU index). In one embodiment, the N297 amino acid substitution is selected from the group consisting of N297A, N297G and N297Q (EU index). In one embodiment, the Fc region further comprises an amino acid substitution at position P331 (EU index). In one embodiment, the P331 amino acid substitution is P331G. In one embodiment, the Fc region does not include a substitution at position P331 (EU index). In one embodiment, the Fc region further comprises an amino acid substitution at position P329 (EU index). In one embodiment, the P329 amino acid substitution is P329G. In one embodiment, the Fc region does not include a substitution at position P329 (EU index). In one embodiment, the Fc region further comprises an amino acid substitution at position I253 (EU index). In one embodiment, the I253 amino acid substitution is I253A. In one embodiment, the Fc region further comprises an amino acid substitution at position H310 (EU index). In one embodiment, the H310 amino acid substitution is H310A.

In another aspect, provided herein is an antibody, or antigen-binding portion thereof, comprising an Fc region, wherein the Fc region comprises an amino acid substitution at positions I253, H310 and H345 (EU index). In one embodiment, the I253 amino acid substitution is I253A. In another embodiment, the H310 amino acid substitution is H310A. In another embodiment, the H435 amino acid substitution is H435A. In another embodiment, the Fc region further comprises an amino acid substitution at position N297 (EU index). In another embodiment, the N297 amino acid substitution is selected from the group consisting of N297A, N297G and N297Q (EU index). In another embodiment, the Fc region further comprises an amino acid substitution at position D265 (EU index). In another embodiment, the D265 amino acid substitution is D265C or D265A (EU index). In another embodiment, the Fc region further comprises an amino acid substitution at position E233 (EU index). In another embodiment, the E233 amino acid substitution is E233P (EU index). In another embodiment, the Fc region further comprises a deletion of G236 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position P331 (EU index). In another embodiment, the P331 amino acid substitution is P331G. In another embodiment, the Fc region does not include a substitution at position P329 (EU index).

In another aspect, provided herein is an antibody, or antigen-binding portion thereof, comprising an Fc region, wherein the Fc region comprises an amino acid substitution at position N297 (EU index). In one embodiment, the Fc region further comprises an amino acid substitution at positions L234 and L235 (EU index). In another embodiment, the L234 amino acid substitution is L234A or L234V. In another embodiment, the L235 amino acid substitution is L235A. In another embodiment, the Fc region does not include a substitution at positions L234 and L235 (EU index). In another embodiment, the N297 amino acid substitution is selected from the group consisting of N297A, N297G and N297Q. In another embodiment, the Fc region further comprises an amino acid substitution at position E233 (EU index). In another embodiment, the E233 amino acid substitution is E233P (EU index). In another embodiment, the Fc region further comprises a deletion of G236 (EU index). In another embodiment, the Fc region further comprises an amino acid substitution at position P331 (EU index). In another embodiment, the P331 amino acid substitution is P331G. In another embodiment, the Fc region does not include a substitution at position P331 (EU index). In another embodiment, the Fc region further comprises an amino acid substitution at position P329 (EU index). In another embodiment, the P329 amino acid substitution is P329G. In another embodiment, the Fc region does not include a substitution at position P329 (EU index). In another embodiment, the Fc region further comprises an amino acid substitution at position I253 (EU index). In another embodiment, the I253 amino acid substitution is I253A. In another embodiment, the Fc region further comprises an amino acid substitution at position H310 (EU index). In another embodiment, the H310 amino acid substitution is H310A.

In some embodiments, the antibody, or antigen-binding portion thereof, comprises any combination of substitutions to the Fc region as described herein.

In some embodiments, the antibody, or antigen-binding portion thereof, further comprises an amino acid substitution at position S239 (EU index). In one embodiment, the S239 amino acid substitution is S239C.

In some embodiments, the antibody, or antigen-binding portion thereof, further comprises an amino acid substitution at position H435 (EU index). In one embodiment, the H435 amino acid substitution is H435A. In another embodiment, the antibody comprises an amino acid substitution H435A and has a decreased half-life relative to an identical intact IgG antibody comprising an unmodified Fc region.

In another aspect, provided herein is an antibody, or antigen-binding portion thereof, comprising an Fc region, wherein the Fc region comprises amino acid substitutions L234A, L235A, S239C and D265A (EU index).

In another aspect, provided herein is an antibody, or antigen-binding portion thereof, comprising an Fc region, wherein the Fc region comprises amino acid substitutions L234A, L235A, S239C and D265C (EU index).

In another aspect, provided herein is an antibody, or antigen-binding portion thereof, comprising an Fc region, wherein the Fc region comprises amino acid substitutions consisting essentially of amino acid substitutions L234A, L235A, and D265C (EU index).

In another aspect, provided herein is an antibody, or antigen-binding portion thereof, comprising an Fc region, wherein the Fc region comprises amino acid substitutions consisting essentially of amino acid substitutions L234A, L235A, and D265A (EU index).

In another aspect, provided herein is an antibody, or antigen-binding portion thereof, comprising an Fc region, wherein the Fc region comprises amino acid substitutions consisting essentially of amino acid substitutions L234A, L235A, S239C and D265A (EU index).

In another aspect, provided herein is an antibody, or antigen-binding portion thereof, comprising an Fc region, wherein the Fc region comprises amino acid substitutions consisting essentially of amino acid substitutions H435A, L234A, L235A, and D265C (EU index). In another aspect, provided herein is an antibody, or antigen-binding portion thereof, comprising an Fc region, wherein the Fc region comprises amino acid substitutions consisting essentially of amino acid substitutions N297A and D265C (EU index).

In another aspect, provided herein is an antibody, or antigen-binding portion thereof, comprising an Fc region, wherein the Fc region comprises amino acid substitutions consisting essentially of amino acid substitutions N297G and D265C (EU index).

In another aspect, provided herein is an antibody, or antigen-binding portion thereof, comprising an Fc region, wherein the Fc region comprises amino acid substitutions consisting essentially of amino acid substitutions N297Q and D265C (EU index).

In another aspect, provided herein is an antibody, or antigen-binding portion thereof, comprising an Fc region, wherein the Fc region comprises amino acid substitutions consisting essentially of amino acid substitutions N297A and D265A (EU index).

In another aspect, provided herein is an antibody, or antigen-binding portion thereof, comprising an Fc region, wherein the Fc region comprises amino acid substitutions consisting essentially of amino acid substitutions N297G and D265A (EU index).

In another aspect, provided herein is an antibody, or antigen-binding portion thereof, comprising an Fc region, wherein the Fc region comprises amino acid substitutions consisting essentially of amino acid substitutions N297Q and D265A (EU index).

In another aspect, provided herein is an antibody, or antigen-binding portion thereof, wherein the antibody has a decrease in an effector function defined as a decrease in binding to an Fc gamma receptor (FcγR) relative to binding of an identical antibody comprising an unmodified Fc region to the FcγR. In some embodiments, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In another embodiment, the antibody does not detectably bind the FcγR. In another embodiment, antibody binding to the FcγR is assessed by biolayer interferometry (BLI). In another embodiment, antibody binding to the FcγR is assessed using assays known to one of ordinary skill in the art. In another embodiment, the FcγR is an FcγR1 receptor. In another embodiment, the FcγR receptor is an FcγR2 receptor or an FcγR3 receptor. In another embodiment, the FcγR2 receptor is FcγR2A, FcγR2B, or FcγR2C. In another embodiment, the FcγR3 receptor is FcγR3A or FcγR3B. In another embodiment, the Fc receptor is a human Fc receptor. In other embodiments, the FcγR receptor is a the FcγR2A 167R receptor. In other embodiments, the FcγR receptor is a the FcγR3A 176V receptor. In other embodiments, the FcγR receptor is a the FcγR3A 176F receptor.

In another aspect, provided herein is an antibody, or antigen-binding portion thereof, wherein the antibody decreases cytokine release in an in vitro cytokine release assay with a decrease in cytokine release of at least 50% relative to cytokine release of an identical antibody comprising an unmodified Fc region. In one embodiment, the decrease in cytokine release is at least a 60% decrease, at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In another embodiment, the antibody does not show detectable cytokine release. In another embodiment, the in vitro cytokine release assay is a Meso Scale Discovery (MSD) tissue culture (TC) proinflammatory assay. In another embodiment, the in vitro cytokine release assay is assessed using assays known to one of ordinary skill in the art. In another embodiment, the antibody decreases mast cell degranulation in an in vitro mast cell degranulation assay with a decrease in mast cell degranulation of at least 50% relative to mast cell degranulation of an identical antibody comprising an unmodified Fc region. In another embodiment, the decrease in mast cell degranulation is at least a 60% decrease, at least a 70% decrease, at least a 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In another embodiment, the antibody does not show detectable mast cell degranulation. In another embodiment, the in vitro mast cell degranulation assay is a beta-hexosaminidase-based mast cell degranulation assay.

In some embodiments, the IgG isotype is an IgG 1 isotype, a IgG2 isotype, a IgG3 isotype, or a IgG4 isotype. In another embodiment, the antibody is a human antibody, a chimeric or a humanized antibody. In yet another embodiment, the antibody is a bispecific antibody. In another embodiment, the antibody is a monoclonal antibody. In another embodiment, the antibody is an intact IgG antibody. In another embodiment, the antibody specifically binds CD117, CD45, CD2, CD5, CD137, or CD252.

In another aspect, provided herein is an antibody drug conjugate (ADC) comprising the antibody, or antigen-binding portion thereof, as set for the herein, wherein the antibody, or antigen-binding portion thereof, is conjugated to a cytotoxin via a linker. In one embodiment, the cytotoxin is an RNA polymerase inhibitor. In another embodiment, the RNA polymerase inhibitor is an amatoxin. In another embodiment, the amatoxin is represented by formula (III)

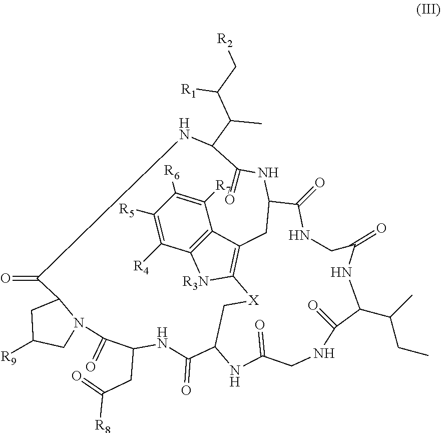

wherein R₁ is H, OH, OR_{A}, or OR_{C}; R2 is H, OH, OR_{B}, or OR_{C}; R_{A} and R_{B}, together with the oxygen atoms to which they are bound, combine to form an optionally substituted 5-membered heterocycloalkyl group; R₃ is H, R_{C}, or R_{D}; R₄, R₅, R₆, and R₇ are each independently H, OH, OR_{C}, OR_{D}, R_{C}, or R_{D}; R₈ is OH, NH₂, OR_{C}, OR_{D}, NHR_{C}, or NR_{C}R_{D}; R₉ is H, OH, OR_{C}, or OR_{D}; X is —S—, —S(O)—, or —SO₂—; R_{C} is -L-Z; R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof; and Z is a chemical moiety formed from a coupling reaction between a reactive substituent present on L and a reactive substituent present within the antibody or antigen-binding fragment thereof, wherein Am comprises exactly one R_{C} substituent. In another embodiment, the amatoxin is represented by formula (IB)

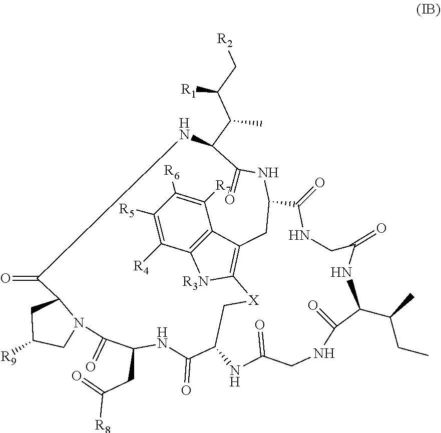

wherein R₁ is H, OH, OR_{A}, or OR_{C}; R₂ is H, OH, OR_{B}, or OR_{C}; R_{A} and R_{B}, together with the oxygen atoms to which they are bound, combine to form an optionally substituted 5-membered heterocycloalkyl group; R₃ is H, R_{C}, or R_{D}; R₄, R₅, R₆, and R₇ are each independently H, OH, OR_{C}, OR_{D}, R_{C}, or R_{D}; R₈ is OH, NH₂, OR_{C}, OR_{D}, NHR_{C}, or NR_{C}R_{D}; R₉ is H, OH, OR_{C}, or OR_{D}; X is —S—, —S(O)—, or —SO₂—; R_{C} is -L-Z; R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof; and Z is a chemical moiety formed from a coupling reaction between a reactive substituent present on L and a reactive substituent present within the antibody or antigen-binding fragment thereof, wherein Am comprises exactly one R_{C} substituent. In another embodiment, the RNA polymerase inhibitor is an amanitin. In another embodiment, the amanitin is selected from the group consisting of α-amanitin, β-amanitin, γ-amanitin, ε-amanitin, amanin, amaninamide, amanullin, amanullinic acid, and proamanullin. In another embodiment, the cytotoxin selected from the group consisting of an pseudomonas exotoxin A, deBouganin, diphtheria toxin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer. In another embodiment, the auristatin is MMAE or MMAF. In another embodiment, the antibody, or antigen-binding portion thereof, is conjugated to the cytotoxin via an interchain conjugation to a native hinge cysteine. In another embodiment, the antibody or antigen-binding portion thereof, is conjugated to the cytotoxin by way of a cysteine residue in the Fc domain of the antibody. In another embodiment, the cysteine residue is introduced by way of an amino acid substitution in the Fc domain of the antibody. In another embodiment, the amino acid substitution is D265C. In another embodiment, the amino acid substitution is S239C.

In another aspect, provided herein is a pharmaceutical composition comprising the antibody or ADC of any one of claims 1 to 218, and a pharmaceutically acceptable carrier.

In another aspect, provided herein is a method of depleting a population of hematopoietic stem cells (HSC) in a human patient, the method comprising administering to the patient an effective amount of the antibody or ADC as described herein. In one embodiment, the method comprises administering to the patient a transplant comprising hematopoietic stem cells. In one embodiment, the transplant is allogeneic. In one embodiment, the transplant is autologous.

In another aspect, provided herein is a method comprising administering to a human patient a transplant comprising hematopoietic stem cells, wherein the patient has been previously administered the antibody or the ADC as described herein, in an amount sufficient to deplete a population of hematopoietic stem cells in the patient. In one embodiment, the hematopoietic stem cell is a CD117+ or CD45+ cell. In another embodiment, the patient has a blood disease, a metabolic disorder, cancer, or an autoimmune disease, or severe combined immunodeficiency disease (SCID).

In another aspect, provided herein is a method of treating leukemia in a human patient, said method comprising administering the antibody or ADC as described herein, to the human patient having leukemia.

In another aspect, provided herein is a method comprising administering to a human patient a transplant comprising hematopoietic stem cells, wherein the patient has been previously administered the antibody or the ADC as described herein, in an amount sufficient to deplete a population of immune cells in the patient. In one embodiment, the immune cell is a CD137+, CD2+, or CD5+ cell. In another embodiment, the immune cell is a T cell.

In another aspect, provided herein is a composition comprising the antibody or ADC as described herein, wherein the composition comprises less than 25% hydrophobic degradant following thermal stress. In one embodiment, the composition comprises less than 20% hydrophobic degradant following thermal stress. In another embodiment, the composition comprises less than 15% hydrophobic degradant following thermal stress. In another embodiment, the composition comprises less than 10% hydrophobic degradant following thermal stress. In another embodiment, the composition comprises less than 5% hydrophobic degradant following thermal stress.

In another aspect, provided herein is a method of treating a stem cell disorder in a human patient, the method comprising administering to the patient a therapeutically effective amount of an antibody, antigen-binding fragment thereof, or ADC as described herein.

In another aspect, provided herein is a method of treating an immunodeficiency disorder in a human patient, the method comprising administering to the patient a therapeutically effective amount of an antibody, antigen-binding fragment thereof, or ADC as described herein. In one embodiment, the immunodeficiency disorder is a congenital immunodeficiency or an acquired immunodeficiency.

In another aspect, provided herein is a method of treating a metabolic disorder in a human patient, the method comprising administering to the patient a therapeutically effective amount of an antibody, antigen-binding fragment thereof, or ADC as described herein. In one embodiment, the metabolic disorder is selected from the group consisting of glycogen storage diseases, mucopolysaccharidoses, Gaucher's Disease, Hurlers Disease, sphingolipidoses, and metachromatic leukodystrophy.

In another aspect, provided herein is a method of treating an autoimmune disorder in a human patient, the method comprising administering to the patient a therapeutically effective amount of an antibody, antigen-binding fragment thereof, or ADC as described herein. In some embodiments, the autoimmune disorder is selected from the group consisting of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis.

In another aspect, provided herein is a method of treating cancer in a human patient, the method comprising administering to the patient a therapeutically effective amount of an antibody, antigen-binding fragment thereof, or ADC as described herein. In some embodiments, the cancer is selected from the group consisting of leukemia, lymphoma, multiple myeloma, and neuroblastoma.

In some embodiments of any of the above aspects, the antibody has a decrease in an effector function defined as a decrease in binding to an Fc gamma receptor (FcγR) relative to binding of an identical antibody comprising an unmodified Fc region to the FcγR. In one embodiment, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In certain embodiments, the antibody does not detectably bind the FcγR. In some embodiments, antibody binding to the FcγR is assessed by biolayer interferometry (BLI). In some embodiments, the FcγR is an FcγR1 receptor, an FcγR2 receptor, or an FcγR3 receptor. In some embodiments, the FcγR1 receptor is FcγR1A, FcγR1B, or FcγR1C. In some embodiments, the FcγR1 receptor is FcγR2A, FcγR2B, or FcγR2C. In some embodiments, the FcγR1 receptor is FcγR3A or FcγR3B. In some embodiments, the Fc receptor is a human Fc receptor.

In some embodiments of any of the above aspects, the IgG isotype is an IgG1 isotype, a IgG2 isotype, a IgG3 isotype, or a IgG4 isotype.

In some embodiments of any of the above aspects, the antibody is a human antibody.

In some embodiments of any of the above aspects, the antibody is a chimeric or humanized antibody.

In some embodiments of any of the above aspects, the antibody is a monoclonal antibody.

In some embodiments of any of the above aspects, the antibody specifically binds CD117, CD45, CD2, CD5, CD137, or CD252.

In another aspect, provided herein is an antibody drug conjugate (ADC) comprising any of the antibodies herein, wherein the antibody is conjugated to a cytotoxin via a linker.

In some embodiments of the conjugates herein, the cytotoxin is an RNA polymerase inhibitor. In some embodiments, the RNA polymerase inhibitor is an amatoxin.

In some embodiments, the amatoxin is represented by formula (IA)

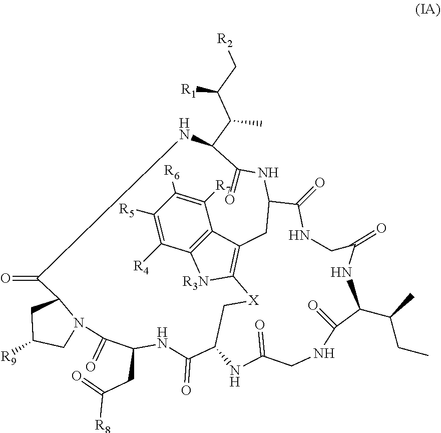

wherein R₁ is H, OH, OR_{A}, or OR_{C};
R₂ is H, OH, OR_{B}, or OR_{C};
R_{A} and R_{B}, together with the oxygen atoms to which they are bound, combine to form an optionally substituted 5-membered heterocyclolalkyl group;
R₃ is H, R_{C}, or R_{D};
R₄, R₅, R₆, and R₇ are each independently H, OH, OR_{C}, OR_{D}, R_{C}, or R_{D};
R₅ is OH, NH₂, OR_{C}, OR_{D}, NHR_{C}, or NR_{C}R_{D};
R₉ is H, OH, OR_{C}, or OR_{D};

### X is —S—, —S(O)—, or —SO₂—;

### R_{C} is -L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;
L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, or optionally substituted heteroarylene; and
Z is a chemical moiety formed from a coupling reaction between a reactive substituent present on L and a reactive substituent present within the antibody or antigen-binding fragment thereof, wherein Am comprises exactly one R_{C} substituent.

In some embodiments, the amatoxin is represented by formula (IB)

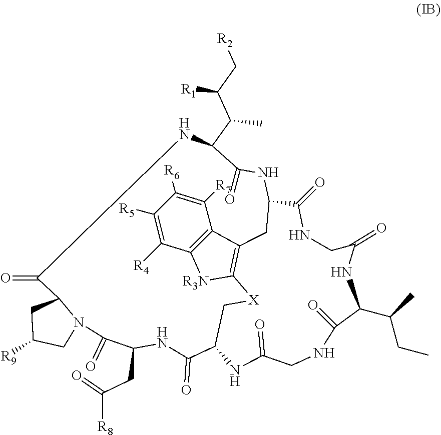

wherein R₁ is H, OH, OR_{A}, or OR_{C};
R₂ is H, OH, OR_{B}, or OR_{C};
R_{A} and R_{B}, together with the oxygen atoms to which they are bound, combine to form an optionally substituted 5-membered heterocyclolalkyl group;
R₃ is H, R_{C}, or R_{D};
R₄, R₅, R₆, and R₇ are each independently H, OH, OR_{C}, OR_{D}, R_{C}, or R_{D};
R₅ is OH, NH₂, OR_{C}, OR_{D}, NHR_{C}, or NR_{C}R_{D};
R₉ is H, OH, OR_{C}, or OR_{D};

### X is —S—, —S(O)—, or —SO₂—;

### R_{C} is -L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, or optionally substituted heteroarylene; and
Z is a chemical moiety formed from a coupling reaction between a reactive substituent present on L and a reactive substituent present within the antibody or antigen-binding fragment thereof,
wherein Am comprises exactly one R_{C} substituent.

In some embodiments, the RNA polymerase inhibitor is an amanitin. In some embodiments, the amanitin is selected from the group consisting of α-amanitin, β-amanitin, γ-amanitin, ε-amanitin, amanin, amaninamide, amanullin, amanullinic acid, and proamanullin.

In some embodiments, the cytotoxin selected from the group consisting of an pseudomonas exotoxin A, deBouganin, diphtheria toxin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, and an indolinobenzodiazepine dimer. In some embodiments, the auristatin is MMAE or MMAF.

In some embodiments of the conjugates herein, the antibody is conjugated to the toxin by way of a cysteine residue in the Fc domain of the antibody. In some embodiments, the cysteine residue is introduced by way of an amino acid substitution in the Fc domain of the antibody. In some embodiments, the amino acid substitution is D265C.

In another aspect, provided herein is a pharmaceutical composition comprising an antibody or ADC described herein, and a pharmaceutically acceptable carrier.

In yet another aspect, provided herein is a method of depleting a population of hematopoietic stem cells (HSC) in a human patient, the method comprising administering to the patient an effective amount of an antibody or ADC described herein,

In some embodiments of the methods described herein, the method further comprises administering to the patient a transplant comprising hematopoietic stem cells. In some embodiments, the transplant is allogeneic. In some embodiments, the transplant is autologous.

In another aspect, provided herein is a method comprising administering to a human patient a transplant comprising hematopoietic stem cells, wherein the patient has been previously administered an antibody or the ADC described herein in an amount sufficient to deplete a population of hematopoietic stem cells in the patient.

In some embodiments of the methods described herein, the patient has a blood disease, a metabolic disorder, cancer, or an autoimmune disease, or severe combined immunodeficiency disease (SCID).

In a further aspect, provided herein is a method of treating leukemia in a human patient, said method comprising administering an antibody or ADC described herein to the human patient having leukemia.

In one aspect, provided herein is a method of depleting a population of CD117+ cells in a human patient in need thereof, the method comprising administering to the patient an effective amount of an anti-CD117 antibody drug conjugate (ADC), wherein the antibody drug conjugate (ADC) is comprises an anti-CD117 antibody conjugated to an amatoxin via a linker and is represented by the formula Ab-Z-L-Am, wherein Ab is an anti-CD117 antibody comprising an H435A mutation (EU index) in the Fc region of the antibody, L is a linker, Z is a chemical moiety, and Am is an amatoxin. In one embodiment, the ADC is administered prior to the patient receiving a transplant comprising hematopoietic stem cells. In another embodiment, the ADC is administered concommittanity with the patient receiving a transplant comprising hematopoietic stem cells.

In another aspect, provided herein is a method for administering to a human patient an anti-CD117 antibody drug conjugate (ADC) in an amount sufficient to deplete a population of CD117+ cells in the patient in need thereof, wherein the antibody drug conjugate (ADC) is comprises an anti-CD117 antibody conjugated to an amatoxin via a linker and is represented by the formula Ab-Z-L-Am, wherein Ab is an anti-CD117 antibody comprising an H435A mutation (EU index) in the Fc region of the antibody, L is a linker, Z is a chemical moiety, and Am is an amatoxin; and subsequently administering to the patient a transplant comprising hematopoietic stem cells. In one embodiment, the transplant comprising hematopoietic stem cells is administered to the patient after the concentration of the ADC has substantially cleared from the blood of the patient. In another embodiment, the hematopoietic stem cells or progeny thereof maintain hematopoietic stem cell functional potential after two or more days following transplantation of the hematopoietic stem cells into the patient. In yet another embodiment, the hematopoietic stem cells or progeny thereof are capable of localizing to hematopoietic tissue and/or reestablishing hematopoiesis following transplantation of the hematopoietic stem cells into the patient. In a further embodiment, the patient has a disorder selected from the group consisting of adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis. In another embodiment, the patient has an autoimmune disorder or a hematological cancer. In another embodiment, the autoimmune disorder is selected from the group consisting of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis.

In another aspect, provided herein is a method of treating a human subject having a hematological cancer comprising administering an effective amount of an anti-CD117 antibody drug conjugate (ADC) to the human subject having the hematological cancer, wherein the antibody drug conjugate (ADC) is comprises an anti-CD117 antibody conjugated to an amatoxin via a linker and is represented by the formula Ab-Z-L-Am, wherein Ab is an anti-CD117 antibody comprising an H435A mutation (EU index) in the Fc region of the antibody, L is a linker, Z is a chemical moiety, and Am is an amatoxin. In one embodiment, the hematological cancer is leukemia. In another embodiment, the Fc region of the anti-CD117 antibody comprises a D265C mutation (EU index). In yet another embodiment, the anti-CD117 antibody comprises a heavy chain variable region comprising a CDR1 comprising the amino acid sequence set forth in SEQ ID NO: 7, a CDR2 comprising the amino acid sequence set forth in SEQ ID NO:8, and a CDR3 comprising the amino acid sequence set forth in SEQ ID NO: 9; and comprising a light chain variable region comprising a CDR1 comprising the amino acid sequence as set forth in SEQ ID NO: 10, a CDR2 comprising the amino acid sequence set forth in SEQ ID NO:11, and a CDR3 comprising the amino acid sequence as set forth in SEQ ID NO: 12. In another embodiment, the anti-CD117 antibody comprises a heavy chain variable region comprising the amino acid sequence set forth in SEQ ID NO: 13, and a light chain variable region comprising the amino acid sequence set forth in SEQ ID NO: 14. In another embodiment, the ADC is internalized by a cancer cell, autoimmune cell, or hematopoietic stem cell following administration to the patient. In another embodiment, the Am-L-Z is represented by formula (I)

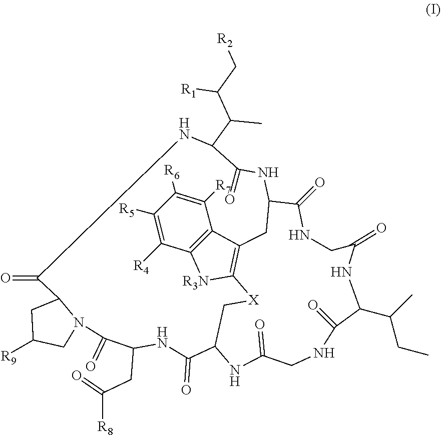

wherein R₁ is H, OH, OR_{A}, or OR_{C}; R₂ is H, OH, OR_{B}, or OR_{C}; R_{A} and R_{B}, when present, together with the oxygen atoms to which they are bound, combine to form an optionally substituted 5-membered heterocycloalkyl group; R₃ is H, R_{C}, or R_{D}; R₄, R₅, R₆, and R₇ are each independently H, OH, OR_{C}, OR_{D}, R_{C}, or R_{D}; R₅ is OH, NH₂, OR_{C}, OR_{D}, NHR_{C}, or NR_{C}R_{D}; R₉ is H, OH, OR_{C}, or OR_{D}; X is —S—, —S(O)—, or —SO₂—; R_{C} is -L-Z; R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a dipeptide, —C(═O)—, a peptide, or a combination thereof; and Z is a chemical moiety formed from a coupling reaction between a reactive substituent present on L and a reactive substituent present within the antibody or antigen-binding fragment thereof, wherein Am comprises exactly one R_{C} substituent. In another embodiment the Am-L-Z is represented by formula (IB).

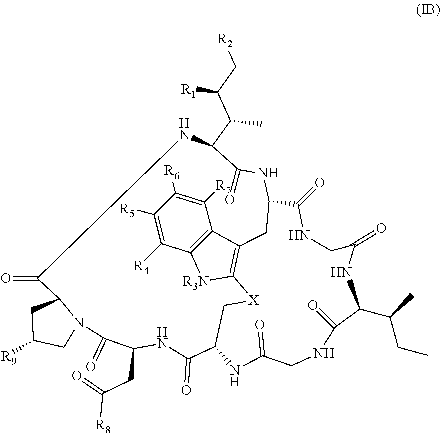

wherein R₁ is H, OH, OR_{A}, or OR_{C}; R₂ is H, OH, OR_{B}, or OR_{C}; R_{A} and R_{B}, when present, together with the oxygen atoms to which they are bound, combine to form an optionally substituted 5-membered heterocycloalkyl group; R₃ is H, R_{C}, or R_{D}; R₄, R₅, R₆, and R₇ are each independently H, OH, OR_{C}, OR_{D}, R_{C}, or R_{D}; R₅ is OH, NH₂, OR_{C}, OR_{D}, NHR_{C}, or NR_{C}R_{D}; R₉ is H, OH, OR_{C}, or OR_{D}; X is —S—, —S(O)—, or —SO₂—; R_{C} is -L-Z; R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl; L is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a dipeptide, —C(═O)—, a peptide. or a combination thereof; and Z is a chemical moiety formed from a coupling reaction between a reactive substituent present on L and a reactive substituent present within the antibody or antigen-binding fragment thereof, wherein Am comprises exactly one R_{C} substituent. In another embodiment, the ADC is administered to the human patient at a dose of about 0.1 mg/kg to about 0.3 mg/kg.

### BRIEF DESCRIPTION OF THE FIGURES

FIGS. 1A-1E graphically depict the results of a standard bio-layer interferometery (BLI) assay to assess the binding response of antibodies having the indicated amino acid substitutions to Fc gamma receptors indicated in the legend. The normalized binding response of each anti-CD117 antibody variant relative to WT IgG1 binding is shown in FIG. 1A and quantification of the normalized binding response of each anti-CD117 antibody variant is shown in FIG. 1B. FIG. 1C depicts the quantification of the normalized binding responses of anti-CD45 antibody variants relative to WT anti-CD45 antibody binding (FIG. 1C; “ic” as used herein refers to interchain conjugates to native hinge cysteines). The normalized binding response of additional anti-CD117 antibody variants relative to WT IgG1 (isotype control) binding is shown in FIG. 1D and quantification of the normalized binding response of each anti-CD117 antibody variant is shown in FIG. 1E.

FIG. 2 graphically depicts the results of a mast cell degranulation assay, in which Beta-hexosaminidase was measured following incubation of mast cells with the indicated antibodies. Beta-hexosaminidase release from mast cells was measured by monitoring para-nitrophenol production from 4-Nitrophenyl N-acetyl-β-D-glucosaminide substrate and is presented as the absorbance of para-nitrophenol at 405 nm on the y-axis.

FIGS. 3A, 3B, 3C, 3D and 3E graphically depicts the results of an in vitro cytokine release assay to measure the release of IL-6 (FIG. 3A), IL-8 (FIG. 3B), TNFa (FIG. 3C), IL-1B (FIG. 3D) and GM-CSF (FIG. 3E) from human peripheral blood mononuclear cells (PBMC) following incubation with the indicated antibodies. The human PBMCs for FIGS. 3A-3D were isolated from one of four donors, as demarcated in the legend.

FIGS. 4A and 4B graphically depict the results of an in vitro antibody-dependent cellular phagocytosis (ADCP) assay that shows a reduction in ADCP activity due to Fc effector silencing in certain Fc variants in comparison to the controls. FIG. 4A depicts results of flow cytometry analysis of the co-expression of CFSE and CD134 staining. FIG. 4B depicts results of incubating a mixture of MDM and Kasumi-1 cells (1:2 molar ratio) for two hours at 37° C. with increasing concentrations of the indicated antibody.

FIGS. 5A, 5B and 5C depict chromatograms demonstrating the results of a thermostability assay in which the melting temperature of each indicated antibody was assessed.

FIGS. 6A and 6B are tables showing the Tm 1 (CH2 Unfolding) and Tm2 (fab/CH3 unfolding) melting temperature for each indicated antibody as determined in the thermostability assay depicted in FIG. 5A (see FIG. 6A) and FIGS. 5B and 5C (see FIG. 6B).

FIGS. 7A and 7B depict chromatograms demonstrating the elution profile of the indicated antibodies at time=0 at room temperature (non-stressed condition) or post 30 minutes incubation at 60 degrees Celsius (stressed condition) after analysis by hydrophobic interaction chromatography (HIC). FIG. 7A depicts chromatograms demonstrating the elution profile of the Ab1 antibody and certain Ab1 Fc variants at time=0 at room temperature (non-stressed condition; FIG. 7A (chromatogram in lower panel)) or post 30 minutes incubation at 60 degrees Celsius (stressed condition; FIG. 7A (chromatogram in upper panel)) after analysis by hydrophobic interaction chromatography (HIC). FIG. 7B depicts a chromatogram demonstrating the elution profile of the Ab2 antibody and certain Ab2 Fc variants at time=0 at room temperature (non-stressed condition) or post 30 minutes incubation at 60 degrees Celsius (stressed condition) after analysis by hydrophobic interaction chromatography (HIC).

FIGS. 8A-8E graphically depicts the results of the HIC assays of FIGS. 7A and 7B, showing the percent area of the antibody monomer (i.e., “Main”) peak (FIGS. 8A and 8D) or hydrophobic degradant peak (FIGS. 8B, 8C and 8E) for the indicated antibodies after exposure to stressed or non-stressed conditions.

FIGS. 9A-9E graphically depict the results of a size exclusion chromatography assay, in which the percent area of the antibody monomer peak (FIGS. 9A and 9D) or percent high molecular aggregate peak (FIGS. 9B, 9C and 9E) was determined for the indicated antibodies after exposure to time=0 at room temperature (non-stressed condition) or post 30 minutes incubation at 60 degrees Celsius (stressed condition).

FIGS. 10A and 10B graphically depict the results of a non-human primate pharmacokinetic assay expressed as the concentration (ng/mL) of an engineered fast half-life anti-CD117-amatoxin ADC at varying doses (0.1 mg/kg and 0.3 mg/kg) as a function time (i.e., hours post-administration; x-axis). FIG. 10B illustrates that the timing of ADC-mediated depletion and clearance provides a window for transplant conditioning post-administration.

FIGS. 11A-11E graphically depict the results of assays detecting the depletion of phenotypic hematopoietic stem cells (i.e., CD34+ CD90+ CD45RA− HSCs) using flow cytometry (FIG. 11A and FIG. 11C) or an assessment of colony forming units from the bone marrow aspirate (FIG. 11B and FIG. 11D) as a function of varying doses of the ADC1 antibody drug conjugate (ADC) versus a control (i.e., PBS) (x-axis). FIGS. 11C and 11D further show data corresponding to the unconjugated anti-CD117 antibody (“anti-CD117”). FIG. 11E shows a phenotypic analysis of bone marrow hematopoietic stem cells (treated versus untreated) using flow cytometry (at day 7 post-dose administration).

FIGS. 12A-12C graphically depict the results of assays detecting (FIG. 12A) the neutrophil count (10³/mL) and (FIGS. 12B and 12C) the lymphocyte count (10³/mL) as a function of days post dose administration of varying doses of the ADC1 antibody drug conjugate (ADC) versus a control (i.e., PBS). FIG. 12C further shows data corresponding to the lymphocyte count for cynomolgus monkeys administered an unconjugated anti-CD117 antibody (“anti-CD117”).

FIGS. 13A-13C graphically depict the results of assays detecting levels of (FIGS. 13A and 13C) plasma alanine aminotransaminase (ALT; in U/mL) and (FIG. 13B) plasma bilirubin (in U/mL) as a function of days post dose administration of varying doses of the ADC1 antibody drug conjugate (ADC) versus a control (i.e., PBS). FIG. 13C further shows data corresponding to the plasma levels of ALT in cynomolgus monkeys administered an unconjugated anti-CD117 antibody (“anti-CD117”).

FIG. 14 shows images of liver and kidney tissue isolated from cynomolgus monkeys 35 days post-administration of ADC1 (0.3 mg/kg) or a control (PBS).

FIG. 15 graphically depicts the results of an assay detecting reticulocyte count (10⁹/mL) as a function of days post dose administration of varying doses (0.1 mg/kg or 0.3 mg/kg) of an ADC1 antibody drug conjugate versus a control (i.e., PBS) or an unconjugated anti-CD117 antibody.

### DETAILED DESCRIPTION

Disclosed herein are antibodies, and conjugates thereof (antibody drug conjugates; ADC) having modified Fc regions, wherein the modifications decrease or substantially eliminate antibody effector functions. The modifications to the Fc region may further permit antibody-drug conjugation and/or decrease the half-life of the antibody. The interaction of antibodies and antibody-antigen complexes with cells of the immune system may effect a variety of responses, including antibody-dependent cell-mediated cytotoxicity (ADCC) and complement dependent cytotoxicity (CDC). Binding of the Fc region of an antibody to Fc receptors on a cell surface may trigger a number of biological responses including engulfment and destruction of antibody-coated particles, clearance of immune complexes, lysis of antibody-coated target cells by killer cells (i.e., ADCC), release of inflammatory mediators, placental transfer and control of immunoglobulin production. By reducing or substantially eliminating antibody effector function, the presently disclosed antibodies can, for example, advantageously avoid triggering various immune system reactions (e.g., avoid cytokine release or avoid mast cell-degranulation) that can be detrimental in certain therapies, e.g., hematopoietic stem cell therapies (for example, hematopoietic stem cell transplant therapy) and depletion of hematopoietic cells (e.g., for treatment of blood cancers, immune system diseases and disorders, autoimmune diseases, graft-versus-host disease, etc.).

Accordingly, included herein are anti-hematopoietic cell antibodies (also referred to as anti-HC antibodies) having modified Fc regions that are useful in therapies. For example, the antibodies or ADCs herein are useful in conditioning procedures, in which a patient is prepared for receipt of a transplant comprising hematopoietic stem cells. Such procedures promote the engraftment of a hematopoietic stem cell transplant. According to the methods described herein, in some embodiments a patient may be conditioned (for example for hematopoietic stem cell transplant therapy or immune system reset) by administration to the patient of an ADC, an antibody, or an antigen-binding fragment thereof, capable of binding an antigen expressed by hematopoietic cells (e.g., hematopoietic stem cells, e.g., hematopoietic stem cells and or mature immune cells (e.g., T cells)), such as CD117 (e.g., GNNK+ CD117), CD45, CD2, CD5, CD137, CD252 and combinations thereof. In some embodiments, the antibodies or ADCs contemplated herein may be used for the treatment of diseases or disorders of the hematopoietic system. For example, in some embodiments, the antibodies or ADCs contemplated herein may be used for the treatment of blood cancers. In another non-limiting example, the antibodies or ADCs contemplated herein may be used for the treatment of graft-versus-host disease (“GvHD”). In certain embodiments, the antibodies or ADCs contemplated herein may be used for the treatment of a T-cell-mediated disease or disorder. As described herein, the antibody may be covalently conjugated to a cytotoxin so as to form an antibody drug conjugate (ADC). Administration of an ADC, an antibody, or an antigen-binding fragment thereof, capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

In one particular aspect, the invention provides isolated anti-CD117 antibodies, specifically isolated human anti-CD117 antibodies, that bind to the ectodomain of human CD117, wherein the isolated anti-CD117 antibodies have modified Fc regions, wherein the modifications decrease or substantially eliminate antibody effector functions. The binding regions of the isolated anti-CD117 antibodies identified herein are described below.

The sections that follow provide a description of the antibodies, or conjugates thereof, that can be administered to a patient, such as a patient suffering from a cancer or autoimmune disease, or a patient in need of hematopoietic stem cell transplant therapy in order to promote engraftment of hematopoietic stem cell grafts, as well as methods of administering such therapeutics to a patient (e.g., prior to hematopoietic stem cell transplantation).

### Definitions

As used herein, the term “about” refers to a value that is within 5% above or below the value being described.

As used herein, the term “allogeneic”, when used in the context of transplantation, is used to define cells (or tissue or an organ) that are transplanted from a donor to a recipient of the same species but who is genetically different. Thus, the term “allogeneic cells” refers to cell types that are genetically distinct between two individuals, yet belong to the same species, e.g., human. Typically, the term “allogeneic” is used to define cells, such as stem cells, that are transplanted from a donor to an unrelated recipient of the same species.

As used herein, the term “autologous” refers to cells or a graft where the donor and recipient are the same subject.

As used herein, the term “xenogeneic” refers to cells where the donor and recipient species are different.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered antibodies, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rIgG, and scFv fragments, so long as they exhibit the desired antigen-binding activity.

The antibodies of the present disclosure are generally isolated or recombinant. “Isolated,” when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities. For instance, an isolated antibody that specifically binds to CD117 is substantially free of antibodies that specifically bind antigens other than CD117.

The term “monoclonal antibody” as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. In one embodiment, an antibody fragment comprises an Fc region.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art. In one embodiment, an antigen-binding fragment of an antibody comprises an Fc region.

As used herein, the term “anti-CD117 antibody” or “an antibody that binds to CD117” refers to an antibody that is capable of binding CD117 with sufficient affinity such that the antibody is useful as a diagnostic and/or therapeutic agent in targeting CD117.

As used herein, the term “anti-CD45 antibody” or “an antibody that binds to CD45” refers to an antibody that is capable of binding CD45 with sufficient affinity such that the antibody is useful as a diagnostic and/or therapeutic agent in targeting CD45.

As used herein, the term “anti-CD2 antibody” or “an antibody that binds to CD2” or an “anti-CD2 ADC” or “an ADC that binds to CD2” refers to an antibody or ADC that specifically binds to human CD2 as CD2 is found on the cell surface of cells, such as T cells.

As used herein, the term “anti-CD5 antibody” or “an antibody that binds to CD5” or an “anti-CD5 ADC” or “an ADC that binds to CD5” refers to an antibody or ADC that specifically binds to human CD5 as CD5 is found on the cell surface of cells, such as T cells.

As used herein, the term “anti-CD137 antibody” or an “antibody that binds to CD137” refers to an antibody that is capable of binding CD137 with sufficient affinity such that the antibody is useful as a diagnostic and/or therapeutic agent in targeting CD137.

As used herein, the term “anti-CD252 antibody” or an “antibody that binds to CD252” refers to an antibody that is capable of binding CD252 with sufficient affinity such that the antibody is useful as a diagnostic and/or therapeutic agent in targeting CD252. In a preferred embodiment, the antibody specifically binds to human CD252 (hCD252). CD252 is found on antigen presenting cells.

As used herein, the term “bispecific antibody” refers to, for example, a monoclonal, e.g., a human or humanized antibody, that is capable of binding at least two different antigens or two different epitopes. For instance, one of the binding specificities can be directed towards an epitope on a hematopoietic stem cell surface antigen, CD117 (e.g., GNNK+ CD117), and the other can specifically bind an epitope on a different hematopoietic stem cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody).

An “intact” or “full length” antibody, as used herein, refers to an antibody having two heavy (H) chain polypeptides and two light (L) chain polypeptides interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

As used herein, the term “thermal stress” refers to stress created by any change in temperature to a molecule, e.g., an antibody, an Fc containing antigen-binding fragment thereof, or an ADC. In one embodiment, thermal stress is incubation of an antibody, an Fc containing antigen-binding fragment thereof, or an ADC at 60 degrees Celsius for 30 minutes.

The term “specifically binds”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., an antigen expressed by hematopoietic stem cells, such as CD117 (e.g., GNNK+ CD117), or CD45; or an antigen expressed by mature immune cells (e.g., T-cells), such as CD45, CD2, CD5, CD137, or CD252, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specifically binds” refers to the ability of a antibody to bind to an antigen with an Kd of at least about 1×10⁻⁶M, 1×10⁻⁷ M, 1×10⁻⁸ M, 1×10⁻⁹ M, 1×10⁻¹⁰ M, 1×10⁻¹¹ M, 1×10⁻¹² M, or more and/or bind to an antigen with an affinity that is at least two-fold greater than its affinity for a nonspecific antigen. In one embodiment, K_{D} is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of an antigen, e.g., CD117 (e.g., GNNK+ CD117), CD45, CD2, CD5, CD137, or CD252.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397.

The terms “Fc”, “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the second constant domain CH2 (e.g., residues at EU positions 231-340 of human IgG1) and the third constant domain CH3 (e.g., residues at EU positions 341-447 of human IgG1). As used herein, the Fc domain includes the “lower hinge region” (e.g., residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively. An example of a “WT” Fc region is provided in SEQ ID NO: 15 (which provides a heavy chain constant region containing an Fc region).

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or Clq as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

The variant Fc domains described herein are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A.L235A mutant can be referred to as “LALA”. As a further example, the E233P.L234V.L235A.delG236 (deletion of 236) mutant can be referred to as “EPLVLAdelG”. As yet another example, the I253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc.gamma.R genes. In humans this family includes but is not limited to Fc.gamma.RI (CD64), including isoforms Fc.gamma.RIa, Fc.gamma.RIb, and Fc.gamma.RIc; Fc.gamma.RII (CD32), including isoforms Fc.gamma.RIIa (including allotypes H131 and R131), Fc.gamma.RIIb (including Fc.gamma.RIIb-1 and Fc.gamma.RIIb-2), and Fc.gamma.RIIc; and Fc.gamma.RIII (CD16), including isoforms Fc.gamma.RIIIa (including allotypes V158 and F158) and Fc.gamma.RIIIb (including allotypes Fc.gamma.RIIIb-NA1 and Fc.gamma.RIIIb-NA2), as well as any undiscovered human Fc.gamma.Rs or Fc.gamma.R isoforms or allotypes. An Fc.gamma.R can be from any organism, including but not limited to humans, mice, rats, rabbits, and monkeys. Mouse Fc.gamma.Rs include but are not limited to Fc.gamma.RI (CD64), Fc.gamma.RII (CD32), Fc.gamma.RIII (CD16), and Fc.gamma.RIII-2 (CD16-2), as well as any undiscovered mouse Fc.gamma.Rs or Fc.gamma.R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and .gamma.delta. T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, L234A, L235A, and/or H435A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC, an antibody or antigen-binding fragment thereof capable of binding an antigen expressed by hematopoietic stem cells, such as CD117 (e.g., GNNK+ CD117), CD45, CD2, CD5, CD137, or CD252. As described herein, the antibody may be covalently conjugated to a cytotoxin so as to form an ADC. Administration of an antibody, antigen-binding fragment thereof, or an ADC capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “effective amount” or “therapeutically effective amount” refers to an amount that is sufficient to achieve the desired result or to have an effect on an autoimmune disease or cancer.

As used herein, the term “half-life” refers to the time it takes for the plasma concentration of the antibody drug in the body to be reduced by one half or 50%. This 50% reduction in serum concentration reflects the amount of drug circulating.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B cells and T cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34-. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, SlamfI/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, SlamfI/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “anti-hematopoietic cell antibody” or “anti-HC antibody” refers to an antibody that specifically binds an antigen expressed by hematopoietic stem cells, such as CD117 (e.g., GNNK+ CD117), or CD45; or an antigen expressed by mature immune cells (e.g., T-cells) such as CD45, CD2, CD5, CD137, or CD252.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, T cells and B cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes VH and VL domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one VH domain and one VL domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

As used herein, the phrase “substantially cleared from the blood” refers to a point in time following administration of a therapeutic agent (such as an anti-CD117 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments, and protein ligands, such as ELISA-based detection assays known in the art or described herein. Additional assays that can be used to detect antibodies, or antibody fragments, include immunoprecipitation techniques and immunoblot assays, among others known in the art.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein “to treat” or “treatment”, refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease, any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting the engraftment of exogenous hematopoietic cells in a patient following antibody conditioning therapy as described herein and subsequent hematopoietic stem cell transplant therapy Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self-antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally, or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally, or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the invention include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “conjugate” or “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the term “microtubule-binding agent” refers to a compound which acts by disrupting the microtubular network that is essential for mitotic and interphase cellular function in a cell. Examples of microtubule-binding agents include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Amatoxins useful in conjunction with the compositions and methods described herein include compounds such, as but not limited to, compounds of Formulas (III), (IIIA), (IIIB), and (IIIC), each as described herein below (e.g., an α-amanitin, β-amanitin, γ-amanitin, ε-amanitin, amanin, amaninamide, amanullin, amanullinic acid, or proamanullin). As described herein, amatoxins may be conjugated to an antibody, or antigen-binding fragment thereof, for instance, by way of a linker moiety (L) (thus forming an ADC). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

The term “acyl” as used herein refers to —C(═O)R, wherein R is hydrogen (“aldehyde”), C₁-C₁₂ alkyl, C₂-C₁₂ alkenyl, C₂-C₁₂ alkynyl, C₃-C₇ carbocyclyl, C₆-C₂₀ aryl, 5-10 membered heteroaryl, or 5-10 membered heterocyclyl, as defined herein. Non-limiting examples include formyl, acetyl, propanoyl, benzoyl, and acryloyl.

The term “C₁-C₁₂ alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp2 double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or spa carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkaryl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or spa carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Unless otherwise constrained by the definition of the individual substituent, the foregoing chemical moieties, such as “alkyl”, “heteroalkyl”, “alkenyl”, “heteroalkenyl”, “alkynyl”, “heteroalkynyl”, “cycloalkyl”, “heterocyclolalkyl”, “aryl,” and “heteroaryl” groups can optionally be substituted with, for example, from 1 to 5 substituents selected from the group consisting of alkyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂— and the like. Other radical naming conventions clearly indicate that the radical is a di-radical such as “alkylene,” “alkenylene,” “arylene,” “heterocycloalkylene,” and the like.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, or antigen-binding fragment thereof, specific for CD117 (such as GNNK+ CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, “drug-to-antibody ratio” or “DAR” refers to the number of cytotoxins, e.g., amatoxin, attached to the antibody of an ADC. The DAR of an ADC can range from 1 to 8, although higher loads are also possible depending on the number of linkage sites on an antibody. Thus, in certain embodiments, an ADC described herein has a DAR of 1, 2, 3, 4, 5, 6, 7, or 8.

Wherever a substituent is depicted as a di-radical (i.e., has two points of attachment to the rest of the molecule), it is to be understood that the substituent can be attached in any directional configuration unless otherwise indicated.

### Fc-Modified Antibodies

The present disclosure is based in part on the discovery that antibodies, or antigen-binding fragments thereof, having Fc modifications that allow Fc silencing, capable of binding an antigen expressed by hematopoietic cells can be used as therapeutic agents. For example, the present disclosure is based in part on the discovery that antibodies, or antigen-binding fragments thereof, having Fc modifications that allow Fc silencing, capable of binding (i) an antigen expressed by hematopoietic cells, including but not limited to CD117 (e.g., GNNK+ CD117), or CD45; or an antigen expressed by mature immune cells (e.g., T-cells), including but not limited CD45, CD2, CD5, CD137, or CD252, can be used as therapeutic agents (e.g., as “naked” antibodies or as ADCs to (i) treat cancers and autoimmune diseases characterized by CD117+ (e.g., GNNK+ CD117) or CD45+ hematopoietic stem cells; or CD45+, CD2+, CD5+, CD137+, or CD252+ immune cells (e.g., T-cells) and (ii) promote the engraftment of transplanted hematopoietic stem cells in a patient in need of transplant therapy. These therapeutic activities can be caused, for instance, by the binding of an anti-hematopoietic cell (HC)-antibody (e.g., anti-CD117 antibody, anti-CD45 antibody, anti-CD2 antibody, anti-CD5 antibody, anti-CD137 antibody, anti-CD252 antibody, etc.) or antigen-binding fragment thereof, that binds to an antigen (e.g., CD117 (e.g., GNNK+ CD117), CD45, CD2, CD5, CD137, CD252, etc.) expressed by a hematopoietic cell (e.g., hematopoietic stem cell leukocyte, immune cell, e.g., mature immune cell (e.g., T cell)), such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

The antibodies, or antigen-binding fragments thereof, described herein may also include modifications and/or mutations that alter the properties of the antibodies and/or fragments, such as those that increase half-life, or increase or decrease ADCC.

In one embodiment, antibodies comprising one or more radiolabeled amino acids are provided. A radiolabeled antibody may be used for both diagnostic and therapeutic purposes (conjugation to radiolabeled molecules is another possible feature). Non-limiting examples of labels for polypeptides include, but are not limited to 3H, 14C, 15N, 35S, 90Y, 99Tc, and 125I, 131I, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, U.S. Pat. No. 5,102,990 (U.S. RE35,500), U.S. Pat. Nos. 5,648,471 and 5,697,902. For example, a radioisotope may be conjugated by a chloramine T method.

In one embodiment, the anti-HC antibody (e.g., anti-CD117 antibody, anti-CD45 antibody, anti-CD2 antibody, anti-CD5 antibody, anti-CD137 antibody, or anti-CD252 antibody), or antigen-binding fragment thereof, comprises a modified Fc region, wherein said modified Fc region comprises at least one amino acid modification relative to a wild-type Fc region, such that said molecule has an altered affinity for or binding to an FcgammaR (FcγR). Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). In some embodiments, the antibodies described herein may comprise variant Fc regions comprising modification of at least one residue that makes a direct contact with an FcγR based on structural and crystallographic analysis. In one embodiment, the Fc region of the anti-HC antibody (e.g., anti-CD117 antibody, anti-CD45 antibody, anti-CD2 antibody, anti-CD5 antibody, anti-CD137 antibody, anti-CD252 antibody, etc.), or antigen-fragment thereof, comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat. In one embodiment, the Fc region comprises a L234A mutation. In some embodiments, the Fc region of the anti-HC antibody (e.g., anti-CD117 antibody, anti-CD45 antibody, anti-CD2 antibody, anti-CD5 antibody, anti-CD137 antibody, or anti-CD252 antibody), or antigen-fragment thereof, comprises an amino acid substitution at amino acid 235 according to the EU index as in Kabat. In one embodiment, the Fc region comprises a L235A mutation. In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A, S239C, L234A, and L235A mutation (also referred to herein as “D265A.S239C.L234A.L235A”). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as “D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and delG236 (deletion of 236) mutation (also referred to herein as “E233P.L234V.L235A.delG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, delG236 (deletion of 236) and H435A mutation (also referred to herein as “E233P.L234V.L235A.delG236.H435A” or as “EPLVLAdelG.H435A”). In another embodiment, the Fc region comprises a E233P, L234V, L235A, delG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR1. In some embodiments, the FcγR is FcγR2A. In some embodiments, the FcγR is FcγR2B. In other embodiments, the FcγR is FcγR2C. In some embodiments, the FcγR is FcγR3A. In some embodiments, the FcγR is FcγR3B. In other embodiments, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In other embodiments, the decrease in binding is at least a 70% to a 100% decrease, at least a 80% to a 100% decrease, at least a 90% to a 100% decrease, at least a 95% to a 100% decrease, or at least a 98% to a 100% decrease, in antibody binding to a FcγR relative to binding of the identical antibody comprising an unmodified Fc region to the FcγR

In some embodiments, the antibody has a modified Fc region such that, the antibody decreases cytokine release in an in vitro cytokine release assay with a decrease in cytokine release of at least 50% relative to cytokine release of an identical antibody comprising an unmodified Fc region. In some embodiments, the decrease in cytokine release is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In some embodiments, the decrease in cytokine release is at least a 70% to a 100% decrease, at least a 80% to a 100% decrease, at least a 90% to a 100% decrease, at least a 95% to a 100% decrease in cytokine release relative to cytokine release of the identical antibody comprising an unmodified Fc region. In preferred embodiments, cytokine release is by immune cells.

In some embodiments, the antibody has a modified Fc region such that, the antibody decreases mast cell degranulation in an in vitro mast cell degranulation assay with a decrease in mast cell degranulation of at least 50% relative to mast cell degranulation of an identical antibody comprising an unmodified Fc region. In some embodiments, the decrease in mast cell degranulation is at least a 70% decrease, at least a 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In some embodiments, the decrease in mast cell degranulation is at least a 70% to a 100% decrease, at least a 80% to a 100% decrease, at least a 90% to a 100% decrease, or at least a 95% to a 100% decrease, in mast cell degranulation relative to mast cell degranulation of the identical antibody comprising an unmodified Fc region.

In some embodiments, the antibody has a modified Fc region such that, the antibody decreases or prevents antibody dependent cell phagocytosis (ADCP) in an in vitro antibody dependent cell phagocytosis assay, with a decrease in ADCP of at least 50% relative to ADCP of an identical antibody comprising an unmodified Fc region. In some embodiments, the decrease in ADCP is at least a 70% decrease, at least a 80% decrease, at least a 90% decrease, at least a 95% decrease, at least a 98% decrease, at least a 99% decrease, or a 100% decrease in antibody dependent cell phagocytosis to antibody dependent cell phagocytosis of the identical antibody comprising an unmodified Fc region.

In some embodiments, the anti-HC antibody (e.g., anti-CD117 antibody, anti-CD45 antibody, anti-CD2 antibody, anti-CD5 antibody, anti-CD137 antibody, or anti-CD252 antibody) described herein comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdelG*), D265C (EPLVLAdelG)/H435A, D265C/N297Q/H435A, D265C/N297Q, EPLVLAdelG/H435A, EPLVLAdelG/D265C, EPLVLAdelG/D265A, N297A, N297G, or N297Q.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least a 75% decrease, at least a 80% decrease, at least a 85% decrease, at least a 90% decrease, at least a 95% decrease, at least a 98% decrease, at least a 99% decrease, or a 100% decrease in binding to a Fc gamma receptor relative to binding of the identical antibody comprising an unmodified Fc region to the Fc gamma receptor (e.g., as assessed by biolayer interferometry (BLI), e.g., as described in Example 1).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in vitro (e.g., as described in Example 2) or for their ability to trigger cytokine release, e.g. by human peripheral blood mononuclear cells (e.g., as described in Example 3).

Thus, in one embodiment, the Fc region comprises a mutation resulting in a decrease in half-life (e.g., relative to an antibody having an unmodified Fc region). An antibody having a short half-life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express a target antigen (e.g., CD117 (e.g., GNNK+ CD117), CD45, CD2, CD5, CD137, or CD252) but are not the target of the anti-HC antibody (e.g., anti-CD117 antibody, anti-CD45 antibody, anti-CD2 antibody, anti-CD5 antibody, anti-CD137 antibody, or anti-CD252 antibody), unlike the endogenous stem cells. In one embodiment, the Fc region comprises a mutation at position 435 (EU index according to Kabat). In one embodiment, the mutation is an H435A mutation.

In one embodiment, the anti-HC antibody (e.g., anti-CD117 antibody, anti-CD45 antibody, anti-CD2 antibody, anti-CD5 antibody, anti-CD137 antibody, or anti-CD252 antibody) described herein has a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-HC antibody (e.g., anti-CD117 antibody, anti-CD45 antibody, anti-CD2 antibody, anti-CD5 antibody, anti-CD137 antibody, or anti-CD252 antibody) described herein has a half-life (e.g., in humans) of about 1-5 hours, about 5-10 hours, about 10-15 hours, about 15-20 hours, or about 20 to 25 hours.

In some aspects, the Fc region comprises two or more mutations that confer reduced half-life and reduce an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-HC antibody (e.g., anti-CD117 antibody, anti-CD45 antibody, anti-CD2 antibody, anti-CD5 antibody, anti-CD137 antibody, or anti-CD252 antibody), or antigen-binding fragment thereof, comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-HC antibody (e.g., anti-CD117 antibody, anti-CD45 antibody, anti-CD2 antibody, anti-CD5 antibody, anti-CD137 antibody, or anti-CD252 antibody), or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

Methods of engineering antibodies to include any of the Fc modifications herein are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

### Anti-CD117 Antibodies

The present disclosure is also based in part on the discovery that antibodies, or antigen-binding fragments thereof, capable of binding CD117, such as GNNK+ CD117, can be used as therapeutic agents alone or as ADCs to (i) treat cancers (such as acute myelogenous leukemia or myelodysplastic syndrome) and autoimmune diseases characterized by CD117+ cells and (ii) promote the engraftment of transplanted hematopoietic stem cells in a patient in need of transplant therapy. These therapeutic activities can be caused, for instance, by the binding of anti-CD117 antibodies, or antigen-binding fragments thereof, to CD117 (e.g., GNNK+ CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+ CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins.

In one embodiment, the anti-CD117 antibody, or antigen binding portion thereof, comprises a heavy chain variable region as set forth in the amino acid sequence of SEQ ID NO: 13, and a light chain variable region as set forth in the amino acid sequence of SEQ ID NO: 14.

In another embodiment, the anti-CD117 antibody, or antigen binding portion thereof, comprises the three CDR sequences of the heavy chain variable region (VH) amino acid sequence and the three CDR sequences of the light chain variable region (LH) amino acid sequence of Ab85.

In another embodiment, the anti-CD117 antibody, or antigen binding portion thereof, comprises the heavy chain variable region (VH) amino acid sequence and the light chain variable region (LH) amino acid sequence of Ab85 (also referred to herein interchangeably as Ab2).

The heavy chain variable region (VH) amino acid sequence provided below as SEQ ID NO: 13. The VH CDR amino acid sequences of Ab85 are underlined below and are as follows: NYWIG (VH CDR1; SEQ ID NO: 7); IINPRDSDTRYRPSFQG (VH CDR2; SEQ ID NO: 8); and HGRGYEGYEGAFDI (VH CDR3; SEQ ID NO: 9).

Ab85 VH sequence
(SEQ ID NO: 13)
EVQLVQSGAEVKKPGESLKISCKGSGYSFTNYWIGWVRQMPGKGLEWMAI
INPRDSDTRYRPSFQGQVTISADKSISTAYLQWSSLKASDTAMYYCARHG
RGYEGYEGAFDIWGQGTLVTVSS

The light chain variable region (VL) amino acid sequence of Ab85 is provided below as SEQ ID NO 14. The VL CDR amino acid sequences of Ab85 are underlined below and are as follows: RSSQGIRSDLG (VL CDR1; SEQ ID NO: 10); DASNLET (VL CDR2; SEQ ID NO: 11; and QQANGFPLT (VL CDR3; SEQ ID NO: 12).

Ab85 VL sequence
(SEQ ID NO: 14)
DIQMTQSPSSLSASVGDRVTITCRSSQGIRSDLGWYQQKPGKAPKLLIYD
ASNLETGVPSRFSGSGSGTDFTLTISSLQPEDFATYYCQQANGFPLTFGG
GTKVEIK

In another embodiment, the anti-CD117 antibody, or antigen binding portion thereof, comprises the heavy chain variable region (VH) amino acid sequence and the light chain variable region (LH) amino acid sequence of Ab249 (also referred to herein interchangeably as Ab3).

The heavy chain variable region (VH) amino acid sequence of Ab249 is provided below as SEQ ID NO: 346. The VH CDR amino acid sequences of Ab249 are underlined below and are as follows: TSWIG (VH CDR1; SEQ ID NO: 340); IIYPGDSDTRYSPSFQG (VH CDR2; SEQ ID NO: 341); and HGLGYNGYEGAFDI (VH CDR3; SEQ ID NO: 342).

Ab249 VH sequence
(SEQ ID NO: 346)
EVQLVQSGAEVKKPGESLKISCKGSGYRFTTSWIGWVRQMPGKGLEWMGI
IYPGDSDTRYSPSFQGQVTISADKSISTAYLQWSSLKASDTAMYYCARHG
LGYNGYEGAFDIWGQGTLVTVSS

The light chain variable region (VL) amino acid sequence of Ab249 is provided below as SEQ ID NO: 347. The VL CDR amino acid sequences of Ab249 are underlined below and are as follows: RASQGIGSALA (VL CDR1; SEQ ID NO: 343); DASNLET (VL CDR2; SEQ ID NO: 344); and QQLNGYPLT (VL CDR3; SEQ ID NO: 345).

Ab249 VL sequence
(SEQ ID NO: 347)
DIQMTQSPSSLSASVGDRVTITCRASQGIGSALAWYQQKPGKAPKLLIYD
ASNLETGVPSRFSGSGSGTDFTLTISSLQPEDFATYYCQQLNGYPLTFGQ
GTRLEIK

Human antibodies Ab85 and Ab249 were both derived from antibody CK6, which is an antagonist anti-CD117 antibody. Ab85 and Ab249 have improved properties, e.g., improved binding characteristics, over CK6.

Thus, in certain embodiments, an anti-CD117 antibody comprises a heavy chain comprising a CDR set (CDR1, CDR2, and CDR3) as set forth in SEQ ID Nos: 7, 8, and 9, and a light chain comprising a CDR set as set forth in SEQ ID Nos: 10, 11, and 12. In other embodiments, an anti-CD117 antibody comprises a heavy chain comprising a CDR set (CDR1, CDR2, and CDR3) as set forth in SEQ ID Nos: 340, 341, and 342, and a light chain comprising a CDR set as set forth in SEQ ID Nos: 343, 344, and 345.

In another embodiment, the anti-CD117 antibody, or antigen binding portion thereof, comprises the heavy chain variable region (VH) amino acid sequence and the light chain variable region (LH) amino acid sequence of Ab67 (a neutral antibody; also referred to herein interchangeably as Ab1).

The heavy chain variable region (VH) amino acid sequence of Ab67 is provided below as SEQ ID NO: 354. The VH CDR amino acid sequences of Ab67 are underlined below and are as follows: FTFSDADMD (VH CDR1; SEQ ID NO: 348); RTRNKAGSYTTEYAASVKG (VH CDR2; SEQ ID NO: 349); and AREPKYWIDFDL (VH CDR3; SEQ ID NO: 350).

Ab67 VH sequence
(SEQ ID NO: 354)
EVQLVESGGGLVQPGGSLRLSCAASGFTFSDADMDWVRQAPGKGLEWVGR
TRNKAGSYTTEYAASVKGRFTISRDDSKNSLYLQMNSLKTEDTAVYYCAR
EPKYWIDFDLWGRGTLVTVSS

The light chain variable region (VL) amino acid sequence of Ab67 is provided below as SEQ ID NO: 355. The VL CDR amino acid sequences of Ab67 are underlined below and are as follows: RASQSISSYLN (VL CDR1; SEQ ID NO: 351); AASSLQS (VL CDR2; SEQ ID NO: 352); and QQSYIAPYT (VL CDR3; SEQ ID NO: 353).

Ab67 VL sequence
(SEQ ID NO: 355)
DIQMTQSPSSLSASVGDRVTITCRASQSISSYLNWYQQKPGKAPKLLIYA
ASSLQSGVPSRFSGSGSGTDFTLTISSLQPEDFATYYCQQSYIAPYTFGG
GTKVEIK

Thus, in certain embodiments, an anti-CD117 antibody comprises a heavy chain comprising a CDR set (CDR1, CDR2, and CDR3) as set forth in SEQ ID Nos: 348, 349, and 350, and a light chain comprising a CDR set as set forth in SEQ ID Nos: 351, 352, and 353.

Additional sequence for anti-CD117 antibodies or binding fragments, described herein, are provided in Table 5.

The anti-CD117 antibodies or binding fragments described herein may also include modifications and/or mutations that alter the properties of the antibodies and/or fragments, such as those that increase half-life, increase or decrease ADCC, etc., as is known in the art.

In one embodiment, the anti-CD117 antibody, or binding fragment thereof, comprises a variant Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). For example, amino acid substitutions at amino acid positions 234 and 235 of the Fc region have been identified as decreasing affinity of an IgG antibody for binding to an Fc receptor, particularly an Fc gamma receptor (FcγR). In one embodiment, an anti-CD117 antibody described herein comprises an Fc region comprising an amino acid substitution at L234 and/or L235, e.g., L234A and L235A (EU index). Thus, the anti-CD117 antibodies described herein may comprise variant Fc regions comprising modification of at least one residue that makes a direct contact with an FcγR based on structural and crystallographic analysis. In one embodiment, the Fc region of the anti-CD117 antibody (or Fc containing fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” or “EU index” refers to the numbering of the human IgG1 EU antibody and is used herein in reference to Fc amino acid positions unless otherwise indicated.

In one embodiment, the Fc region comprises a D265A mutation. In one embodiment, the Fc region comprises a D265C mutation.

In some embodiments, the Fc region of the anti-CD117 antibody (or fragment thereof) comprises an amino acid substitution at amino acid 234 according to the EU index as in Kabat. In one embodiment, the Fc region comprises a L234A mutation. In some embodiments, the Fc region of the anti-CD117 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In certain aspects a variant IgG Fc domain comprises one or more amino acid substitutions resulting in decreased or ablated binding affinity for an FcgammaR and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an anti-CD117 antibody described herein comprises an Fc region comprising L235A, L235A, and D265C (EU index). The antibodies of the invention may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life (also referred to herein as a “fast” half life) may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express CD117 but are not the target of the anti-CD117 antibody, unlike the endogenous stem cells. In one embodiment, the Fc region comprises a mutation at position 435 (EU index according to Kabat). In one embodiment, the mutation is an H435A mutation. In another embodiment, the mutation is a D265C mutation. In yet another embodiment, the mutations are an H435A mutation and a D265C mutation.

In one embodiment, the anti-CD117 antibody described herein has a half life of equal to or less than 24 hours, equal to or less than 22 hours, equal to or less than 20 hours, equal to or less than 18 hours, equal to or less than 16 hours, equal to or less than 14 hours, equal to or less than 13 hours, equal to or less than 12 hours, equal to or less than 11 hours, equal to or less than 10 hours, equal to or less than 9 hours, equal to or less than 8 hours, equal to or less than 7 hours, equal to or less than 6 hours, or equal to or less than 5 hours. In one embodiment, the half life of the antibody is 5 hours to 7 hours; is 5 hours to 9 hours; is 15 hours to 11 hours; is 5 hours to 13 hours; is 5 hours to 15 hours; is 5 hours to 20 hours; is 5 hours to 24 hours; is 7 hours to 24 hours; is 9 hours to 24 hours; is 11 hours to 24 hours; 12 hours to 22 hours; 10 hours to 20 hours; 8 hours to 18 hours; or 14 hours to 24 hours.

Anti-CD117 antibodies that can be used in conjunction with the patient conditioning methods described herein include, for instance, antibodies produced and released from ATCC Accession No. 10716 (deposited as BA7.3C.9), such as the SR-1 antibody, which is described, for example, in U.S. Pat. No. 5,489,516, the disclosure of which is incorporated herein by reference as it pertains to anti-CD117 antibodies.

In one embodiment, an anti-CD117 antibody described herein comprises an Fc region comprising L235A, L235A, D265C, and H435A (EU index).

Additional anti-CD117 antibodies that can be used in conjunction with the patient conditioning methods described herein include those described in U.S. Pat. No. 7,915,391, which describes, e.g., humanized SR-1 antibodies; U.S. Pat. No. 5,808,002, which describes, e.g., the anti-CD117 A3C6E2 antibody, as well as those described in, for example, WO 2015/050959, which describes anti-CD117 antibodies that bind epitopes containing Pro317, Asn320, Glu329, Val331, Asp332, Lus358, Glue360, Glue376, His378, and/or Thr380 of human CD117; and US 2012/0288506 (also published as U.S. Pat. No. 8,552,157), which describes, e.g., the anti-CD117 antibody CK6 (also referred to herein interchangeably as Ab4), having the CDR sequences of:

| a CDR-H1 having the amino acid sequence | |
|---|---|
| SYWIG; | (SEQ ID NO: 1) |
| a CDR-H2 having the amino acid sequence | |
| IIYPGDSDTRYSPSFQG; | (SEQ ID NO: 2) |
| a CDR-H3 having the amino acid sequence | |
| HGRGYNGYEGAFDI; | (SEQ ID NO: 3) |
| a CDR-L1 having the amino acid sequence | |
| RASQGISSALA; | (SEQ ID NO: 4) |
| a CDR-L2 having the amino acid sequence | |
| DASSLES; | (SEQ ID NO: 5) |
| and | |
| a CDR-L3 having the amino acid sequence | |
| CQQFNSYPLT | (SEQ ID NO: 6) |

The heavy chain variable region amino acid sequence of CK6 is provided in SEQ ID NO: 27):

QVQLVQSGAAVKKPGESLKISCKGSGYRFTSYWIGWVRQMPGKGLEWMGI
IYPGDSDTRYSPSFQGQVTI SAGKSISTAYLQWSSLKASDTAMYYCARH
GRGYNGYEGAFDIWGQGTMVTVSS (SEQ ID NO: 27; CDRs are
underlined are in bold).

The light chain amino acid variable sequence of CK6 is provided in SEQ ID NO: 28:

AIQLTQSPSSLSASVGDRVTITCRASOGISSALAWYQQKPGKAPKLLIYD
ASSLESGVPSRFSGSGSGTD FTLTISSLQPEDFATYYCQQFNSYPLTFG
GGTKVEIK (SEQ ID NO: 28; CDRs are underlined and
in bold).

Additional anti-CD117 antibodies and antigen-binding fragments thereof that may be used in conjunction with the compositions and methods described herein include those described in US 2015/0320880, such as the clones 9P3, NEG024, NEG027, NEG085, NEG086, and 20376.

The disclosures of each of the foregoing publications are incorporated herein by reference as they pertain to anti-CD117 antibodies. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD117 binding assay.

Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

Antibodies may be produced using recombinant methods and compositions, e.g., as described in U.S. Pat. No. 4,816,567. In one embodiment, isolated nucleic acid encoding an anti-CD117 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD117 antibody, or antigen binding fragment thereof, comprises variable regions having an amino acid sequence that is at least 95%, 96%, 97% or 99% identical to the SEQ ID Nos disclosed herein. Alternatively, the anti-CD117 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

In one embodiment, the anti-CD117 antibody, or antigen binding fragment thereof, comprises a heavy chain variable region and a heavy chain constant region having an amino acid sequence that is disclosed herein. In another embodiment, the anti-CD117 antibody, or antigen binding fragment thereof, comprises a light chain variable region and a light chain constant region having an amino acid sequence that is disclosed herein. In yet another embodiment, the anti-CD117 antibody, or antigen binding fragment thereof, comprises a heavy chain variable region, a light chain variable region, a heavy chain constant region and a light chain constant region having an amino acid sequence that is disclosed herein.

Additional anti-CD117 antibodies are described in US 2019/0153114 A1 and US 2019/0144558 A1, the content of both applications are hereby expressly incorporated by reference in their entirety.

The anti-CD117 antibodies and ADCs described herein can be used in methods of treating a variety of disorders, such as diseases of a cell type in the hematopoietic lineage, cancers, autoimmune diseases, metabolic disorders, and stem cell disorders, among others. The compositions and methods described herein may (i) directly deplete a population of cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of an ADC, antibody, or antigen-binding fragment thereof, capable of binding an antigen expressed by an endogenous disease-causing cell, an autoimmune cell or a hematopoietic stem cell. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. The invention is based in part on the discovery that ADCs, antibodies, or antigen-binding fragments thereof, capable of binding CD117 (such as GNNK+ CD117) can be administered to a patient to affect both of the above activities. ADCs, antibodies, or antigen-binding fragments thereof, that bind CD117 can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

Engraftment of hematopoietic stem cell transplants due to the administration of anti-CD117 ADCs, antibodies, or antigen-binding fragments thereof, can manifest in a variety of empirical measurements. For instance, engraftment of transplanted hematopoietic stem cells can be evaluated by assessing the quantity of competitive repopulating units (CRU) present within the bone marrow of a patient following administration of an ADC, antibody or antigen-binding fragment thereof capable of binding CD117 and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

### Anti-CD2 Antibodies

Human CD2 is also referred to as T-cell Surface Antigen T11/Leu-5, T11, CD2 antigen (p50), and Sheep Red Blood Cell Receptor (SRBC). CD2 is expressed on T cells. Two isoforms of human CD2 have been identified. Isoform 1 contains 351 amino acids is described in Seed, B. et al. (1987) 84: 3365-69 (see also Sewell et al. (1986) 83: 8718-22) and below (NCBI Reference Sequence: NP_001758.2):

(SEQ ID NO: 29)
msfpckfvas fllifnvssk gayskeitna letwgalgqd
inldipsfqm sddiddikwe ktsdkkkiaq frkeketfke
kdtyklfkng tlkikhlktd dqdiykvsiy dtkgknvlek
ifdlkiqerv skpkiswtci nttltcevmn gtdpelnlyq
dgkhlklsqr vithkwttsl sakfkctagn kvskessvep
vscpekgldi yliigicggg sllmvfvall vfyitkrkkq
rsrrndeele trahrvatee rgrkphqipa stpqnpatsq
hpppppghrs qapshrpppp ghrvqhqpqk rppapsgtqv
hqqkgpplpr prvqpkpphg aaenslspss n

A second isoform of CD2 is 377 amino acids and is identified herein as NCBI Reference Sequence: NP_001315538.1.

In one embodiment, an anti-CD2 antibody that may be used in conjunction with the compositions and methods described herein include those that have one or more, or all, of the following CDRs:

(SEQ ID NO: 30)
a. a CDR-H1 having the amino acid sequence EYYMY;
(SEQ ID NO: 31)
b. a CDR-H2 having the amino acid sequence
RIDPEDGSIDYVEKFKK;
(SEQ ID NO: 32)
c. a CDR-H3 having the amino acid sequence
GKFNYRFAY;
(SEQ ID NO: 33)
d. a CDR-L1 having the amino acid sequence
RSSQSLLHSSGNTYLN;
(SEQ ID NO: 34)
e. a CDR-L2 having the amino acid sequence
LVSKLES;
and
(SEQ ID NO: 35)
f. a CDR-L3 having the amino acid sequence
MQFTHYPYT.

In one embodiment, an anti-CD2 antibody, or antigen binding portion thereof, comprises a heavy chain variable region as set forth in the amino acid sequence of SEQ ID NO: 36, and a light chain variable region as set forth in the amino acid sequence of SEQ ID NO: 37.

In one embodiment, an anti-CD2 antibody that may be used in conjunction with the compositions and methods described herein include those that have one or more, or all, of the following CDRs:

(SEQ ID NO: 38)
a. a CDR-H1 having the amino acid sequence
GFTFSSY;
(SEQ ID NO: 39)
b. a CDR-H2 having the amino acid sequence SGGGF;
(SEQ ID NO: 40)
c. a CDR-H3 having the amino acid sequence
SSYGEIMDY;
(SEQ ID NO: 42)
d. a CDR-L1 having the amino acid sequence
RASQRIGTSIH;
(SEQ ID NO: 43)
e. a CDR-L2 having the amino acid sequence
YASESIS;
and
(SEQ ID NO: 44)
f. a CDR-L3 having the amino acid sequence
QQSHGWPFTF.

In one embodiment, an anti-CD2 antibody, or antigen binding portion thereof, comprises a heavy chain variable region as set forth in the amino acid sequence of SEQ ID NO: 45, and a light chain variable region as set forth in the amino acid sequence of SEQ ID NO: 47.

In another embodiment, an anti-CD2 antibody that may be used in conjunction with the compositions and methods described herein include those that have one or more, or all, of the following CDRs:

(SEQ ID NO: 38)
a. a CDR-H1 having the amino acid sequence
GFTFSSY;
(SEQ ID NO: 39)
b. a CDR-H2 having the amino acid sequence SGGGF;
(SEQ ID NO: 41)
c. a CDR-H3 having the amino acid sequence
SSYGELMDY;
(SEQ ID NO: 42)
d. a CDR-L1 having the amino acid sequence
RASQRIGTSIH;
(SEQ ID NO: 43)
e. a CDR-L2 having the amino acid sequence
YASESIS;
and
f. a CDR-L3 having the amino acid sequence
QQSHGWPFTF.

In one embodiment, an anti-CD2 antibody, or antigen binding portion thereof, comprises a heavy chain variable region as set forth in the amino acid sequence of SEQ ID NO: 46, and a light chain variable region as set forth in the amino acid sequence of SEQ ID NO: 47.

Antibodies and antigen-binding fragments thereof containing the foregoing CDR sequences are described, e.g., in U.S. Pat. No. 6,849,258, the disclosure of which is incorporated herein by reference as it pertains to anti-CD2 antibodies and antigen-binding fragments thereof.

Further, in certain embodiments the anti-CD2 ADC has a serum half-life in a human subject of 3 days or less.

Additional sequences for anti-CD2 antibodies or binding fragments, described herein, are provided in Table 5.

Additional anti-CD2 antibodies, antigen-binding fragments thereof, or ADCs thereof that can be used in the compositions and methods as described herein can be identified using techniques known in the art, such as hybridoma production. Hybridomas can be prepared using a murine system. Protocols for immunization and subsequent isolation of splenocytes for fusion are known in the art. Fusion partners and procedures for hybridoma generation are also known. Alternatively, anti-CD2 antibodies can be generated using the HuMAb-Mouse® or XenoMouse™. In making additional anti-CD2 antibodies, the CD2 antigen is isolated and/or purified. The CD2 antigen may be a fragment of CD2 from the extracellular domain of CD2. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD2 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD2 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD2 antibodies for use in the anti-CD2 ADCs described herein can also be identified using high throughput screening of libraries of antibodies or antibody fragments for molecules capable of binding CD2. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules).

In addition to in vitro display techniques, computational modeling techniques can be used to design and identify anti-CD2 antibodies or antibody fragments in silico, for instance, using the procedures described in US 2013/0288373, the disclosure of which is incorporated herein as it pertains to molecular modeling methods for identifying anti-CD2 antibodies. For example, using computational modeling techniques, one of skill in the art can screen libraries of antibodies or antibody fragments in silico for molecules capable of binding specific epitopes on CD2, such as extracellular epitopes of CD2.

In one embodiment, the anti-CD2 antibody used in the ADCs described herein are able to internalize into the cell. In identifying an anti-CD2 antibody (or fragment thereof) additional techniques can be used to identify antibodies or antigen-binding fragments that bind CD2 on the surface of a cell (e.g., a T cell) and further are able to be internalized by the cell, for instance, by receptor-mediated endocytosis. For example, the in vitro display techniques described above can be adapted to screen for antibodies or antigen-binding fragments thereof that bind CD2 on the surface of a hematopoietic stem cell and that are subsequently internalized. Phage display represents one such technique that can be used in conjunction with this screening paradigm. To identify anti-CD2 antibodies or fragments thereof that bind CD2 and are subsequently internalized a CD2+ cell, one of skill in the art can use the phage display techniques described in Williams et al., Leukemia 19:1432-1438, 2005, the disclosure of which is incorporated herein by reference in its entirety.

The internalizing capacity of an anti-CD2 antibody or fragment thereof can be assessed, for instance, using radionuclide internalization assays known in the art. For example, an anti-CD2 antibody or fragment thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with hematopoietic stem cells for a time sufficient to permit internalization. Internalized antibodies, or fragments thereof, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

In some embodiments, the anti-CD2 antibody (or fragment thereof) has a defined serum half-life. For example, an anti-CD2 antibody (or fragment thereof) may have a serum half-life of about 1-24 hours in the human patient. ADCs containing such anti-CD2 antibodies can also, for example, have a serum half-life of about 1-24 hours in a human patient. Pharmacokinetic analysis by measurement of serum levels can be performed by assays known in the art.

For recombinant production of an anti-CD2 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Anti-CD5 Antibodies

Human CD5 is also referred to as Lymphocyte Antigen T1, T1, Leu-1, and LEU1. CD5 is expressed on human T cells. Two isoforms of human CD5 have been identified. Isoform 1 contains 495 amino acids and is described in Gladkikh et al (2017) Cancer Med. 6(12):2984 and Jones et al. (1986) Nature 323 (6086): 346). The amino acid sequence of CD5 (isoform 1) is provided below (NCBI Reference Sequence: NP_055022.2):

(SEQ ID NO: 48)
mpmgslqpla tlyllgmlva sclgrlswyd pdfqarltrs
nskcqgqlev ylkdgwhmvc sqswgrsskq wedpsqaskv
cqrlncgvpl slgpflvtyt pqssiicygq lgsfsncshs
rndmchslgl tclepqkttp pttrpppttt peptapprlq
lvaqsggqhc agvvefysgs lggtisyeaq dktqdlenfl
cnnlqcgsfl khlpeteagr aqdpgepreh gplpiqwkiq
nssctslehc frkikpqksg rvlallcsgf qpkvqsrlvg
gssicegtve vrqgaqwaal cdsssarssl rweevcreqq
cgsvnsyrvl dagdptsrgl fcphqklsqc helwernsyc
kkvfvtcqdp npaglaagtv asiilalvll vvllvvcgpl
aykklvkkfr qkkqrqwigp tgmnqnmsfh rnhtatvrsh
aenptashvd neysqpprns hlsaypaleg alhrssmqpd
nssdsdydlh gaqrl

A second isoform (SEQ ID NO: 339) of human CD5 is 438 amino acids (see underlined portion above) and is identified as NCBI Reference Sequence: NP_001333385.1. Unlike isoform 1, CD5 isoform 2 is an intracellular protein. Isoform 2 contains a distinct 5′ UTR and lacks an in-frame portion of the 5′ coding region, compared to isoform 1. The resulting isoform 2 has a shorter N-terminus, compared to isoform 1. The CD5 isoform 2 lacks the leader peptide, compared to isoform 1 and represents an intracellular isoform found in a subset of B lymphocytes. The ADCs described herein are specific for human CD5 isoform 1 which represents the extracellular version of human CD5.

In one embodiment, an anti-CD5 antibody that may be used in the methods and compositions described herein is Antibody 5D7v (Ab5D7v). The heavy chain variable region (VH) amino acid sequence of Ab5D7v is provided below as SEQ ID NO: 49.

(SEQ ID NO: 49)
QVTLKESGPVLVKPTETLTLTCTFSGFSLSTSGMGVGWIRQAPGKGLEWV
AHIWWDDDVYYNPSLKSRLTITKDASKDQVSLKLSSVTAADTAVYYCVRR
RATGTGFDYWGQGTLVTVSS

The VH CDR amino acid sequences of Ab5D7v are underlined above and are as follows:

(VH CDR1; SEQ ID NO: 51)
FSLSTSGMG;
(VH CDR2; SEQ ID NO: 52)
WWDDD;
and
(VH CDR3; SEQ ID NO: 53)
RRATGTGFDY.

The light chain variable region (VL) amino acid sequence of Ab5D7v is provided below as SEQ ID NO 50.

(SEQ ID NO: 50)
NIVMTQSPSSLSASVGDRVTITCQASQDVGTAVAWYQQKPDQSPKLL
IYWTSTRHTGVPDRFTGSGSGTDFTLTISSLQPEDIATYFCHQYNSY
NTFGSGTKLEIK

The VL CDR amino acid sequences of Ab5D7v are underlined above and are as follows:

(VL CDR1; SEQ ID NO: 54)
QDVGTA;
(VL CDR2; SEQ ID NO: 55)
WTSTRHT;
and
(VL CDR3; SEQ ID NO: 56)
YNSYNT.

In one embodiment, an anti-CD5 ADC comprises an anti-CD5 antibody comprising a heavy chain comprising a CDR1 domain comprising the amino acid sequence set forth in SEQ ID NO: 51, a CDR2 domain comprising the amino acid sequence set forth in SEQ ID NO: 52, and a CDR3 domain comprising the amino acid sequence set forth in SEQ ID NO: 53, and comprises a light chain comprising a CDR1 domain comprising the amino acid sequence set forth in SEQ ID NO: 54, a CDR2 domain comprising the amino acid sequence set forth in SEQ ID NO: 55, and a CDR3 domain comprising the amino acid sequence set forth in SEQ ID NO: 56, wherein the antibody is conjugated to a cytotoxin via a linker.

In one embodiment, an anti-CD5 ADC comprises an anti-CD5 antibody comprising a heavy chain comprising a variable region comprising an amino acid sequence as set forth in SEQ ID NO:49, and a light chain comprising a variable region comprising an amino acid sequence as set forth in SEQ ID NO: 50, wherein the antibody is conjugated to a cytotoxin via a linker.

In another embodiment, an anti-CD5 antibody used in the ADCs described herein is the 5D7 antibody (see, e.g., US 20080254027, the disclosure of which is incorporated herein by reference). In another embodiment, an anti-CD5 antibody that may be used in the methods and compositions (including ADCs) described herein is a variant of the 5D7 antibody (see, e.g., US 20080254027, the disclosure of which is incorporated herein by reference).

Further, in certain embodiments the anti-CD5 ADC has a serum half-life in a human subject of 3 days or less.

Additional sequence for anti-CD5 antibodies or binding fragments, described herein, are provided in Table 5.

Additional anti-CD5 antibodies that can be used in the ADCs described herein can be identified using techniques known in the art, such as hybridoma production. Hybridomas can be prepared using a murine system. Protocols for immunization and subsequent isolation of splenocytes for fusion are known in the art. Fusion partners and procedures for hybridoma generation are also known. Alternatively, anti-CD5 antibodies can be generated using the HuMAb-Mouse® or XenoMouse™. In making additional anti-CD5 antibodies, the CD5 antigen is isolated and/or purified. The CD5 antigen may be a fragment of CD5 from the extracellular domain of CD5. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD5 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD5 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD5 antibodies for use in the anti-CD5 ADCs described herein can also be identified using high throughput screening of libraries of antibodies or antibody fragments for molecules capable of binding CD5. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules).

In addition to in vitro display techniques, computational modeling techniques can be used to design and identify anti-CD5 antibodies or antibody fragments in silico, for instance, using the procedures described in US 2013/0288373, the disclosure of which is incorporated herein as it pertains to molecular modeling methods for identifying anti-CD5 antibodies. For example, using computational modeling techniques, one of skill in the art can screen libraries of antibodies or antibody fragments in silico for molecules capable of binding specific epitopes on CD5, such as extracellular epitopes of CD5.

In one embodiment, the anti-CD5 antibody used in the ADCs described herein are able to internalize into the cell. In identifying an anti-CD5 antibody (or fragment thereof) additional techniques can be used to identify antibodies or antigen-binding fragments that bind CD5 on the surface of a cell (e.g., a T cell) and further are able to be internalized by the cell, for instance, by receptor-mediated endocytosis. For example, the in vitro display techniques described above can be adapted to screen for antibodies or antigen-binding fragments thereof that bind CD5 on the surface of a hematopoietic stem cell and that are subsequently internalized. Phage display represents one such technique that can be used in conjunction with this screening paradigm. To identify anti-CD5 antibodies or fragments thereof that bind CD5 and are subsequently internalized a CD5+ cell, one of skill in the art can use the phage display techniques described in Williams et al., Leukemia 19:1432-1438, 2005, the disclosure of which is incorporated herein by reference in its entirety.

The internalizing capacity of an anti-CD5 antibody or fragment thereof can be assessed, for instance, using radionuclide internalization assays known in the art. For example, an anti-CD5 antibody or fragment thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with hematopoietic stem cells for a time sufficient to permit internalization. Internalized antibodies, or fragments thereof, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

In some embodiments, the anti-CD5 antibody (or fragment thereof) has a defined serum half-life. For example, an anti-CD5 antibody (or fragment thereof) may have a serum half-life of about 1-24 hours in the human patient. ADCs containing such anti-CD5 antibodies can also, for example, have a serum half-life of about 1-24 hours in a human patient. Pharmacokinetic analysis by measurement of serum levels can be performed by assays known in the art.

For recombinant production of an anti-CD5 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

In some embodiments, the anti-CD5 antibodies that can be used in conjunction with the compositions and methods described herein include those that contain a combination of CDR-H1, CDR-H2, CDR-H3, CDR-L1, CDR-L2, and CDR-L3 regions set forth in Tables 1 and 2, below.

TABLE 1

| Ab | | | SEQ | | SEQ | | SEQ |
|---|---|---|---|---|---|---|---|
| No. | Name | CDRH1 | ID NO: | CDRH2 | ID NO: | CDRH3 | ID NO: |
| 1 | 1D8 | SGYSFTGYTM | 57 | LINPYNGGTT | 94 | CARDYYGSSPDFDYW | 131 |
| 2 | 3I21 | SGYSFTDYTM | 58 | LINPYNGGTM | 95 | CARDNYGSSPDFDYW | 132 |
| 3 | 4H10 | SGYSFTGYTM | 59 | LINPYNGGTM | 96 | CARDNYGSSPYFDYW | 133 |
| 4 | 8J23 | SGYSFTGYTM | 60 | LINPYNGGTM | 97 | CARDNYGSSPYFDYW | 134 |
| 5 | 5O4 | SGYSFTGYTM | 61 | LINPYNGGTT | 98 | CARDYYGSSPDFDYW | 135 |
| 6 | 4H2 | SGFTFSNYAM | 62 | SISSGGNTF | 99 | CVRYYYGVTYWYFDVW | 136 |
| 7 | 5G2 | SGFTFSSYAM | 63 | SISSGGSTY | 100 | CVRYYYGIRYWYFDVW | 137 |
| 8 | 8G8 | SGYSFTAYNI | 64 | SIDPYYGDTK | 101 | CARRMITMGDWYFDVW | 138 |
| 9 | 6M4 | SGYSFTAYSM | 65 | SIDPYYGDTK | 102 | CARRMITTGDWYFDVW | 139 |
| 10 | 2E3 | SGYTFTNFAI | 66 | LISSNSGDVS | 103 | CARHYGAHNYFDYW | 140 |
| 11 | 4E24 | SGYTFTNFAI | 67 | LISTSSGDVS | 104 | CARHYGANNYFDYW | 141 |
| 12 | 4F10 | SGYTFTNFAI | 68 | LISSNSGDVS | 105 | CARHYGAHNYFDYW | 142 |
| 13 | 7J9 | SGYTFTNFAI | 69 | LISSNSGDVS | 106 | CARHYGAHNYFDYW | 143 |
| 14 | 7P9 | SGFNIKDTYM | 70 | RIDPANGNTK | 107 | CAREENYYGTYYFDYW | 144 |
| 15 | 8E24 | SGYSFTSYWM | 71 | MIHPSDSETR | 108 | CARWGDHDDAMDFW | 145 |
| 16 | 6L18 | SGFSLTNYDV | 72 | VIWSGGNTD | 109 | CARNHGDGYFNWYFDVW | 146 |
| 17 | 7H7 | SGFSLTNYDV | 73 | VIWSGGNTD | 110 | CARNHGDGYYNWYFDVW | 147 |
| 18 | 1E7 | SGFTFSNYGM | 74 | AINSNGDITY | 111 | CARGTAWFTYW | 148 |
| 19 | 8J21 | SGYSFTGYTM | 75 | LINPYNGGTR | 112 | CARDGDDGWDIDVW | 149 |
| 20 | 7I11 | SGYIFANYGM | 76 | WINTYTGEPT | 113 | CARRGTYWHFDVW | 150 |
| 21 | 8M9 | SGYNFTNYGM | 77 | WINTYTGEPT | 114 | CARRGSYWHFDVW | 151 |
| 22 | 1P21 | SGYTFTNYGM | 78 | WINTYTGEPT | 115 | CARRSTLVFDYW | 152 |
| 23 | 2H11 | SGYTFTDYYI | 79 | WIYPGGGNTR | 116 | CARNGYWYFDVW | 153 |
| 24 | 3M22 | SGYTFTDYYI | 80 | WIYPGGGNTR | 117 | CARNGYWYFDVW | 154 |
| 25 | 5M6 | SGNTFTNFYL | 81 | CIYPGNVKTK | 118 | CAKEGDYDGTAYFDYW | 155 |
| 26 | 5H8 | SGYTFTNYGM | 82 | WINTYTGEPT | 119 | CARRRDGNFDYW | 156 |
| 27 | 7I19 | SEFTFSNYAM | 83 | TISSGGSYTY | 120 | CVRHGYFDVW | 157 |
| 28 | 1A20 | SGYTFTSYRM | 84 | RIDPYDSGTH | 121 | CAFYDGAYW | 158 |
| 29 | 8E15 | SGFNIKDTYM | 85 | RIDPANGNTK | 122 | CASYDPDYW | 159 |
| 30 | 8C10 | SGYSFTDYTM | 86 | LINPYNGGTR | 123 | CARDTTATYYFDYW | 160 |
| 31 | 3P16 | SGYMFTNHGM | 87 | WINTYTGEPT | 124 | CARRVATYFDVW | 161 |
| 32 | 4F3 | SGYMFTNYGM | 88 | WINTYTGEPT | 125 | CTRRSHITLDYW | 162 |
| 33 | 5M24 | SGYIFTNYGM | 89 | WINTYTGEPT | 126 | CARRRTTAFDYW | 163 |
| 34 | 5O24 | SGFNIKDYYI | 90 | WIDPENGRTE | 127 | CNNGNYVRHYYFDYW | 168 |
| 35 | 7B16 | SGYTFINYGM | 91 | WINTYTGEPT | 128 | CTRRREITFDYW | 164 |
| 36 | 1E8 | SGYTFTDYFI | 92 | EIYPGSSNTY | 129 | CARSGISPFTYW | 165 |
| 37 | 2H16 | SGYIFTGYNI | 93 | AVYPGNGDTS | 130 | CAKYDRFFASW | 166 |

TABLE 2

| Ab | | | SEQ | | SEQ | | SEQ |
|---|---|---|---|---|---|---|---|
| No. | Name | CDRL1 | ID NO: | CDRL2 | ID NO: | CDRL3 | ID NO: |
| 1 | 1D8 | SQGISNHL | 167 | YFTSS | 204 | CQQYSNLPYTF | 241 |
| 2 | 3I21 | SQGIRNYL | 185 | YFTSS | 205 | CQQYSNLPYTF | 242 |
| 3 | 4H10 | SQGISNHL | 169 | YFTSS | 206 | CQQYSNLPYTF | 243 |
| 4 | 8J23 | SQGINNYL | 170 | YYTSS | 207 | CQQYSKIPYTC | 244 |
| 5 | 5O4 | SQGISNHL | 171 | YFTSS | 208 | CQQYSNLPYTF | 245 |
| 6 | 4H2 | SQSVDHDGDSYM | 172 | YAASN | 209 | CQQNYEDPTF | 246 |
| 7 | 5G2 | SQSVDYDGDSYM | 173 | YAASN | 210 | CQQSNEDPTF | 247 |
| 8 | 8G8 | SQDISNYL | 174 | YYTSR | 211 | CQQGDALPWTF | 248 |
| 9 | 6M4 | SQDISTYL | 175 | FYTSR | 212 | CQQGNSLPFTF | 249 |
| 10 | 2E3 | TSSISSSYL | 176 | YGTSN | 213 | CQQWSSRPPTF | 250 |
| 11 | 4E24 | NSSVSSSYL | 177 | YGTSN | 214 | CQQYSGYPLTF | 251 |
| 12 | 4F10 | TSSISSSYL | 178 | YGTSN | 215 | CQQYSDYPLTF | 252 |
| 13 | 7J9 | TSSISSSYL | 179 | YGTSN | 216 | CQQRSYFPFTF | 253 |
| 14 | 7P9 | SENIYYNL | 180 | YNANS | 217 | CKQVYDVPFTF | 254 |
| 15 | 8E24 | SENIYGYF | 181 | YNAKT | 218 | CQHHYGTPFTF | 255 |
| 16 | 6L18 | SQDINNYI | 182 | HYTST | 219 | CLQYDNLWTF | 256 |
| 17 | 7H7 | SQDINKYI | 183 | HYTST | 220 | CLQYDNLWTF | 257 |
| 18 | 1E7 | SENIYSYL | 184 | YNAKT | 221 | CQHHYGYPYTF | 258 |
| 19 | 8J21 | SQGIRNYL | 185 | YHTST | 222 | CQQYSNLPLTF | 259 |
| 20 | 7I11 | SQDVRTDV | 186 | YSASF | 223 | CQQHYTSPWTF | 260 |
| 21 | 8M9 | SQDVITAV | 187 | YSASY | 224 | CQQHYSTPWTF | 261 |
| 22 | 1P21 | SQSIGTSI | 188 | KSASE | 225 | CQQSNRWPLTF | 262 |
| 23 | 2H11 | SSQSLLNQKNYL | 189 | YWAST | 226 | CQNDYDYPYTF | 263 |
| 24 | 3M22 | SSSVSSSYL | 190 | YSTSN | 227 | CHQYHRSPLTF | 264 |
| 25 | 5M6 | SENIYYNL | 191 | YNANS | 228 | CQQTFDVPWTF | 265 |
| 26 | 5H8 | SQTIGTSI | 192 | KNASE | 229 | CQQSNSWPLTY | 266 |
| 27 | 7I19 | SQSLLYSSDQKNYL | 193 | YWAST | 230 | CQQYYNYPLTF | 267 |
| 28 | 1A20 | NSSVSYM | 194 | YDTSK | 231 | CQQWSSNPFTF | 268 |
| 29 | 8E15 | SENIYYNL | 195 | YNANS | 232 | CKQAYDVPWTF | 269 |
| 30 | 8C10 | SSSLSYM | 196 | YDTSN | 233 | CQQWSSFPPTF | 270 |
| 31 | 3P16 | SQRIGTSM | 197 | KSASE | 234 | CQQSNSWPLTF | 271 |
| 32 | 4F3 | SQSIGTSI | 198 | KSASE | 235 | CQQSNSWPLTF | 272 |
| 33 | 5M24 | SQNIGTSI | 199 | KDASE | 236 | CQQSDSWPLTF | 273 |
| 34 | 5O24 | ISSVSYM | 200 | YATSN | 237 | CQQWSSNPRTF | 274 |
| 35 | 7B16 | SQTIATSI | 201 | KNASE | 238 | CQQSNSWPLTF | 275 |
| 36 | 1E8 | SQSLVHSNGNTYL | 202 | YKVSN | 239 | CWQNTHFPQTF | 276 |
| 37 | 2H16 | NESVEYSGTSLM | 203 | SAASN | 240 | CQQSRQVPLTF | 277 |

### Anti-CD137 Antibodies

CD137 is also referred to as CDw137, TNFRSF9, 4-1 BB, and ILA. Anti-CD137 antibodies, antigen-binding fragments thereof and ADCs thereof can be used as therapeutic agents to prevent and treat GVHD from hematopoietic stem cells in a patient suffering from or at risk for GVHD or an autoimmune disease. Additionally, it has been discovered that ligands that bind CD137, such as human CD137L, can be used as a therapeutic agent to prevent or treat patient suffering from or at risk for GVHD. These ligands, such as soluble human CD137, can be covalently bound to an effector domain, such as an Fc domain, for instance, in order to promote antibody-dependent cell-mediated cytotoxicity (ADCC).

T cells have been shown to express CD137, as this antigen is a transmembrane TNF receptor superfamily of costimulatory molecules and is expressed on a variety of hematopoietic cells and promotes T cell activation and regulates proliferation and survival of T cells (see, e.g., Cannons et al., J. Immunol. 167:1313-1324, 2001, the disclosure of which is incorporated herein by reference as it pertains to the expression of CD137 by T cells). Antibodies, and antigen-binding fragments thereof, can be identified using techniques known in the art and described herein, such as by immunization, computational modeling techniques, and in vitro selection methods, such as the phage display and cell-based display platforms described below.

Anti-CD137 antibodies that can be used to prevent and treat GVHD or an autoimmune disease by the methods disclosed herein include those that have one or more, or all, of the following CDRs:

a. a CDR-H1 having the amino acid sequence
STYWIS (SEQ ID NO: 278);
b. a CDR-H2 having the amino acid sequence
KIYPGDSYTNYSPSFQG (SEQ ID NO: 279);
c. a CDR-H3 having the amino acid sequence
RGYGIFDY (SEQ ID NO: 280);
d. a CDR-L1 having the amino acid sequence
SGDNIGDQYAH (SEQ ID NO: 281)
e. a CDR-L2 having the amino acid sequence
QDKNRPS (SEQ ID NO: 282);
and
f. a CDR-L3 having the amino acid sequence
ATYTGFGSLAV (SEQ ID NO: 283)

Additional anti-CD137 antibodies that can be used to prevent and treat GVHD and autoimmune diseases by the methods disclosed herein include those that have one or more, or all, of the following CDRs:

a. a CDR-H1 having the amino acid sequence
STYWIS (SEQ ID NO: 278);
b. a CDR-H2 having the amino acid sequence
KIYPGDSYTNYSPSFQG (SEQ ID NO: 279);
c. a CDR-H3 having the amino acid sequence
RGYGIFDY (SEQ ID NO: 280);
d. a CDR-L1 having the amino acid sequence
SGDNIGDQYAH (SEQ ID NO: 281)
e. a CDR-L2 having the amino acid sequence
QDKNRPS (SEQ ID NO: 282);
and
f. a CDR-L3 having the amino acid sequence
STYTFVGFTTV (SEQ ID NO: 284)

Additional anti-CD137 antibodies include those that have one or more, or all, of the following CDRs:

a. a CDR-H1 having the amino acid sequence
NSYAIS (SEQ ID NO: 285);
b. a CDR-H2 having the amino acid sequence
GIIPGFGTANYAQKFQG (SEQ ID NO: 286);
c. a CDR-H3 having the amino acid sequence
RKNEEDGGFDH (SEQ ID NO: 287);
d. a CDR-L1 having the amino acid sequence
SGDNLGDYYAS (SEQ ID NO: 288)
e. a CDR-L2 having the amino acid sequence
DDSNRPS (SEQ ID NO: 289);
and
f. a CDR-L3 having the amino acid sequence
QTWDGTLHFV (SEQ ID NO: 290)

Additional anti-CD137 antibodies or ADCs include those that have one or more, or all, of the following CDRs:

a. a CDR-H1 having the amino acid sequence
SDYYMH (SEQ ID NO: 291);
b. a CDR-H2 having the amino acid sequence
VISGSGSNTYYADSVKG (SEQ ID NO: 292);
c. a CDR-H3 having the amino acid sequence
RLYAQFEGDF (SEQ ID NO: 293);
d. a CDR-L1 having the amino acid sequence
SGDNIGSKYVS (SEQ ID NO: 294)
e. a CDR-L2 having the amino acid sequence
SDSERPS (SEQ ID NO: 295);
and
f. a CDR-L3 having the amino acid sequence
QSWDGSISRV (SEQ ID NO: 296)

The foregoing antibodies are described, e.g., in U.S. Pat. No. 9,468,678, the disclosure of which is incorporated herein by reference as it pertains to anti-CD137 antibodies and antigen-binding fragments thereof. The antibodies and fragments thereof disclosed in U.S. Pat. No. 9,468,678 can be used in conjunction with the methods disclosed herein.

In another embodiment, an anti-CD137 antibody that may be used in the methods and compositions (including ADCs) described herein is the murine anti-CD137 antibody BBK2 (Thermo Fisher; MS621PABX) or an anti-CD137 antibody comprising antigen binding regions corresponding to the BBK2 antibody. The BBK2 antibody (which may also be referred to as a BBK-2 antibody or an anti-4-1BB antibody), is a mouse monoclonal antibody (IgG1, kappa) that binds to the ectodomain of human 4-1 BB recombinant protein (4-1BB is also known as CD137). In certain embodiments, the methods and compositions of the disclosure include an anti-CD137 antibody comprising the binding regions (e.g., the CDRs) of the BBK2 antibody. In another embodiment, the methods and compositions of the disclosure comprise an antibody that competitively inhibits the binding of the BBK2 antibody to its epitope on CD137. In certain embodiments, the anti-CD137 antibody is humanized BBK2 or chimeric BBK2.

In one embodiment, the methods and compositions described herein include a chimeric anti-CD137 (ch-BBK2) antibody comprising the variable heavy and light chain regions of BBK2. In certain embodiments, the chimeric BBK2 antibody is an IgG1 antibody comprising human constant regions. The heavy chain amino acid sequence of ch-BBK2 is described in SEQ ID NO: 297, and the light chain amino acid sequence of ch-BBK2 is described in SEQ ID NO: 298. The CDR regions (CDR1, CDR2, and CDR3) of each of the heavy and light chain sequences are described in bold below. The variable regions are italicized.

(SEQ ID NO: 297)
QVQLQQPGAELVRPGASVKLSCKASGYTFTSYWINWVKQRPGQGLEW
IGNIYPSDSYTNYNQKFKDKATLTVDKSSNTVYMQLNSPTSEDSAVY
YCTRNGVEGYPHYYAMEYWGQGTSVIVSSASTKGPSVFPLAPSSKST
SGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSRDELTKNQVSLTCLVK
GFYPSDIAVEWESNGQPENNYKTTPPVLDSDGSFFLYSKLTVDKSRW
QQGNVFSCSVMHEALHNHYTQKSLSLSPGK
(SEQ ID NO: 298)
DIQMTQTTSALSASLGDRVTIGCRASQDLSNHLYWYQQKPDGTVKLL
IYYTSRLHSGVPSRFSGSGSGTDYSLTIRNLEQEDVATYFCQQGYTL
PYTFGGGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

The foregoing CDR regions (and BBK2 antibody) are described in Lee et al. (2002) European J of Immunogenetics 29(5):449-452. Thus, in one embodiment, the VH CDR amino acid sequences of anti-CD137 antibody BBK2 (including ch-BBK2) are as follows: SGYTFTSYW (VH CDR1; SEQ ID NO: 299); NIYPSDSYT (VH CDR2; SEQ ID NO: 300) and TRNGVEGYPHYYAME (VH CDR3; SEQ ID NO: 301). The VL CDR amino acid sequences of anti-CD137 antibody BBK2 (including ch-BBK2) are as follows: SQDLSNH (VL CDR1; SEQ ID NO: 302); YYTS (VL CDR2; SEQ ID NO: 303) and CQQGYTLPY (VL CDR3; SEQ ID NO: 304).

Alternatively, the CDR regions of BBK2 can be defined according to Kabat numbering. CDRs as defined by Kabat numbering are described below for each of the heavy and light chain sequences (described in bold below). The variable regions of BBK2 are italicized.

(ch-BBK2 heavy chain; SEQ ID NO: 297)
QVQLQQPGAELVRPGASVKLSCKASGYTFT [symbol] WVKQRPGQGLEW
IG [symbol] [symbol] KATLTVDKSSNTVYMQLNSPTSEDSAVY
YCTR [symbol] WGQGTSVTVSSASTKGPSVFPLAPSSKST
SGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSRDELTKNQVSLTCLVK
GFYPSDIAVEWESNGQPENNYKTTPPVLDSDGSFFLYSKLTVDKSRW
QQGNVFSCSVMHEALHNHYTQKSLSLSPGK
(ch-BBK2 light chain; SEQ ID NO: 298)
DIQMTQTTSALSASLGDRVTIGC [symbol] WYQQKPDGTVKLL
IY [symbol] GVPSRFSGSGSGTDYSLTIRNLEQEDVATYFC [symbol]
FGGGTKLEIKRTVAAPSVFIFPPSDEQLKSGTASVVCLL
NNFYPREAKVQWKVDNALQSGNSQESVTEQDSKDSTYSLSSTLTLSK
ADYEKHKVYACEVTHQGLSSPVTKSFNRGEC

Thus, in one embodiment, the VH CDR amino acid sequences of anti-CD137 antibody BBK2 (including ch-BBK2) are as follows: SYWIN (VH CDR1; SEQ ID NO: 305); NIYPSDSYTNYNQKFKD (VH CDR2; SEQ ID NO: 306) and NGVEGYPHYYAMEY (VH CDR3; SEQ ID NO: 307), and the VL CDR amino acid sequences of anti-CD137 antibody BBK2 (including ch-BBK2) are as follows: RASQDLSNHLY (VL CDR1; SEQ ID NO: 308); YTSRLHS (VL CDR2; SEQ ID NO: 309) and QQGYTLPYT (VL CDR3; SEQ ID NO: 310).

The heavy chain variable region of BBK2 is set forth in SEQ ID NO: 311 as QVQLQQPGAELVRPGASVKLSCKASGYTFTSYWINWVKQRPGQGLEWIGNIYPSDSYTNYNQKF KDKATLTVDKSSNTVYMQLNSPTSEDSAVYYCTRNGVEGYPHYYAMEYWGQGTSVTVSS. The light chain variable region of BBK2 is set forth in SEQ ID NO: 312 as DIQMTQTTSALSASLGDRVTIGCRASQDLSNHLYWYQQKPDGTVKLLIYYTSRLHSGVPSRFSGS GSGTDYSLTIRNLEQEDVATYFCQQGYTLPYTFGGGTKLEIK. Anti-CD137 antibodies (including anti-CD137 ADCs) can comprise the heavy and light chain variable region amino acid sequences as set forth in SEQ ID Nos: 311 and 312, respectively.

In one embodiment, the anti-CD137 antibody, e.g., a chimeric (ch-BBK2) antibody or a humanized BBK2 antibody, comprises a heavy chain variable region comprising a CDR1 comprising the amino acid sequence of SEQ ID NO: 305, a CDR2 comprising the amino acid sequence of SEQ ID NO: 306, and a CDR3 comprising the amino acid sequence of SEQ ID NO: 307; and comprises a light chain variable region comprising a CDR1 comprising the amino acid sequence of SEQ ID NO: 308, a CDR2 comprising the amino acid sequence of SEQ ID NO: 309, and a CDR3 comprising the amino acid sequence of SEQ ID NO: 310.

In one embodiment, the anti-CD137 antibody, e.g., a chimeric (ch-BBK2) antibody or a humanized BBK2 antibody, comprises a heavy chain variable region comprising a CDR1 comprising the amino acid sequence of SEQ ID NO: 299, a CDR2 comprising the amino acid sequence of SEQ ID NO: 300, and a CDR3 comprising the amino acid sequence of SEQ ID NO: 301; and comprises a light chain variable region comprising a CDR1 comprising the amino acid sequence of SEQ ID NO: 302, a CDR2 comprising the amino acid sequence of SEQ ID NO: 303, and a CDR3 comprising the amino acid sequence of SEQ ID NO: 304.

Thus, BBK2, humanized BBK2, or chimeric BBK2 antibodies can be used in the anti-CD137 ADCs and methods described herein. Each of these antibodies can be conjugated to any of the cytotoxin described below using methods known in the art and those described herein.

Additional sequence for anti-CD137 antibodies or binding fragments, described herein, are provided in Table 5.

Other anti-CD137 antibodies that can be used in conjunction with a cytotoxin described herein can be identified using techniques known in the art (e.g., hybridoma production). Hybridomas can be prepared using a murine system. Protocols for immunization and subsequent isolation of splenocytes for fusion are known in the art. Fusion partners and procedures for hybridoma generation are also known. Human anti-CD137 antibodies can also be generated in the HuMAb-Mouse® or XenoMouse™. In making anti-CD137 antibodies, the CD137 antigen is isolated and/or purified. The CD137 antigen may be a fragment of CD137 from the extracellular domain of CD137. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD137 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD137 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD137 antibodies can be generated from an isolated nucleic acid molecule that comprises a nucleotide sequence encoding an amino acid sequence of a CD137 binding molecule provided by the present disclosure. The amino acid sequence encoded by the nucleotide sequence may be any portion of an antibody, such as a CDR, a sequence comprising one, two, or three CDRs, a variable region of a heavy chain, variable region of a light chain, or may be a full-length heavy chain or full length light chain. A nucleic acid of the disclosure can be, for example, DNA or RNA, and may or may not contain intronic sequences. Typically, the nucleic acid is a cDNA molecule.

In addition to antibodies, and antigen-binding fragments, soluble CD137 ligands, such as human CD137 ligand, can be administered to a patient according to the methods described herein to condition a patient prior to hematopoietic stem cell transplant therapy. For instance, CD137 ligands, such as human CD137 ligand, can be conjugated to a cytotoxin (e.g., according to the methods described below or known in the art) or another effector molecule, such as an Fc domain. Maytansine cytotoxins for use with the methods described herein include, for example, human CD137 ligand-IgG1 Fc conjugates, human CD137 ligand-IgG2 Fc conjugates, human CD137 ligand-IgG3 Fc conjugates, human CD137 ligand-IgG4 Fc conjugates, human CD137 ligand-IgA Fc conjugates, human CD137 ligand-IgE Fc conjugates, human CD137 ligand-IgM Fc conjugates, and human CD137 ligand-IgD Fc conjugates.

Antibodies and ligands for use in conjunction with the compositions and methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, antibody fragment, or soluble ligand described herein.

### Anti-CD252 Antibodies

The present invention also provides antibodies, or antigen-binding fragments thereof, capable of binding CD252 (also referred to as OX40 ligand (OX40L), Protein NCBI Reference Sequence: NP_003317.1; Uniprot Accession No: P23510; SEQ ID NOs: 313 or 314) can be used as a therapeutic agent to prevent and treat GVHD. Such antibodies can be used alone or conjugated to a cytotoxin as an antibody drug conjugate (ADC).

In one embodiment, methods and compositions (e.g., ADCs) described herein include an anti-CD252 antibody whose heavy and light chain amino acid sequences are set forth in SEQ ID NOs. 315 and 316, respectively. In one embodiment, an anti-CD252 antibody, or antigen binding portion thereof, comprises a heavy chain variable region as set forth in the amino acid sequence of SEQ ID NO: 315, and a light chain variable region as set forth in the amino acid sequence of SEQ ID NO: 316. In one embodiment, an anti-CD252 antibody, or antigen binding portion thereof, comprises a heavy chain variable region comprising CDRs as set forth in the amino acid sequence of SEQ ID NO: 315, and a light chain variable region comprising CDRs as set forth in the amino acid sequence of SEQ ID NO: 316. The amino acid sequences of SEQ ID NOs: 315 and 316 are provided below.

In certain embodiments, an anti-CD252 antibody, or antigen binding portion thereof, comprises a heavy chain variable region comprising CDRs as set forth in the amino acid sequence of SEQ ID NOs: 317-319, and a light chain variable region comprising CDRs as set forth in the amino acid sequence of SEQ ID NO: 320-322. The amino acid sequences of SEQ ID NOs: 3-8 are provided below.

Anti-CD252 VH amino acid sequence (the following CDR sequences are defined by IMGT)

(SEQ ID NO: 315)
EVQLVESGGGLVQPGGSLRLSCAASGFTFSNYAMNWVRQAPGKGLEW
VSTISGSGGATRYADSVKGRFTISRDNSRNTVYLQMNSLRVEDTAVF
YCTKDRLIMATVRGPYYYGMDVWGQGTTVTVSS
CDR-H1:
(SEQ ID NO: 317)
GFTFSNYA
CDR-H2:
(SEQ ID NO: 318)
ISGSGGAT
CDR-H3:
(SEQ ID NO: 319)
TKDRLIMATVRGPYYYGMDV

Anti-CD252 VL amino acid sequence (the following CDR sequences are defined by IMGT)

(SEQ ID NO: 316)
DIQMTQSPSSLSASVGDRVTITCRASQSISSYLNWYQQKPGKAPNLL
IYAASSLQSGVPSRFSGSGSETDFTLTISSLQPEDFATYYCQQSHSV
SFTFGPGTKVDIK
CDR-L1:
(SEQ ID NO: 320)
QSISSY
CDR-L2:
(SEQ ID NO: 321)
AAS
CDR-L3:
(SEQ ID NO: 322)
QQSHSVSFT

In one embodiment, an anti-CD252 antibody used in the methods and compositions disclosed herein is an intact antibody comprising a heavy chain variable region as set forth in the amino acid sequence of SEQ ID NO: 315, and a light chain variable region as set forth in the amino acid sequence of SEQ ID NO: 316. In one embodiment, the anti-CD252 antibody is engineered to have a short half life.

In one embodiment, an anti-CD252 antibody that may be used in the methods and compositions (including ADCs) described herein is an antibody selected from 11C3.1 (Biolegend, Catalog #326302), 159403 (R&D Systems, Catalog # MAB10541), 159408 (R&D Systems, Catalog # MAB1054), MM0505-8523 (Novus, Catalog # NBP2-11969), or oxelumab (Novus Catalog # NBP2-52687-0.1).

In one embodiment, an anti-CD252 antibody that may be used in the methods and compositions (including ADCs) described herein is the murine monoclonal anti-CD252 antibody 11C3.1 or an anti-CD252 antibody comprising antigen binding regions corresponding to the 11C3.1 antibody. 11C3.1 (sold by Biolegend Cat. No. 326302 (date Feb. 27, 2019)).

In one embodiment, an anti-CD252 antibody comprises a heavy chain comprising a CDR1, CDR2 and CDR3 of anti-CD252 antibody 11C3.1, and a light chain variable region comprising a CDR1, CDR2 and CDR3 of anti-CD252 antibody 11C3.1. In another embodiment, an anti-CD252 antibody used in the compositions and methods disclosed herein is a humanized 11C3.1 antibody.

In one embodiment, an anti-CD252 antibody that may be used in the methods and compositions (including ADCs) described herein is the murine monoclonal anti-CD252 antibody 159403 or an anti-CD252 antibody comprising antigen binding regions corresponding to the 159403 antibody. 159403 (sold by R&D Systems, Catalog # MAB10541 (date Feb. 27, 2019)).

In one embodiment, an anti-CD252 antibody comprises a heavy chain comprising a CDR1, CDR2 and CDR3 of anti-CD252 antibody 159403, and a light chain variable region comprising a CDR1, CDR2 and CDR3 of anti-CD252 antibody 159403. In another embodiment, an anti-CD252 antibody used in the compositions and methods disclosed herein is a humanized 159403 antibody.

In one embodiment, an anti-CD252 antibody that may be used in the methods and compositions (including ADCs) described herein is the murine monoclonal anti-CD252 antibody 159408 or an anti-CD252 antibody comprising antigen binding regions corresponding to the 159408 antibody. 159408 (sold by R&D Systems, Catalog # MAB1054 (date Feb. 27, 2019)).

In one embodiment, an anti-CD252 antibody comprises a heavy chain comprising a CDR1, CDR2 and CDR3 of anti-CD252 antibody 159408, and a light chain variable region comprising a CDR1, CDR2 and CDR3 of anti-CD252 antibody 159408. In another embodiment, an anti-CD252 antibody used in the compositions and methods disclosed herein is a humanized 159408 antibody.

In one embodiment, an anti-CD252 antibody that may be used in the methods and compositions (including ADCs) described herein is the murine monoclonal anti-CD252 antibody MM0505-8S23 or an anti-CD252 antibody comprising antigen binding regions corresponding to the MM0505-8S23 antibody. MM0505-8S23 (sold by Novus, Catalog # NBP2-11969 (date Feb. 27, 2019)). This antibody was produced from a hybridoma (mouse myeloma fused with spleen cells from a mouse immunized with human TNFSF4, also called OX40 ligand.

In one embodiment, an anti-CD252 antibody comprises a heavy chain comprising a CDR1, CDR2 and CDR3 of anti-CD252 antibody MM0505-8S23, and a light chain variable region comprising a CDR1, CDR2 and CDR3 of anti-CD252 antibody MM0505-8S23. In another embodiment, an anti-CD252 antibody used in the compositions and methods disclosed herein is a humanized MM0505-8S23 antibody.

In one embodiment, an anti-CD252 antibody that may be used in the methods and compositions (including ADCs) described herein is the rabbit monoclonal anti-CD252 antibody oxelumab or an anti-CD252 antibody comprising antigen binding regions corresponding to the oxelumab antibody. Oxelumab (sold by Novus, Catalog # NBP2-52687-0.1 (date Feb. 27, 2019)).

In one embodiment, an anti-CD252 antibody comprises a heavy chain comprising a CDR1, CDR2 and CDR3 of anti-CD252 antibody oxelumab, and a light chain variable region comprising a CDR1, CDR2 and CDR3 of anti-CD252 antibody oxelumab. In another embodiment, an anti-CD252 antibody used in the compositions and methods disclosed herein is a humanized oxelumab antibody. In some embodiment, the anti-CD252 antibody, or antigen binding portion thereof, comprises a heavy chain as set forth in the amino acid sequence of SEQ ID NO: 323, and a light chain as set forth in the amino acid sequence of SEQ ID NO: 324. In some embodiment, the anti-CD252 antibody, or antigen binding portion thereof, comprises a heavy chain variable region as set forth in the amino acid sequence of SEQ ID NO: 331, and a light chain variable region as set forth in the amino acid sequence of SEQ ID NO: 332. In one embodiment, an anti-CD252 antibody, or antigen binding portion thereof, comprises a heavy chain variable region comprising CDRs as set forth in the amino acid sequence of SEQ ID NO: 325-327, and a light chain variable region comprising CDRs as set forth in the amino acid sequence of SEQ ID NO: 328-330. In one embodiment, the antibody is an intact antibody comprising a heavy chain variable region as set forth in the amino acid sequence of SEQ ID NO: 331, and a light chain variable region as set forth in the amino acid sequence of SEQ ID NO: 332. The amino acid sequences of SEQ ID NOs: 323-330 are provided below.

oxelumab full length heavy chain sequence (the following CDR sequences are defined by IMGT;
the heavy chain variable region (SEQ ID NO: 331) has been underlined):

(SEQ ID NO: 323)
EVQLLESGGGLVQPGGSLRLSCAASGFTFNSYAMSWVRQAPGKGLEW
VSIISGSGGFTYYADSVKGRFTISRDNSRTTLYLQMNSLRAEDTAVY
YCAKDRLVAPGTFDYWGQGALVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTIPPVLDSDGSFFLYSKLTVDKSRWQQG
NVFSCSVMHEALHNHYTQKSLSLSPG
CDR-H1:
(SEQ ID NO: 325)
GFTFNSYA
CDR-H2:
(SEQ ID NO: 326)
ISGSGGFT
CDR-H3:
(SEQ ID NO: 327)
AKDRLVAPGTFDY

oxelumab full length light chain sequence (the following CDR sequences are defined by IMGT; the light chain variable region (SEQ ID NO: 332) has been underlined):

(SEQ ID NO: 324)
DIQMTQSPSSLSASVGDRVTITCRASQGISSWLAWYQQKPEKAPKSL
IYAASSLQSGVPSRFSGSGSGTDFTLTISSLQPEDFATYYCQQYNSY
PYTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
CDR-L1:
(SEQ ID NO: 328)
QGISSW
CDR-L2:
(SEQ ID NO: 329)
AAS
CDR-L3:
(SEQ ID NO: 330)
QQYNSYPYT

The anti-CD252 antibodies or binding fragments described herein may also include modifications and/or mutations that alter the properties of the antibodies and/or fragments, such as those that increase half-life, increase or decrease ADCC, etc., as is known in the art.

In one embodiment, an anti-CD252 antibody, or binding fragment thereof, used in the methods and compositions disclosed herein comprises a variant Fc region, wherein said variant Fc region comprises at least one amino acid modification relative to a wild-type Fc region, such that said molecule has an altered affinity for an FcgammaR. Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). Thus, the anti-CD252 antibodies described herein may comprise variant Fc regions comprising modification of at least one residue that makes a direct contact with an Fc.γ.R based on structural and crystallographic analysis. In one embodiment, the Fc region of the anti-CD252 antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the anti-CD252 antibody (or fragment thereof) comprises an amino acid substitution at amino acid 234 according to the EU index as in Kabat. In one embodiment, the Fc region comprises a L234A mutation. In some embodiments, the Fc region of the anti-CD252 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In certain aspects a variant IgG Fc domain comprises one or more amino acid substitutions resulting in decreased or ablated binding affinity for an Fc.gamma.R and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an anti-CD252 antibody comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™. analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

The antibodies of the invention may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, I253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. An antibody having a short half life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express CD252 but are not the target of the anti-CD252 antibody, unlike the endogenous stem cells. In one embodiment, the Fc regions comprises a mutation at position 435 (EU index according to Kabat). In one embodiment, the mutation is an H435A mutation.

In one embodiment, the anti-CD252 antibody described herein has a half life of equal to or less than about 14 hours, equal to or less than about 13 hours, equal to or less than about 12 hours, or equal to or less than about 11 hours. In one embodiment, the anti-CD252 antibody described herein has a half life of equal to or less than about 24 hours, a half life of equal to or less than about 22 hours, a half life of equal to or less than about 20 hours, a half life of equal to or less than about 18 hours, a half life of equal to or less than about 16 hours, a half life of equal to or less than about 14 hours, equal to or less than about 13 hours, equal to or less than about 12 hours, or equal to or less than about 11 hours. In one embodiment, the half life of the antibody is between about 1 hour to about 20 hours, between about 2 hours to about 18 hours, between about 4 hours to about 16 hours, between about 6 hours to about 14 hours, between about 8 hours to about 12 hours, between about 11 hours to about 12 hours, between about 11 hours to about 24 hours; between about 12 hours to about 22 hours; between about 10 hours to about 20 hours; between about 8 hours to about 18 hours; between about 1 hours to about 6 hours, between about 2 hours to about 5 hours, between about 3 hours to about 4 hours, or between about 14 hours to about 24 hours.

In some aspects, the Fc region comprises two or more mutations that confer reduced half-life and greatly diminish or completely abolish an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD252 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation.

In some embodiments of these aspects, the cysteine residue is naturally occurring in the Fc domain of the antibody or antigen-binding fragment thereof. For instance, the Fc domain may be an IgG Fc domain, such as a human IgG1 Fc domain, and the cysteine residue may be selected from the group consisting of Cys261, Cys321, Cys367, and Cys425.

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary.

In one embodiment, the anti-CD252 antibody, or antigen binding fragment thereof, comprises variable regions having an amino acid sequence that is at least 95%, 96%, 97% or 99% identical to the SEQ ID Nos disclosed herein. Alternatively, the anti-CD252 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

In certain embodiments, an anti-CD252 antibody, or antigen binding fragment thereof, has a certain dissociation rate which is particularly advantageous when used as a part of a conjugate. For example, an anti-CD252 antibody has, in certain embodiments, an off rate constant (Koff) for human CD252 and/or rhesus CD252 of 1×10⁻² to 1×10⁻³, 1×10⁻³ to 1×10⁻⁴, 1×10⁻⁵ to 1×10⁻⁶, 1×10⁻⁶ to 1×10⁻⁷ or 1×10⁻⁷ to 1×10⁻⁸, as measured by bio-layer interferometry (BLI). In some embodiments, the antibody or antigen-binding fragment thereof binds CD252 (e.g., human CD252 and/or rhesus CD252) with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay. In some embodiments, the antibody or antigen-binding fragment thereof binds CD252 (e.g., human CD252 and/or rhesus CD252) with a K_{D} of between about 90 nM-100 nM, between about 80 nM-90 nM, between about 70 nM-80 nM, between about 60 nM-70 nM, between about 50 nM-60 nM, between about 40 nM-50 nM, between about 30 nM-40 nM, between about 20 nM-30 nM, between about 10 nM-20 nM, between about 8 nM-10 nM, between about 6 nM-8 nM, between about 4 nM-6 nM, between about 2 nM-4 nM, between about 1 nM-2 nM, or about 1 nM or less as determined by a Bio-Layer Interferometry (BLI) assay.

The antibodies, and binding fragments thereof, disclosed herein can be used in conjugates, as described in more detail below.

Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others. The anti-CD252 antibodies described herein can be in the form of full-length antibodies, bispecific antibodies, dual variable domain antibodies, multiple chain or single chain antibodies, and/or binding fragments that specifically bind human CD252, including but not limited to Fab, Fab′, (Fab′)₂, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the anti-CD252 antibody is an IgG (e.g. IgG1, IgG2, IgG3 or IgG4).

In one embodiment, the anti-CD252 antibody, or antigen binding fragment thereof, comprises variable regions having an amino acid sequence that is at least 95%, 96%, 97% or 99% identical to the SEQ ID Nos disclosed herein. Alternatively, the anti-CD252 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

### Anti-CD45 Antibodies

Antibodies and antigen-binding fragments capable of binding human CD45 (mRNA NCBI Reference Sequence: NM_080921.3, Protein NCBI Reference Sequence: NP_563578.2), including those capable of binding the isoform CD45RO, can be used in conjunction with the compositions and methods disclosed herein, such as to promote engraftment of hematopoietic stem cell grafts in a patient in need of hematopoietic stem cell transplant therapy. In one embodiment, the compositions and methods disclosed herein include an anti-CD45 antibody or ADC that binds to human CD45RO as set forth in the amino acid sequence of SEQ ID NO: 336. Antibodies that bind to the various isoforms of CD45 disclosed herein are also contemplated for use in the methods and compositions disclosed herein. Multiple isoforms of CD45 arise from the alternative splicing of 34 exons in the primary transcript. Splicing of exons 4, 5, 6, and potentially 7 give rise to multiple CD45 variations. Selective exon expression is observed in the CD45 isoforms described in Table 3, below.

TABLE 3
Exon expression in various CD45 isoforms

| CD45 Isoform | Exon Expression Pattern |
|---|---|
| CD45RA (SEQ ID NO: | Expresses exon 4 only |
| 333) | |
| CD45RB (SEQ ID NO: | Expresses exon 5 only |
| 334) | |
| CD45RC (SEQ ID NO: | Expresses exon 6 only |
| 335) | |
| CD45RO (SEQ ID NO: | Does not express exons 4-6 |
| 336) | |

Alternative splicing can result in individual exons or combinations of exons expressed in various isoforms of the CD45 protein (for example, CD45RA, CD45RAB, CD45RABC). In contrast, CD45RO lacks expression of exons 4-6 and is generated from a combination of exons 1-3 and 7-34. There is evidence that exon 7 can also be excluded from the protein, resulting in splicing together of exons 1-3 and 8-34. This protein, designated E3-8, has been detected at the mRNA level but has not been currently identified by flow cytometry.

CD45RO is currently the only known CD45 isoform expressed on hematopoietic stem cells. CD45RA and CD45RABC have not been detected or are excluded from the phenotype of hematopoietic stem cells. There is evidence from studies conducted in mice that CD45RB is expressed on fetal hematopoietic stem cells, but it is not present on adult bone marrow hematopoietic stem cells. Notably, CD45RC has a high rate of polymorphism in exon 6 found within Asian populations (a polymorphism at exon 6 in CD45RC is found in approximately 25% of the Japanese population). This polymorphism leads to high expression of CD45RO and decreased levels of CD45RA, CD45RB, and CD45RC. Additionally, CD45RA variants (such as CD45RAB and CD45RAC) exhibit a polymorphism in exon 4 that has been associated with autoimmune disease.

The presence of CD45RO on hematopoietic stem cells and its comparatively limited expression on other immune cells (such as T and B lymphocyte subsets and various myeloid cells) renders CD45RO a particularly well-suited target for conditioning therapy for patients in need of a hematopoietic stem cell transplant. As CD45RO only lacks expression of exons 4, 5, and 6, its use as an immunogen enables the screening of pan CD45 Abs and CD45RO-specific antibodies.

Anti-CD45 antibodies that can be used in conjunction with the patient conditioning methods described herein include anti-CD45 antibodies, and antigen-binding portions thereof. Antigen-binding portions of antibodies are well known in the art, and can readily be constructed based on the antigen-binding region of the antibody. In exemplary embodiments, the anti-CD45 antibody used in conjunction with the conditioning methods described herein can be a monoclonal antibody or antigen-binding fragment thereof, a polyclonal antibody or antigen-binding fragment thereof, a humanized antibody or antigen-binding fragment thereof, a fully human antibody or antigen-binding fragment thereof, a chimeric antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)2 molecule, or a tandem di-scFv. Exemplary anti-CD45 antibodies which may be used in whole or in part in the ADCs or methods described herein are provided below.

In one embodiment, the anti-CD45 antibody is or is derived from clone HI30, which is commercially available from BIOLEGEND® (San Diego, Calif.), or a humanized variant thereof. Humanization of antibodies can be performed by replacing framework residues and constant region residues of a non-human antibody with those of a germline human antibody according to procedures known in the art (as described, for instance, in Example 7, below). Additional anti-CD45 antibodies that can be used in conjunction with the methods described herein include the anti-CD45 antibodies ab10558, EP322Y, MEM-28, ab10559, 0.N.125, F10-89-4, Hle-1, 2611, YTH24.5, PD7/26/16, F10-89-4, 1B7, ab154885, B-A11, phosphor S1007, ab170444, EP350, Y321, GA90, D3/9, X1 6/99, and LT45, which are commercially available from ABCAM® (Cambridge, Mass.), as well as humanized variants thereof. Further anti-CD45 antibodies that may be used in conjunction with the patient conditioning procedures described herein include anti-CD45 antibody HPA000440, which is commercially available from SIGMA-ALDRICH® (St. Louis, Mo.), and humanized variants thereof. Additional anti-CD45 antibodies that can be used in conjunction with the patient conditioning methods described herein include murine monoclonal antibody BC8, which is described, for instance, in Matthews et al., Blood 78:1864-1874, 1991, the disclosure of which is incorporated herein by reference as it pertains to anti-CD45 antibodies, as well as humanized variants thereof. Further anti-CD45 antibodies that can be used in conjunction with the methods described herein include monoclonal antibody YAML568, which is described, for instance, in Glatting et al., J. Nucl. Med. 8:1335-1341, 2006, the disclosure of which is incorporated herein by reference as it pertains to anti-CD45 antibodies, as well as humanized variants thereof. Additional anti-CD45 antibodies that can be used in conjunction with the patient conditioning procedures described herein include monoclonal antibodies YTH54.12 and YTH25.4, which are described, for instance, in Brenner et al., Ann. N.Y. Acad. Sci. 996:80-88, 2003, the disclosure of which is incorporated herein by reference as it pertains to anti-CD45 antibodies, as well as humanized variants thereof. Additional anti-CD45 antibodies for use with the patient conditioning methods described herein include UCHL1, 2H4, SN130, MD4.3, MBI, and MT2, which are described, for instance, in Brown et al., Immunology 64:331-336, 1998, the disclosure of which is incorporated herein by reference as it pertains to anti-CD45 antibodies, as well as humanized variants thereof. Additional anti-CD45 antibodies that can be used in conjunction with the methods described herein include those produced and released from American Type Culture Collection (ATCC) Accession Nos. RA3-6132, RA3-2C2, and TIB122, as well as monoclonal antibodies C363.16A, and 13/2, which are described, for instance, in Johnson et al., J. Exp. Med. 169:1179-1184, 1989, the disclosure of which is incorporated herein by reference as it pertains to anti-CD45 antibodies, as well as humanized variants thereof. Further anti-CD45 antibodies that can be used in conjunction with the patient conditioning methods described herein include the monoclonal antibodies AHN-12.1, AHN-12, AHN-12.2, AHN-12.3, AHN-12.4, HLe-1, and KC56(T200), which are described, for instance, in Harvath et al., J. Immunol. 146:949-957, 1991, the disclosure of which is incorporated herein by reference as it pertains to anti-CD45 antibodies, as well as humanized variants thereof.

Additional anti-CD45 antibodies that can be used in conjunction with the patient conditioning methods described herein include those described, for example, in U.S. Pat. No. 7,265,212 (which describes, e.g., anti-CD45 antibodies 39E11, 16C9, and 1G10, among other clones); U.S. Pat. No. 7,160,987 (which describe, e.g., anti-CD45 antibodies produced and released by ATCC Accession No. HB-11873, such as monoclonal antibody 6G3); and U.S. Pat. No. 6,099,838 (which describes, e.g., anti-CD45 antibody MT3, as well as antibodies produced and released by ATCC Accession Nos. HB220 (also designated MB23G2) and HB223), as well as US 2004/0096901 and US 2008/0003224 (which describes, e.g., anti-CD45 antibodies produced and released by ATCC Accession No. PTA-7339, such as monoclonal antibody 17.1), the disclosures of each of which are incorporated herein by reference as they pertain to anti-CD45 antibodies.

Further anti-CD45 antibodies that can be used in conjunction with the patient conditioning methods described herein include antibodies produced and released from ATCC Accession Nos. MB4B4, MB23G2, 14.8, GAP 8.3, 74-9-3, I/24.D6, 9.4, 4B2, M1/9.3.4.HL.2, as well as humanized and/or affinity-matured variants thereof. Affinity maturation can be performed, for instance, using in vitro display techniques described herein or known in the art, such as phage display, as described in Example 6, below.

Additional anti-CD45 antibodies that can be used in conjunction with the patient conditioning methods described herein include anti-CD45 antibody T29/33, which is described, for instance, in Morikawa et al., Int. J. Hematol. 54:495-504, 1991, the disclosure of which is incorporated herein by reference as it pertains to anti-CD45 antibodies.

In certain embodiments, the anti-CD45 antibody is selected from apamistamab (also known 90Y-BC8, Iomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003/048327, WO2016/016442, US2017/0226209, US2016/0152733, U.S. Pat. No. 9,701,756; US2011/0076270, or U.S. Pat. No. 7,825,222, each of which is incorporated by reference in its entirety.

For example, in one embodiment, the anti-CD45 antibody, or antigen-binding fragment thereof, comprising binding regions, e.g., CDRs, variable regions, corresponding to those of apamistamab. The heavy chain variable region (VH) amino acid sequence of apamistamab is set forth in SEQ ID NO: 337. The light chain variable region (VL) amino acid sequence of apamistamab is described in SEQ ID NO: 338. In other embodiments, an anti-CD45 antibody, or antigen-binding portion thereof, comprises a variable heavy chain comprising the amino acid residues set forth in SEQ ID NO: 337, and a light chain variable region as set forth in SEQ ID NO: 338. In one embodiment, the anti-CD45 antibody comprises a heavy chain comprising a CDR1, CDR2 and CDR3 of apamistamab, and a light chain variable region comprising a CDR1, CDR2 and CDR3 of apamistamab.

In one embodiment, the anti-CD45 antibody comprises a heavy chain of an anti-CD45 antibody described herein, and a light chain variable region of anti-CD45 antibody described herein. In one embodiment, the anti-CD45 antibody comprises a heavy chain comprising a CDR1, CDR2 and CDR3 of an anti-CD45 antibody described herein, and a light chain variable region comprising a CDR1, CDR2 and CDR3 of an anti-CD45 antibody described herein.

In another embodiment, the antibody, or antigen-binding fragment thereof, comprises a heavy chain variable region that comprises an amino acid sequence having at least 95% identity to an anti-CD45 antibody herein, e.g., at least 95%, 96%, 97%, 98%, 99%, or 100% identity to an anti-CD45 antibody herein. In certain embodiments, an antibody comprises a modified heavy chain (HC) variable region comprising an HC variable domain of an anti-CD45 antibody herein, or a variant thereof, which variant (i) differs from the anti-CD45 antibody in 1, 2, 3, 4 or 5 amino acids substitutions, additions or deletions; (ii) differs from the anti-CD45 antibody in at most 5, 4, 3, 2, or 1 amino acids substitutions, additions or deletions; (iii) differs from the anti-CD45 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD45 antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-CD45 antibody, while retaining the CD45 binding specificity of the antibody.

The disclosures of each of the foregoing publications are incorporated herein by reference in their entirety. Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD45 binding assay.

### Methods of Identifying Antibodies

Methods for high throughput screening of antibody, or antibody fragment libraries for molecules capable of binding an antigen (e.g., CD117 (e.g., GNNK+ CD117), or CD45) expressed by hematopoietic stem cells or an antigen (e.g., CD2, CD5, CD137, or CD252) expressed by mature immune cells (e.g., T-cells) can be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate antibodies, or antigen-binding fragments, that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). Human anti-HC antibodies (e.g., anti-CD117 antibody, anti-CD45 antibody, anti-CD2 antibody, anti-CD5 antibody, anti-CD137 antibody, or anti-CD252 antibody) can also be generated, for example, in the HuMAb-Mouse® or XenoMouse™. These techniques, among others, can be used to identify and improve the affinity of antibodies, antibody or fragments, capable of binding an antigen (e.g., CD117 (e.g., GNNK+ CD117), or CD45) expressed by hematopoietic stem cells or an antigen (e.g., CD2, CD5, CD137, or CD252) expressed by mature immune cells (e.g., T-cells) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies capable of binding an antigen (e.g., CD117 (e.g., GNNK+ CD117), or CD45) expressed by hematopoietic stem cells or an antigen (e.g., CD2, CD5, CD137, or CD252) expressed by mature immune cells (e.g., T-cells), or antibody fragments in silico. For example, using computational modeling techniques, one of skill in the art can screen libraries of antibodies, or antibody fragments, in silico for molecules capable of binding specific epitopes on an antigen expressed by hematopoietic stem cells (e.g., CD117 (e.g., GNNK+ CD117) or CD45) or an antigen expressed by mature immune cells, such as T-cells (e.g., CD2, CD5, CD137, or CD252), such as extracellular epitopes of the antigen. The antibodies, or antigen-binding fragments thereof, identified by these computational techniques can be used in conjunction with the therapeutic methods described herein, such as, e.g., the cancer and autoimmune disease treatment methods described herein and the patient conditioning procedures described herein.

Additional techniques can be used to identify antibodies, or antibody fragments, capable of binding an antigen expressed by hematopoietic stem cells (e.g., CD117 (e.g., GNNK+ CD117) or CD45) or an antigen expressed by mature immune cells, such as T-cells (e.g., CD2, CD5, CD137, or CD252) and that are internalized by the cell, for instance, by receptor-mediated endocytosis. For example, the in vitro display techniques described above can be adapted to screen for antibodies, or antibody fragments, that bind an antigen expressed by hematopoietic stem cells (e.g., CD117 (e.g., GNNK+ CD117) or CD45) or an antigen expressed by mature immune cells, such as T-cells (e.g., CD2, CD5, CD137, or CD252) and that are subsequently internalized. Phage display represents one such technique that can be used in conjunction with this screening paradigm. To identify an anti-HC antibody (e.g., anti-CD117 antibody, anti-CD45 antibody, anti-CD2 antibody, anti-CD5 antibody, anti-CD137 antibody, or anti-CD252 antibody), or antibody fragment, and are subsequently internalized by hematopoietic stem cells (or immune cells), one of skill in the art can use the phage display techniques described, for example. in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with an antigen (e.g., CD117 (e.g., GNNK+ CD117), CD45, CD2, CD5, CD137, or CD252), for instance, by first incubating the phage library with blocking agents (such as, for instance, milk protein, bovine serum albumin, and/or IgG so as to remove phage encoding antibodies, or antibody fragments, that exhibit non-specific protein binding and phage that encode antibodies or fragments thereof that bind Fc domains, and then incubating the phage library with a population of hematopoietic stem cells or mature immune cells (e.g., T-cells), which express, e.g., CD117 (e.g., GNNK+ CD117), CD45, CD2, CD5, CD137, or CD252 The phage library can be incubated with the target cells, such as cancer cells, autoimmune cells, or hematopoietic stem cells for a time sufficient to allow anti-HC antibodies (e.g., anti-CD117 antibody, anti-CD45 antibody, anti-CD2 antibody, anti-CD5 antibody, anti-CD137 antibody, or anti-CD252 antibody) or antibody fragments thereof, to bind the cognate cell-surface antigen (e.g., CD117 (e.g., GNNK+ CD117), CD45, CD2, CD5, CD137, or CD252) and to subsequently be internalized by the hematopoietic stem cells (e.g., from 30 minutes to 6 hours at 4° C., such as 1 hour at 4° C.). Phage containing antibodies, or antibody fragments thereof, that do not exhibit sufficient affinity for the antigen (CD117 (e.g., GNNK+ CD117), CD45, CD2, CD5, CD137, or CD252) so as to permit binding to, and internalization by, the target cells, such as cancer cells, autoimmune cells, or hematopoietic stem cells, can subsequently be removed by washing the cells, for instance, with cold (4° C.) 0.1 M glycine buffer at pH 2.8. Phage bound to antibodies, or antibody fragments thereof, that have been internalized by the target cells, such as cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or antibody fragments, inserted within the phage genome. The encoded antibodies, or antibody fragments thereof, can subsequently be prepared de novo by chemical synthesis (for instance, of antibody fragments thereof, such as scFv fragments) or by recombinant expression (for instance, of full-length antibodies).

The internalizing capacity of the prepared antibodies, or antibody fragments thereof, can be assessed, for instance, using radionuclide internalization assays known in the art. For example, anti-HC antibodies (e.g., anti-CD117 antibody, anti-CD45 antibody, anti-CD2 antibody, anti-CD5 antibody, anti-CD137 antibody, or anti-CD252 antibody) or antibody fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or antibody fragments, using beads, such as polystyrene beads, containing electrophilic halogen reagents (e.g., Iodination Beads, Thermo Fisher Scientific, Inc., Cambridge, Mass.). Radiolabeled antibodies, fragments thereof, or ADCs, can be incubated with target cells, such as cancer cells, autoimmune cells, or hematopoietic stem cells, for a time sufficient to permit internalization (e.g., from 30 minutes to 6 hours at 4° C., such as 1 hour at 4° C.). The cells can then be washed to remove non-internalized antibodies or fragments thereof, (e.g., using cold (4° C.) 0.1 M glycine buffer at pH 2.8). Internalized antibodies, or antibody fragments thereof, can be identified by detecting the emitted radiation (e.g., γ-radiation) of the resulting target cells, such as cancer cells, autoimmune cells, or hematopoietic stem cells in comparison with the emitted radiation (e.g., γ-radiation) of the recovered wash buffer. The foregoing internalization assays can also be used to characterize ADCs.

Antibodies may be produced using recombinant methods and compositions, e.g., as described in U.S. Pat. No. 4,816,567. In one embodiment, isolated nucleic acid encoding an anti-HC antibody (e.g., anti-CD117 antibody, anti-CD45 antibody, anti-CD2 antibody, anti-CD5 antibody, anti-CD137 antibody, or anti-CD252 antibody) described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-HC antibody (e.g., an anti-CD117 antibody, an anti-CD45 antibody, an anti-CD2 antibody, an anti-CD5 antibody, an anti-CD137 antibody, or an anti-CD252 antibody),) nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Antibody Drug Conjugates (ADCs)

Antibodies (including anti-CD117 antibodies) and antigen-binding fragments thereof described herein can be conjugated (linked) to a cytotoxin via a linker. In some embodiments, the cytotoxic molecule is conjugated to a cell internalizing antibody, or antigen-binding fragment thereof as disclosed herein such that following the cellular uptake of the antibody, or fragment thereof, the cytotoxin may access its intracellular target and mediate hematopoietic cell death. Any number of cytotoxins can be conjugated to the anti-CD117 antibody, e.g., 1, 2, 3, 4, 5, 6, 7, or 8.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

### Cytotoxins

Various cytotoxins can be conjugated to an anti-HC antibody (e.g., an anti-CD117 antibody, an anti-CD45 antibody, an anti-CD2 antibody, an anti-CD5 antibody, an anti-CD137 antibody, or an anti-CD252 antibody) via a linker for use in the therapies described herein. In particular, the anti-HC ADCs (e.g., anti-CD117 ADC, anti-CD45 ADC, anti-CD2 ADC, anti-CD5 ADC, anti-CD137 ADC, or anti-CD252 ADC) include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

ADCs of the present disclosure present disclosure therefore may be of the general formula Ab-(Z-L-D)ₙ, wherein an antibody or antigen-binding fragment thereof (Ab) is conjugated (covalently linked) to linker (L), through a chemical moiety (Z), to a cytotoxic moiety (“drug,” D), each as disclosed herein.

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-HC ADCs (e.g., anti-CD117 ADC, anti-CD45 ADC, anti-CD2 ADC, anti-CD5 ADC, anti-CD137 ADC, or anti-CD252 ADC) may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, an indolinobenzodiazepine pseudodimer, or a variant thereof, or another cytotoxic compound described herein or known in the art.

In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof.

Additional details regarding cytotoxins that can be used in the anti-HC ADCs (e.g., anti-CD117 ADC, anti-CD45 ADC, anti-CD2 ADC, anti-CD5 ADC, anti-CD137 ADC, or anti-CD252 ADC) useful in the methods of the invention are described below.

Amatoxins

The methods and compositions disclosed herein include ADCs comprising an RNA polymerase inhibitor, e.g., an amatoxin, as the cytotoxin conjugated to an anti-HC antibody (e.g., an anti-CD117 antibody). In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin of the antibody-drug conjugate as disclosed herein is an amatoxin or derivative thereof. such as an α-amanitin, β-amanitin, γ-amanitin, ε-amanitin, amanin, amaninamide, amanullin, amanullinic acid, proamanullin or a derivative thereof. Suitable amatoxins are disclosed in, e.g., Zanotti et al., Int. J. Peptide Protein Res. 30, 1987, 450-459.

Amatoxins useful in conjunction with the compositions and methods described herein include compounds according to, but are not limited to, formula (III), including α-amanitin, β-amanitin, γ-amanitin, ε-amanitin, amanin, amaninamide, amanullin, amanullinic acid, or proamanullin. Formula (III) is as follows:

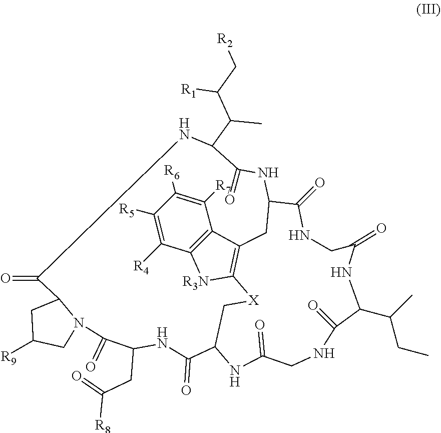

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₅ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

For instance, in one embodiment, amatoxins useful in conjunction with the compositions and methods described herein include compounds according to formula (IIIA)

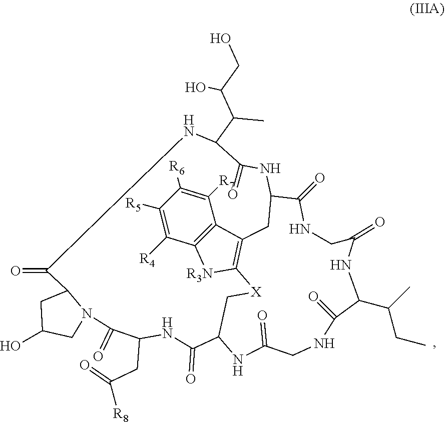

wherein R₄, R₅, X, and R₅ are each as defined above.

For instance, in one embodiment, amatoxins useful in conjunction with the compositions and methods described herein include compounds according to formula (IIIB), below:

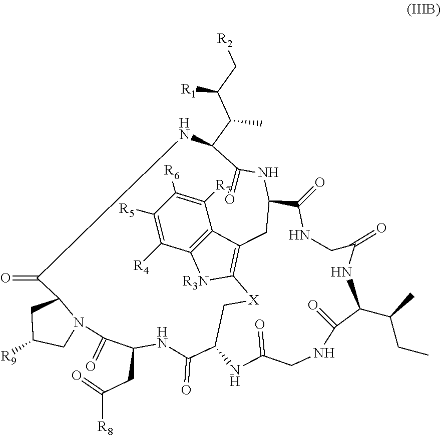

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₅ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, amatoxins useful in conjunction with the compositions and methods described herein also include compounds according to formula (IIIC), below:

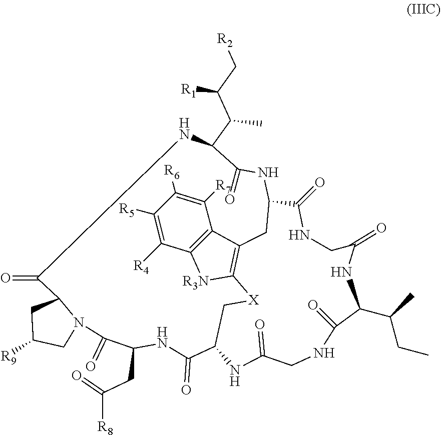

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₅ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, the cytotoxin is an amanitin. For instance, the antibodies, and antigen-binding fragments, described herein may be bound to an amatoxin so as to form a conjugate represented by the formula Ab-Z-L-Am, wherein Ab is the antibody, or antigen-binding fragment thereof, L is a linker, Z is a chemical moiety and Am is an amatoxin. Many positions on amatoxins or derivatives thereof can serve as the position to covalently bond the linking moiety L, and, hence the antibodies or antigen-binding fragments thereof. Exemplary methods of amatoxin conjugation and linkers useful for such processes are described below. Exemplary linker-containing amatoxins useful for conjugation to an antibody, or antigen-binding fragment, in accordance with the compositions and methods described herein are shown in structural formulas (I), (IA), (IB), (II), (IIA), and (IIB), recited herein.

In some embodiments, the amatoxin-linker conjugate Am-L-Z is represented by formula (I)

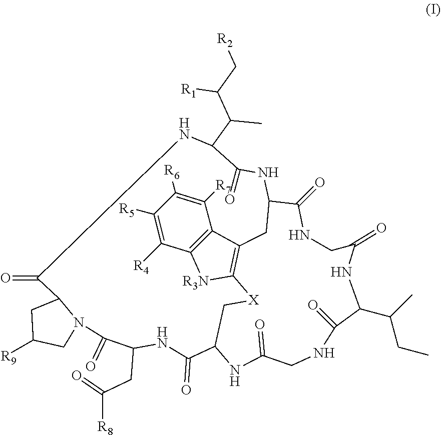

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₅ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof;

and

Z is a chemical moiety formed from a coupling reaction between a reactive substituent present on L and a reactive substituent present within an antibody, or antigen-binding fragment thereof, that binds a target antigen (e.g., CD117).

In some embodiments, Am contains exactly one R_{C} substituent.

In some embodiments, L-Z is

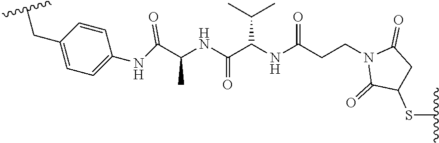

where S is a sulfur atom which represents the reactive substituent present within an antibody, or antigen-binding fragment thereof, that binds a target antigen (e.g., from the —SH group of a cysteine residue).

In some embodiments, the conjugate Am-L-Z-Ab is represented by one of formulas IV, IVA, or IVB:

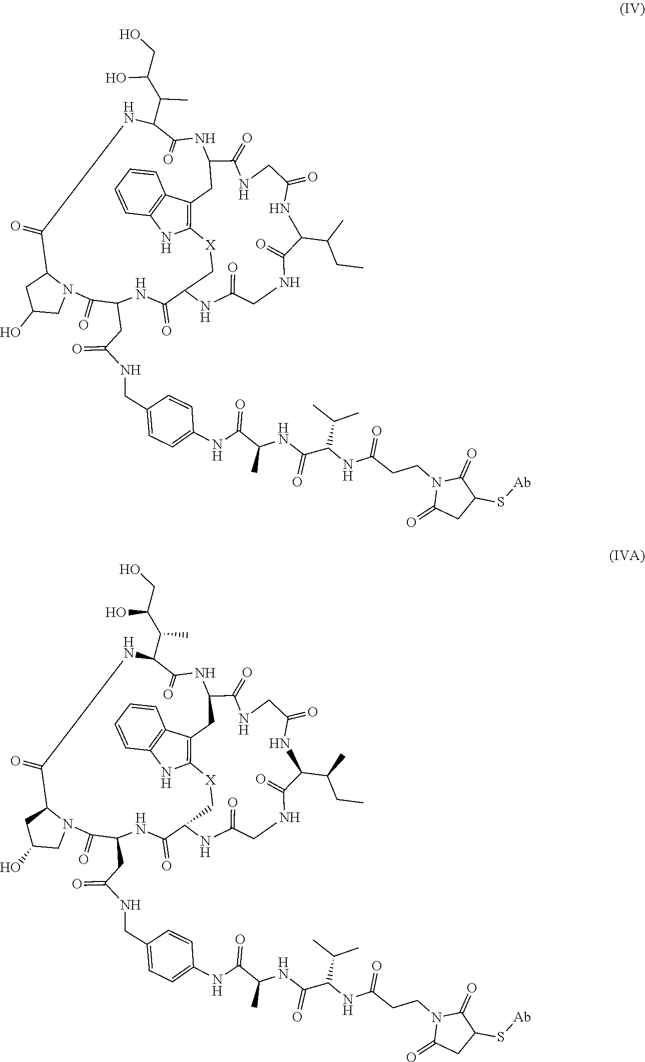

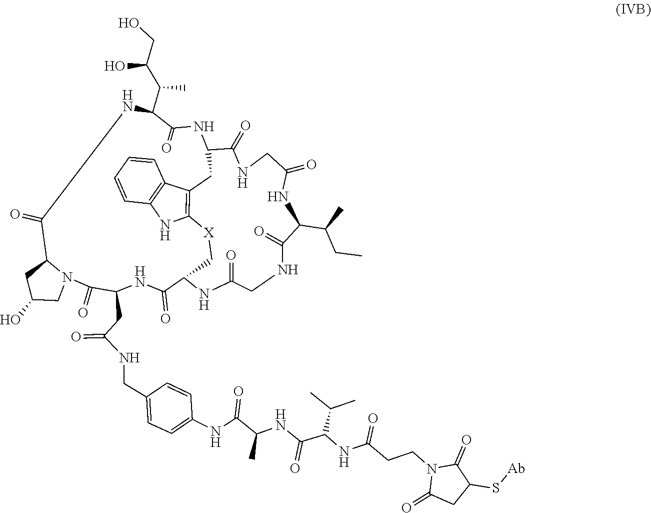

where X is S, SO or SO₂, and the Ab is shown to indicate the point of Ab attachment.

In some embodiments, Am-L-Z-Ab is

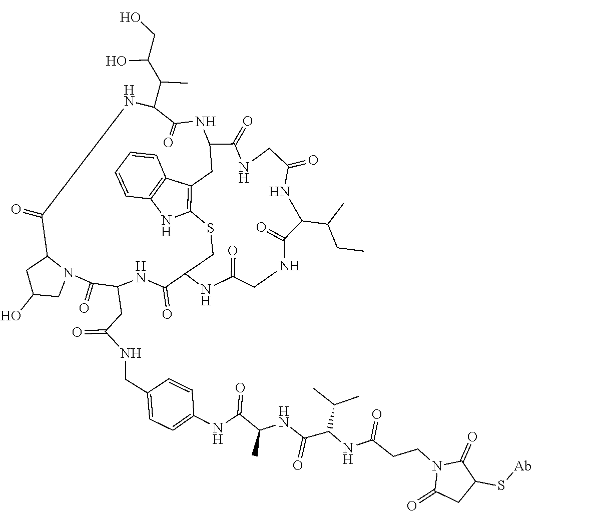

where Ab is shown to indicate the point of Ab attachment.

In some embodiments, Am-L-Z-Ab is

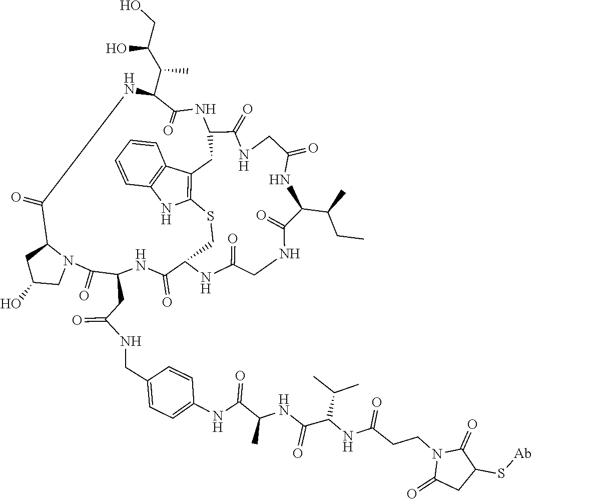

where Ab is shown to indicate the point of Ab attachment.

In some embodiments, Am-L-Z-Ab is

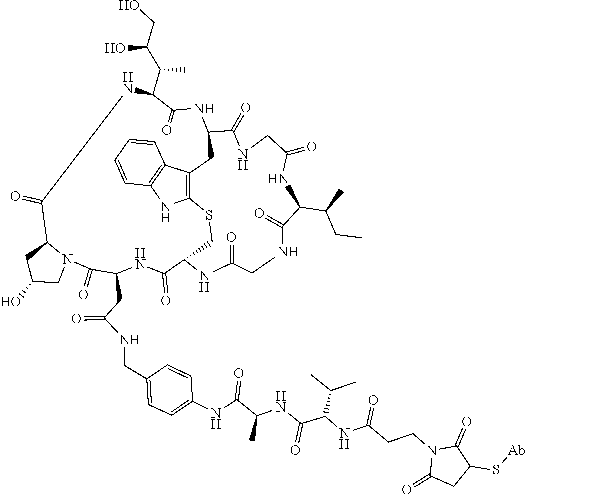

where Ab is shown to indicate the point of Ab attachment.

In some embodiments, the Am-L-Z-Ab precursor, Am-L-Z′, is

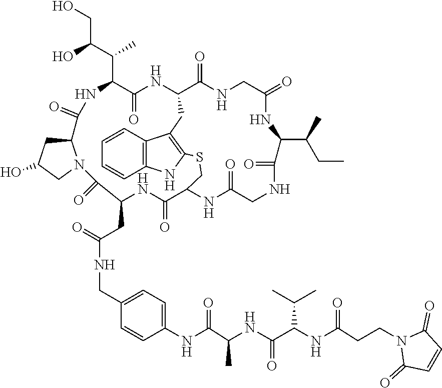

wherein the maleimide reacts with a thiol group found on a cysteine in the antibody.

In some embodiments, the Am-L-Z-Ab precursor, Am-L-Z′, is

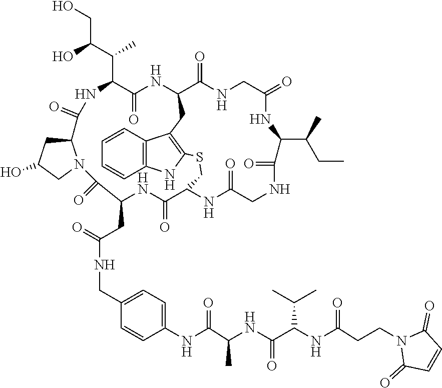

wherein the maleimide reacts with a thiol group found on a cysteine in the antibody.

In some embodiments, Am-L-Z is represented by formula (IA)

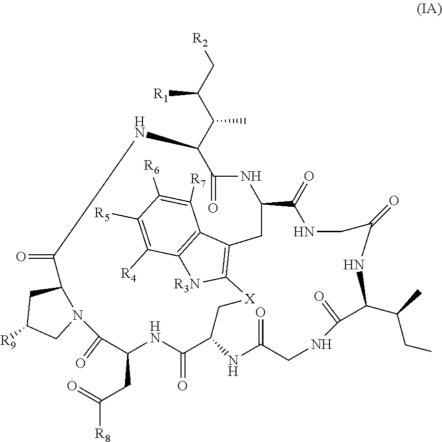

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₅ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof;

Z is a chemical moiety formed from a coupling reaction between a reactive substituent present on L and a reactive substituent present within an antibody, or antigen-binding fragment thereof, that binds an HC antigen (i.e., an anti-HC antibody, e.g., anti-CD117 antibody, anti-CD45 antibody, anti-CD2 antibody, anti-CD5 antibody, anti-CD137 antibody, or anti-CD252 antibody); and

wherein Am contains exactly one R_{C} substituent.

In some embodiments, L-Z is

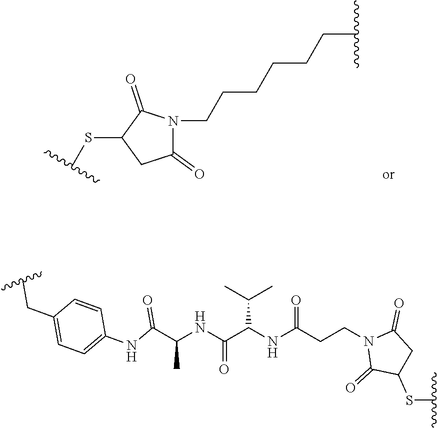

In some embodiments, L-Z is

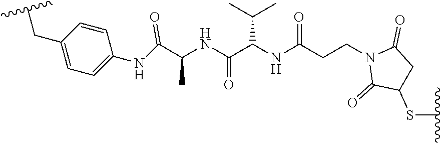

In some embodiments, Am-L-Z is represented by formula (IB)

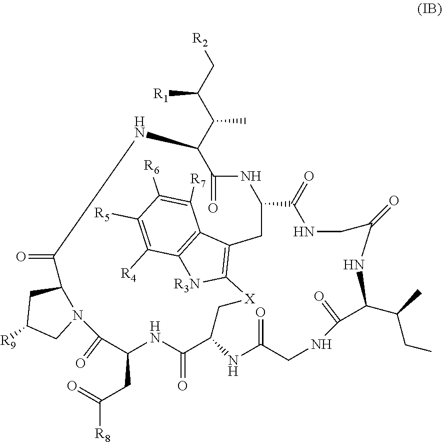

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₅ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof;

Z is a chemical moiety formed from a coupling reaction between a reactive substituent present on L and a reactive substituent present within an antibody, or antigen-binding fragment thereof, that binds an HC antigen (i.e., an anti-HC antibody, e.g., anti-CD117 antibody, anti-CD45 antibody, anti-CD2 antibody, anti-CD5 antibody, anti-CD137 antibody, or anti-CD252 antibody); and

wherein Am contains exactly one R_{C} substituent.

In some embodiments, L-Z is

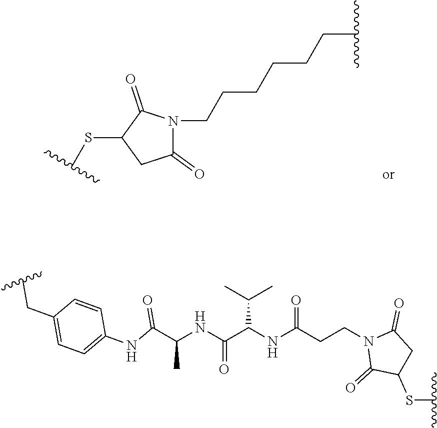

In some embodiments, L-Z is

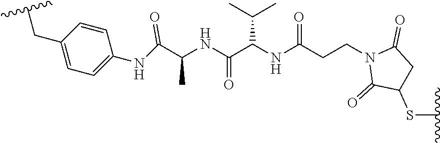

In some embodiments, R_{A} and R_{B}, when present, together with the oxygen atoms to which they are bound, combine to form a 5-membered heterocycloalkyl group of

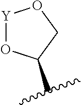

formula:

wherein Y is —(C═O)—, —(C═S)—, —(C═NR_{E})—, or —(CR_{E}R_{E′})—; and

R_{E} and R_{E′} are each independently optionally substituted C₁-C₆ alkylene-R_{C}, optionally substituted C₁-C₆ heteroalkylene-R_{C}, optionally substituted C₂-C₆ alkenylene-R_{C}, optionally substituted C₂-C₆ heteroalkenylene-R_{C}, optionally substituted C₂-C₆ alkynylene-R_{C}, optionally substituted C₂-C₆ heteroalkynylene-R_{C}, optionally substituted cycloalkylene-R_{C}, optionally substituted heterocycloalkylene-R_{C}, optionally substituted arylene-R_{C}, or optionally substituted heteroarylene-R_{C}.

In some embodiments, Am-L-Z is represented by formula (IA) or formula (IB),

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

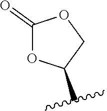 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₅ is OH, NH₂, OR_{C}, or NHR_{C};

R₉ is H or OH;

X is —S—, —S(O)—, or —SO₂—; and

wherein R_{C} and RD are each as defined above.

In some embodiments, Am-L-Z is represented by formula (IA) or formula (IB),

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

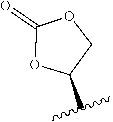 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₅ is OH, NH₂, OR_{C}, or NHR_{C};

R₉ is H or OH;

X is —S—, —S(O)—, or —SO₂—; and

wherein R_{C} is as defined above.

In some embodiments, Am-L-Z is represented by formula (IA) or formula (IB),

wherein R₁ is H, OH, or OR_{A};

R₂ is H, OH, or OR_{B};

R_{A} and R_{B}, when present, together with the oxygen atoms to which they are bound, combine to form:

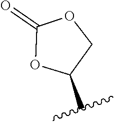 [SMILES: *[C@@H]1COC(=O)O1]

R₃, R₄, R₆, and R₇ are each H;

R₅ is OR_{C};

R₅ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2016/0002298, the disclosure of which is incorporated herein by reference in its entirety.

In some embodiments, Am-L-Z is represented by formula (IA) or formula (IB),

wherein R₁ and R₂ are each independently H or OH;

R₃ is R_{C};

R₄, R₆, and R₇ are each H;

R₅ is H, OH, or OC₁-C₆ alkyl;

R₅ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2014/0294865, the disclosure of which is incorporated herein by reference in its entirety.

In some embodiments, Am-L-Z is represented by formula (IA) or formula (IB),

wherein R₁ and R₂ are each independently H or OH;

R₃, R₆, and R₇ are each H;

R₄ and R₅ are each independently H, OH, OR_{C}, or R_{C};

R₅ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2015/0218220, the disclosure of which is incorporated herein by reference in its entirety.

In some embodiments, Am-L-Z is represented by formula (IA) or formula (IB),

wherein R₁ and R₂ are each independently H or OH;

R₃, R₆, and R₇ are each H;

R₄ and R₅ are each independently H or OH;

R₅ is OH, NH₂, OR_{C}, or NHR_{C};

R₉ is H or OH;

X is —S—, —S(O)—, or —SO₂—; and

wherein R_{C} is as defined above. Such amatoxin conjugates are described, for example, in U.S. Pat. Nos. 9,233,173 and 9,399,681, as well as in US 2016/0089450, the disclosures of each of which are incorporated herein by reference in their entirety.

In some embodiments, Am-L-Z′ is

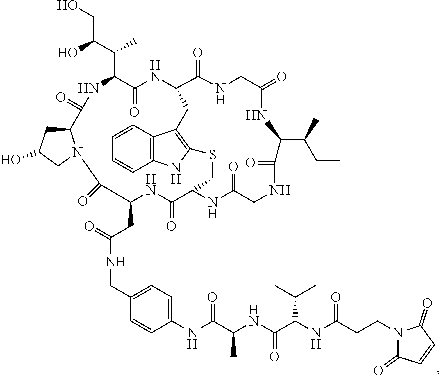

Additional amatoxins that may be used for conjugation to an antibody, or antigen-binding fragment thereof, in accordance with the compositions and methods described herein are described, for example, in WO 2016/142049; WO 2016/071856; WO 2017/149077; WO 2018/115466; and WO 2017/046658, the disclosures of each of which are incorporated herein by reference in their entirety.

In some embodiments, Am-L-Z is represented by formula (II), formula (IIA), or formula (IIB)

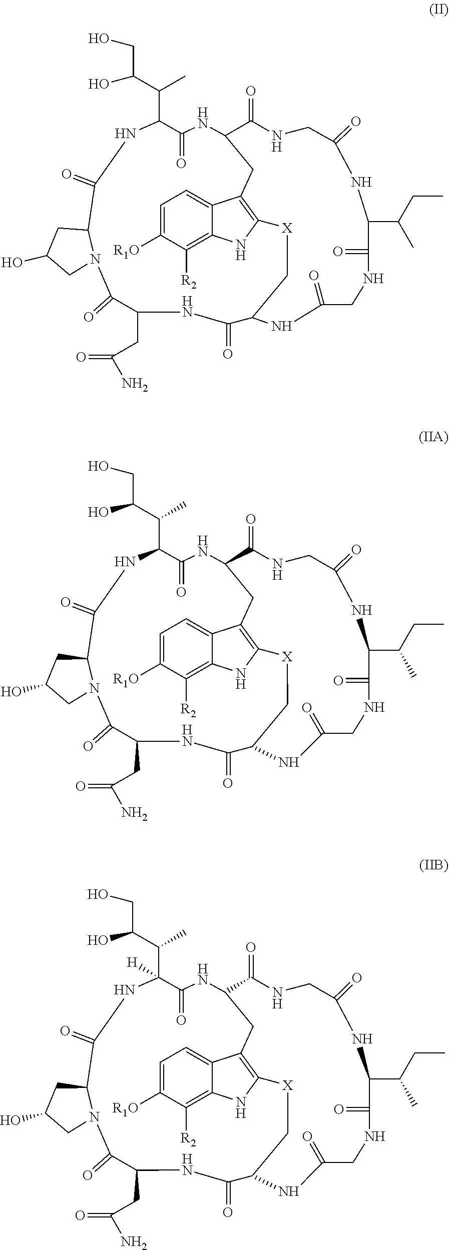

wherein X is S, SO, or SO₂; R₁ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; and R₂ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; wherein when R₁ is H, R₂ is the linker, and when R₂ is H, R₁ is the linker. In some embodiments, R₁ is the linker and R₂ is H, and the linker and chemical moiety, together as L-Z, is

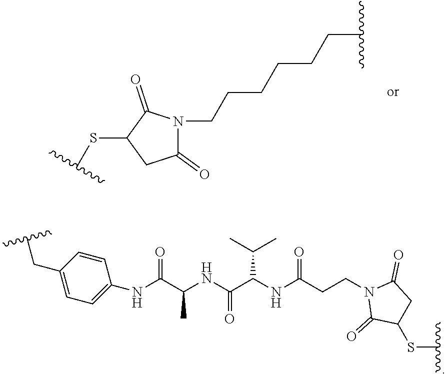

In some embodiments, L-Z is

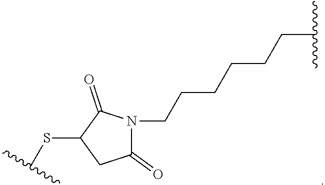 [SMILES: *CCCCCCN1C(=O)CC(S*)C1=O ; "."]

In one embodiment, Am-L-Z-Ab is:

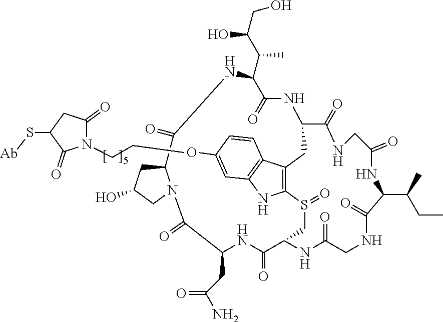

In one embodiment, Am-L-Z-Ab is:

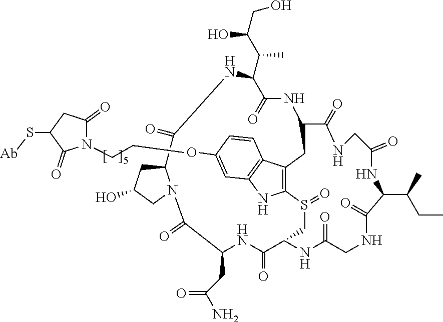

In some embodiments, the Am-L-Z-Ab precursor (i.e., Am-L-Z′) is one of:

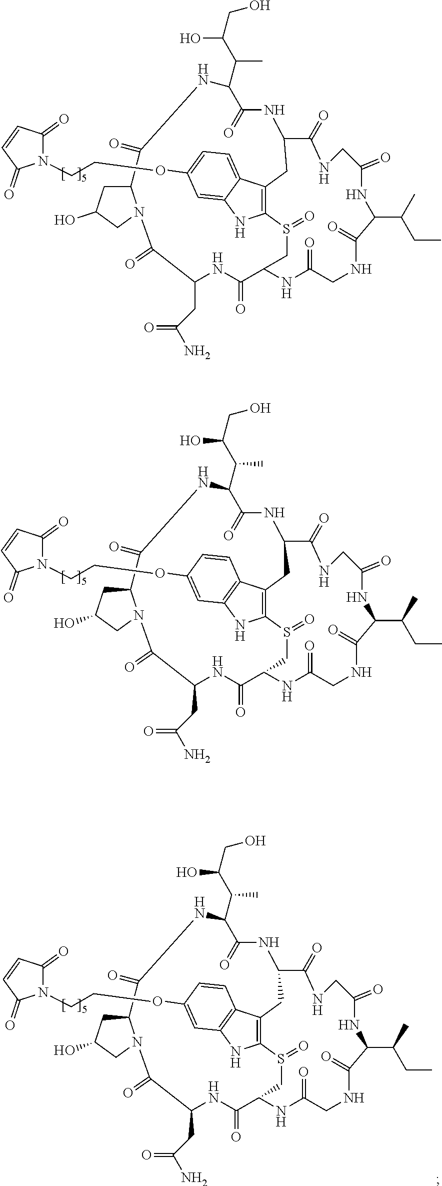

wherein the maleimide reacts with a thiol group found on a cysteine in the antibody.

In some embodiments, the cytotoxin is an α-amanitin. In some embodiments, the α-amanitin is a compound of formula III. In some embodiments, the α-amanitin of formula III is attached to an anti-HC antibody via a linker L. The linker L may be attached to the α-amanitin of formula III at any one of several possible positions (e.g., any of R¹-R⁹) to provide an α-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

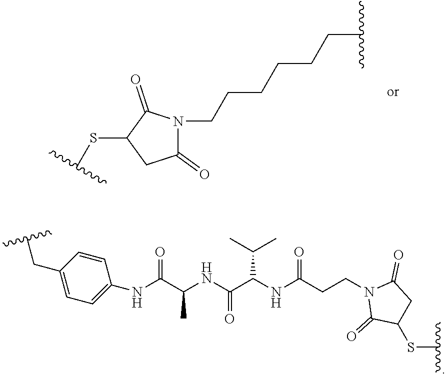

In some embodiments, the cytotoxin is a β-amanitin. In some embodiments, the α-amanitin is a compound of formula III. In some embodiments, the β-amanitin of formula III is attached to an anti-HC antibody via a linker L. The linker L may be attached to the β-amanitin of formula III at any one of several possible positions (e.g., any of R¹-R⁹) to provide an β-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

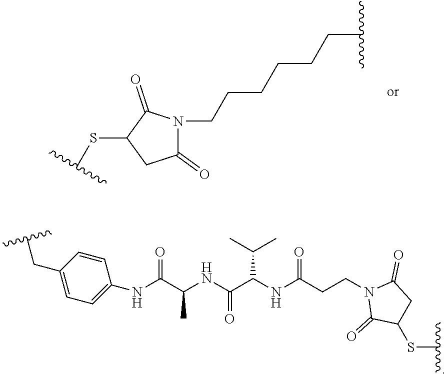

In some embodiments, the cytotoxin is a γ-amanitin. In some embodiments, the γ-amanitin is a compound of formula III. In some embodiments, the γ-amanitin of formula III is attached to an anti-HC antibody via a linker L. The linker L may be attached to the γ-amanitin of formula III at any one of several possible positions (e.g., any of R¹-R⁹) to provide an γ-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

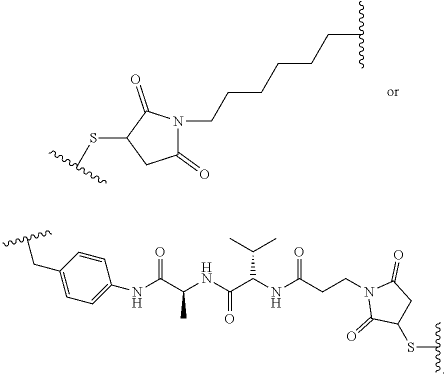

In some embodiments, the cytotoxin is a ε-amanitin. In some embodiments, the ε-amanitin is a compound of formula III. In some embodiments, the ε-amanitin of formula III is attached to an anti-HC antibody via a linker L. The linker L may be attached to the ε-amanitin of formula III at any one of several possible positions (e.g., any of R¹-R⁹) to provide an ε-amanitin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

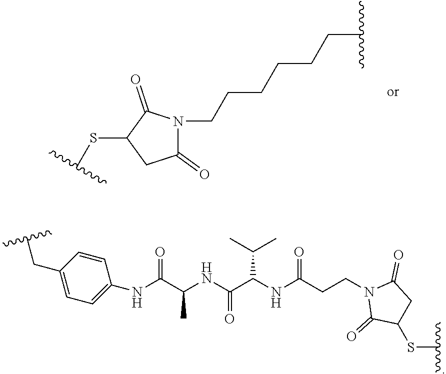

In some embodiments, the cytotoxin is an amanin. In some embodiments, the amanin. is a compound of formula III. In some embodiments, the amanin of formula III is attached to an anti-HC antibody via a linker L. The linker L may be attached to the amanin of formula III at any one of several possible positions (e.g., any of R¹-R⁹) to provide an amanin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

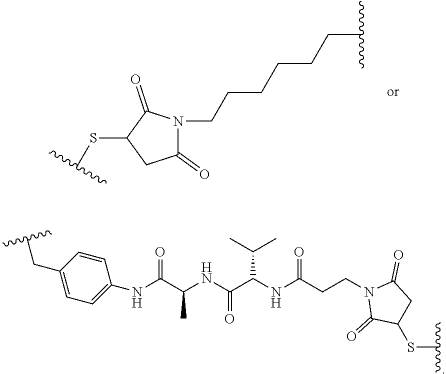

In some embodiments, the cytotoxin is an amaninamide. In some embodiments, the amaninamide is a compound of formula III. In some embodiments, the amaninamide of formula III is attached to an anti-HC antibody via a linker L. The linker L may be attached to the amaninamide of formula III at any one of several possible positions (e.g., any of R¹-R⁹) to provide an amaninamide-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

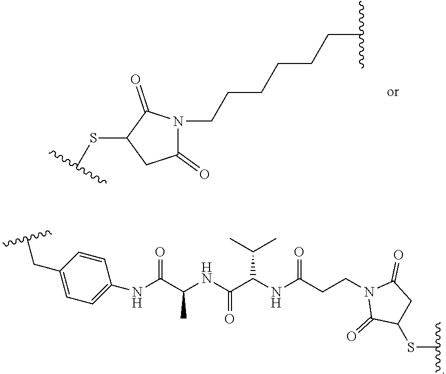

In some embodiments, the cytotoxin is an amanullin. In some embodiments, the amanullin is a compound of formula III. In some embodiments, the amanullin of formula III is attached to an anti-HC antibody via a linker L. The linker L may be attached to the amanullin of formula III at any one of several possible positions (e.g., any of R¹-R⁹) to provide an amanullin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

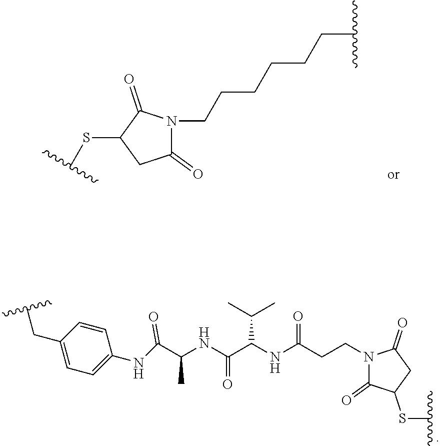

In some embodiments, the cytotoxin is an amanullinic acid. In some embodiments, the amanullinic acid is a compound of formula III. In some embodiments, the amanullinic acid of formula III is attached to an anti-HC antibody via a linker L. The linker L may be attached to the amanullinic acid of formula III at any one of several possible positions (e.g., any of R¹-R⁹) to provide an amanullinic acid-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

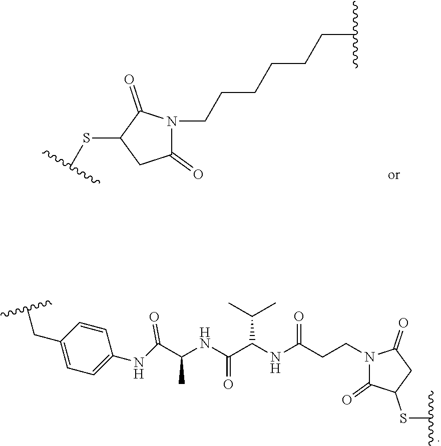

In some embodiments, the cytotoxin is a proamanullin. In some embodiments, the proamanullin is a compound of formula III. In some embodiments, the proamanullin of formula III is attached to an anti-HC antibody via a linker L. The linker L may be attached to the proamanullin of formula III at any one of several possible positions (e.g., any of R¹-R⁹) to provide an proamanullin-linker conjugate of formula I, IA, IB, II, IIA, or IIB. In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

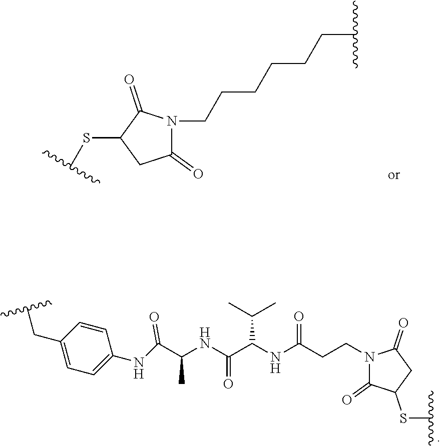

Synthetic methods of making amatoxin are described in U.S. Pat. No. 9,676,702, which is incorporated by reference herein.

Antibodies, or antigen-binding fragments, for use with the compositions and methods described herein can be conjugated to an amatoxin, such as α-amanitin or a variant thereof, using conjugation techniques known in the art or described herein. For instance, antibodies, or antigen-binding fragments thereof, that recognize and bind a target antigen (an anti-HC antibody, e.g., anti-CD117 antibody, anti-CD45 antibody, anti-CD2 antibody, anti-CD5 antibody, anti-CD137 antibody, or anti-CD252 antibody) can be conjugated to an amatoxin, such as α-amanitin or a variant thereof, as described in US 2015/0218220, the disclosure of which is incorporated herein by reference as it pertains, for example, to amatoxins, such as α-amanitin and variants thereof, as well as covalent linkers that can be used for covalent conjugation.

Auristatins

Anti-HC antibodies (e.g., anti-CD117 antibody, anti-CD45 antibody, anti-CD2 antibody, anti-CD5 antibody, anti-CD137 antibody, or anti-CD252 antibody) and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE, wherein the wavy line indicates the point of covalent attachment to the linker of an antibody-linker conjugate (-L-Z-Ab or -L-Z′, as described herein).

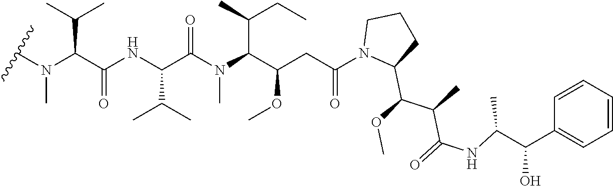 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Another exemplary auristatin embodiment is MMAF, wherein the wavy line indicates the point of covalent attachment to the linker of an antibody-linker conjugate (-L-Z-Ab or -L-Z′, as described herein), as disclosed in US 2005/0238649:

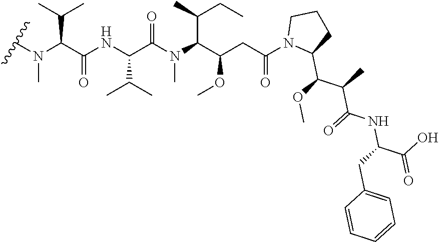 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N²¹-deacetyl-N²¹-(3-mercapto-1-oxopropyl)-maytansine, as the cytotoxic agent. DM1 is represented by the following structural formula V:

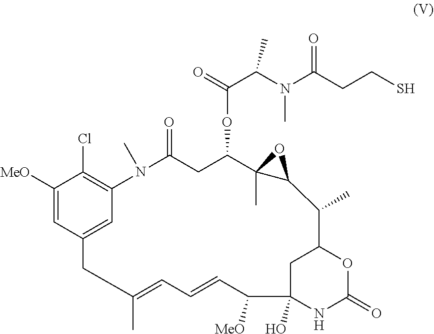 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present disclosure utilize the thiol-containing maytansinoid N²′-deacetyl-N²′(4-methyl-4-mercapto-1-oxopentyl)-maytansine (e.g., DM4) as the cytotoxic agent. DM4 is represented by the following structural formula VI:

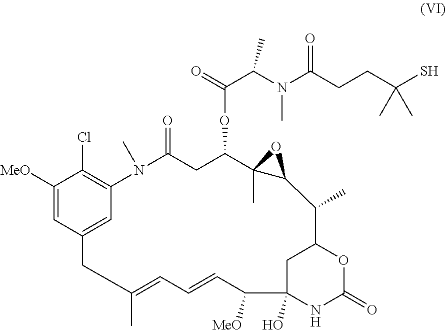 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N²′-deacetyl-N-^{2′}(4-mercapto-1-oxopentyl)-maytansine (termed DM3), represented by the following structural formula VII:

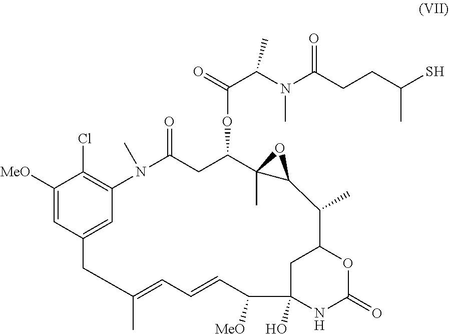 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugates of the present disclosure. In this regard, the entire disclosure of U.S. Pat. Nos. 5,208,020 and 7,276,697 is incorporated herein by reference.

Many positions on maytansinoids can serve as the position to covalently bond the linking moiety and, hence the antibodies or antigen-binding fragments thereof (-L-Z-Ab or -L-Z′, as described herein). For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to covalently bond the linker moiety, and in some particular embodiments, the C-3 position of maytansinol serves as the position to covalently bond the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present disclosure also includes various isomers and mixtures of maytansinoids and conjugates. Certain compounds and conjugates of the present disclosure may exist in various stereoisomeric, enantiomeric, and diastereomeric forms. Several descriptions for producing such antibody-maytansinoid conjugates are provided in U.S. Pat. Nos. 5,208,020; 5,416,064; 6,333,410; 6,441,163; 6,716,821; and 7,368,565, each of which is incorporated herein in its entirety.

Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy; N. R. Bachur, “Free Radical Damage” id. at pp. 97-102]. Because of their cytotoxic potential anthracyclines have been used in the treatment of numerous cancers such as leukemia, breast carcinoma, lung carcinoma, ovarian adenocarcinoma and sarcomas [see e.g., P. H-Wiernik, in Anthracycline: Current Status And New Developments p 11]. Commonly used anthracyclines include doxorubicin, epirubicin, idarubicin and daunomycin.

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

Commonly used anthracyclines include doxorubicin, epirubicin, idarubicin and daunomycin. In some embodiments, the cytotoxin is an anthracycline selected from the group consisting of daunorubicin, doxorubicin, epirubicin, and idarubicin

Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.) The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by the structural formula:

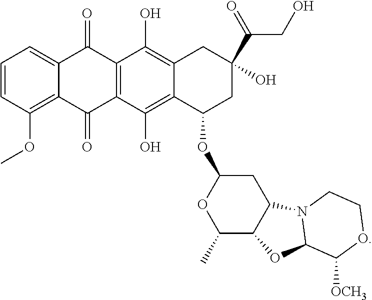

Multiple positions on anthracyclines such as PNU can serve as the position to covalently bond the linking moiety and, hence the anti-CD117 antibodies or antigen-binding fragments thereof as described herein. For example, linkers may be introduced through modifications to the hydroxymethyl ketone side chain.

In some embodiments, the cytotoxin is a PNU derivative represented by the structural formula:

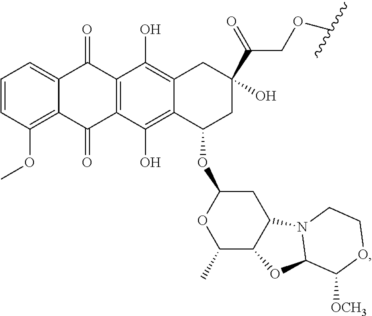

wherein the wavy line indicates the point of covalent attachment to the linker of the ADC as described herein.

In some embodiments, the cytotoxin is a PNU derivative represented by the structural formula:

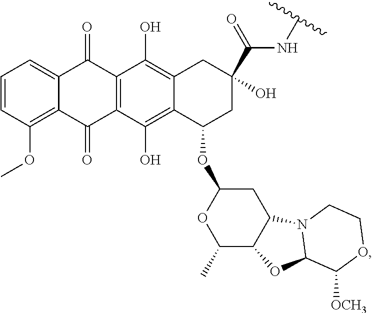

wherein the wavy line indicates the point of covalent attachment to the linker of the ADC as described herein.

Pyrrolobenzodiazepines (PBDs)

In other embodiments, the anti-HC antibodies (e.g., anti-CD117 antibody, anti-CD45 antibody, anti-CD2 antibody, anti-CD5 antibody, anti-CD137 antibody, or anti-CD252 antibody) or antigen-binding fragments thereof described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, J A (2011) The development of pyrrolobenzodiazepines as antitumour agents. Expert Opin Inv Drug, 20(6), 733-744 and Antonow D, Thurston D E (2011) Synthesis of DNA-interactive pyrrolo[2,1-c][1,4]benzodiazepines (PBDs). Chem Rev 111: 2815-2864.

In some embodiments, the cytotoxin is a pyrrolobenzodiazepine dimer represented by the structural formula:

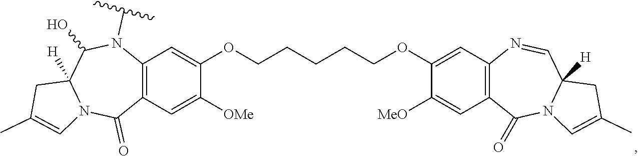

wherein the wavy line indicates the attachment point of the linker.

In some embodiments, the cytotoxin is conjugated to the antibody, or the antigen-binding fragment thereof, by way of a maleimidocaproyl linker.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula:

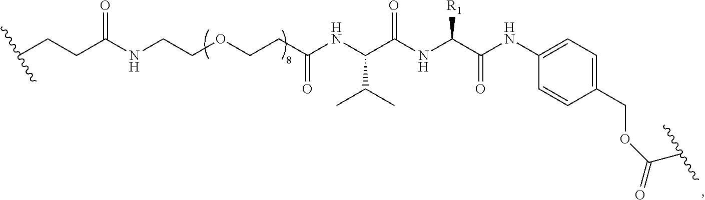

wherein R₁ is CH₃ (Ala) or (CH₂)₃NH(CO)NH₂ (Cit).

In some embodiments, the linker, prior to conjugation to the antibody and including the reactive substituent Z′, taken together as L-Z′, has the structure:

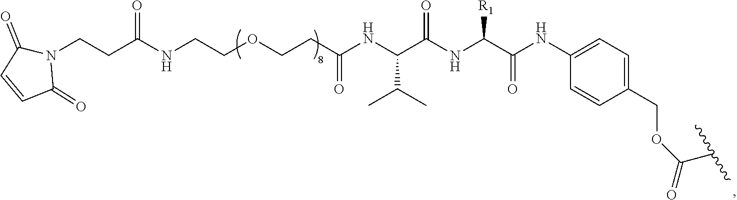

wherein the wavy line indicates the attachment point to the cytotoxin (e.g., a PBD). In certain embodiments, R₁ is CH₃.

In some embodiments, the cytotoxin-linker conjugate, prior to conjugation to the antibody and including the reactive substituent Z′, taken together as Cy-L-Z′, has the structural formula:

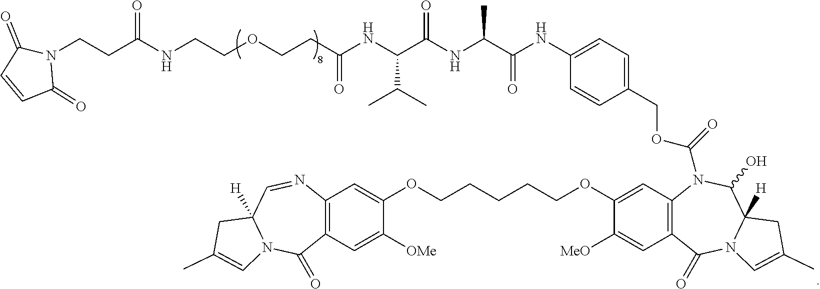

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In some embodiments, the cytotoxin is a pyrrolobenzodiazepine dimer represented by the structural formula:

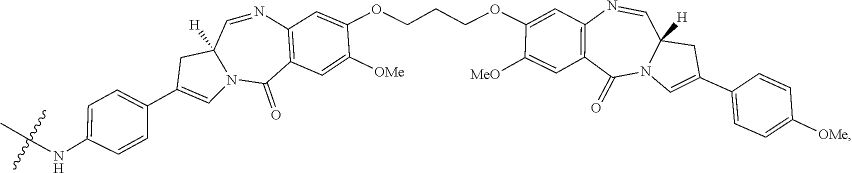 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

wherein the wavy line indicates the attachment point of the linker.

In some embodiments, the cytotoxin-linker conjugate, prior to conjugation to the antibody and including the reactive substituent Z′, taken together as Cy-L-Z′, has the structural formula:

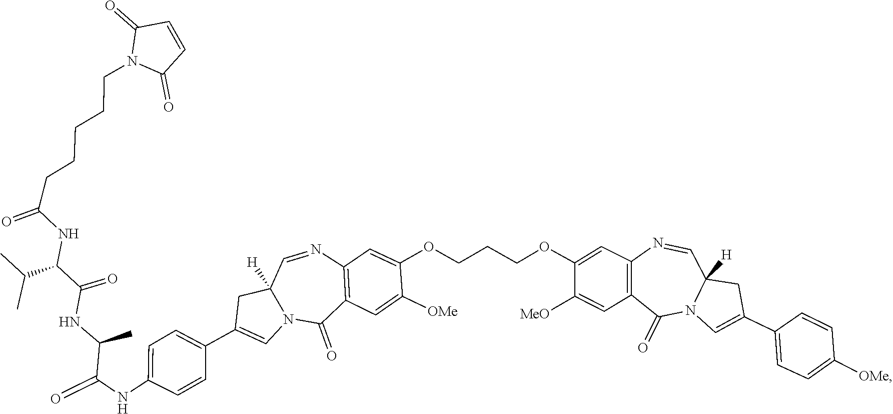 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in connection with the ADC Vadastuximab talirine (SGN-CD33A), Mantaj et al., Angewandte Chemie International Edition English 2017, 56, 462-488, the disclosure of which is incorporated by reference herein in its entirety.

In some embodiments, the cytotoxin is an indolinobenzodiazepine pseudodimer having the structural formula:

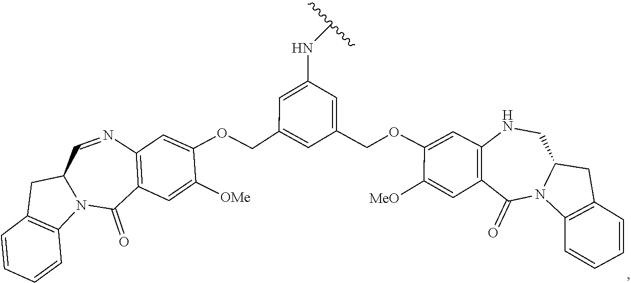 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

In some embodiments, the cytotoxin-linker conjugate, prior to conjugation to the antibody and including the reactive substituent Z′, taken together as Cy-L-Z′, has the structural formula:

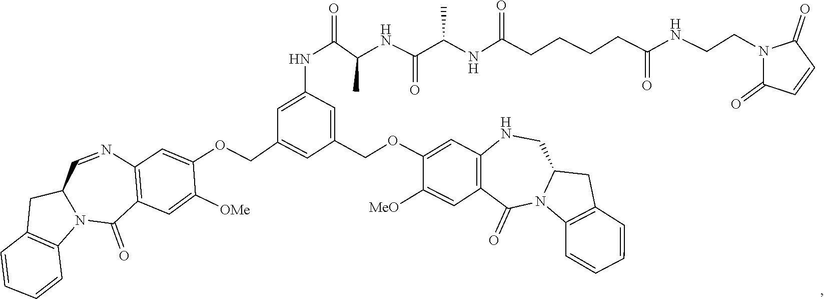 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

Calicheamicin

In other embodiments, the antibodies and antigen-binding fragments thereof described herein can be conjugated to a cytotoxin that is an enediyne antitumor antibiotic (e.g., calicheamicins, ozogamicin). The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

An exemplary calicheamicin is designated γ₁, which is herein referenced simply as gamma, and has the structural formula:

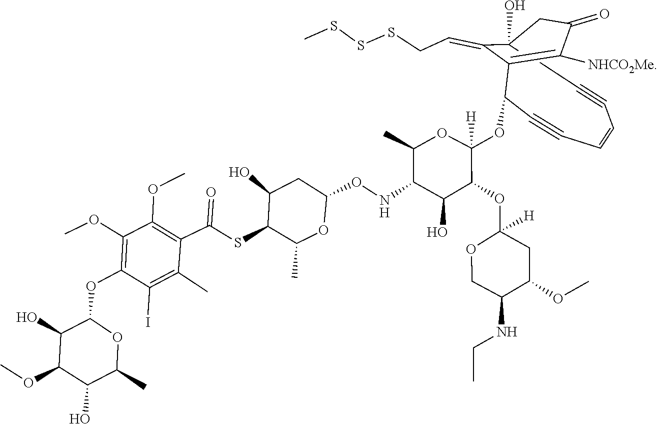

In some embodiments, the calicheamicin is a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD117 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein is a calicheamicin disulfide derivative represented by the structural formula:

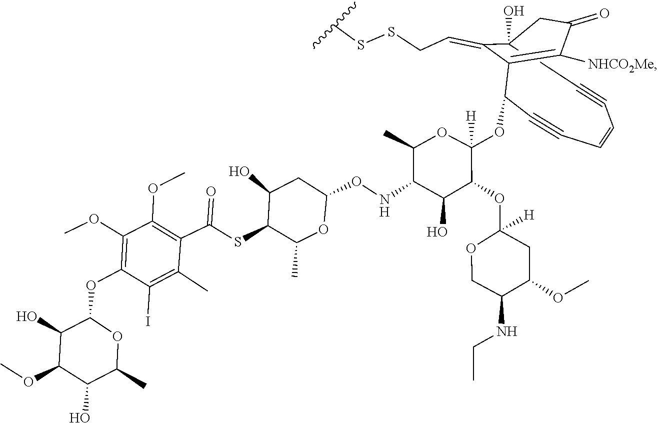

wherein the wavy line indicates the attachment point of the linker.

Additional Cytotoxins

In other embodiments, the antibodies and antigen-binding fragments thereof described herein can be conjugated to a cytotoxin other than or in addition to those cytotoxins disclosed herein above. Additional cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

Linkers

A variety of linkers can be used to conjugate the antibodies, or antibody fragments thereof, described herein (e.g., an anti-CD117 antibody, an anti-CD45 antibody, an anti-CD2 antibody, an anti-CD5 antibody, an anti-CD137 antibody, or an anti-CD252 antibody) to a cytotoxic molecule.

The term “Linker” as used herein means a divalent chemical moiety comprising a covalent bond or a chain of atoms that covalently attaches an anti-HC antibody (e.g., an anti-CD117 antibody, an anti-CD45 antibody, an anti-CD2 antibody, an anti-CD5 antibody, an anti-CD137 antibody, or an anti-CD252 antibody) drug conjugates (ADC) of the present disclosure (ADCs; Ab-Z-L-D, where D is a cytotoxin). Suitable linkers have two reactive termini, one for conjugation to an antibody and the other for conjugation to a cytotoxin. The antibody conjugation reactive terminus of the linker (reactive moiety, Z′) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z′, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, or antibody fragments thereof, described herein to a cytotoxic molecule include those capable of releasing a cytotoxin by a 1,6-elimination process (a “self-immolative” group). Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

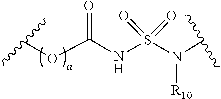

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₚ— unit, wherein p is an integer from 1-6.

In some embodiments, the linker comprises a —(CH₂)ₙ— unit, wherein n is an integer from 2 to 6.

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises

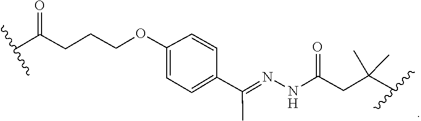 [SMILES: *C(=O)CCCOc1ccc(/C(C)=N/NC(=O)CC(*)(C)C)cc1]

In some embodiments, the linker comprises MCC (4-[N-maleimidomethyl]cyclohexane-1-carboxylate).

In one specific embodiment, the linker comprises the structure

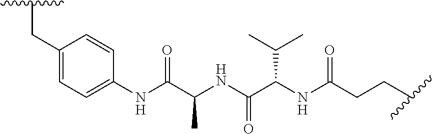 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises the structure

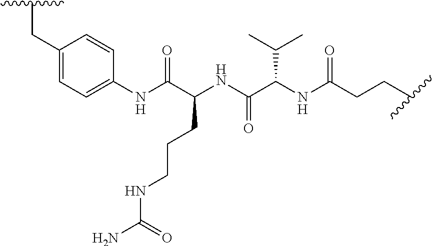 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z′. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

Linkers that can be used to conjugate an antibody, or antigen-binding fragment thereof, to a cytotoxic agent include those that are covalently bound to the cytotoxic agent on one end of the linker and, on the other end of the linker, contain a chemical moiety formed from a coupling reaction between a reactive substituent present on the linker and a reactive substituent present within the antibody, or antigen-binding fragment thereof, that binds CD117 (such as GNNK+ CD117). Reactive substituents that may be present within an antibody, or antigen-binding fragment thereof, that binds CD117 (such as GNNK+CD117) include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids.

Examples of linkers useful for the synthesis of drug-antibody conjugates conjugates include those that contain electrophiles, such as Michael acceptors (e.g., maleimides), activated esters, electron-deficient carbonyl compounds, and aldehydes, among others, suitable for reaction with nucleophilic substituents present within antibodies or antigen-binding fragments, such as amine and thiol moieties. For instance, linkers suitable for the synthesis of drug-antibody conjugates include, without limitation, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are particularly useful for the conjugation of microtubule-disrupting agents such as auristatins, are described by Doronina et al., Bioconjugate Chem. 17:14-24, 2006, the disclosure of which is incorporated herein by reference as it pertains to linkers for chemical conjugation.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 4, below. Curved lines designate points of attachment to the antibody or antigen-binding fragment, and the cytotoxic molecule, respectively.

TABLE 4
Exemplary chemical moieties Z formed by coupling reactions in the
formation of antibody-drug conjugates

| Exemplary | |
|---|---|
| Coupling Reactions | Chemical Moiety Z Formed by Coupling Reactions |
| [3 + 2] Cycloaddition | 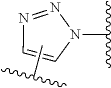 |
| [3 + 2] Cycloaddition | 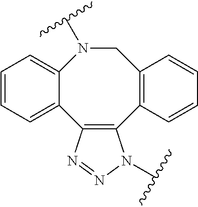 [SMILES: *N1Cc2ccccc2-c2c(nnn2*)-c2ccccc21] |
| [3 + 2] Cycloaddition, Esterification | 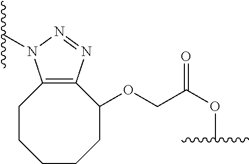 [SMILES: *OC(=O)COC1CCCCCc2c1nnn2*] |
| [3 + 2] Cycloaddition, Esterification | 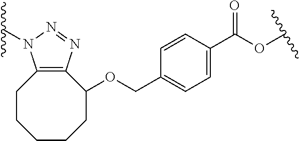 [SMILES: *OC(=O)c1ccc(COC2CCCCCc3c2nnn3*)cc1] |
| [3 + 2] Cycloaddition, Esterification | 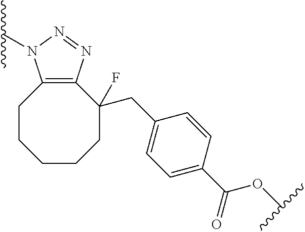 [SMILES: *OC(=O)c1ccc(CC2(F)CCCCCc3c2nnn3*)cc1] |
| [3 + 2] Cycloaddition, Esterification | 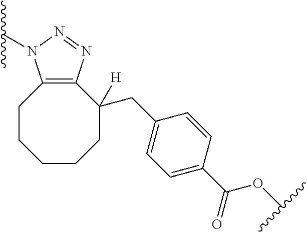 [SMILES: *OC(=O)c1ccc(CC2CCCCCc3c2nnn3*)cc1] |
| [3 + 2] Cycloaddition, Esterification | 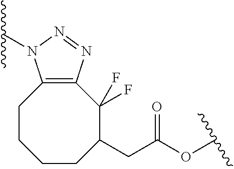 [SMILES: *OC(=O)CC1CCCCc2c(nnn2*)C1(F)F] |
| [3 + 2] Cycloaddition, Esterification | 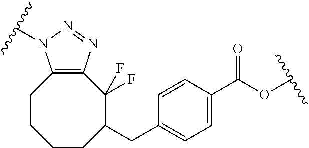 [SMILES: *OC(=O)c1ccc(CC2CCCCc3c(nnn3*)C2(F)F)cc1] |
| [3 + 2] Cycloaddition, Esterification | 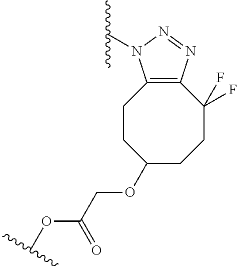 [SMILES: *OC(=O)COC1CCc2c(nnn2*)C(F)(F)CC1] |
| [3 + 2] Cycloaddition, Esterification | 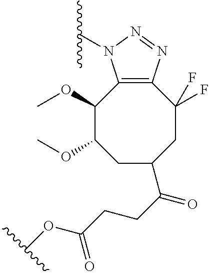 [SMILES: *OC(=O)CCC(=O)C1C[C@H](OC)[C@@H](OC)c2c(nnn2*)C(F)(F)C1] |
| [3 + 2] Cycloaddition, Esterification | 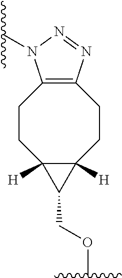 [SMILES: *OC[C@@H]1[C@@H]2CCc3c(nnn3*)CC[C@@H]21] |
| [3 + 2] Cycloaddition, Esterification | 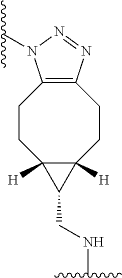 [SMILES: *NC[C@@H]1[C@@H]2CCc3c(nnn3*)CC[C@@H]21] |
| [3 + 2] Cycloaddition, Esterification | 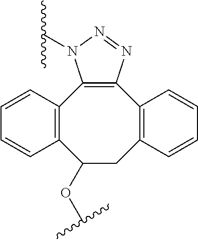 [SMILES: *OC1Cc2ccccc2-c2nnn(*)c2-c2ccccc21] |
| [3 + 2] Cycloaddition, Etherification | 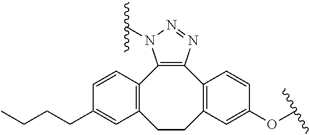 [SMILES: *Oc1ccc2c(c1)CCc1cc(CCCC)ccc1-c1c-2nnn1*] |
| [3 + 2] Cycloaddition | 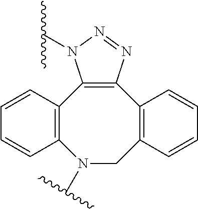 [SMILES: *N1Cc2ccccc2-c2nnn(*)c2-c2ccccc21] |
| Michael addition | 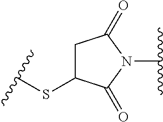 [SMILES: *SC1CC(=O)N(*)C1=O] |
| Michael addition | 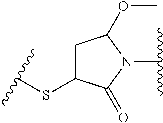 [SMILES: *SC1CC(OC)N(*)C1=O] |
| Imine condensation, Amidation | 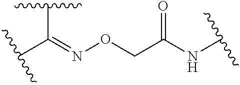 [SMILES: *NC(=O)CON=C(*)*] |
| Imine condensation | 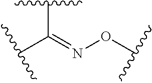 [SMILES: *ON=C(*)*] |
| Disulfide formation | 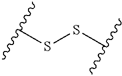 [SMILES: *SS*] |
| Thiol alkylation | 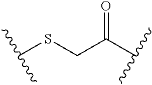 [SMILES: *SCC(*)=O] |
| Condensation, Michael addition | 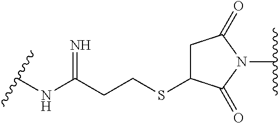 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

As depicted in Table 4, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others.

For instance, linkers suitable for the synthesis of ADCs include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

Non-limiting examples of amatoxin-linker conjugates containing a reactive substituent Z′ suitable for reaction with a reactive residue on the antibody or antigen-binding fragment thereof include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-1-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-1yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyl)aziridin-1-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin.

One of skill in the art will recognize the linker-reactive substituent group structure, prior to conjugation with the antibody or antigen binding fragment thereof, includes a maleimide as the group Z′. The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In some embodiments, the linker-reactive substituent group structure L-Z′, prior to conjugation with the antibody or antigen binding fragment thereof, is:

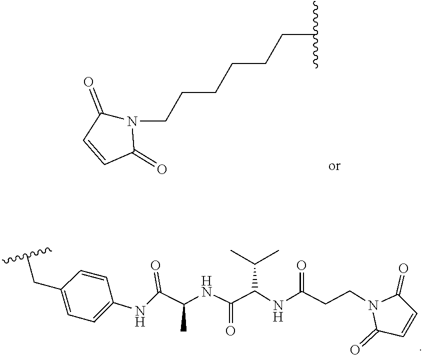

In some embodiments, an amatoxin as disclosed herein is conjugated to a linker-reactive moiety -L-Z′ having the following formula:

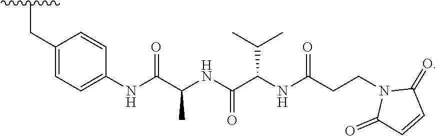 [SMILES: *Cc1ccc(NC(=O)[C@H](C)NC(=O)[C@@H](NC(=O)CCN2C(=O)C=CC2=O)C(C)C)cc1]

In some embodiments, an amatoxin as disclosed herein is conjugated to a linker-reactive moiety -L-Z′ having the following formula:

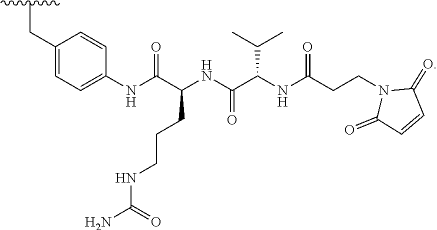 [SMILES: *Cc1ccc(NC(=O)[C@H](CCCNC(N)=O)NC(=O)[C@@H](NC(=O)CCN2C(=O)C=CC2=O)C(C)C)cc1]

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In some embodiments, the ADC comprises an anti-CD117 antibody conjugated to an amatoxin, including but not limited to an amatoxin of any one of formulae I, IA, IB, II, IIA, or IIB as disclosed herein, via a linker and a chemical moiety Z. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker is —(CH₂)ₙ—. In some embodiments, the linker is —((CH₂)ₙ—, wherein n is 6.

In some embodiments, the chemical moiety Z is selected from Table 4. In some embodiments, the chemical moiety Z is

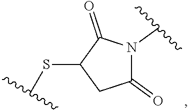

where S is a sulfur atom which represents the reactive substituent present within an antibody, or antigen-binding fragment thereof, that binds CD117 (e.g., from the —SH group of a cysteine residue).

In some embodiments, the linker L and the chemical moiety Z, taken together as L-Z, is

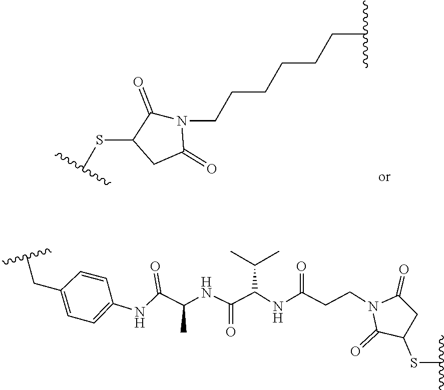

### Preparation of Antibody-Drug Conjugates

In the ADCs of the present disclosure (ADCs; D-L-Z-Ab, where D is a cytotoxin) as disclosed herein, an anti-HC antibody (e.g., an anti-CD117 antibody, an anti-CD45 antibody, an anti-CD2 antibody, an anti-CD5 antibody, an anti-CD137 antibody, or an anti-CD252 antibody) or an antigen binding fragment thereof, is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above to form an ADC of formula D-L-Z-Ab, such as Am-Z-L-Ab. Additional methods for preparing ADC are described herein.

In another aspect, the anti-HC antibody (e.g., anti-CD117 antibody, anti-CD45 antibody, anti-CD2 antibody, anti-CD5 antibody, anti-CD137 antibody, or anti-CD252 antibody), or an antigen-binding fragment thereof, has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-HC antibody (e.g., anti-CD117 antibody, anti-CD45 antibody, anti-CD2 antibody, anti-CD5 antibody, anti-CD137 antibody, or anti-CD252 antibody), or an antigen-binding fragment thereof, can have one or more carbohydrate groups that can be chemically modified to have one or more sulfhydryl groups. The ADC is then formed by conjugation through the sulfhydryl group's sulfur atom as described herein above.

In yet another aspect, the anti-HC antibody (e.g., anti-CD117 antibody, anti-CD45 antibody, anti-CD2 antibody, anti-CD5 antibody, anti-CD137 antibody, or anti-CD252 antibody), or an antigen-binding fragment thereof, can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising anti-HC ADCs (e.g., anti-CD117 ADC, anti-CD45 ADC, anti-CD2 ADC, anti-CD5 ADC, anti-CD137 ADC, or anti-CD252 ADC) as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### Methods of Treatment

Further disclosed herein are compositions and methods of treating a variety of disorders, such as diseases of a cell type in the hematopoietic lineage, cancers, autoimmune diseases, metabolic disorders, stem cell disorders, graft versus host disease, among others. The compositions and methods described herein may (i) directly deplete a population of cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of an ADC, antibody, or antigen-binding fragment thereof, capable of binding an antigen expressed by an endogenous disease-causing cell or a hematopoietic stem cell. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. The invention is based in part on the discovery that ADCs, antibodies, or antigen-binding fragments thereof, capable of binding an antigen expressed by hematopoietic stem cells (e.g., CD117 (e.g., GNNK+ CD117), or CD45) or an antigen expressed by mature immune cells, such as T-cells (e.g., CD45, CD2, CD5, CD137, or CD252) can be administered to a patient to effect both of the above activities. ADCs, antibodies, or antigen-binding fragments thereof, that bind an antigen expressed by hematopoietic stem cells (e.g., CD117 (e.g., GNNK+ CD117), or CD45) or an antigen expressed by immune cells (e.g., mature immune cells), such as T-cells (e.g., CD45, CD2, CD5, CD137, or CD252) can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

As described herein, hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally, or alternatively, the compositions and methods described herein can be used to treat an immunodeficiency, such as a congenital immunodeficiency. Additionally, or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additionally, or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The antibodies, or antigen-binding fragments thereof, and conjugates described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In addition, the compositions and methods described herein can be used to treat cancers directly, such as cancers characterized by cells that are CD117+ (e.g., GNNK+ CD117), CD45+, CD2+, CD5+, CD137+, or CD252+. For instance, the compositions and methods described herein can be used to treat leukemia, such as in patients that exhibit CD117+ leukemic cells. By depleting CD117+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

Acute myeloid leukemia (AML) is a cancer of the myeloid line of blood cells, characterized by the rapid growth of abnormal white blood cells that build up in the bone marrow and interfere with the production of normal blood cells. AML is the most common acute leukemia affecting adults, and its incidence increases with age. The symptoms of AML are caused by replacement of normal bone marrow with leukemic cells, which causes a drop in red blood cells, platelets, and normal white blood cells. As an acute leukemia, AML progresses rapidly and may be fatal within weeks or months if left untreated. In one embodiment, the anti-CD117 ADCs described herein are used to treat AML in a human patient in need thereof. In certain embodiments the anti-CD117 ADC treatment depletes AML cells in the treated subjects. In some embodiments 50% or more of the AML cells are depleted. In other embodiments, 60% or more of the AML cells are depleted, or 70% or more of the AML cells are depleted, or 80% of more or 90% or more, or 95% or more of the AML cells are depleted. In certain embodiments the anti-CD117 ADC treatments is a single dose treatment. In certain embodiments the single dose anti-CD117 ADC treatment depletes 60%, 70%, 80%, 90% or 95% or more of the AML cells.

In addition, the compositions and methods described herein can be used to treat autoimmune disorders. For instance, an antibody, or antigen-binding fragment thereof, can be administered to a subject, such as a human patient suffering from an autoimmune disorder, so as to kill CD45+, CD2+, CD5+, CD137+, or CD252+ immune cell. For example, a CD45+, CD2+, CD5+, CD137+, or CD252+ immune cell may be an autoreactive lymphocyte, such as a T-cell that expresses a T-cell receptor that specifically binds, and mounts an immune response against, a self-antigen. By depleting self-reactive, CD45+, CD2+, CD5+, CD137+, or CD252+ cells, the compositions and methods described herein can be used to treat autoimmune pathologies, such as those described below. Additionally, or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

Further provided herein are methods of preventing and treating graft-vs-host-disease (GVHD) and autoimmune diseases by administration of an antibody, antigen-binding fragment thereof, or ADC, capable of binding an antigen (e.g., CD137, CD2, or CD5) expressed by hematopoietic cells, wherein the antibody comprises an Fc region comprising a D265C, L234A, L235A, and/or H435A mutation. This administration can cause the selective depletion of a population of T cells that are reactive against the host. For example, an antibody, antigen-binding fragment thereof, or ADC capable of binding CD137 can be administered to a patient in in order to prevent and treat GVHD and autoimmune diseases, such as those arising from hematopoietic stem cell transplant therapy. Anti-CD137 antibodies and conjugates thereof, and related methods of use are found, for example, in U.S. Pat. Application. No. 62/448,741, and PCT Publication NO. WO 2018/134787, which are hereby expressly incorporated by reference in their entirety.

The compositions and methods described herein may be used to deplete activated T cells (e.g., expressing CD137, CD252, CD134, etc.) that are associated with graft failure and autoimmune diseases in order to achieve transplant tolerance. The compositions and methods described herein are particularly useful for preventing and treating GVHD and autoimmune diseases. The methods and compositions disclosed herein are also useful in reducing the risk of transplant failure in a human patient receiving an allogenic transplant. The preferred subject is human. The amount of antibody, antibody-drug conjugate, or ligand-drug conjugate administered should be sufficient to deplete cells, e.g., activated T cells, that promote GVHD or autoimmune disease.

The antibody or antibody-drug conjugate can be administered to the human patient in need prior to, concomitantly with, or after transplantation of cells or a solid organ to the patient. In one embodiment, an anti-CD137 ADC is administered to the human patient in need thereof prior to (e.g., about 3 days before, about 2 days before, about 12 hours before, about 12 hours to 3 days before, about 1 to 3 days before, about 12 hours to 2 days before, or about 1-2 days before) transplantation of cells or a solid organ. In one embodiment, an anti-CD137 ADC is administered to the human patient in need thereof after (e.g., about 1 days after, about 2 days after, about 3 days after, or about 4 days after) transplantation of cells or a solid organ. A single dose of an anti-CD137 ADC may be administered to the human patient either prior to, concomitantly with, or after transplantation of cells or an organ, where such single dose is sufficient to treat or prevent GVHD or graft failure.

The methods and compositions disclosed herein may be used to prevent or treat graft failure. Graft failure or graft rejection, including failure after allogeneic hematopoietic stem cell transplantation, may be manifested generally as either lack of initial engraftment of donor cells, or loss of donor cells after initial engraftment (for review see Mattsson et al. (2008) Biol Blood Marrow Transplant. 14(Suppl 1): 165-170). For example, the compositions and methods disclosed herein may be used to deplete CD137 expressing activated T cells in a graft or transplantation scenario where graft failure is of concern, e.g., where the human patient is at risk of developing graft failure following transplantation of a solid organ or cells, particularly where the transplanted cells or organ is allogeneic.

In one embodiment, an anti-CD137 antibody, antibody-drug conjugate, or ligand-drug conjugate is used to deplete CD137 expressing donor cells, e.g., activated T cells expressing CD137, by contacting the cells, graft or solid organ with the anti-CD137 antibody, antibody-drug conjugate, or ligand-drug conjugate prior to transplantation of the cells, graft or organ to a human patient. In one embodiment, the cells, graft or organ are allogeneic.

The risk of GVHD remains high following transplantation with current therapies. The methods and compositions disclosed herein may be used to inhibit graft versus host disease (GVHD) in a human patient. The anti-CD137 ADCs may be used to selectively target activated T cells in a patient who will be receiving a transplant, such as a stem cell transplant. Anti-CD137 ADCs, as described herein, may also be used to reduce the risk of GVHD by targeting and depleting CD137 positive cells in a human patient who is going to be or has already received a transplant, such as but not limited to, an HSC transplant. Anti-CD252 ADCs, as described herein, may also be used to reduce the risk of GVHD by targeting and depleting CD137 positive cells in a human patient who is going to be or has already received a transplant, such as but not limited to, an HSC transplant. In certain embodiments, the compositions and methods disclosed herein are for treating GVHD prior to appearance of symptoms of GVHD in a patient following a transplantation therapy, e.g., allogeneic HSCs.

The methods described herein are also useful for preventing host versus graft (HvG) reactions. For example, an anti-CD137-ADC can also be used as an immunosuppressant to prevent host versus graft (HvG) reactions thereby preventing or reducing the risk of allogeneic graft failure. Use of an anti-CD137 ADC in a patient at risk for a HvG reaction would enable engraftment of donor cells with a greater degree of HLA-mismatch. Additional uses include tolerance induction in solid organ transplant, where host versus graft reactions are prevented or dampened by the CD137-ADC. These would include solid organ transplants done with or without hematopoietic stem cell transplants, including xeno-transplants where the organ is non-human in origin and/or genetically modified.

In one embodiment, an anti-CD137-ADC is used to prevent graft versus graft (GvG) in the context of allogeneic transplants where two donors are used. Examples include the use of 2 cord blood stem cell donors in adult and pediatric patients. Prevention of GvG would enable more rapid hematopoietic (e.g. neutrophil and platelet) reconstitution post-transplant as both stem cell sources would successfully engraft.

Other methods of treatment using anti-CD137 antibodies or ADCs are described in U.S. Pat. No. 10,434,185, which is incorporated by reference herein.

Other methods of treatment using anti-CD252 antibodies or ADCs, including prevention of graft versus host disease or induction of graft tolerance post transplant, are described in US WO 2019/173780, which is incorporated by reference herein.

Other methods of treatment using anti-CD2 antibodies or ADCs are described in WO 2019/108860, which is incorporated by reference herein.

Other methods of treatment using anti-CD5 antibodies or ADCs are described in WO 2019/108863, which is incorporated by reference herein.

In some embodiments, the transplant is allogeneic. In some embodiments, the transplant is autologous.

In some embodiments, the transplant is a bone marrow transplant, a peripheral blood transplant, or a cord blood transplant.

In some embodiments, the transplant includes hematopoietic cells (e.g., hematopoietic stem cells).

In any of the embodiments described herein, the transplant may be any solid organ or skin transplant. In some embodiments, the transplant is selected from the group consisting of kidney transplant, heart transplant, liver transplant, pancreas transplant, lung transplant, intestine transplant and skin transplant.

Additionally, disclosed herein are methods of treating or preventing rejection of transplanted allogeneic cells in a human subject, i.e., treatment or prevention of host versus graft (HvG) disease using the antibodies, or conjugates thereof, having the modified Fc regions disclosed herein. For example, the methods disclosed herein include administration of both an anti-CD137 antibody drug conjugate (ADC) (that binds to endogenous CD137+ immune cells, e.g., activated T cells) and allogeneic cell therapy, wherein the anti-CD137 antibody comprises an Fc region comprising a D265C, L234A, L235A, and/or H435A mutation. By administering an anti-CD137 ADC to a human patient receiving an allogeneic cell transplant, endogenous CD137+ T cells are depleted, thus reducing the risk of a reaction by the endogenous cells against the allogeneic cell therapy.

### Routes of Administration and Dosing

Antibodies, antigen-binding fragments thereof, or ADCs described herein can be administered to a patient (e.g., a human patient suffering from cancer, an autoimmune disease, or in need of hematopoietic stem cell transplant therapy) in a variety of dosage forms. For instance, antibodies, antigen-binding fragments thereof, or ADCs described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

Pharmaceutical formulations comprising an anti-HC antibody (e.g., an anti-CD117 antibody, an anti-CD45 antibody, an anti-CD2 antibody, an anti-CD5 antibody, an anti-CD137 antibody, or an anti-CD252 antibody), or conjugates thereof (e.g., ADCs as described herein) are prepared by mixing such antibody or ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The antibodies, antigen-binding fragments, or ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 μg/mL) of the antibody, or antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant.

In one embodiment, the dose of an anti-HC ADC (e.g, an anti-CD117 antibody conjugated via a linker to an amatoxin) administered to the human patient is about 0.1 mg/kg to about 0.3 mg/kg.

In one embodiment, the dose of an anti-HC ADC (e.g, an anti-CD117 antibody conjugated via a linker to an amatoxin) administered to the human patient is about 0.15 mg/kg to about 0.3 mg/kg.

In one embodiment, the dose of an anti-HC ADC (e.g, an anti-CD117 antibody conjugated via a linker to an amatoxin) administered to the human patient is about 0.15 mg/kg to about 0.25 mg/kg.

In one embodiment, the dose of an anti-HC ADC (e.g, an anti-CD117 antibody conjugated via a linker to an amatoxin) administered to the human patient is about 0.2 mg/kg to about 0.3 mg/kg.

In one embodiment, the dose of an anti-HC ADC (e.g, an anti-CD117 antibody conjugated via a linker to an amatoxin) administered to the human patient is about 0.25 mg/kg to about 0.3 mg/kg.

In one embodiment, the dose of an anti-HC ADC (e.g, an anti-CD117 antibody conjugated via a linker to an amatoxin) administered to the human patient is about 0.1 mg/kg.

In one embodiment, the dose of an anti-HC ADC (e.g, an anti-CD117 antibody conjugated via a linker to an amatoxin) administered to the human patient is about 0.2 mg/kg.

In one embodiment, the dose of an anti-HC ADC (e.g, an anti-CD117 antibody conjugated via a linker to an amatoxin) administered to the human patient is about 0.3 mg/kg.

In one embodiment, the dose of an anti-HC ADC described herein administered to the human patient is about 0.001 mg/kg to 10 mg/kg, about 0.01 mg/kg to 9.5 mg/kg, about 0.1 mg/kg to 9 mg/kg, about 0.1 mg/kg to 8.5 mg/kg, about 0.1 mg/kg to 8 mg/kg, about 0.1 mg/kg to 7.5 mg/kg, about 0.1 mg/kg to 7 mg/kg, about 0.1 mg/kg to 6.5 mg/kg, about 0.1 mg/kg to 6 mg/kg, about 0.1 mg/kg to 5.5 mg/kg, about 0.1 mg/kg to 5 mg/kg, about 0.1 mg/kg to 4.5 mg/kg, about 0.1 mg/kg to 4 mg/kg, about 0.5 mg/kg to 3.5 mg/kg, about 0.5 mg/kg to 3 mg/kg, about 1 mg/kg to 10 mg/kg, about 1 mg/kg to 9 mg/kg, about 1 mg/kg to 8 mg/kg, about 1 mg/kg to 7 mg/kg, about 1 mg/kg to 6 mg/kg, about 1 mg/kg to 5 mg/kg, about 1 mg/kg to 4 mg/kg, or about 1 mg/kg to 3 mg/kg.

In one embodiment, anti-HC ADC described herein that is administered to a human patient for treatment or conditioning has a half life of equal to or less than 24 hours, equal to or less than 22 hours, equal to or less than 20 hours, equal to or less than 18 hours, equal to or less than 16 hours, equal to or less than 14 hours, equal to or less than 13 hours, equal to or less than 12 hours, equal to or less than 11 hours, equal to or less than 10 hours, equal to or less than 9 hours, equal to or less than 8 hours, equal to or less than 7 hours, equal to or less than 6 hours, or equal to or less than 5 hours. In one embodiment, the half life of the anti-HC ADC is 5 hours to 7 hours; is 5 hours to 9 hours; is 15 hours to 11 hours; is 5 hours to 13 hours; is 5 hours to 15 hours; is 5 hours to 20 hours; is 5 hours to 24 hours; is 7 hours to 24 hours; is 9 hours to 24 hours; is 11 hours to 24 hours; 12 hours to 22 hours; 10 hours to 20 hours; 8 hours to 18 hours; or 14 hours to 24 hours.

In one embodiment, the methods disclosed herein minimize liver toxicity in the patient receiving the ADC for conditioning. For example, in certain embodiments, the methods disclosed herein result in a liver marker level remaining below a known toxic level in the patient for more than 24 hours, 48 hours, 72 hours, or 96 hours. In other embodiments, the methods disclosed herein result in a liver marker level remaining within a reference range in the patient for more than 24 hours, 48 hours, 72 hours, or 96 hours. In certain embodiments, the methods disclosed herein result in a liver marker level rising not more than 1.5-fold above a reference range, not more than 3-fold above a reference range, not more than 5-fold above a reference range, or not more than 10-fold above a reference range for more than 24 hours, 48 hours, 72 hours, or 96 hours. Examples of liver markers that can be used to test for toxicity include alanine aminotransaminase (ALT), lactate dehydrogenase (LDH), and aspartate aminotransaminase (AST). In certain embodiments, administration of an ADC as described herein, i.e., where two doses are administered instead of a single dose, results in a transient increase in a liver marker, e.g., AST, LDH, and/or ALT. In some instances, an elevated level of a liver marker indicating toxicity may be reached, but within a certain time period, e.g., about 12 hours, about 18 hours, about 24 hours, about 36 hours, about 48 hours, about 72 hours, above 3 days, about 3.5 days, about 4 days, about 4.5 days, about 5 days, about 5.5 days, about 6 days, about 6.5 days, about 7 days, about 7.5 days, or less than a week, the liver marker level returns to a normal level not associated with liver toxicity. For example, in a human (average adult male), a normal, non-toxic level of ALT is 7 to 55 units per liter (U/L); and a normal, non-toxic level of AST is 8 to 48 U/L. In certain embodiments, at least one of the patient's blood AST, ALT, or LDH levels does not reach a toxic level between administration of a first dose of the ADC and 14 days after administration of the first dose to the patient. For example, the patient may be administered a first dose and subsequently a second dose, a third dose, a fourth dose, or more doses within, e.g., 5, 10, or 14 days of being administered the first dose, yet at least one of the patient's blood AST, ALT, or LDH levels does not reach a toxic level between administration of a first dose of the ADC and 14 days after administration of the first dose to the patient.

In certain embodiments, at least one of the patient's blood AST, ALT, or LDH levels does not rise above normal levels, does not rise more than 1.5-fold above normal levels, does not rise more than 3-fold above normal levels, does not rise more than 5-fold above normal levels, or does not rise more than 10-fold above normal levels.

The present invention includes dosing regimens that reduce adverse events and toxicity using ADCs that are capable of binding an antigen expressed by a hematopoietic cell, such as a hematopoietic stem cell, an immune cell or a cancer cell. Examples of such antigens include, but are not limited to, CD117, CD2, CD5, CD45, CD252, CD134, and CD137.

In the case of a conditioning procedure prior to hematopoietic stem cell transplantation, the antibody, or antigen-binding fragment thereof can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days) or more prior to administration of the exogenous hematopoietic stem cell transplant. Ranges including the numbers recited herein are also included in the contemplated methods.

Dosing ranges described above may be combined with anti-HC ADCs having half lives recited herein.

Using the methods disclosed herein, a physician of skill in the art can administer to a human patient in need of hematopoietic stem cell transplant therapy an ADC, an antibody or an antigen-binding fragment thereof capable of binding an antigen expressed by hematopoietic stem cells (e.g., CD117 (e.g., GNNK+ CD117), or CD45) or an antigen expressed by mature immune cells, such as T-cells (e.g., CD45, CD2, CD5, CD137, or CD252). In this fashion, a population of endogenous hematopoietic stem cells can be depleted prior to administration of an exogenous hematopoietic stem cell graft so as to promote engraftment of the hematopoietic stem cell graft. The antibody may be covalently conjugated to a toxin, such as a cytotoxic molecule described herein or known in the art. For instance, an anti-CD117 antibody or antigen-binding fragment thereof (such as an anti-HC antibody (e.g., anti-CD117 antibody, anti-CD45 antibody, anti-CD2 antibody, anti-CD5 antibody, anti-CD137 antibody, or anti-CD252 antibody) or antigen-binding fragment thereof) can be covalently conjugated to a cytotoxin, such as pseudomonas exotoxin A, deBouganin, diphtheria toxin, an amatoxin, such as γ-amanitin, α-amanitin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof. This conjugation can be performed using covalent bond-forming techniques described herein or known in the art. The antibody, antigen-binding fragment thereof, or drug-antibody conjugate can subsequently be administered to the patient, for example, by intravenous administration, prior to transplantation of exogenous hematopoietic stem cells (such as autologous, syngeneic, or allogeneic hematopoietic stem cells) to the patient.

The anti-HC antibody (e.g., anti-CD117 antibody, anti-CD45 antibody, anti-CD2 antibody, anti-CD5 antibody, anti-CD137 antibody, or anti-CD252 antibody), an antigen-binding fragment thereof, or ADC can be administered in an amount sufficient to reduce the quantity of endogenous hematopoietic stem cells, for example, by about 10%, about 20%, about 30%, about 40%, about 50%, about 60%, about 70%, about 80%, about 90%, about 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of hematopoietic stem cells has reached a minimum value in response to conditioning therapy with an anti-HC antibody (e.g., an anti-CD117 antibody, an anti-CD45 antibody, an anti-CD2 antibody, an anti-CD5 antibody, an anti-CD137 antibody, or an anti-CD252 antibody), an antigen-binding fragment thereof, or ADC, the physician may conclude the conditioning therapy, and may begin preparing the patient for hematopoietic stem cell transplant therapy.

The anti-HC antibody (e.g., anti-CD117 antibody, anti-CD45 antibody, anti-CD2 antibody, anti-CD5 antibody, anti-CD137 antibody, or anti-CD252 antibody), antigen-binding fragment thereof, or ADC can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, or drug-antibody conjugate can be administered to the patient at a dosage of, for example, from about 0.001 mg/kg to about 100 mg/kg, from about 0.001 mg/kg to about 10 mg/kg, about 0.01 mg/kg to 9.5 mg/kg, about 0.1 mg/kg to 9 mg/kg, about 0.1 mg/kg to 8.5 mg/kg, about 0.1 mg/kg to 8 mg/kg, about 0.1 mg/kg to 7.5 mg/kg, about 0.1 mg/kg to 7 mg/kg, about 0.1 mg/kg to 6.5 mg/kg, about 0.1 mg/kg to 6 mg/kg, about 0.1 mg/kg to 5.5 mg/kg, about 0.1 mg/kg to 5 mg/kg, about 0.1 mg/kg to 4.5 mg/kg, about 0.1 mg/kg to 4 mg/kg, about 0.5 mg/kg to 3.5 mg/kg, about 0.5 mg/kg to 3 mg/kg, about 1 mg/kg to 10 mg/kg, about 1 mg/kg to 9 mg/kg, about 1 mg/kg to 8 mg/kg, about 1 mg/kg to 7 mg/kg, about 1 mg/kg to 6 mg/kg, about 1 mg/kg to 5 mg/kg, about 1 mg/kg to 4 mg/kg, or about 1 mg/kg to 3 mg/kg, prior to administration of a hematopoietic stem cell graft to the patient. The antibody, antigen-binding fragment thereof, or drug-antibody conjugate can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

Following the conclusion of conditioning therapy, the patient may then receive an infusion (e.g., an intravenous infusion) of exogenous hematopoietic stem cells, such as from the same physician that performed the conditioning therapy or from a different physician. The physician may administer the patient an infusion of autologous, syngeneic, or allogeneic hematopoietic stem cells, for instance, at a dosage of from 1×10³ to 1×10⁹ hematopoietic stem cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from about 1 hour to about 6 months, or more, following hematopoietic stem cell transplant therapy (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or more). A finding that the concentration of hematopoietic stem cells or cells of the hematopoietic lineage has increased (e.g., by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 100%, about 200%, about 500%, or more) following the transplant therapy relative to the concentration of the corresponding cell type prior to transplant therapy provides one indication that treatment with the anti-HC antibody (e.g., anti-CD117 antibody, anti-CD45 antibody, anti-CD2 antibody, anti-CD5 antibody, anti-CD137 antibody, or anti-CD252 antibody), an antigen-binding fragment thereof, or an ADC has successfully promoted engraftment of the transplanted hematopoietic stem cell graft.

Engraftment of hematopoietic stem cell transplants due to the administration of an anti-HC antibody (e.g., an anti-CD117 antibody, an anti-CD45 antibody, an anti-CD2 antibody, an anti-CD5 antibody, an anti-CD137 antibody, or an anti-CD252 antibody), antigen-binding fragments thereof, or ADCs, can manifest in a variety of empirical measurements. For instance, engraftment of transplanted hematopoietic stem cells can be evaluated by assessing the quantity of competitive repopulating units (CRU) present within the bone marrow of a patient following administration of an antibody or antigen-binding fragment thereof capable of binding capable of binding an antigen expressed by hematopoietic stem cells (e.g., CD117 (e.g., GNNK+ CD117), or CD45) or an antigen expressed by mature immune cells, such as T-cells (e.g., CD2, CD5, CD137, or CD252) and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention. Ab1 refers to an anti-CD117 antibody having the variable regions of the anti-CD117 antibody Ab67, the sequences of which are provided in Table 5. Ab2 refers to an anti-CD117 antibody having the variable regions of the anti-CD117 antibody Ab85, the sequences of which are provided in Table 5. Ab3 refers to an anti-CD117 antibody having the variable regions of the anti-CD117 antibody Ab249, the sequences of which are provided in Table 5. Ab4 refers to an anti-CD117 antibody having the variable regions of the anti-CD117 antibody CK6, the sequences of which are provided in Table 5. Ab5 refers to a monoclonal antibody directed against CD45 (i.e., an anti-CD45 antibody)). Further, “ADC1” and “ADC2” refer to two antibody drug conjugates comprising an antibody (as disclosed herein) conjugated to at least one drug, wherein the drug is an amatoxin variant disclosed herein, wherein the amatoxin variant of ADC1 is different from the amatoxin variant of ADC2. The anti-CD117 ADC used in Example 7 through Example 11 is referred to as ADC1 and is anti-CD117 antibody Ab85 (i.e., Ab2) having Fc mutations D265C and H435A (defined by the EU index)) conjugated to an amatoxin via a cleavable linker.

### Example 1. In Vitro Fc-Binding Studies with Fc Variants

To identify Fc modifications that abrogate Fc gamma receptor binding and thereby silence antibody effector functions, IgG antibodies containing one or more amino acid substitution in the Fc region were assessed in an Octet binding assay to measure their ability to bind to different Fc gamma receptors. The following amino acid substitutions within the Fc region of an IgG1 were tested (amino acid positions refer to the Fc region according to the EU index):

D265A
D265C
D265C/H435A
D265C/LALA
D265A/LALA
D265C/LALA/H435A
D265C/N297G
D265C/N297G/H435A
D265C (EPLVLAdelG; also referred to herein as “IgG2*”)
D265C (EPLVLAdelG; also referred to herein as “IgG2*”)/H435A
D265C/N297Q/H435A
D265C/N297Q
EPLVLAdelG/H435A
N297A
N297G
N297Q
D265C/N297A/H435A
LALA/P329G
EPLVLAdelG
LALA/P331G
D265A/H435A
D265C/LALA/P331G/H435A
N297A.IHH (i.e., N297A.I253A.H310A.H435A)
CH2 deleted
D265C/LALA
D265C/LALA/P329A/H435A

“IgG2*,” as used in the context of Fc modifications in these examples, refers to an Fc region having the following mutations: E233P, L234V, L235A, deletion of G236 in an hIgG1 backbone (i.e., EPLVLAdelG).

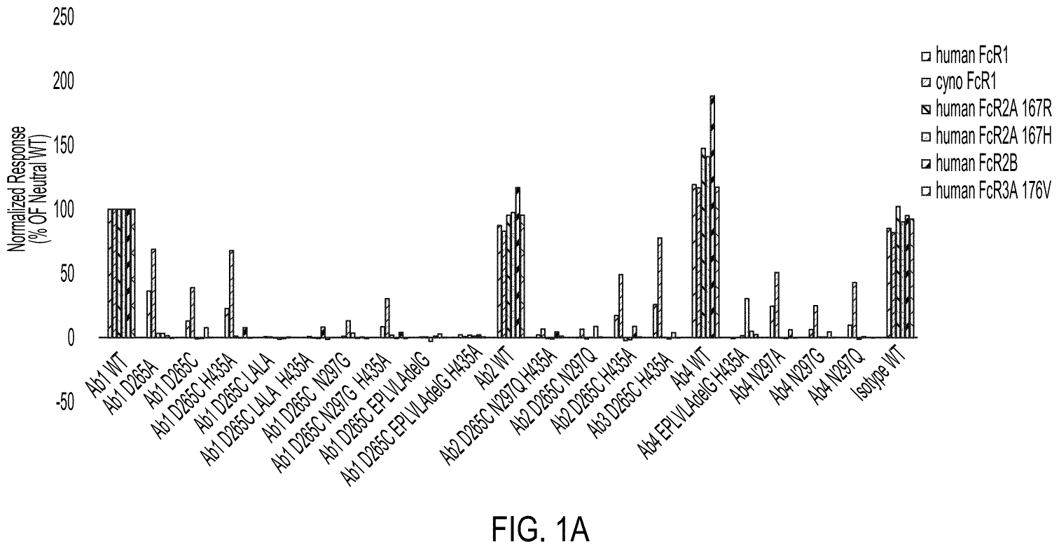

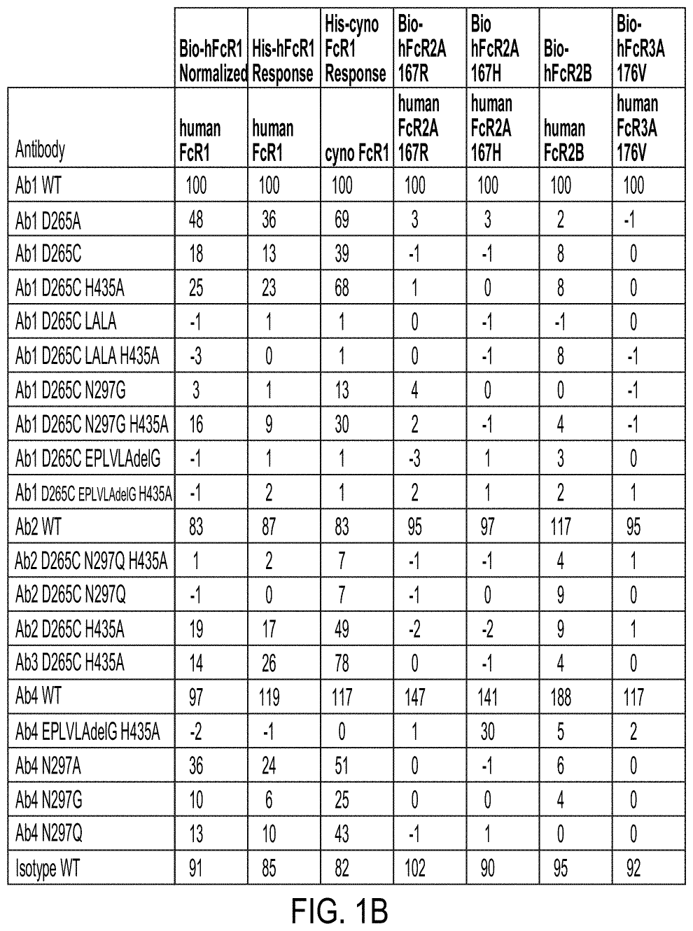

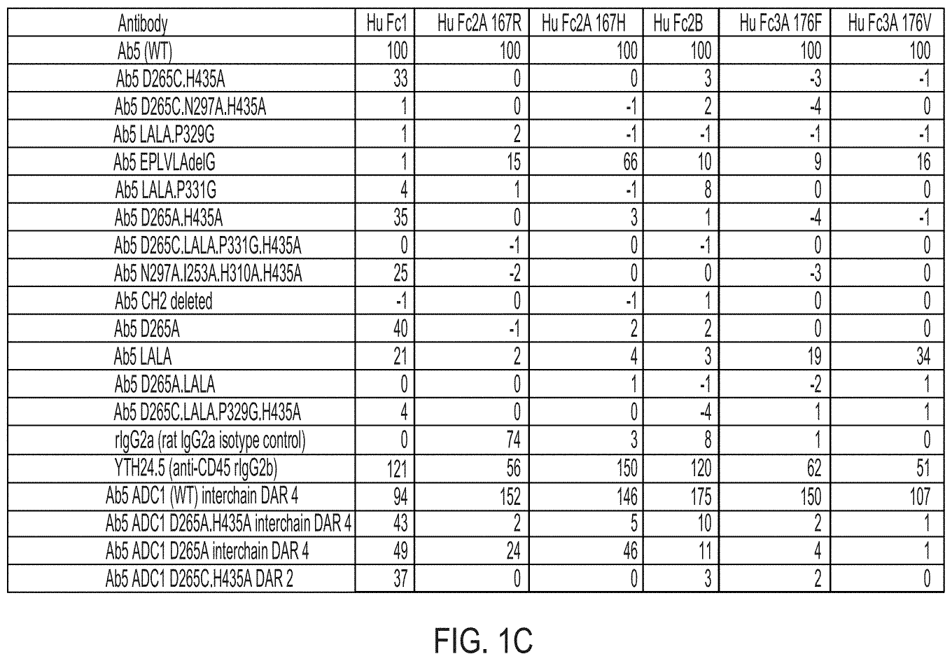

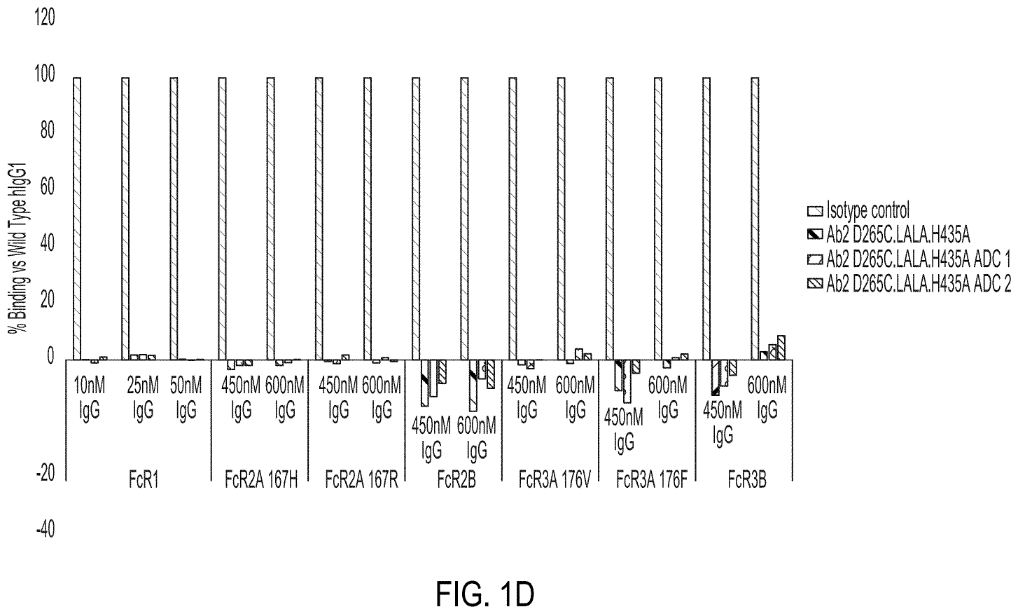

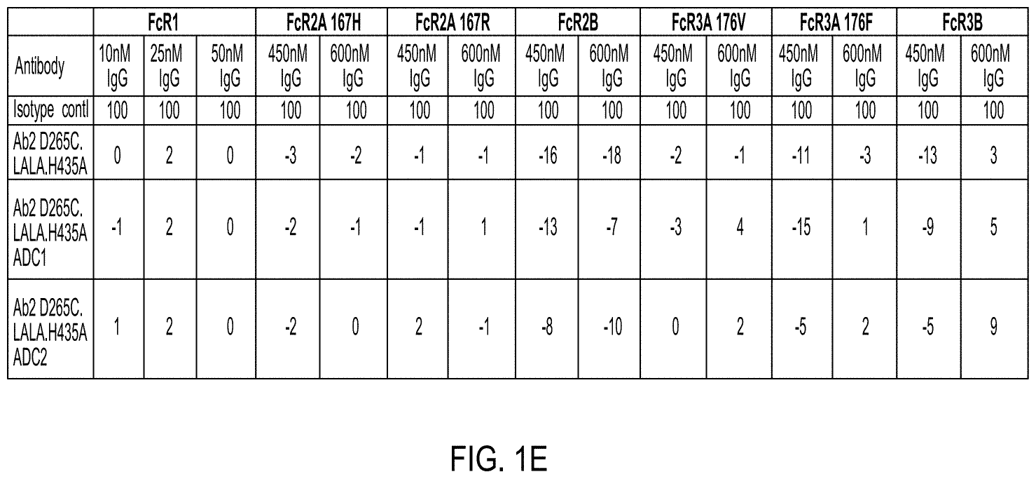

Wild type (WT) as used in these examples refers to an IgG1 Fc region that does not have any substitutions (unless otherwise specified), including those substitutions described herein. “LALA” as used throughout the examples refers to two amino acid substitutions at positions 234 and 235, specifically L234A and L235A. In some embodiments, the anti-CD117 antibody (or anti-CD45 antibody) herein comprises an Fc region comprising one of the following modifications or combinations of modifications: D265A, D265C, D265C/H435A, D265C/LALA, D265C/LALA/H435A, D265C/N297G, D265C/N297G/H435A, D265C (EPLVLAdelG in IgG1), D265C (EPLVLAdelG in IgG1)/H435A, D265C/N297Q/H435A, D265C/N297Q, EPLVLAdelG/H435A, N297A, N297G, N297Q, D265C/N297A/H435A, LALA/P329G, EPLVLAdelG, LALA/P331G, D265A/H435A, D265C/LALA/P331G/H435A, N297A.IHH, CH₂ deleted, D265C/LALA, or D265C/LALA/P329A/H435A. The various substitutions and combinations of substitutions were tested in a number of different anti-CD117 antibodies (see FIGS. 1A-1E), including a neutral anti-CD117 human antibody (i.e., Ab1), CK6 (also referred to as 3100 (i.e., Ab4)), antagonistic anti-CD117 human antibodies (i.e., Ab2 and Ab3; both Ab2 and Ab3 are different antagonistic antibodies than CK6), and a monoclonal antibody directed against CD45 (i.e., Ab5 (an anti-CD45 antibody)).

The binding assays were performed at 25° C. in phosphate buffered saline with (0.1% BSA, 0.02% Tween-20) using Bio-Layer Interferometry Device (ForteBio). Various Fc gamma receptors (i.e., human FcγR1 (hFcγR1), cynomolgus FcγRI (cyno FcγR1), hFcγR2A 167R, hFcγR2A 167H, hFcγR2B, hFcγR2A 167F, hFcγR3A 176V or hFcγR3B) were tagged with biotin (“Bio”) or histidine (“His”) and immobilized on streptavidin biosensors or anti-histidine biosensors, respectively, at a concentration of 10-25 nM. For the association step, the indicated antibody was added at a concentration of 25 nM for the FcγR1 binding studies or at a concentration of 300 nM for the FcγR2A, 2B, or 3A binding studies. The measured binding response for each antibody variant was normalized to the level of WT IgG1 binding under similar conditions.

The normalized binding response of each antibody variant relative to WT IgG1 binding is shown in FIG. 1A and quantification of the normalized binding response of each antibody variant is shown in FIG. 1B.

Additional binding assays were performed as described above to test various Fc mutations and combinations of Fc mutations based on an anti-CD45 antibody (i.e., Ab5) and corresponding anti-CD45 ADCs (see FIG. 1C) as well as an anti-CD117 antibody (i.e., Ab2) and corresponding anti-CD117 ADCs (see FIGS. 1D and 1E) normalized to the level of WT IgG1 binding under similar conditions.

These results demonstrate that D265C alone does not completely abrogate Fc gamma receptor binding (see FIGS. 1A and 1B). The data suggests that in order to abolish Fc gamma receptor binding, the D265C mutation must be combined with other mutations, such as the amino acid substitutions L234A and L235A (“LALA”) (see FIGS. 1A and 1B). Notably, antibodies containing the mutations D265C LALA could not substantially bind Fc gamma receptors and were therefore silenced in regard to effector functions (see FIGS. 1A and 1B). Indeed, the level of binding for an anti-CD117 antibody (see FIGS. 1A and 1B) having an Fc region with the D265C LALA mutations, was essentially zero or undetectable. In addition, the data in FIG. 1C indicate that while the D265A variant (alone) or the LALA variant (alone) show some observable binding to Fc gamma R1 (i.e., “Hu Fc1”), the combination of D265A and LALA, as well as the combination of D265C, N297A and H435A (i.e., “D265C.N297A.H435A”) could not substantially bind Fc gamma receptors and were therefore silenced in regard to effector functions. The antibody “YTH24.5 rIgG2b” is an anti-CD45 antibody control known to have effector function. The data show that the order of silencing on human Fc gamma R1 (i.e., “Hu Fc1”) is most significant for the combination of D265A and LALA (i.e., “D265A.LALA”) and the combination of D265C and N297A (data not provided), followed by LALA (alone), N297A (alone; data not provided), D265A (alone) and wild type (WT) (see FIG. 1C). The data also suggest that both the binding of the naked (un-conjugated) antibody Ab5 and the corresponding ADC (i.e., conjugated antibody) were similar (see FIG. 1C), indicating that the toxin did not significantly affect Fc silencing and further suggesting the corresponding Fc silent ADCs minimize off-target toxicity. The data in FIGS. 1D and 1E indicate that the combination of D265C, LALA and H435A (i.e., “D265C.LALA.H435A”) could not substantially bind Fc gamma receptors and were therefore silenced in regard to effector functions. The data also demonstrates that both the naked (un-conjugated) antibody (i.e., the isotype control) and the Ab2 ADCs (i.e., conjugated antibody) could not substantially bind the Fc gamma receptors tested and were therefore silenced in regard to effector functions (see FIGS. 1D and 1E), indicating that the toxin did not affect Fc silencing and further suggesting the corresponding Fc silent ADCs minimize off-target toxicity.

### Example 2. In Vitro Analysis of Fc Variants Using Mast Cell Degranulation Assay

To analyze the degree to which Fc modifications can reduce antibody-triggered mast cell degranulation, antibodies having the Fc mutations described in Example 1 were assessed using an in vitro mast cell degranulation assay.

Mast cells were derived from mobilized peripheral blood CD34+ cells following 8-12 weeks of in vitro culture in the presence of IL-6 and SCF. Cells were cultured overnight in the absence of IL-6 and SCF and in the presence of 150 ng/ml interferon gamma (IFNγ). 100 nM of each indicated antibody was incubated alongside mast cells at 37° C. for 30 minutes. Mast cell degranulation was assessed by measuring the release of beta-hexosaminidase into culture supernatants after treating mast cells with positive control antibodies (i.e., “NEG085” and “104D2”), a negative control antibody (h IgG1), or each of the indicated neutral or antagonist antibody variants.

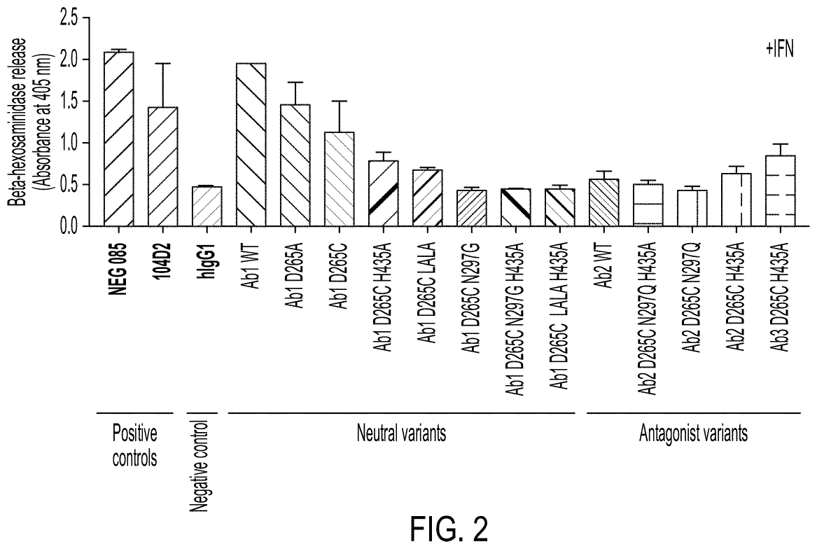

Beta-hexosaminidase release was measured by combining supernatants with p-nitrophenyl N-acetyl-β-D-glucosamide (PNAG) for 60-90 minutes at 37° C. followed by the addition of glycine and is presented as absorbance at 405 nm in FIG. 2. These results show that the D265C LALA combination of mutations (i.e., “D265C.LALA”) that were identified as Fc silencing mutations in Example 1 also were able to reduce activation of mast cell degranulation in the context of a neutral antibody (i.e., Ab1), as the determined levels were similar to those of the negative control (IgG1 matched isotype).

### Example 3. In Vitro Analysis of Fc Variants Using Cytokine Release Assay

Antibodies with modified Fc regions were assessed for the ability of each antibody (e.g., Ab1, Ab2, Ab4, and Ab5) to trigger cytokine release using an in vitro human peripheral blood mononuclear cell (PBMC) cytokine release (CRA) assay.

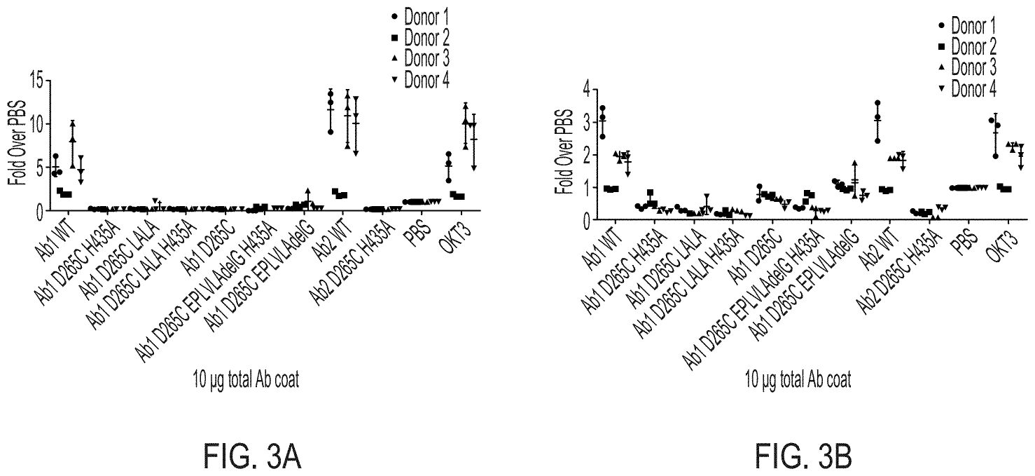

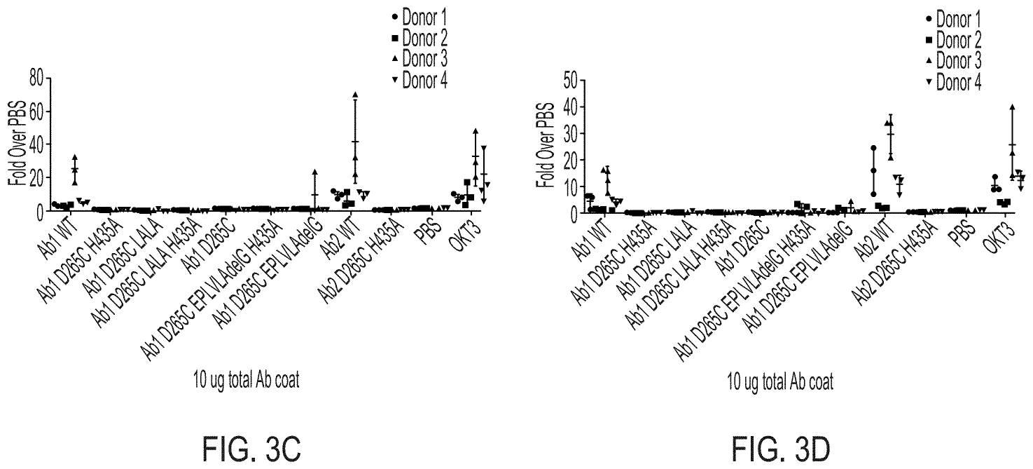

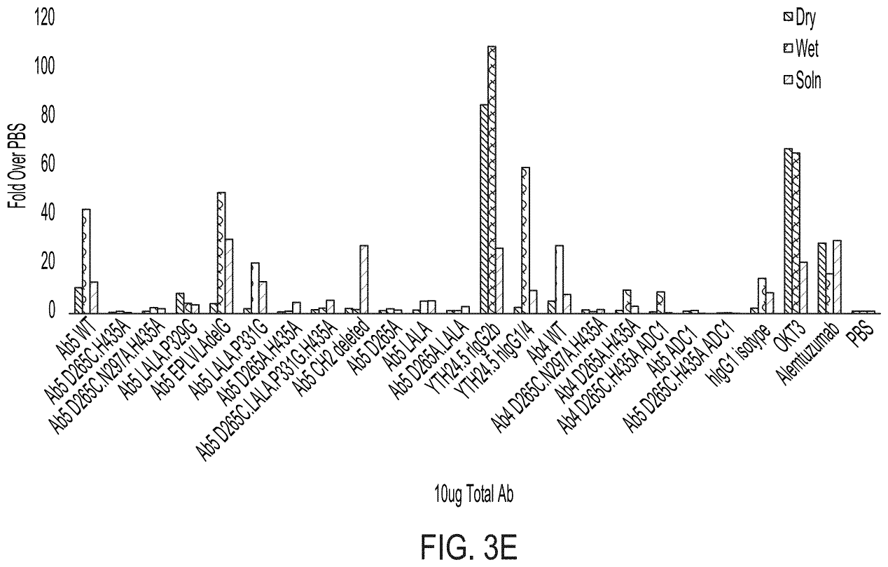

Human PBMCs isolated from four donors were resuspended in Roswell Park Memorial Institute (RPMI) medium with 2% autologous serum (FIGS. 3A-3D). The indicated anti-CD117 antibodies (i.e., Ab1 and Ab2) and variants (i.e., Ab1 and Ab2 variants) or a positive control (OKT3) was immobilized by wet-coating (procedure described below) the antibody at 37° C. for 1 hr onto a non-tissue culture plate prior to the addition of human PBMCs (FIGS. 3A-3D). The PBMCs were incubated with the coated antibody overnight. Supernatants were harvested and analyzed with Meso Scale Discovery (MSD) tissue culture (IC) proinflammatory kit to assess cytokine release in comparison to the positive controls (i.e., isotype h IgG1, OKT3 (positive control), alemtuzumab (positive control)).

The cytokines tested were IL-6 (FIG. 3A), IL-8 (FIG. 3B), TNFa (FIG. 3C), IL-1B (FIG. 3D), IL12p70 (data not shown), IL-10 (data not shown), and IFNg (data not shown). IL12p70, IL10, and IFNg levels were below the limit of quantitation.

For all cytokines tested (FIGS. 3A-3D), significant reduction in cytokine release was observed in the Fc silenced anti-CD117 antibodies (e.g., D265C LALA).

Additional in vitro cytokine release assays were performed to assess the ability of additional antibody variants (i.e., Ab4 and Ab5) to trigger cytokine release (e.g., granulocyte-macrophage colony-stimulating factor (GM-CSF)) using an in vitro human peripheral blood mononuclear cell (PBMC) cytokine release (CRA) assay (i.e., FIG. 3E).

For these in vitro cytokine assays, three different antibody presentation methods were used. For the wet coat method, 10 μg of the antibody in 100-150 μL of PBS were used to wet coat non-tissue culture treated round bottom 96-well plate for one to two hours at 37° C. After wet coating the antibody, excess antibody was removed and washed with 1× of 200 μL of PBS. Fresh human PBMCs were isolated from the whole blood of four donors. The PBMCs were resuspended in RPMI media with 2% autologous serum. 250,000 PBMCs in 200 μL of media were then added to each well and incubated with the wet coated antibodies overnight at 37° C. in a 5% CO₂ tissue culture incubator. The 96-well plate was then centrifuged at 500G for 10 min and 100 μL of the supernatant was collected and analyzed using a mesoscale discovery multiplexed assay (MSD) Human IC Proinflammatory kit (Meso Scale Diagnostics, LLC; Product No. K15008B). The anti-CD3 mAb clone OKT3 was used as a positive control for the cytokine release assay.

For the dry coat method, 1 or 10 μg of the antibody in 20 μL of PBS were added to a non-tissue culture treated round bottom 96-well plate and incubated overnight at room temperature to evaporate solution and dry coat the antibody. Fresh human PBMCs were isolated from the whole blood of donors and resuspended. 250,000 PBMC in 200 μL of 2% autologous serum with 1% Pen-strep in RPMI 1640 media were added to each well in the plate and was incubated overnight at 37° C. in 5% a CO₂ tissue culture incubator. The 96-well plate was then centrifuged at 500G for 10 min and 100 μL of the supernatant was collected and analyzed using a mesoscale discovery multiplexed assay (MSD) Human TC Proinflammatory kit (Meso Scale Diagnostics, LLC; Product No. K15008B). The anti-CD3 mAb clone OKT3 was used as a positive control for the cytokine release assay.

For the solution coat method, fresh human PBMCs were isolated from the whole blood of donors. 250,000 cells were resuspended in 200 μL of 2% autologous serum with 1% Pen-strep in RPMI 1640 media. The cells were then plated in non-tissue culture treated round bottom 96-well plate. 10 μg of antibody was added to the wells containing PBMCs and incubated overnight at 37° C. in 5% a CO₂ tissue culture incubator. The plate was then centrifuged at 500G for 10 min and 100 μL of the supernatant was collected and analyzed using a mesoscale discovery multiplexed assay (MSD) Human TC Proinflammatory kit (Meso Scale Diagnostics, LLC; Product No. K15008B). The anti-CD3 mAb clone OKT3 was used as a positive control for the cytokine release assay.

The results in FIG. 3E demonstrate a significant reduction in release of the cytokine GM-CSF in the Fc silenced anti-CD45 antibody (i.e., Ab5) (e.g., D265C.H435A) and Ab4 for each of the three antibody presentation methods. Similar results showing Fc silenced anti-CD45 antibody (i.e., Ab5) prevents in vitro cytokine release were observed for TNFα, IL-1β, IFNγ and IL-6 (data not shown).

### Example 4. In Vitro Analysis of Fc Variants Using Phagocytosis Assay

To analyze the degree to which Fc modifications can reduce antibody-dependent cellular phagocytosis, anti-CD117 antibodies (i.e., Ab2) having the Fc mutations D265C.LALA.H435A or D265C.H435A were assessed using an in vitro antibody-dependent cellular phagocytosis assay in comparison to controls.

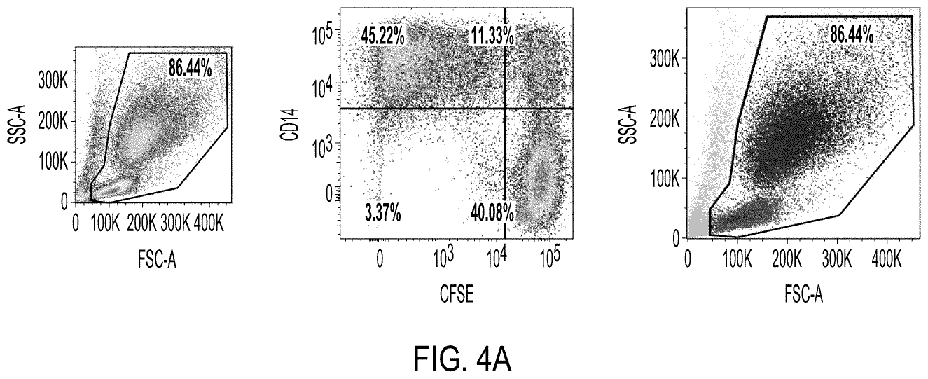

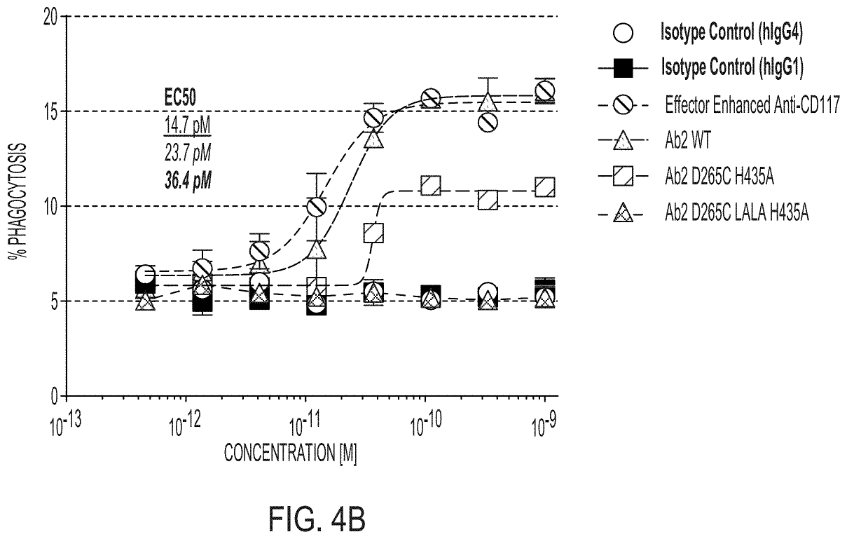

Monocytes were isolated from freshly drawn human whole blood using a RosetteSep kit (StemCell Technologies), followed by incubation for 6 days in the presence of macrophage colony-stimulating factor (M-CSF) to generate monocyte-derived macrophages (MDMs). The resulting MDMs were labeled by staining surface-exposed CD14 and incubated for 2 hours with CFSE-labeled Kasumi-1 cells and a 1:2 molar ratio of MDM to Kasumi-1 cells. The resulting mixture was incubated with increasing concentrations of the indicated antibody for two hours at 37° C. Antibody-dependent cellular phagocytosis (ADCP) was assessed using flow cytometry by determining co-expression of CFSE and CD134 staining (FIG. 4A) after incubation with the indicated antibody (i.e., Ab2 (WT), Ab2 D265C.LALA.H435A, and Ab2 D265C.H435A), positive control (i.e., effector enhanced anti-CD117 antibody) or negative control (i.e., isotype hIgG4 and isotype hIgG1).

The results indicate that the effector enhanced anti-CD117 antibody (positive control) and Ab2 (WT) facilitate robust ADCP activity (FIG. 4B). In addition, a partial reduction in ADCP activity due to increased Fc effector silencing was observed for Ab2 D265C.H435A (i.e., EC₅₀ increases from an EC₅₀ of 14.7 pM for effector enhanced anti-CD117 antibody and an EC₅₀ of 23.7 pM for Ab2 (WT) to an EC₅₀ of 36.4 pM for Ab2 D265C.H435A), while the results for Ab2 D265C.LALA.H435A indicate a significant reduction in ADCP activity due to robust Fc effector silencing when compared with the appropriate controls (FIG. 4B). Together these data demonstrate that ADCP activity can be reduced to baseline levels with antibodies engineered to be Fc silent.

### Example 5. In Vitro Analysis of Thermostability of Fc Variants

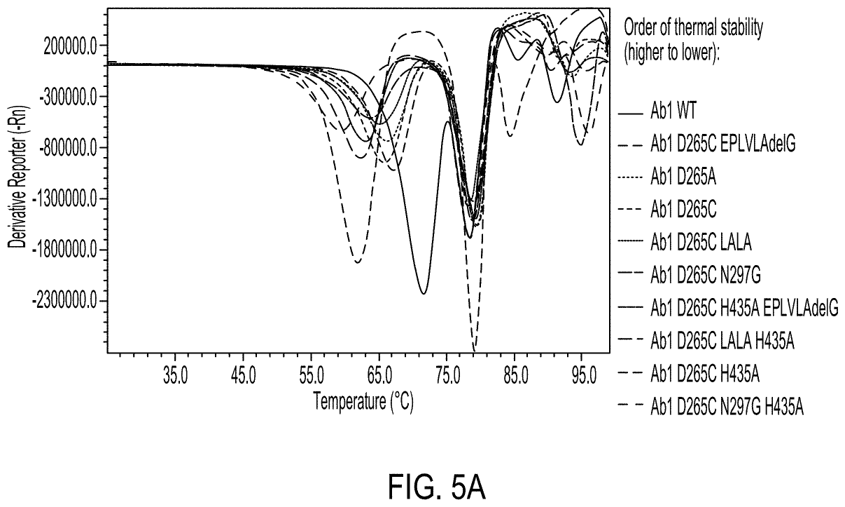

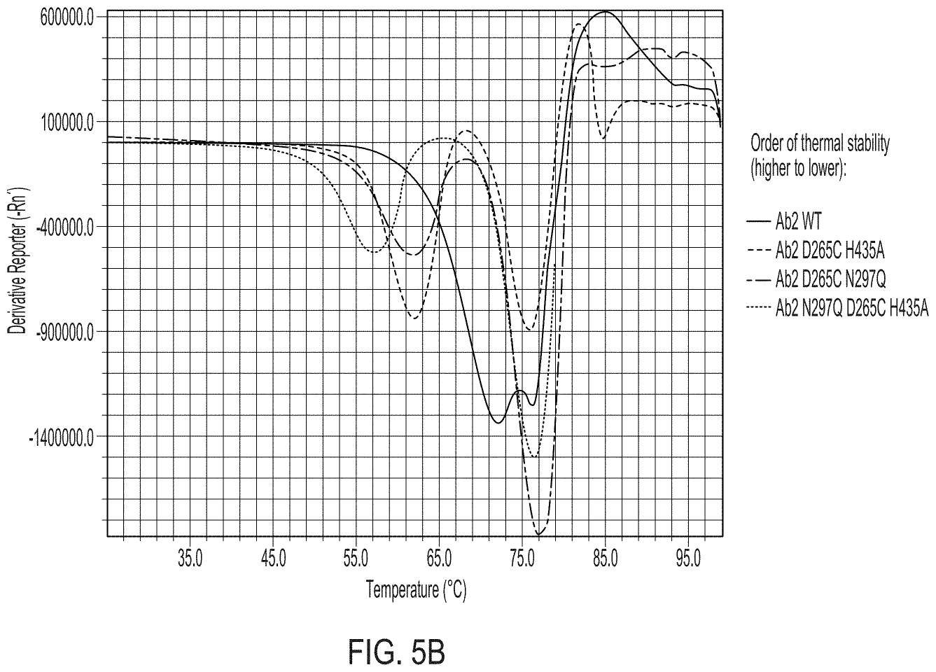

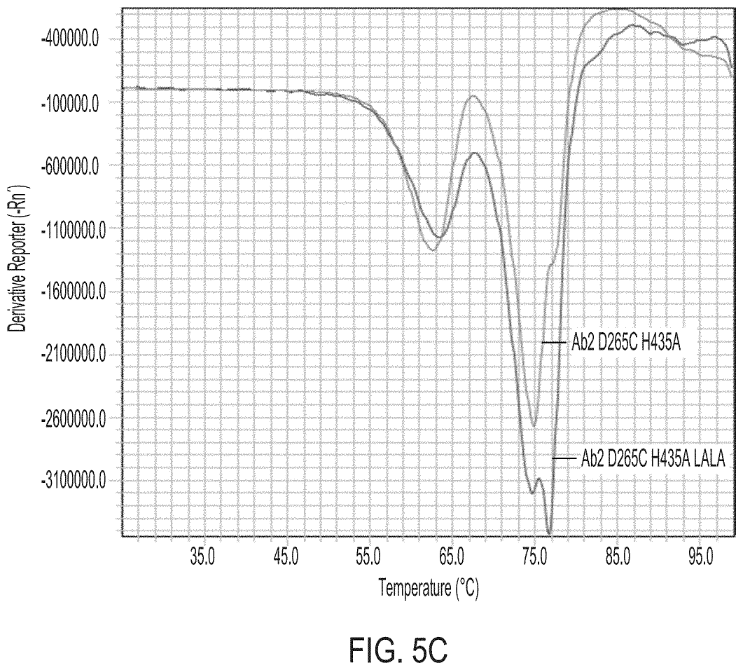

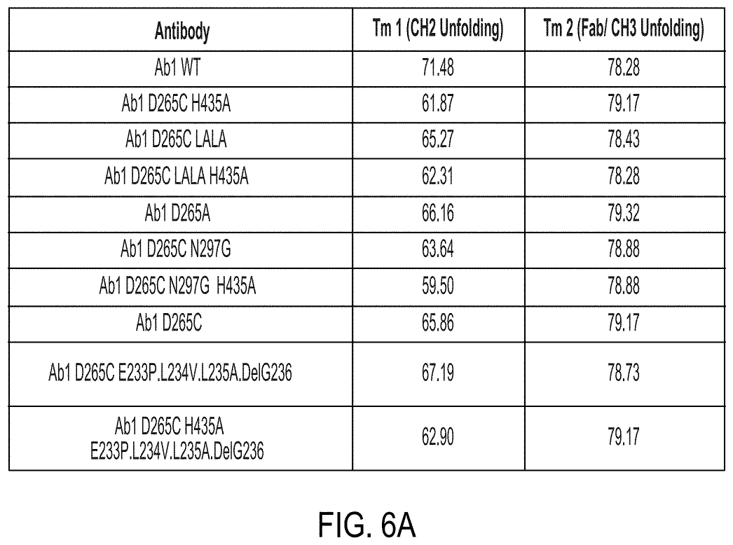

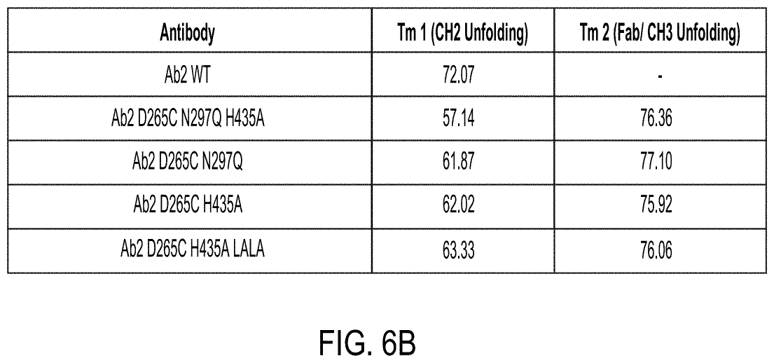

Fc-modified antibodies were evaluated by differential scanning fluorimetry (DSF) to assess the thermostability of each antibody having the indicated amino acid substitutions. 20 micrograms of the antibody combined with the protein thermal shift buffer and dye as per protein thermal shift kit specifications (Applied biosystems, Protein Thermal Shift Dye kit (Part #4461146) were analyzed using Applied Biosystems Quant Studio 7 Flex instrument by Life Technologies and the melting temperature (Tm) of each antibody was determined (see FIGS. 5A, 5B and 5C and FIGS. 6A and 6B).

Mutations on the same antibody backbone caused changes only to the unfolding temperatures of the CH₂ domain. Fab unfolding temperatures remained constant (FIGS. 6A and 6B). Introduction of D265C lowered thermal stability of the WT (FIG. 6A). H435A also caused further decline in stability (FIG. 6A). However, introduction of the LALA mutation into the modified Fc region did not cause additional instability (FIGS. 6A and 6B).

### Example 6: In Vitro Analysis of the Accelerated Stability of the Fc Variants

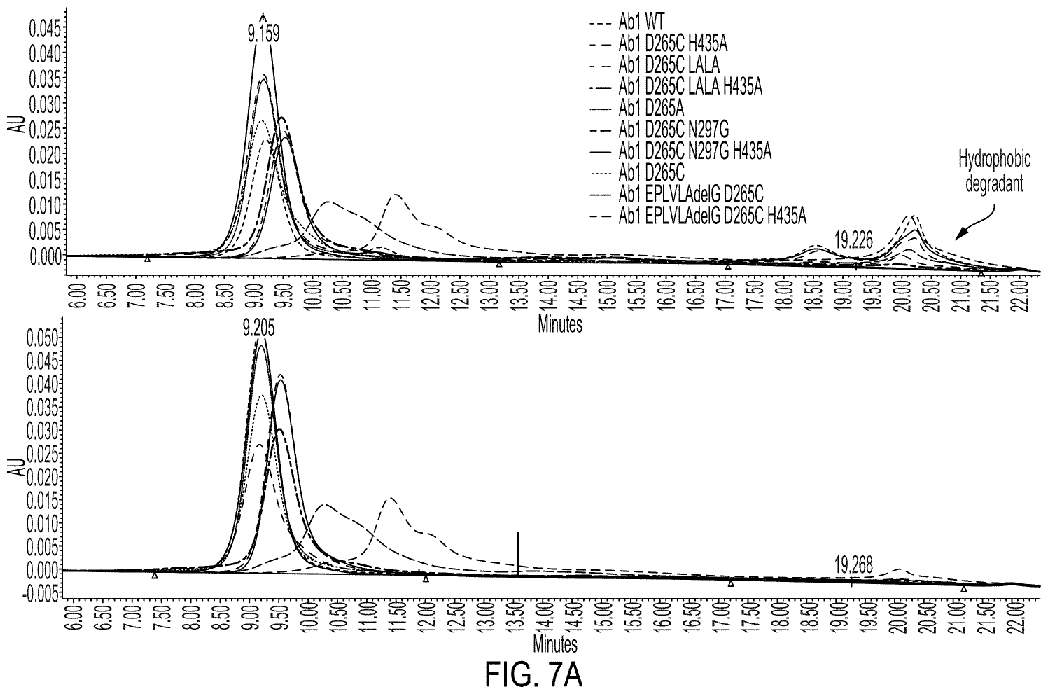

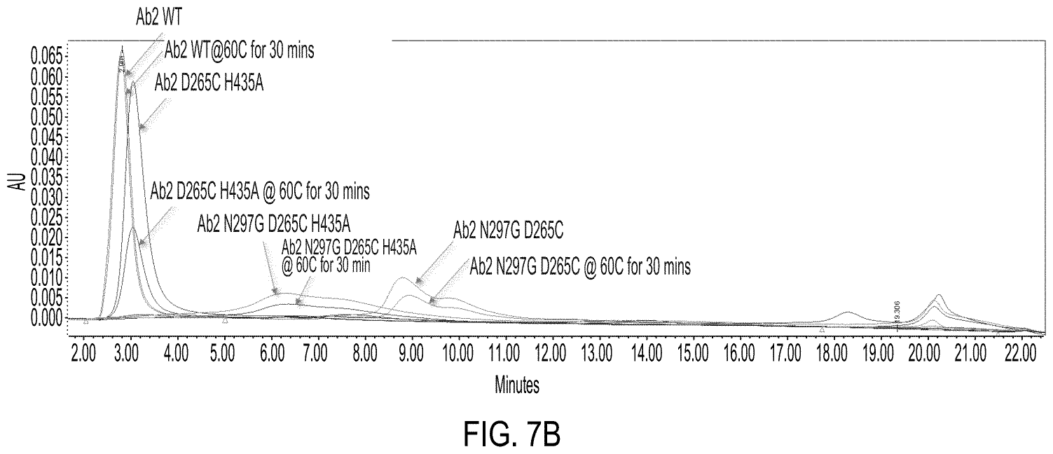

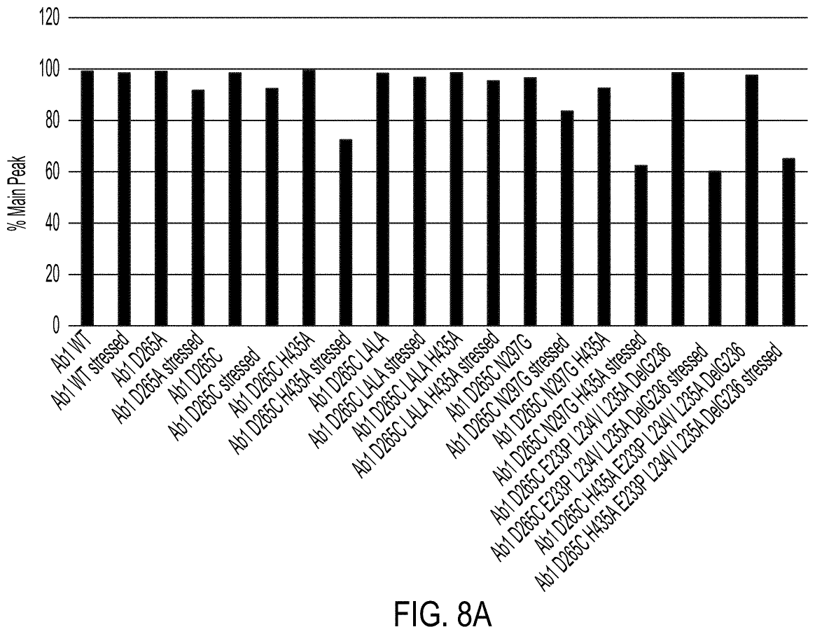

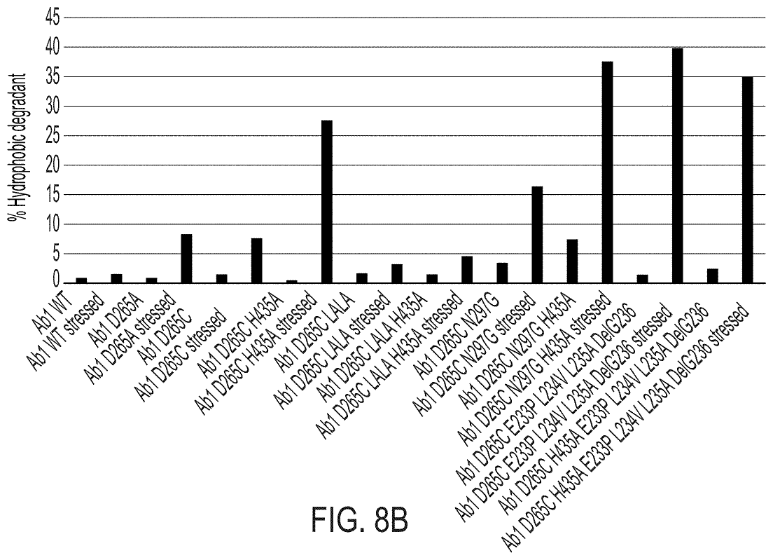

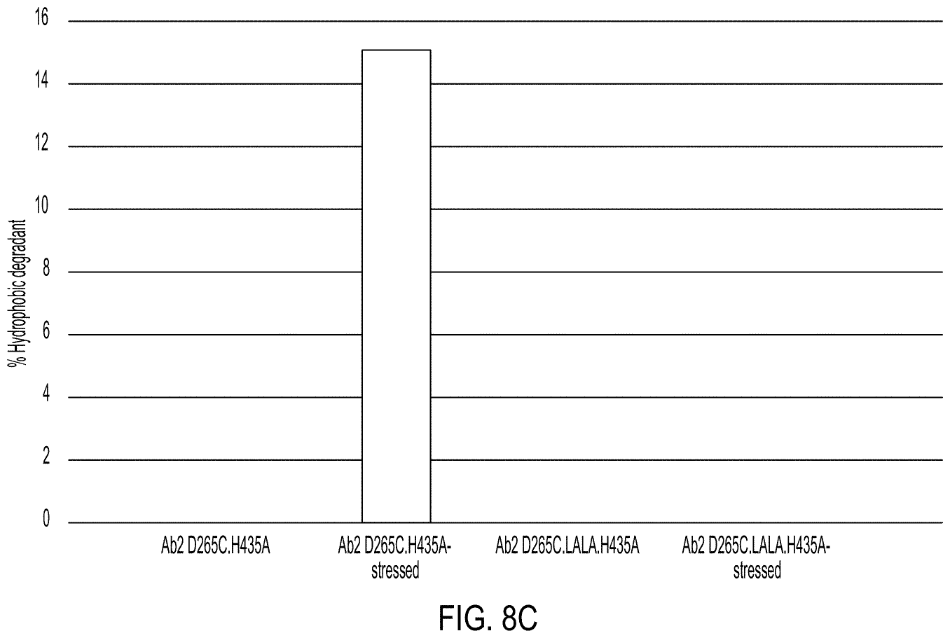

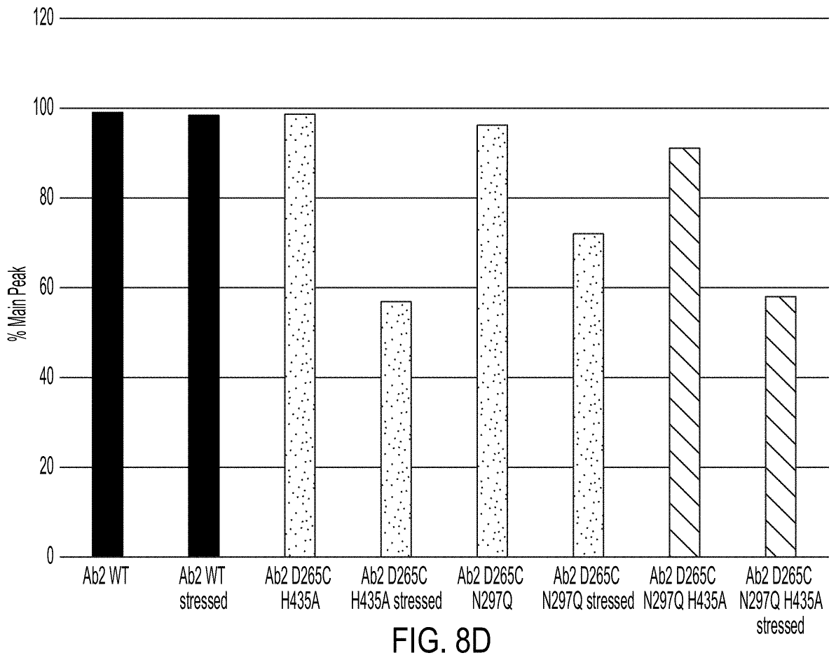

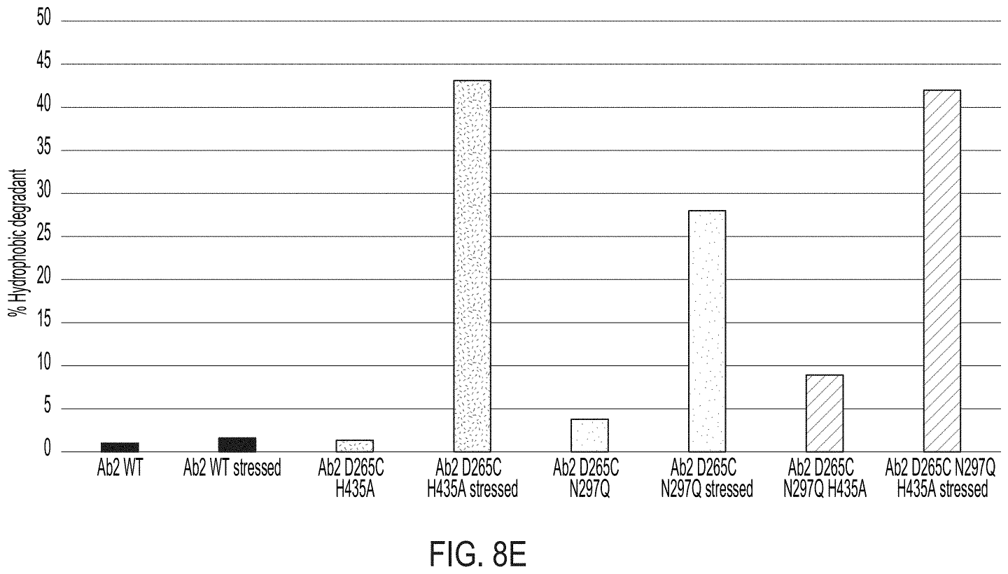

The accelerated stability of each indicated antibody (and antibody variants) was evaluated after incubation at 60 degrees Celsius for 30 minutes by hydrophobic interaction chromatography (HIC) and size exclusion chromatography (SEC). To assess the level of hydrophobic degradants following treatment of each indicated antibody 30 minutes at 60 degrees Celsius, 25 μg of the indicated antibody was injected onto a Tosoh TSKgel Phenyl-5PW, 7.5 mm ID x 7.5 cm, 10 μM column on a Waters ACQUITY Arc HPLC system. The eluted protein was detected using UV absorbance at 280 nm and the results were reported as the area percent of the antibody monomer peak (FIGS. 7A, 7B, 8A and 8D) or hydrophobic degradant peak (FIGS. 7A, 7B, 8B, 8C and 8E). The D265C LALA and D265C H435A LALA variants displayed low levels of hydrophobic degradants following thermal stress (FIG. 8A). In addition, the D265C.H435A.LALA variant demonstrates significant improved stability compared to the D265C.H435A variant (FIG. 8C).

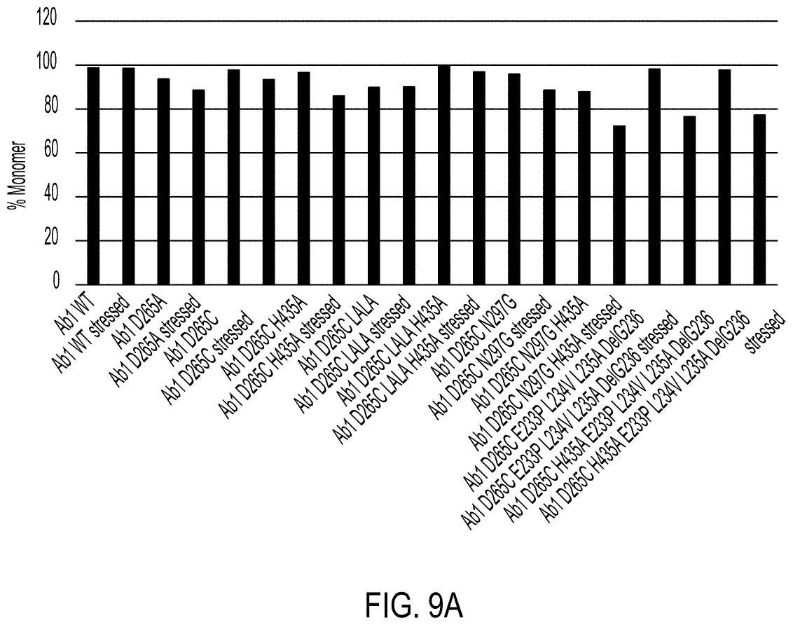

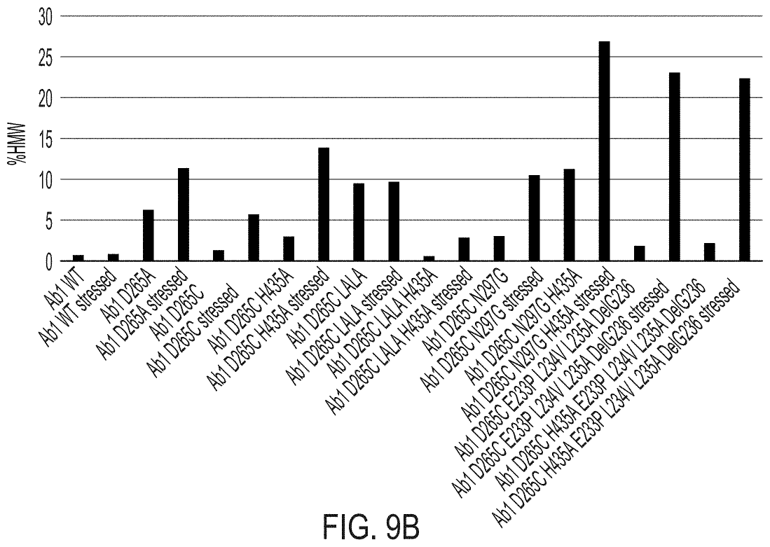

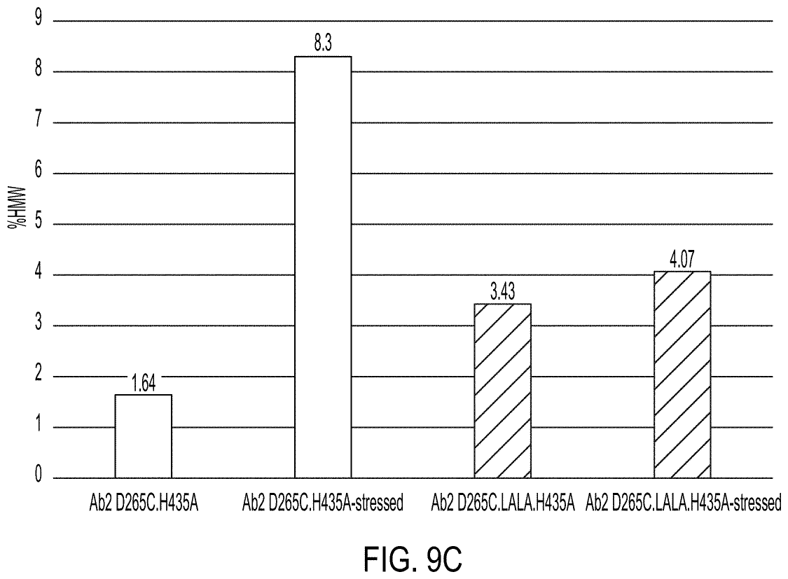

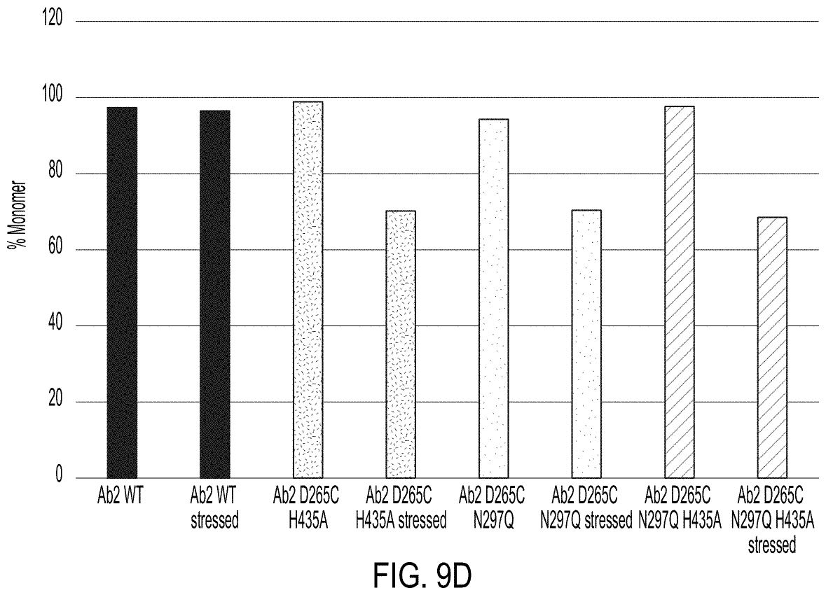

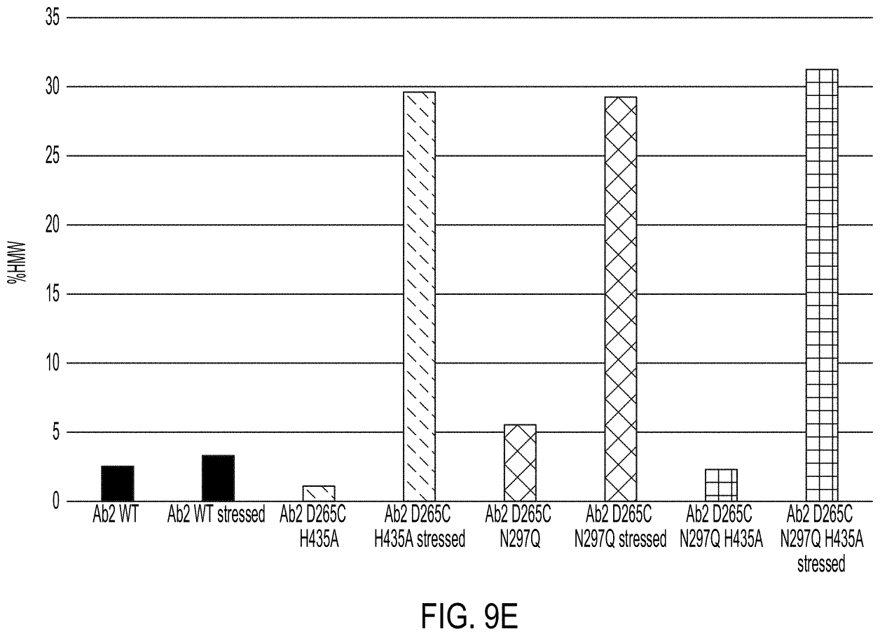

Each antibody variant was also tested for percent high molecular weight (HMW) species formation, an indication of aggregation propensity, using SEC following treatment of each antibody 30 minutes at 60 degrees Celsius. SEC analysis was performed on a Waters ACQUITY Arc HPLC system with a Waters ACQUITY UPLC Protein BEH SEC Column, 200A, 1.7 μm, 4.6 mm×150 mm column. 25 μg of the indicated antibody was injected on the column, and the eluted protein was detected using UV absorbance at 280 nm. The results were reported as the area percent of the antibody monomer peak (FIGS. 9A and 9D) or percent HMW aggregate peak (FIGS. 9B, 9C and 9E). The D265C LALA and D265C H435A LALA variants displayed a low level of aggregates following thermal stress (FIG. 9B). In addition, the D265C.H435A.LALA variant demonstrates improved stability with lower level of aggregates compared to the D265C.H435A variant (FIG. 9C).

### Example 7. Analysis of Non-Human Primate Pharmacokinetics

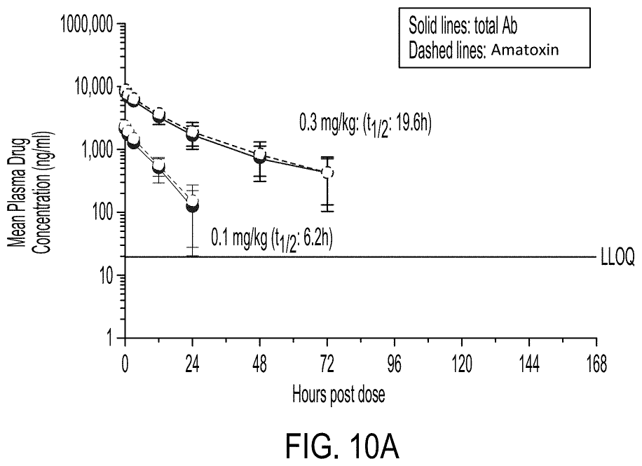

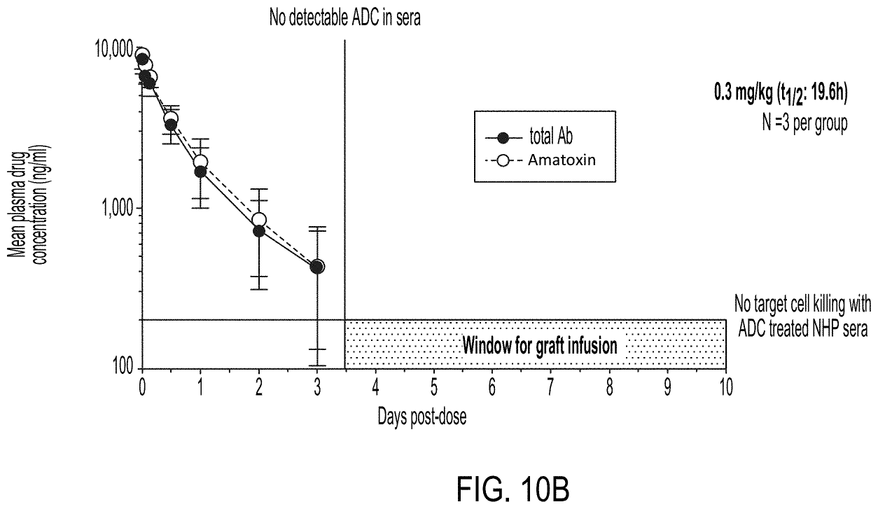

A non-human primate pharmacokinetic assay was performed to determine the clearance rates of an anti-CD117 ADC comprising the anti-CD117 antibody Ab85 (also referred to herein interchangeably as Ab2) having Fc mutations D265C and H435A (defined by the EU index, i.e., Ab2 D265C.H435A) conjugated via a linker (cleavable) to amatoxin. The results in FIGS. 10A and 10B show that the ADC with the Fc-modified Ab85 (i.e., Ab2) antibody showed rapid clearance in cynomolgus monkeys with a half-life suitable for patient preparation for transplant (n=3/group). The clearance rates were similar for the total ADC (solid lines) and amatoxin (dashed lines) detection. FIG. 10A shows the clearance rates for the total ADC (solid lines) and amatoxin (dashed lines). As shown in FIGS. 10A and 10B, the ADC is no longer detectable after three days post-administration. This provides a window following target cell depletion and rapid elimination of the ADC for safe graft infusion.

### Example 8. Analysis of Target Cell Population Depletion in an In Vivo Dose Escalation Study

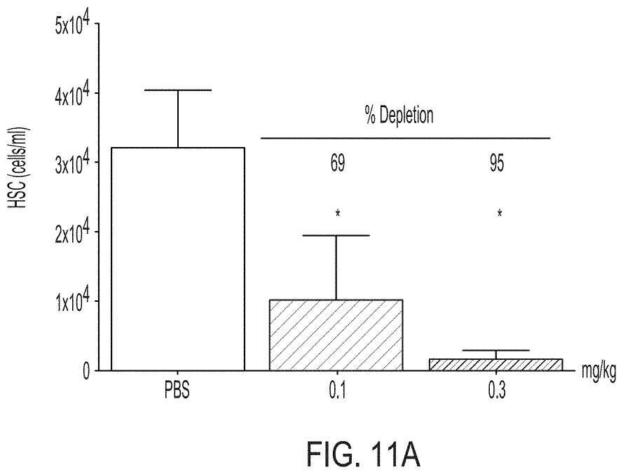

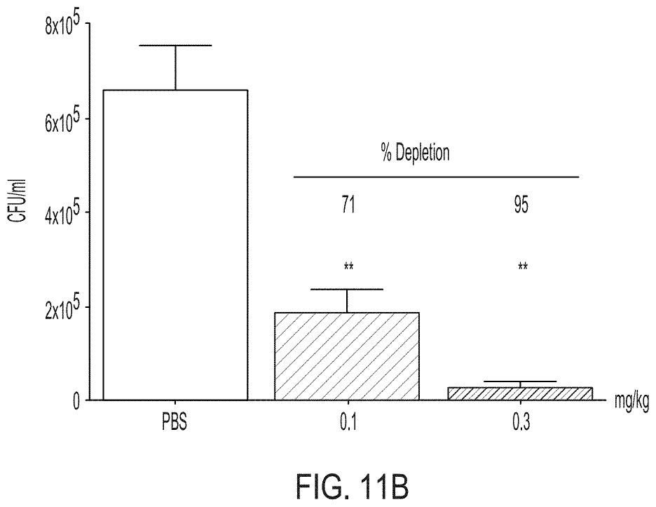

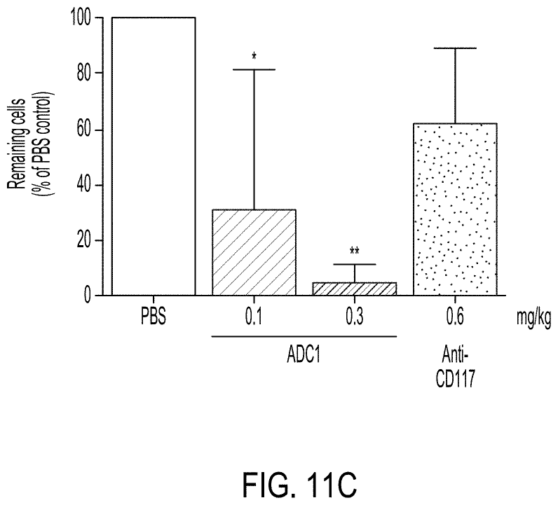

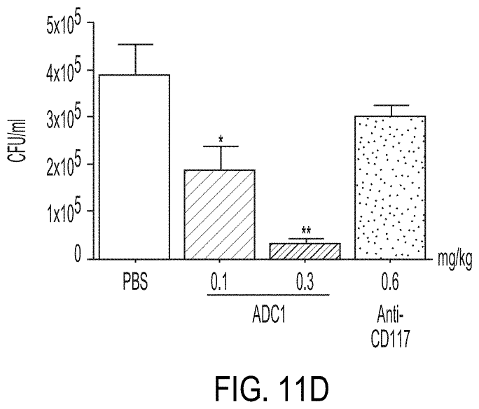

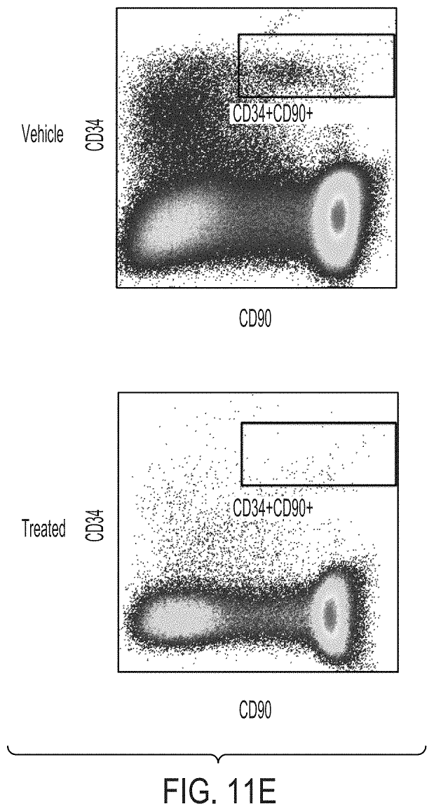

An anti-CD117 ADC comprising the anti-CD117 antibody Ab2 conjugated via a linker to amatoxin was engineered to possess D265C and H435A mutations (i.e., ADC1), resulting in a fast half-life anti-CD117 ADC. Cohorts of cynomolgus monkeys (3 monkeys per cohort) were administered varying doses of ADC1 or a control (i.e., 0.1 mg/kg; 0.3 mg/kg; or a control (PBS)) on day 0. Bone marrow aspirates were collected on day 7 post-dose. Phenotypic HSCs were quantified by flow cytometry (resulting data provided in FIGS. 11A and 11C) and by assessment of colony forming units (CFU) from the bone marrow aspirate (resulting data provided in FIGS. 11B and 11D). The data were graphically represented as a function of varying doses of the ADC1 antibody drug conjugate (ADC) versus a control (i.e., PBS) (x-axis) as shown in FIGS. 11A-11D. FIGS. 11C and 11D further show data corresponding to the unconjugated anti-CD117 antibody (“anti-CD117”). FIG. 11E shows phenotypic analysis of bone marrow collected on day 7 and examined by flow cytometry (FIG. 11E).

The results in FIGS. 11A-11D indicate engineered fast half-life anti-CD117-amatoxin ADC (i.e., ADC1) selectively depletes target cell populations in cynomolgus monkeys. Specifically, the data show that on-target dose-dependent depletion of HSC (FIGS. 11A and 11C) and CFU (FIGS. 11B and 11D) were observed. Therefore, these data demonstrate that the ADC1 exhibited potent selective elimination of NHP HSCs and progenitors in vivo, whereby the fast half life ADC1 provides a model for target cell depletion and rapid clearance prior to bone marrow transplant, that potentially provides a significant improvement in standard-of-care approaches to patient preparation prior to bone marrow transplant and allow more patients to receive a transplant.

### Example 9. Analysis of Neutrophil Cell Count and Lymphocyte Cell Count in an In Vivo Dose Escalation Study

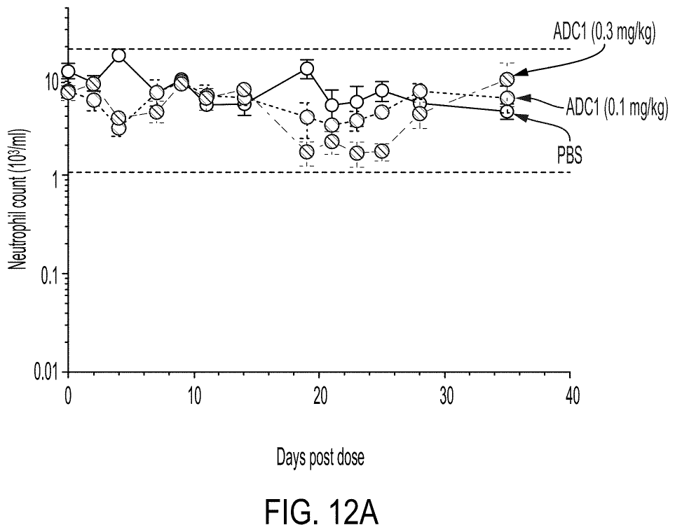

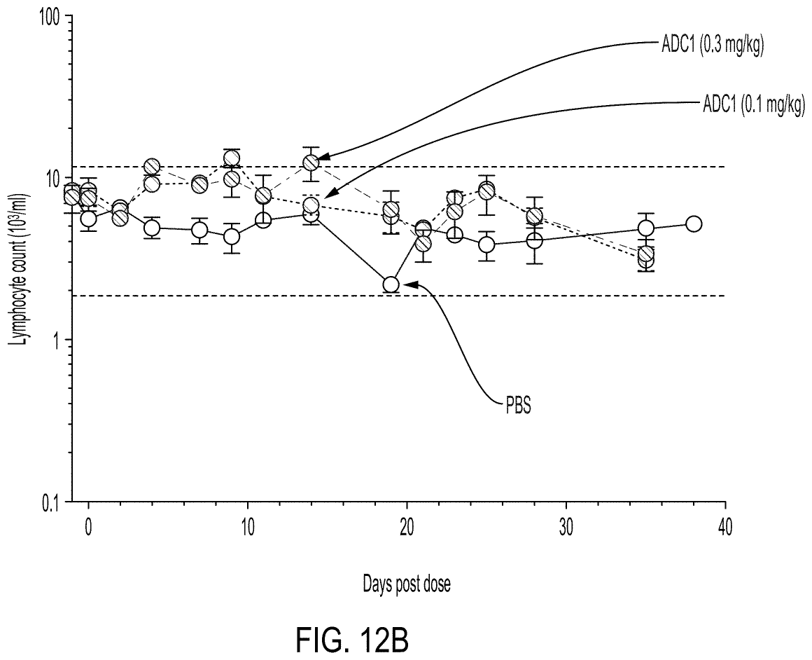

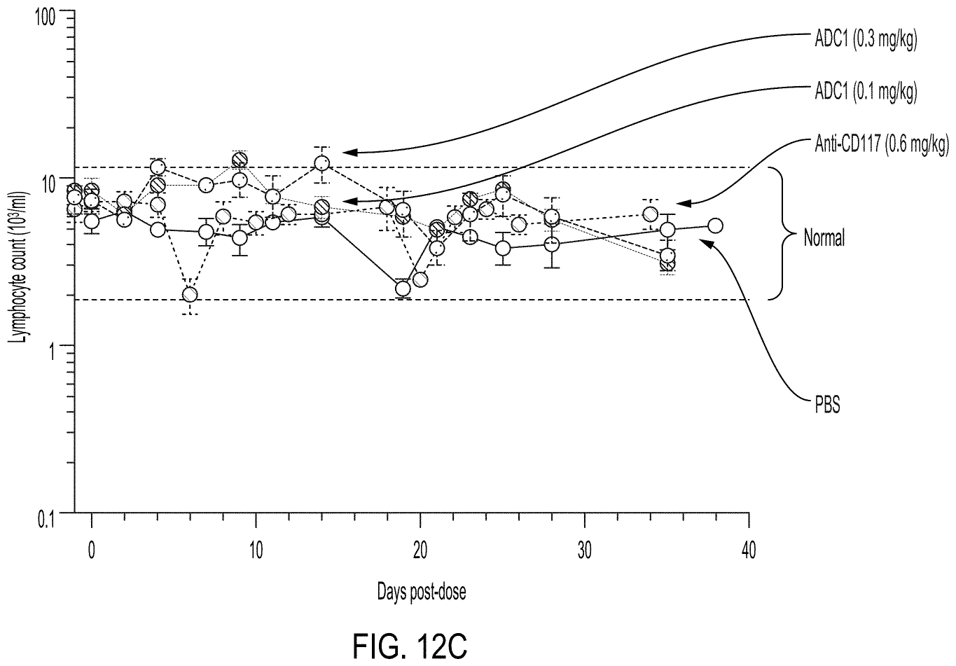

Cohorts of cynomolgus monkeys (3 monkeys per cohort) were administered varying doses of the ADC1 (i.e., 0.1 mg/kg; 0.3 mg/kg; or a control (PBS)) on day 0. Peripheral blood was collected through the course of the study. Hematology was evaluated throughout the course of the study. Neutrophil cell count (10³/mL) and lymphocyte cell count (10³/mL) was measured and was graphically represented as a function of days post dose administration as shown in FIGS. 12A-12C. FIG. 12C further shows data corresponding to the lymphocyte count for cynomolgus monkeys administered an unconjugated anti-CD117 antibody (“anti-CD117”).

The results in FIGS. 12A-12C show on-target dose-dependent depletion observed for neutrophils while the lymphocytes are spared (i.e., lymphocyte counts remained within the normal range). These data also indicate the preservation of the adaptive immune system with delayed onset of neutrophil nadir (18 days), potentially shortening the period of neutropenia.

### Example 10. Analysis of Plasma ALT and Bilirubin in an In Vivo Dose Escalation Study

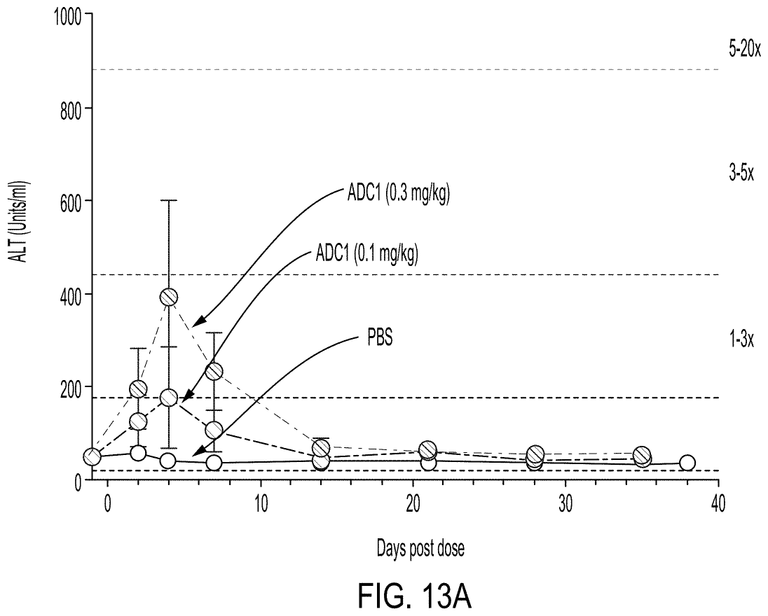

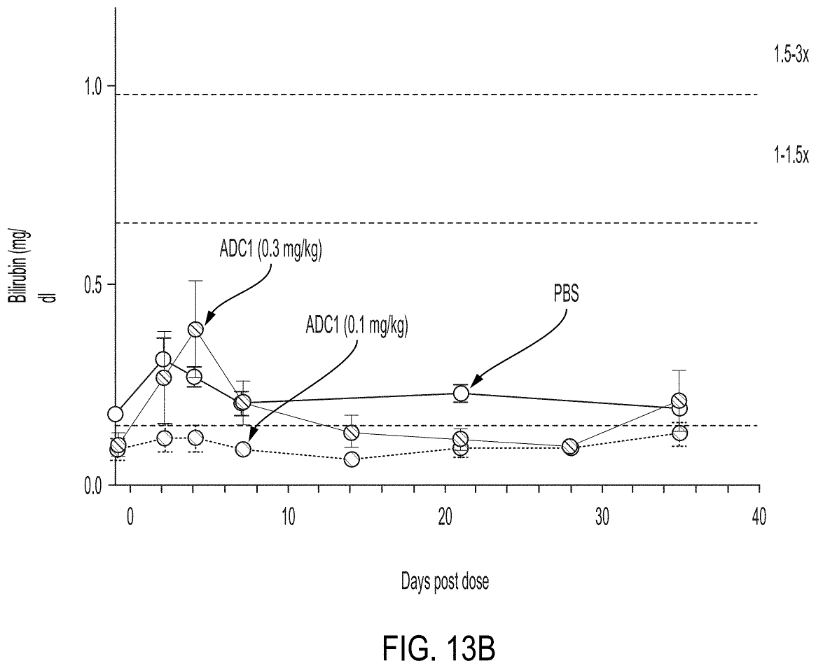

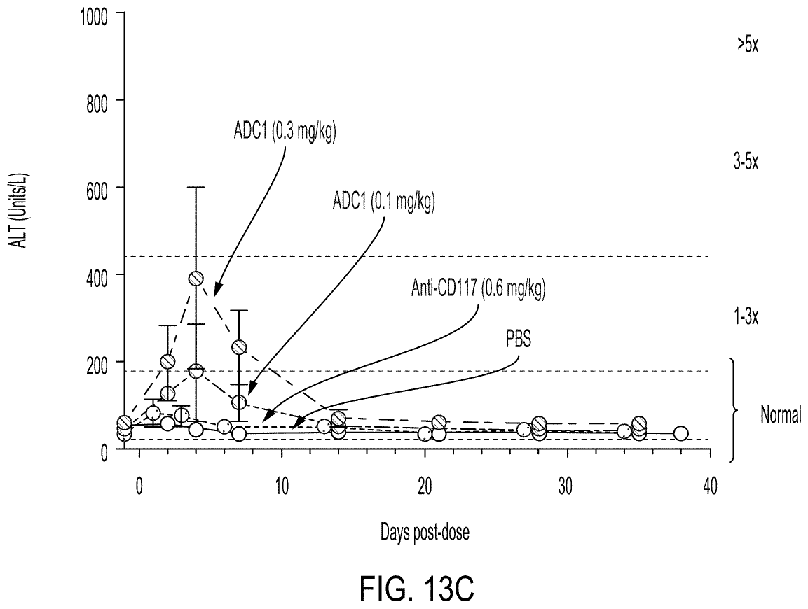

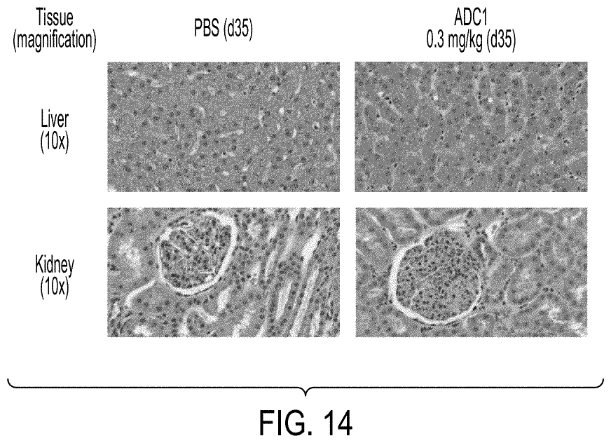

Cohorts of cynomolgus monkeys (3 monkeys per cohort) were administered varying doses of the ADC1 engineered to possess D265C and H435A mutations, resulting in a fast half-life anti-CD117 ADC (i.e., 0.1 mg/kg; 0.3 mg/kg; or a control (PBS)) on day 0. Clinical chemistries were evaluated throughout the course of the study. Plasma levels of ALT (alanine aminotransaminase) and Bilirubin were measured were graphically represented as a function of days post dose administration as shown in FIGS. 13A-13C. FIG. 13C further shows data corresponding to the plasma levels of ALT in cynomolgus monkeys administered an unconjugated anti-CD117 antibody (“anti-CD117”). Liver and kidney tissues were additionally evaluated 35 days post-treatment (FIG. 14). Tissues were formalin-fixed, embedded in paraffin, stained with hematoxylin and eosin (H&E), and imaged with an Aperio AT2 high throughput scanner (FIG. 14).

The results in FIGS. 13A-13C show a transient dose-dependent elevation of liver enzymes and bilirubin was observed in groups treated with the highest doses of isotype-AM (data not shown) and the various doses of ADC1 (* p<0.05, ** p<0.01 when comparing ADC1 against vehicle). No change above the upper limit normal (ULN) for the additional parameters, i.e., GGT, albumin, BUN, PT, ALP, creatinine, glucose, LDH, or PTT, was observed. Further, no histopathological changes were observed in liver and kidney tissues 35 days post-treatment, as shown in FIG. 14.

### Example 11. Analysis of Reticulocyte Cell Count in an In Vivo Study

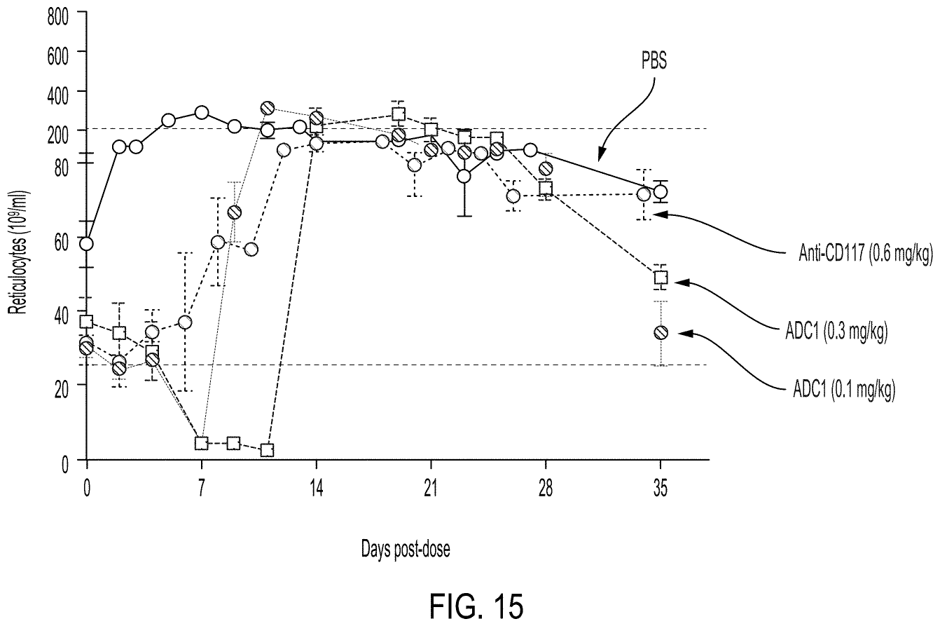

Cohorts of cynomolgus monkeys were administered varying doses of the ADC1 engineered to possess D265C and H435A mutations, resulting in a fast half-life anti-CD117 ADC (0.1 mg/kg, 0.3 mg/kg), an unconjugated CD117 antibody, or a control (PBS) on day 0. Reticulocyte cell count (10⁹/L) was measured using a hematology analyzer and was graphically represented as a function of days post dose administration as shown in FIG. 15.

The results in FIG. 15 indicate a dose dependent depletion of reticulocytes upon administration of the fast half-life ADC 1 (0.1 mg/kg dose-0.3 mg/kg dose) compared to the baseline (i.e., PBS) reticulocyte count. No depletion was observed for an isotype-AM control (data not shown) indicating on-target depletion of reticulocytes.

TABLE 5
AMINO ACID SEQUENCE SUMMARY

| Sequence | | |
|---|---|---|
| Identifier | Description | Amino Acid Sequence |
| SEQ ID NO: 1 | CK6 CDR-H1 | SYWIG |
| SEQ ID NO: 2 | CK6 CDR-H2 | IIYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Ab85 CDR-H1 | NYWIG |
| SEQ ID NO: 8 | Ab85 CDR-H2 | IINPRDSDTRYRPSFQG |
| SEQ ID NO: 9 | Ab85 CDR-H3 | HGRGYEGYEGAFDI |
| SEQ ID | Ab85 CDR-L1 | RSSQGIRSDLG |
| NO: 10 | | |
| SEQ ID | Ab85 CDR-L2 | DASNLET |
| NO: 11 | | |
| SEQ ID | Ab85 CDR-L3 | QQANGFPLT |
| NO: 12 | | |
| SEQ ID | Heavy chain | EVQLVQSGAEVKKPGESLKISCKGSGYSFTNYWIG |
| NO: 13 | variable region of | WVRQMPGKGLEWMAIINPRDSDTRYRPSFQGQVTI |
| | Ab 85 | SADKSISTAYLQWSSLKASDTAMYYCARHGRGYEG |
| | | YEGAFDIWGQGTLVTVSS |
| SEQ ID | Light chain variable | DIQMTQSPSSLSASVGDRVTITCRSSQGIRSDLGWY |
| NO: 14 | region of Ab 85 | QQKPGKAPKLLIYDASNLETGVPSRFSGSGSGTDFT |
| | | LTISSLQPEDFATYYCQQANGFPLTFGGGTKVEIK |
| SEQ ID | Heavy chain | ASTKGPSVFPLAPSSKSTSGGTAALGCLVKDYFPEP |
| NO: 15 | constant region | VTVSWNSGALTSGVHTFPAVLQSSGLYSLSSVVTVP |
| | (Wild type (WT)) | SSSLGTQTYICNVNHKPSNTKVDKKVEPKSCDKTHT |
| | | CPPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 16 | constant region | VTVSWNSGALTSGVHTFPAVLQSSGLYSLSSVVTVP |
| | with L234A, L235A | SSSLGTQTYICNVNHKPSNTKVDKKVEPKSCDKTHT |
| | (LALA) mutations | CPPCPAPEAAGGPSVFLFPPKPKDTLMISRTPEVTC |
| | (mutations in bold)* | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 17 | constant region | VTVSWNSGALTSGVHTFPAVLQSSGLYSLSSVVTVP |
| | with D265C | SSSLGTQTYICNVNHKPSNTKVDKKVEPKSCDKTHT |
| | mutation | CPPCPAPELLGGPSVFLFPPKPKDTLMISRTPEVTC |
| | (mutation in bold)* | VVVCVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQKS |
| | | LSLSPGK |
| SEQ ID | Heavy chain | ASTKGPSVFPLAPSSKSTSGGTAALGCLVKDYFPEP |
| NO: 18 | constant region | VTVSWNSGALTSGVHTFPAVLQSSGLYSLSSVVTVP |
| | with H435A | SSSLGTQTYICNVNHKPSNTKVDKKVEPKSCDKTHT |
| | mutation | CPPCPAPELLGGPSVFLFPPKPKDTLMISRTPEVTC |
| | (mutation in bold)* | VVVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNAYTQKS |
| | | LSLSPGK |
| SEQ ID | Heavy chain | ASTKGPSVFPLAPSSKSTSGGTAALGCLVKDYFPEP |
| NO: 19 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C mutations | VVVCVSHEDPEVKFNWYVDGVEVHNAKTKPREEQY |
| | (mutations in bold)* | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 20 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C, H435A | VVVCVSHEDPEVKFNWYVDGVEVHNAKTKPREEQY |
| | mutations | NSTYRVVSVLTVLHQDWLNGKEYKCKVSNKALPAPI |
| | (mutations in bold)* | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNAYTQ |
| | | KSLSLSPGK |
| SEQ ID | Ab85 full length | EVQLVQSGAEVKKPGESLKISCKGSGYSFTNYWIG |
| NO: 21 | heavy chain | WVRQMPGKGLEWMAIINPRDSDTRYRPSFQGQVTI |
| | sequence; constant | SADKSISTAYLQWSSLKASDTAMYYCARHGRGYEG |
| | region underlined | YEGAFDIWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNWY |
| | | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGK |
| SEQ ID | Ab85 full length | EVQLVQSGAEVKKPGESLKISCKGSGYSFTNYWIG |
| NO: 22 | heavy chain | WVRQMPGKGLEWMAIINPRDSDTRYRPSFQGQVTI |
| | sequence; constant | SADKSISTAYLQWSSLKASDTAMYYCARHGRGYEG |
| | region underlined; | YEGAFDIWGQGTLVTVSSASTKGPSVFPLAPSSKST |
| | modified Fc region | SGGTAALGCLVKDYFPEPVTVSWNSGALTSGVHTF |
| | with L234A, L235A | PAVLQSSGLYSLSSVVTVPSSSLGTQTYICNVNHKP |
| | mutations | SNTKVDKKVEPKSCDKTHTCPPCPAPEAAGGPSVF |
| | (mutations in bold)* | LFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID | Ab85 full length | EVQLVQSGAEVKKPGESLKISCKGSGYSFTNYWIG |
| NO: 23 | heavy chain | WVRQMPGKGLEWMAIINPRDSDTRYRPSFQGQVTI |
| | sequence: constant | SADKSISTAYLQWSSLKASDTAMYYCARHGRGYEG |
| | region underlined; | YEGAFDIWGQGTLVTVSSASTKGPSVFPLAPSSKST |
| | modified Fc region | SGGTAALGCLVKDYFPEPVTVSWNSGALTSGVHTF |
| | with L234A, L235A, | PAVLQSSGLYSLSSVVTVPSSSLGTQTYICNVNHKP |
| | D265C mutations | SNTKVDKKVEPKSCDKTHTCPPCPAPEAAGGPSVF |
| | (mutations in bold)* | LFPPKPKDTLMISRTPEVTCVVVCVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNHYTQKSLSLSPGK |
| SEQ ID | Ab85 full length | EVQLVQSGAEVKKPGESLKISCKGSGYSFTNYWIG |
| NO: 24 | heavy chain | WVRQMPGKGLEWMAIINPRDSDTRYRPSFQGQVTI |
| | sequence (LALA- | SADKSISTAYLQWSSLKASDTAMYYCARHGRGYEG |
| | D265C-H435A | YEGAFDIWGQGTLVTVSSASTKGPSVFPLAPSSKST |
| | mutant); constant | SGGTAALGCLVKDYFPEPVTVSWNSGALTSGVHTF |
| | region underlined | PAVLQSSGLYSLSSVVTVPSSSLGTQTYICNVNHKP |
| | | SNTKVDKKVEPKSCDKTHTCPPCPAPEAAGGPSVF |
| | | LFPPKPKDTLMISRTPEVTCVVVCVSHEDPEVKFNW |
| | | YVDGVEVHNAKTKPREEQYNSTYRVVSVLTVLHQD |
| | | WLNGKEYKCKVSNKALPAPIEKTISKAKGQPREPQV |
| | | YTLPPSRDELTKNQVSLTCLVKGFYPSDIAVEWESN |
| | | GQPENNYKTTPPVLDSDGSFFLYSKLTVDKSRWQQ |
| | | GNVFSCSVMHEALHNAYTQKSLSLSPGK |
| SEQ ID | Light chain | RTVAAPSVFIFPPSDEQLKSGTASVVCLLNNFYPREA |
| NO: 25 | constant region | KVQWKVDNALQSGNSQESVTEQDSKDSTYSLSSTL |
| | | TLSKADYEKHKVYACEVTHQGLSSPVTKSFNRGEC |
| SEQ ID | Ab85 full length | DIQMTQSPSSLSASVGDRVTITCRSSQGIRSDLGWY |
| NO: 26 | light chain; | QQKPGKAPKLLIYDASNLETGVPSRFSGSGSGTDFT |
| | constant region | LTISSLQPEDFATYYCQQANGFPLTFGGGTKVEIKRT |
| | underlined | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID | CK6 heavy chain | QVQLVQSGAAVKKPGESLKISCKGSGYRFTSYWIG |
| NO: 27 | variable region | WVRQMPGKGLEWMGIIYPGDSDTRYSPSFQGQVTI |
| | amino acid | SAGKSISTAYLQWSSLKASDTAMYYCARHGRGYNG |
| | sequence (CDRs | YEGAFDIWGQGTMVTVSS |
| | bold) | |
| SEQ ID | CK6 light chain | AIQLTQSPSSLSASVGDRVTITCRASQGISSALAWY |
| NO: 28 | variable region | QQKPGKAPKLLIYDASSLESGVPSRFSGSGSGTD |
| | amino acid | FTLTISSLQPEDFATYYCQQFNSYPLTFGGGTKVEIK |
| | sequence (CDRs | |
| | bold) | |
| SEQ ID NO: | Human CD2 | MSFPCKFVAS FLLIFNVSSK GAVSKEITNA |
| 29 | sequence | LETWGALGQD INLDIPSFQM SDDIDDIKWE |
| | | KTSDKKKIAQ FRKEKETFKE KDTYKLFKNG |
| | | TLKIKHLKTD DQDIYKVSIY DTKGKNVLEK |
| | | IFDLKIQERV SKPKISWICI NTTLTCEVMN |
| | | GTDPELNLYQ DGKHLKLSQR VITHKWTTSL |
| | | SAKFKCTAGN KVSKESSVEP VSCPEKGLDI |
| | | YLIIGICGGG SLLMVFVALL VFYITKRKKQ |
| | | RSRRNDEELE TRAHRVATEE RGRKPHQIPA |
| | | STPQNPATSQ HPPPPPGHRS QAPSHRPPPP |
| | | GHRVQHQPQK RPPAPSGTQV HQQKGPPLPR |
| | | PRVQPKPPHG AAENSLSPSS N |
| SEQ ID NO: | Anti-CD2 antibody | EYYMY |
| 30 | CDR-H1 | |
| SEQ ID NO: | Anti-CD2 antibody | RIDPEDGSIDYVEKFKK |
| 31 | CDR-H2 | |
| SEQ ID NO: | Anti-CD2 antibody | GKFNYRFAY |
| 32 | CDR-H3 | |
| SEQ ID NO: | Anti-CD2 antibody | RSSQSLLHSSGNTYLN |
| 33 | CDR-L1 | |
| SEQ ID NO: | Anti-CD2 antibody | LVSKLES |
| 34 | CDR-L2 | |
| SEQ ID NO: | Anti-CD2 antibody | MQFTHYPYT |
| 35 | CDR-L3 | |
| SEQ ID NO: | Anti-CD2 Heavy | QVQLVQSGAEVKKPGASVKVSCKASGYTFTEYYMY |
| 36 | chain variable | WVRQAPGQGLELMGRIDPEDGSIDYVEKFKKKVTLT |
| | region | ADTSSSTAYMELSSLTSDDTAVYYCARGKFNYRFAY |
| | | WGQGTLVTVSS |
| SEQ ID NO: | Anti-CD2 Light | DVVMTQSPPSLLVTLGQPASISCRSSQSLLHSSGNT |
| 37 | chain variable | YLNWLLQRPGQSPQPLIYLVSKLESGVPDRFSGSGS |
| | region | GTDFTLKISGVEAEDVGVYYCMQFTHYPYTFGQGTK |
| | | LEIK |
| SEQ ID NO: | Anti-CD2 (RPA- | GFTFSSY |
| 38 | 2.10) CDR-H1 | |
| SEQ ID NO: | Anti-CD2 (RPA- | SGGGF |
| 39 | 2.10) CDR-H2 | |
| SEQ ID NO: | Anti-CD2 (RPA- | SSYGEIMDY |
| 40 | 2.10) CDR-H3 | |
| | Variant 1 | |
| SEQ ID NO: | Anti-CD2 (RPA- | SSYGELMDY |
| 41 | 2.10) CDR-H3 | |
| | Variant 2 | |
| SEQ ID NO: | Anti-CD2 (RPA- | RASQRIGTSIH |
| 42 | 2.10) CDR-L1 | |
| SEQ ID NO: | Anti-CD2 (RPA- | YASESIS |
| 43 | 2.10) CDR-L2 | |
| SEQ ID NO: | Anti-CD2 (RPA- | QQSHGWPFTF |
| 44 | 2.10) CDR-L3 | |
| SEQ ID NO: | Anti-CD2 (RPA- | EVKLVESGGGLVKPGGSLKLSCAASGFTFSSYDMS |
| 45 | 2.10) Heavy chain | WVRQTPEKRLEWVASISGGGFLYYLDSVKGRFTISR |
| | variable region | DNARNILYLHMTSLRSEDTAMYYCARSSYGEIMDYW |
| | Variant 1 | GQGTSVTVSS |
| SEQ ID NO: | Anti-CD2 (RPA- | EVKLVESGGGLVKPGGSLKLSCAASGFTFSSYDMS |
| 46 | 2.10) Heavy chain | WVRQTPEKRLEWVASISGGGFLYYLDSVKGRFTISR |
| | variable region | DNARNILYLHMTSLRSEDTAMYYCARSSYGELMDY |
| | Variant 2 | WGQGTSVTVSS |
| SEQ ID NO: | Anti-CD2 (RPA- | DILLTQSPAILSVSPGERVSFSCRASQRIGTSIHWYQ |
| 47 | 2.10) Light chain | QRTTGSPRLLIKYASESISGIPSRFSGSGSGTDFTLSI |
| | variable region | NSVESEDVADYYCQQSHGWPFTFGGGTKLEIE |
| SEQ ID NO: | Human CD5 amino | MPMGSLQPLATLYLLGMLVASCLGRLSWYDPDFQA |
| 48 | acid sequence | RLTRSNSKCQGQLEVYLKDGWH MVCSQSWGRS |
| | (isoform 1) | SKQWEDPSQA SKVCQRLNCG VPLSLGPFLV |
| | | TYTPQSSIIC YGQLGSFSNCSHSRNDMCHS |
| | | LGLTCLEPQK TTPPTTRPPP TTTPEPTAPP |
| | | RLQLVAQSGG QHCAGVVEFYSGSLGGTISY |
| | | EAQDKTQDLE NFLCNNLQCG SFLKHLPETE |
| | | AGRAQDPGEP REHQPLPIQWKIQNSSCTSL |
| | | EHCFRKIKPQ KSGRVLALLC SGFQPKVQSR |
| | | LVGGSSICEG TVEVRQGAQWAALCDSSSAR |
| | | SSLRWEEVCR EQQCGSVNSY RVLDAGDPTS |
| | | RGLFCPHQKL SQCHELWERNSYCKKVFVTC |
| | | QDPNPAGLAA GTVASIILAL VLLVVLLVVC |
| | | GPLAYKKLVK KFRQKKQRQWIGPTGMNQNM |
| | | SFHRNHTATV RSHAENPTAS HVDNEYSQPP |
| | | RNSHLSAYPA LEGALHRSSMQPDNSSDSDY |
| | | DLHGAQRL |
| SEQ ID NO: | Anti-CD5 | QVTLKESGPVLVKPTETLTLTCTFSGFSLSTSGMGV |
| 49 | (humanized 5D7) | GWIRQAPGKGLEWVAHIWWDDDVYYNPSLKSRLTI |
| | Heavy chain | TKDASKDQVSLKLSSVTAADTAVYYCVRRRATGTGF |
| | variable region | DYWGQGTLVTVSS |
| | (CDRs in bold) | |
| SEQ ID NO: | Anti-CD5 | NIVMTQSPSSLSASVGDRVTITCQASQDVGTAVAWY |
| 50 | (humanized 5D7) | QQKPDQSPKLLIYWTSTRHTGVPDRFTGSGSGTDF |
| | Light chain | TLTISSLQPEDIATYFCHQYNSYNTFGSGTKLEIK |
| | variable region | |
| | (CDRs in bold) | |
| SEQ ID NO: | Anti-CD5 | FSLSTSGMG |
| 51 | (humanized 5D7) | |
| | CDR-H1 | |
| SEQ ID NO: | Anti-CD5 | WWDDD |
| 52 | (humanized 5D7) | |
| | CDR-H2 | |
| SEQ ID NO: | Anti-CD5 | RRATGTGFDY |
| 53 | (humanized 5D7) | |
| | CDR-H3 | |
| SEQ ID NO: | Anti-CD5 | QDVGTA |
| 54 | (humanized 5D7) | |
| | CDR-L1 | |
| SEQ ID NO: | Anti-CD5 | WTSTRHT |
| 55 | (humanized 5D7) | |
| | CDR-L2 | |
| SEQ ID NO: | Anti-CD5 | YNSYNT |
| 56 | (humanized 5D7) | |
| | CDR-L3 | |
| SEQ ID NOs: | CDR amino acid sequences for various anti-CD5 antibodies | |
| 57-277 | | |
| SEQ ID NO: | Anti-CD137 | STYWIS |
| 278 | antibody CDR-H1 | |
| SEQ ID NO: | Anti-CD137 | KIYPGDSYTNYSPSFQG |
| 279 | antibody CDR-H2 | |
| SEQ ID NO: | Anti-CD137 | RGYGIFDY |
| 280 | antibody CDR-H3 | |
| SEQ ID NO: | Anti-CD137 | SGDNIGDQYAH |
| 281 | antibody CDR-L1 | |
| SEQ ID NO: | Anti-CD137 | QDKNRPS |
| 282 | antibody CDR-L2 | |
| SEQ ID NO: | Anti-CD137 | ATYTGFGSLAV |
| 283 | antibody CDR-L3 | |
| SEQ ID NO: | Anti-CD137 | STYTFVGFTTV |
| 284 | antibody CDR-L3 | |
| SEQ ID NO: | Anti-CD137 | NSYAIS |
| 285 | antibody CDR-H1 | |
| SEQ ID NO: | Anti-CD137 | GIIPGFGTANYAQKFQG |
| 286 | antibody CDR-H2 | |
| SEQ ID NO: | Anti-CD137 | RKNEEDGGFDH |
| 287 | antibody CDR-H3 | |
| SEQ ID NO: | Anti-CD137 | SGDNLGDYYAS |
| 288 | antibody CDR-L1 | |
| SEQ ID NO: | Anti-CD137 | DDSNRPS |
| 289 | antibody CDR-L2 | |
| SEQ ID NO: | Anti-CD137 | QTWDGTLHFV |
| 290 | antibody CDR-L3 | |
| SEQ ID NO: | Anti-CD137 | SDYYMH |
| 291 | antibody CDR-H1 | |
| SEQ ID NO: | Anti-CD137 | VISGSGSNTYYADSVKG |
| 292 | antibody CDR-H2 | |
| SEQ ID NO: | Anti-CD137 | RLYAQFEGDF |
| 293 | antibody CDR-H3 | |
| SEQ ID NO: | Anti-CD137 | SGDNIGSKYVS |
| 294 | antibody CDR-L1 | |
| SEQ ID NO: | Anti-CD137 | SDSERRS |
| 295 | antibody CDR-L2 | |
| SEQ ID NO: | Anti-CD137 | QSWDGSISRV |
| 296 | antibody CDR-L3 | |
| SEQ ID NO: | Anti-CD137 (ch- | QVQLQQPGAELVRPGASVKLSCKASGYTFTSYWIN |
| 297 | BBK2) Heavy chain | WVKQRPGQGLEWIGNIYPSDSYTNYNQKFKDKATL |
| | sequence (CDRs in | TVDKSSNTVYMQLNSPTSEDSAVYYCTRNGVEGYP |
| | bold) | HYYAMEYWGQGTSVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNWY |
| | | VDGVEVHNAKTKPREEQYNSTYRVVSVLTVLHQDW |
| | | LNGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGK |
| SEQ ID NO: | Anti-CD137 (ch- | DIQMTQTTSALSASLGDRVTIGCRASQDLSNHLYWY |
| 298 | BBK2) Light chain | QQKPDGTVKLLIYYTSRLHSGVPSRFSGSGSGTDYS |
| | sequence (CDRs in | LTIRNLEQEDVATYFCQQGYTLPYTFGGGTKLEIKRT |
| | bold) | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: | Anti-CD137 (ch- | SGYTFTSYW |
| 299 | BBK2) CDR-H1 | |
| SEQ ID NO: | Anti-CD137 (ch- | NIYPSDSYT |
| 300 | BBK2) CDR-H2 | |
| SEQ ID NO: | Anti-CD137 (ch- | TRNGVEGYPHYYAME |
| 301 | BBK2) CDR-H3 | |
| SEQ ID NO: | Anti-CD137 (ch- | SQDLSNH |
| 302 | BBK2) CDR-L1 | |
| SEQ ID NO: | Anti-CD137 (ch- | YYTS |
| 303 | BBK2) CDR-L2 | |
| SEQ ID NO: | Anti-CD137 (ch- | CQQGYTLPY |
| 304 | BBK2) CDR-L3 | |
| SEQ ID NO: | Anti-CD137 (ch- | SYWIN |
| 305 | BBK2) CDR-H1 | |
| SEQ ID NO: | Anti-CD137 (ch- | NIYPSDSYTNYNQKFKD |
| 306 | BBK2) CDR-H2 | |
| SEQ ID NO: | Anti-CD137 (ch- | NGVEGYPHYYAMEY |
| 307 | BBK2) CDR-H3 | |
| SEQ ID NO: | Anti-CD137 (ch- | RASQDLSNHLY |
| 308 | BBK2) CDR-L1 | |
| SEQ ID NO: | Anti-CD137 (ch- | YTSRLHS |
| 309 | BBK2) CDR-L2 | |
| SEQ ID NO: | Anti-CD137 (ch- | QQGYTLPYT |
| 310 | BBK2) CDR-L3 | |
| SEQ ID NO: | Anti-CD137 Heavy | QVQLQQPGAELVRPGASVKLSCKASGYTFTSYWIN |
| 311 | chain variable | WVKQRPGQGLEWIGNIYPSDSYTNYNQKFKDKATL |
| | region sequence | TVDKSSNTVYMQLNSPTSEDSAVYYCTRNGVEGYP |
| | (BBK2) | HYYAMEYWGQGTSVTVSS |
| SEQ ID NO: | Anti-CD137 Light | DIQMTQTTSALSASLGDRVTIGCRASQDLSNHLYWY |
| 312 | chain variable | QQKPDGTVKLLIYYTSRLHSGVPSRFSGSGSGTDYS |
| | region sequence | LTIRNLEQEDVATYFCQQGYTLPYTFGGGTKLEIK |
| | (BBK2) | |
| SEQ ID NO: | Human CD252 | MERVQPLEENVGNAARPRFERNKLLLVASVIQGLGL |
| 313 | (i.e., OX40L)- | LLCFTYICLHFSALQVSHRYPRIQSIKVQFTEYKKEKG |
| | Isoform 1 | FILTSQKEDEIMKVQNNSVIINCDGFYLISLKGYFSQE |
| | | VNISLHYQKDEEPLFQLKKVRSVNSLMVASLTYKDK |
| | | VYLNVTTDNTSLDDFHVNGGELILIHQNPGEFCVL |
| SEQ ID NO: | Human CD252 | MVSHRYPRIQSIKVQFTEYKKEKGFILTSQKEDEIMK |
| 314 | (i.e., OX40L)- | VQNNSVIINCDGFYLISLKGYFSQEVNISLHYQKDEE |
| | Isoform 2 | PLFQLKKVRSVNSLMVASLTYKDKVYLNVTTDNTSL |
| | | DDFHVNGGELILIHQNPGEFCVL |
| SEQ ID NO: | Anti-CD252 VH | EVQLVESGGGLVQPGGSLRLSCAASGFTFSNYAMN |
| 315 | amino acid | WVRQAPGKGLEWVSTISGSGGATRYADSVKGRFTI |
| | sequence | SRDNSRNTVYLQMNSLRVEDTAVFYCTKDRLIMATV |
| | | RGPYYYGMDVWGQGTTVTVSS |
| SEQ ID NO: | Anti-CD252 VL | DIQMTQSPSSLSASVGDRVTITCRASQSISSYLNWY |
| 316 | amino acid | QQKPGKAPNLLIYAASSLQSGVPSRFSGSGSETDFT |
| | sequence | LTISSLQPEDFATYYCQQSHSVSFTFGPGTKVDIK |
| SEQ ID NO: | Anti-CD252 CDR- | GFTFSNYA |
| 317 | H1 amino acid | |
| | sequence | |
| SEQ ID NO: | Anti-CD252 CDR- | ISGSGGAT |
| 318 | H2 amino acid | |
| | sequence | |
| SEQ ID NO: | Anti-CD252 CDR- | TKDRLIMATVRGPYYYGMDV |
| 319 | H3 amino acid | |
| | sequence | |
| SEQ ID NO: | Anti-CD252 CDR- | QSISSY |
| 320 | L1 amino acid | |
| | sequence | |
| SEQ ID NO: | Anti-CD252 CDR- | AAS |
| 321 | L2 amino acid | |
| | sequence | |
| SEQ ID NO: | Anti-CD252 CDR- | QQSHSVSFT |
| 322 | L3 amino acid | |
| | sequence | |
| SEQ ID NO: | oxelumab full | EVQLLESGGGLVQPGGSLRLSCAASGFTFNSYAMS |
| 323 | length heavy chain | WVRQAPGKGLEWVSIISGSGGFTYYADSVKGRFTIS |
| | amino acid | RDNSRTTLYLQMNSLRAEDTAVYYCAKDRLVAPGTF |
| | sequence | DYWGQGALVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKV |
| | | DKKVEPKSCDKTHTCPPCPAPELLGGPSVFLFPPKP |
| | | KDTLMISRTPEVTCVVVDVSHEDPEVKFNWYVDGVE |
| | | VHNAKTKPREEQYNSTYRVVSVLTVLHQDWLNGKE |
| | | YKCKVSNKALPAPIEKTISKAKGQPREPQVYTLPPSR |
| | | DELTKNQVSLTCLVKGFYPSDIAVEWESNGQPENNY |
| | | KTTPPVLDSDGSFFLYSKLTVDKSRWQQGNVFSCS |
| | | VMHEALHNHYTQKSLSLSPG |
| SEQ ID NO: | oxelumab full | DIQMTQSPSSLSASVGDRVTITCRASQGISSWLAWY |
| 324 | length light chain | QQKPEKAPKSLIYAASSLQSGVPSRFSGSGSGTDFT |
| | amino acid | LTISSLQPEDFATYYCQQYNSYPYTFGQGTKLEIKRT |
| | sequence | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: | oxelumab CDR-H1 | GFTFNSYA |
| 325 | amino acid | |
| | sequence | |
| SEQ ID NO: | oxelumab CDR-H2 | ISGSGGFT |
| 326 | amino acid | |
| | sequence | |
| SEQ ID NO: | oxelumab CDR-H3 | AKDRLVAPGTFDY |
| 327 | amino acid | |
| | sequence | |
| SEQ ID NO: | oxelumab CDR-L1 | QGISSW |
| 328 | amino acid | |
| | sequence | |
| SEQ ID NO: | oxelumab CDR-L2 | AAS |
| 329 | amino acid | |
| | sequence | |
| SEQ ID NO: | oxelumab CDR-L3 | QQYNSYPYT |
| 330 | amino acid | |
| | sequence | |
| SEQ ID NO: | oxelumab VH | EVQLLESGGGLVQPGGSLRLSCAASGFTFNSYAMS |
| 331 | amino acid | WVRQAPGKGLEWVSIISGSGGFTYYADSVKGRFTIS |
| | sequence | RDNSRTTLYLQMNSLRAEDTAVYYCAKDRLVAPGTF |
| | | DYWGQGALVTVSS |
| SEQ ID NO: | oxelumab VL | DIQMTQSPSSLSASVGDRVTITCRASQGISSWLAWY |
| 332 | amino acid | QQKPEKAPKSLIYAASSLQSGVPSRFSGSGSGTDFT |
| | sequence | LTISSLQPEDFATYYCQQYNSYPYTFGQGTKLEIK |
| SEQ ID NO: | CD45RA (Human | MTMYLWLKLLAFGFAFLDTEVFVTGQSPTPSPTGLT |
| 333 | CD45 Isoform) | TAKMPSVPLSSDPLPTHTTAFSPASTFERENDFSET |
| | | TTSLSPDNTSTQVSPDSLDNASAFNTTDAYLNASET |
| | | TTLSPSGSAVISTTTIATTPSKPTCDEKYANITVDYLY |
| | | NKETKLFTAKLNVNENVECGNNTCTNNEVHNLTECK |
| | | NASVSISHNSCTAPDKTLILDVPPGVEKFQLHDCTQV |
| | | EKADTTICLKWKNIETFTCDTQNITYRFQCGNMIFDN |
| | | KEIKLENLEPEHEYKCDSEILYNNHKFTNASKIIKTDF |
| | | GSPGEPQIIFCRSEAAHQGVITWNPPQRSFHNFTLC |
| | | YIKETEKDCLNLDKNLIKYDLQNLKPYTKYVLSLHAYII |
| | | AKVQRNGSAAMCHFTTKSAPPSQVWNMTVSMTSD |
| | | NSMHVKCRPPRDRNGPHERYHLEVEAGNTLVRNES |
| | | HKNCDFRVKDLQYSTDYTFKAYFHNGDYPGEPFILH |
| | | HSTSYNSKALIAFLAFLIIVTSIALLVVLYKIYDLHKKRS |
| | | CNLDEQQELVERDDEKQLMNVEPIHADILLETYKRKI |
| | | ADEGRLFLAEFQSIPRVFSKFPIKEARKPFNQNKNRY |
| | | VDILPYDYNRVELSEINGDAGSNYINASYIDGFKEPR |
| | | KYIAAQGPRDETVDDFWRMIWEQKATVIVMVTRCEE |
| | | GNRNKCAEYWPSMEEGTRAFGDVVVKINQHKRCP |
| | | DYIIQKLNIVNKKEKATGREVTHIQFTSWPDHGVPED |
| | | PHLLLKLRRRVNAFSNFFSGPIVVHCSAGVGRIGTYI |
| | | GIDAMLEGLEAENKVDVYGYVVKLRRQRCLMVQVE |
| | | AQYILIHQALVEYNQFGETEVNLSELHPYLHNMKKRD |
| | | PPSEPSPLEAEFQRLPSYRSWRTQHIGNQEENKSK |
| | | NRNSNVIPYDYNRVPLKHELEMSKESEHDSDESSDD |
| | | DSDSEEPSKYINASFIMSYWKPEVMIAAQGPLKETIG |
| | | DFWQMIFQRKVKVIVMLTELKHGDQEICAQYWGEG |
| | | KQTYGDIEVDLKDTDKSSTYTLRVFELRHSKRKDSR |
| | | TVYQYQYTNWSVEQLPAEPKELISMIQVVKQKLPQK |
| | | NSSEGNKHHKSTPLLIHCRDGSQQTGIFCALLNLLES |
| | | AETEEVVDIFQVVKALRKARPGMVSTFEQYQFLYDVI |
| | | ASTYPAQNGQVKKNNHQEDKIEFDNEVDKVKQDAN |
| | | CVNPLGAPEKLPEAKEQAEGSEPTSGTEGPEHSVN |
| | | GPASPALNQGS |
| SEQ ID NO: | CD45RB (Human | MTMYLWLKLLAFGFAFLDTEVFVTGQSPTPSPTGVS |
| 334 | CD45 Isoform) | SVQTPHLPTHADSQTPSAGTDTQTFSGSAANAKLNP |
| | | TPGSNAISDAYLNASETTTLSPSGSAVISTTTIATTPS |
| | | KPTCDEKYANITVDYLYNKETKLFTAKLNVNENVECG |
| | | NNTCTNNEVHNLTECKNASVSISHNSCTAPDKTLILD |
| | | VPPGVEKFQLHDCTQVEKADTTICLKWKNIETFTCDT |
| | | QNITYRFQCGNMIFDNKEIKLENLEPEHEYKCDSEIL |
| | | YNNHKFTNASKIIKTDFGSPGEPQIIFCRSEAAHQGVI |
| | | TWNPPQRSFHNFTLCYIKETEKDCLNLDKNLIKYDLQ |
| | | NLKPYTKYVLSLHAYIIAKVQRNGSAAMCHFTTKSAP |
| | | PSQVWNMTVSMTSDNSMHVKCRPPRDRNGPHERY |
| | | HLEVEAGNTLVRNESHKNCDFRVKDLQYSTDYTFKA |
| | | YFHNGDYPGEPFILHHSTSYNSKALIAFLAFLIIVTSIA |
| | | LLVVLYKIYDLHKKRSCNLDEQQELVERDDEKQLMN |
| | | VEPIHADILLETYKRKIADEGRLFLAEFQSIPRVFSKFP |
| | | IKEARKPFNQNKNRYVDILPYDYNRVELSEINGDAGS |
| | | NYINASYIDGFKEPRKYIAAQGPRDETVDDFWRMIW |
| | | EQKATVIVMVTRCEEGNRNKCAEYWPSMEEGTRAF |
| | | GDVVVKINQHKRCPDYIIQKLNIVNKKEKATGREVTHI |
| | | QFTSWPDHGVPEDPHLLLKLRRRVNAFSNFFSGPIV |
| | | VHCSAGVGRTGTYIGIDAMLEGLEAENKVDVYGYVV |
| | | KLRRQRCLMVQVEAQYILIHQALVEYNQFGETEVNL |
| | | SELHPYLHNMKKRDPPSEPSPLEAEFQRLPSYRSW |
| | | RTQHIGNQEENKSKNRNSNVIPYDYNRVPLKHELEM |
| | | SKESEHDSDESSDDDSDSEEPSKYINASFIMSYWKP |
| | | EVMIAAQGPLKETIGDFWQMIFQRKVKVIVMLTELKH |
| | | GDQEICAQYWGEGKQTYGDIEVDLKDTDKSSTYTLR |
| | | VFELRHSKRKDSRTVYQYQYTNWSVEQLPAEPKELI |
| | | SMIQVVKQKLPQKNSSEGNKHHKSTPLLIHCRDGSQ |
| | | QTGIFCALLNLLESAETEEVVDIFQVVKALRKARPGM |
| | | VSTFEQYQFLYDVIASTYPAQNGQVKKNNHQEDKIE |
| | | FDNEVDKVKQDANCVNPLGAPEKLPEAKEQAEGSE |
| | | PTSGTEGPEHSVNGPASPALNQGS |
| SEQ ID NO: | CD45RC (Human | MTMYLWLKLLAFGFAFLDTEVFVTGQSPTPSPTDVP |
| 335 | CD45 Isoform) | GERSTASTFPTDPVSPLTTTLSLAHHSSAALPARTSN |
| | | TTITANTSDAYLNASETTTLSPSGSAVISTTTIATTPSK |
| | | PTCDEKYANITVDYLYNKETKLFTAKLNVNENVECG |
| | | NNTCTNNEVHNLTECKNASVSISHNSCTAPDKTLILD |
| | | VPPGVEKFQLHDCTQVEKADTTICLKWKNIETFTCDT |
| | | QNITYRFQCGNMIFDNKEIKLENLEPEHEYKCDSEIL |
| | | YNNHKFTNASKIIKTDFGSPGEPQIIFCRSEAAHQGVI |
| | | TWNPPQRSFHNFTLCYIKETEKDCLNLDKNLIKYDLQ |
| | | NLKPYTKYVLSLHAYIIAKVQRNGSAAMCHFTTKSAP |
| | | PSQVWNMTVSMTSDNSMHVKCRPPRDRNGPHERY |
| | | HLEVEAGNTLVRNESHKNCDFRVKDLQYSTDYTFKA |
| | | YFHNGDYPGEPFILHHSTSYNSKALIAFLAFLIIVTSIA |
| | | LLVVLYKIYDLHKKRSCNLDEQQELVERDDEKQLMN |
| | | VEPIHADILLETYKRKIADEGRLFLAEFQSIPRVFSKFP |
| | | IKEARKPFNQNKNRYVDILPYDYNRVELSEINGDAGS |
| | | NYINASYIDGFKEPRKYIAAQGPRDETVDDFWRMIW |
| | | EQKATVIVMVTRCEEGNRNKCAEYWPSMEEGTRAF |
| | | GDVVVKINQHKRCPDYIIQKLNIVNKKEKATGREVTHI |
| | | QFTSWPDHGVPEDPHLLLKLRRRVNAFSNFFSGPIV |
| | | VHCSAGVGRTGTYIGIDAMLEGLEAENKVDVYGYVV |
| | | KLRRQRCLMVQVEAQYILIHQALVEYNQFGETEVNL |
| | | SELHPYLHNMKKRDPPSEPSPLEAEFQRLPSYRSW |
| | | RTQHIGNQEENKSKNRNSNVIPYDYNRVPLKHELEM |
| | | SKESEHDSDESSDDDSDSEEPSKYINASFIMSYWKP |
| | | EVMIAAQGPLKETIGDFWQMIFQRKVKVIVMLTELKH |
| | | GDQEICAQYWGEGKQTYGDIEVDLKDTDKSSTYTLR |
| | | VFELRHSKRKDSRTVYQYQYTNWSVEQLPAEPKELI |
| | | SMIQVVKQKLPQKNSSEGNKHHKSTPLLIHCRDGSQ |
| | | QTGIFCALLNLLESAETEEVVDIFQVVKALRKARPGM |
| | | VSTFEQYQFLYDVIASTYPAQNGQVKKNNHQEDKIE |
| | | FDNEVDKVKQDANCVNPLGAPEKLPEAKEQAEGSE |
| | | PTSGTEGPEHSVNGPASPALNQGS |
| SEQ ID NO: | CD45RO (Human | MTMYLWLKLLAFGFAFLDTEVFVTGQSPTPSPTDAY |
| 336 | CD45 Isoform) | LNASETTTLSPSGSAVISTTTIATTPSKPTCDEKYANI |
| | | TVDYLYNKETKLFTAKLNVNENVECGNNTCTNNEVH |
| | | NLTECKNASVSISHNSCTAPDKTLILDVPPGVEKFQL |
| | | HDCTQVEKADTTICLKWKNIETFTCDTQNITYRFQCG |
| | | NMIFDNKEIKLENLEPEHEYKCDSEILYNNHKFTNAS |
| | | KIIKTDFGSPGEPQIIFCRSEAAHQGVITWNPPQRSF |
| | | HNFTLCYIKETEKDCLNLDKNLIKYDLQNLKPYTKYVL |
| | | SLHAYIIAKVQRNGSAAMCHFTTKSAPPSQVWNMTV |
| | | SMTSDNSMHVKCRPPRDRNGPHERYHLEVEAGNTL |
| | | VRNESHKNCDFRVKDLQYSTDYTFKAYFHNGDYPG |
| | | EPFILHHSTSYNSKALIAFLAFLIIVTSIALLVVLYKIYDL |
| | | HKKRSCNLDEQQELVERDDEKQLMNVEPIHADILLE |
| | | TYKRKIADEGRLFLAEFQSIPRVFSKFPIKEARKPFNQ |
| | | NKNRYVDILPYDYNRVELSEINGDAGSNYINASYIDG |
| | | FKEPRKYIAAQGPRDETVDDFWRMIWEQKATVIVMV |
| | | TRCEEGNRNKCAEYWPSMEEGTRAFGDVVVKINQH |
| | | KRCPDYIIQKLNIVNKKEKATGREVTHIQFTSWPDHG |
| | | VPEDPHLLLKLRRRVNAFSNFFSGPIVVHCSAGVGR |
| | | TGTYIGIDAMLEGLEAENKVDVYGYVVKLRRQRCLM |
| | | VQVEAQYILIHQALVEYNQFGETEVNLSELHPYLHNM |
| | | KKRDPPSEPSPLEAEFQRLPSYRSWRTQHIGNQEE |
| | | NKSKNRNSNVIPYDYNRVPLKHELEMSKESEHDSDE |
| | | SSDDDSDSEEPSKYINASFIMSYWKPEVMIAAQGPL |
| | | KETIGDFWQMIFQRKVKVIVMLTELKHGDQEICAQY |
| | | WGEGKQTYGDIEVDLKDTDKSSTYTLRVFELRHSKR |
| | | KDSRTVYQYQYTNWSVEQLPAEPKELISMIQVVKQK |
| | | LPQKNSSEGNKHHKSTPLLIHCRDGSQQTGIFCALL |
| | | NLLESAETEEVVDIFQVVKALRKARPGMVSTFEQYQ |
| | | FLYDVIASTYPAQNGQVKKNNHQEDKIEFDNEVDKV |
| | | KQDANCVNPLGAPEKLPEAKEQAEGSEPTSGTEGP |
| | | EHSVNGPASPALNQGS |
| SEQ ID NO: | Apamistamab | EVKLLESGGGLVQPGGSLKLSCAASGFDFSRYWMS |
| 337 | Heavy Chain | WVRQAPGKGLEWIGEINPTSSTINFTPSLKDKVFISR |
| | Variable Region | DNAKNTLYLQMSKVRSEDTALYYCARGNYYRYGDA |
| | | MDYWGQGTSVTVSSA |
| SEQ ID NO: | Apamistamab Light | DIALTQSPASLAVSLGQRATISCRASKSVSTSGYSYL |
| 338 | Chain Variable | HWYQQKPGQPPKLLIYLASNLESGVPARFSGSGSG |
| | Region | TDFTLNIHPVEEEDAATYYCQHSRELPFTFGSGTKLE |
| | | IKR |
| SEQ ID NO: | Human CD5 amino | MVCSQSWGRSSKQWEDPSQASKVCQRLNCGVPLS |
| 339 | acid sequence | LGPFLVTYTPQSSIICYGQLGSFSNCSHSRNDMCHS |
| | (isoform 2) | LGLTCLEPQKTTPPTTRPPPTTTPEPTAPPRLQLVAQ |
| | | SGGQHCAGVVEFYSGSLGGTISYEAQDKTQDLENF |
| | | LCNNLQCGSFLKHLPETEAGRAQDPGEPREHQPLPI |
| | | QWKIQNSSCTSLEHCFRKIKPQKSGRVLALLCSGFQ |
| | | PKVQSRLVGGSSICEGTVEVRQGAQWAALCDSSSA |
| | | RSSLRWEEVCREQQCGSVNSYRVLDAGDPTSRGLF |
| | | CPHQKLSQCHELWERNSYCKKVFVTCQDPNPAGLA |
| | | AGTVASIILALVLLVVLLVVCGPLAYKKLVKKFRQKKQ |
| | | RQWIGPTGMNQNMSFHRNHTATVRSHAENPTASH |
| | | VDNEYSQPPRNSHLSAYPALEGALHRSSMQPDNSS |
| | | DSDYDLHGAQRL |
| SEQ ID | Ab249 CDR-H1 | TSWIG |
| NO: 340 | | |
| SEQ ID | Ab249 CDR-H2 | IIYPGDSDTRYSPSFQG |
| NO: 341 | | |
| SEQ ID | Ab249 CDR-H3 | HGLGYNGYEGAFDI |
| NO: 342 | | |
| SEQ ID | Ab249 CDR-L1 | RASQGIGSALA |
| NO: 343 | | |
| SEQ ID | Ab249 CDR-L2 | DASNLET |
| NO: 344 | | |
| SEQ ID | Ab249 CDR-L3 | QQLNGYPLT |
| NO: 345 | | |
| SEQ ID | Heavy chain | EVQLVQSGAEVKKPGESLKISCKGSGYRFTTSWIGW |
| NO: 346 | variable region of | VRQMPGKGLEWMGIIYPGDSDTRYSPSFQGQVTISA |
| | Ab 249 | DKSISTAYLQWSSLKASDTAMYYCARHGLGYNGYE |
| | | GAFDIWGQGTLVTVSS |
| SEQ ID | Light chain variable | DIQMTQSPSSLSASVGDRVTITCRASQGIGSALAWY |
| NO: 347 | region of Ab 249 | QQKPGKAPKLLIYDASNLETGVPSRFSGSGSGTDFT |
| | | LTISSLQPEDFATYYCQQLNGYPLTFGQGTRLEIK |
| SEQ ID | Ab67 CDR-H1 | FTFSDADMD |
| NO: 348 | | |
| SEQ ID | Ab67 CDR-H2 | RTRNKAGSYTTEYAASVKG |
| NO: 349 | | |
| SEQ ID | Ab67 CDR-H3 | AREPKYWIDFDL |
| NO: 350 | | |
| SEQ ID | Ab67 CDR-L1 | RASQSISSYLN |
| NO: 351 | | |
| SEQ ID | Ab67 CDR-L2 | AASSLQS |
| NO: 352 | | |
| SEQ ID | Ab67 CDR-L3 | QQSYIAPYT |
| NO: 353 | | |
| SEQ ID | Heavy chain | EVQLVESGGGLVQPGGSLRLSCAASGFTFSDADMD |
| NO: 354 | variable region of | WVRQAPGKGLEWVGRTRNKAGSYTTEYAASVKGR |
| | Ab 67 | FTISRDDSKNSLYLQMNSLKTEDTAVYYCAREPKYW |
| | | IDFDLWGRGTLVTVSS |
| SEQ ID | Light chain variable | DIQMTQSPSSLSASVGDRVTITCRASQSISSYLNWY |
| NO: 355 | region of Ab 67 | QQKPGKAPKLLIYAASSLQSGVPSRFSGSGSGTDFT |
| | | LTISSLQPEDFATYYCQQSYIAPYTFGGGTKVEIK |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. An antibody, or antigen-binding portion thereof, comprising an Fc region, wherein the Fc region comprises amino acid substitutions consisting essentially of amino acid substitutions L234A, L235A, and D265C (EU index.

2. An antibody, or antigen-binding portion thereof, comprising an Fc region, wherein the Fc region comprises amino acid substitutions consisting essentially of amino acid substitutions L234A, L235A, S239C and D265A (EU index).

3. An antibody, or antigen-binding portion thereof, comprising an Fc region, wherein the Fc region comprises amino acid substitutions consisting essentially of amino acid substitutions H435A, L234A, L235A, and D265C (EU index).

4. The antibody, or antigen-binding portion of claim 1, wherein the antibody, or the antigen-binding region, is conjugated to a cytotoxin.

5. The antibody, or antigen-binding portion of claim 2, wherein the antibody, or the antigen-binding region, is conjugated to a cytotoxin.

6. The antibody, or antigen-binding portion of claim 3, wherein the antibody, or the antigen-binding region, is conjugated to a cytotoxin.


SEQUENCE LISTING (355 sequences)

SEQ ID NO: 1 - protein, 5 aa, Artificial Sequence
  Description of Artificial Sequence Synthetic peptide
SYWIG

SEQ ID NO: 2 - protein, 17 aa, Artificial Sequence
  Description of Artificial Sequence Synthetic peptide
IIYPGDSDTRYSPSFQG

SEQ ID NO: 3 - protein, 14 aa, Artificial Sequence
  Description of Artificial Sequence Synthetic peptide
HGRGYNGYEGAFDI

SEQ ID NO: 4 - protein, 11 aa, Artificial Sequence
  Description of Artificial Sequence Synthetic peptide
RASQGISSALA

SEQ ID NO: 5 - protein, 7 aa, Artificial Sequence
  Description of Artificial Sequence Synthetic peptide
DASSLES

SEQ ID NO: 6 - protein, 10 aa, Artificial Sequence
  Description of Artificial Sequence Synthetic peptide
CQQFNSYPLT

SEQ ID NO: 7 - protein, 5 aa, Artificial Sequence
  Description of Artificial Sequence Synthetic peptide
NYWIG

SEQ ID NO: 8 - protein, 17 aa, Artificial Sequence
  Description of Artificial Sequence Synthetic peptide
IINPRDSDTRYRPSFQG

SEQ ID NO: 9 - protein, 14 aa, Artificial Sequence
  Description of Artificial Sequence Synthetic peptide
HGRGYEGYEGAFDI

SEQ ID NO: 10 - protein, 11 aa, Artificial Sequence
  Description of Artificial Sequence Synthetic peptide
RSSQGIRSDLG

SEQ ID NO: 11 - protein, 7 aa, Artificial Sequence
  Description of Artificial Sequence Synthetic peptide
DASNLET

SEQ ID NO: 12 - protein, 9 aa, Artificial Sequence
  Description of Artificial Sequence Synthetic peptide
QQANGFPLT

SEQ ID NO: 13 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 14 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 15 - protein, 330 aa, Homo sapiens [sequence omitted]

SEQ ID NO: 16 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 19 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 21 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 25 - protein, 107 aa, Artificial Sequence [sequence omitted]

SEQ ID NO: 26 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 28 - protein, 107 aa, Artificial Sequence [sequence omitted]

SEQ ID NO: 29 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 30 - protein, 5 aa, Artificial Sequence
  Description of Artificial Sequence Synthetic peptide
EYYMY

SEQ ID NO: 31 - protein, 17 aa, Artificial Sequence
  Description of Artificial Sequence Synthetic peptide
RIDPEDGSIDYVEKFKK

SEQ ID NO: 32 - protein, 9 aa, Artificial Sequence
  Description of Artificial Sequence Synthetic peptide
GKFNYRFAY

SEQ ID NO: 33 - protein, 16 aa, Artificial Sequence
  Description of Artificial Sequence Synthetic peptide
RSSQSLLHSSGNTYLN

SEQ ID NO: 34 - protein, 7 aa, Artificial Sequence
  Description of Artificial Sequence Synthetic peptide
LVSKLES

SEQ ID NO: 35 - protein, 9 aa, Artificial Sequence
  Description of Artificial Sequence Synthetic peptide
MQFTHYPYT

SEQ ID NO: 36 - protein, 118 aa, Artificial Sequence
  Description of Artificial Sequence Synthetic polypeptide
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 37 - protein, 112 aa, Artificial Sequence
  Description of Artificial Sequence Synthetic polypeptide
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 38 - protein, 7 aa, Artificial Sequence
  Description of Artificial Sequence Synthetic peptide
GFTFSSY

SEQ ID NO: 39 - protein, 5 aa, Artificial Sequence
  Description of Artificial Sequence Synthetic peptide
SGGGF

SEQ ID NO: 40 - protein, 9 aa, Artificial Sequence
  Description of Artificial Sequence Synthetic peptide
SSYGEIMDY

SEQ ID NO: 41 - protein, 9 aa, Artificial Sequence
  Description of Artificial Sequence Synthetic peptide
SSYGELMDY

SEQ ID NO: 42 - protein, 11 aa, Artificial Sequence
  Description of Artificial Sequence Synthetic peptide
RASQRIGTSIH

SEQ ID NO: 43 - protein, 7 aa, Artificial Sequence
  Description of Artificial Sequence Synthetic peptide
YASESIS

SEQ ID NO: 44 - protein, 10 aa, Artificial Sequence
  Description of Artificial Sequence Synthetic peptide
QQSHGWPFTF

SEQ ID NO: 45 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSS

SEQ ID NO: 46 - protein, 117 aa, Artificial Sequence
  Description of Artificial Sequence Synthetic polypeptide
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGELMDYWGQGTSVTVSS

SEQ ID NO: 47 - protein, 107 aa, Artificial Sequence
  Description of Artificial Sequence Synthetic polypeptide
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIE

SEQ ID NO: 48 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 49 - protein, 120 aa, Artificial Sequence
  Description of Artificial Sequence Synthetic polypeptide
QVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 50 - protein, 106 aa, Artificial Sequence
  Description of Artificial Sequence Synthetic polypeptide
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIK

SEQ ID NO: 51 - protein, 9 aa, Artificial Sequence
  Description of Artificial Sequence Synthetic peptide
FSLSTSGMG

SEQ ID NO: 52 - protein, 5 aa, Artificial Sequence
  Description of Artificial Sequence Synthetic peptide
WWDDD

SEQ ID NO: 53 - protein, 10 aa, Artificial Sequence
  Description of Artificial Sequence Synthetic peptide
RRATGTGFDY

SEQ ID NO: 54 - protein, 6 aa, Artificial Sequence
  Description of Artificial Sequence Synthetic peptide
QDVGTA

SEQ ID NO: 55 - protein, 7 aa, Artificial Sequence
  Description of Artificial Sequence Synthetic peptide
WTSTRHT

SEQ ID NO: 56 - protein, 6 aa, Artificial Sequence
  Description of Artificial Sequence Synthetic peptide
YNSYNT

SEQ ID NO: 57 - protein, 10 aa, Artificial Sequence
  Description of Artificial Sequence Synthetic peptide
SGYSFTGYTM

SEQ ID NO: 58 - protein, 10 aa, Artificial Sequence
  Description of Artificial Sequence Synthetic peptide
SGYSFTDYTM

SEQ ID NO: 59 - protein, 10 aa, Artificial Sequence
  Description of Artificial Sequence Synthetic peptide
SGYSFTGYTM

SEQ ID NO: 60 - protein, 10 aa, Artificial Sequence
  Description of Artificial Sequence Synthetic peptide
SGYSFTGYTM

SEQ ID NO: 61 - protein, 10 aa, Artificial Sequence
  Description of Artificial Sequence Synthetic peptide
SGYSFTGYTM

SEQ ID NO: 62 - protein, 10 aa, Artificial Sequence
  Description of Artificial Sequence Synthetic peptide
SGFTFSNYAM

SEQ ID NO: 63 - protein, 10 aa, Artificial Sequence
  Description of Artificial Sequence Synthetic peptide
SGFTFSSYAM

SEQ ID NO: 64 - protein, 10 aa, Artificial Sequence
  Description of Artificial Sequence Synthetic peptide
SGYSFTAYNI

SEQ ID NO: 65 - protein, 10 aa, Artificial Sequence
  Description of Artificial Sequence Synthetic peptide
SGYSFTAYSM

SEQ ID NO: 66 - protein, 10 aa, Artificial Sequence
  Description of Artificial Sequence Synthetic peptide
SGYTFTNFAI

SEQ ID NO: 67 - protein, 10 aa, Artificial Sequence
  Description of Artificial Sequence Synthetic peptide
SGYTFTNFAI

SEQ ID NO: 68 - protein, 10 aa, Artificial Sequence
  Description of Artificial Sequence Synthetic peptide
SGYTFTNFAI

SEQ ID NO: 69 - protein, 10 aa, Artificial Sequence
  Description of Artificial Sequence Synthetic peptide
SGYTFTNFAI

SEQ ID NO: 70 - protein, 10 aa, Artificial Sequence
  Description of Artificial Sequence Synthetic peptide
SGFNIKDTYM

SEQ ID NO: 71 - protein, 10 aa, Artificial Sequence
  Description of Artificial Sequence Synthetic peptide
SGYSFTSYWM

SEQ ID NO: 72 - protein, 10 aa, Artificial Sequence
  Description of Artificial Sequence Synthetic peptide
SGFSLTNYDV

SEQ ID NO: 73 - protein, 10 aa, Artificial Sequence
  Description of Artificial Sequence Synthetic peptide
SGFSLTNYDV

SEQ ID NO: 74 - protein, 10 aa, Artificial Sequence
  Description of Artificial Sequence Synthetic peptide
SGFTFSNYGM

SEQ ID NO: 75 - protein, 10 aa, Artificial Sequence
  Description of Artificial Sequence Synthetic peptide
SGYSFTGYTM

SEQ ID NO: 76 - protein, 10 aa, Artificial Sequence
  Description of Artificial Sequence Synthetic peptide
SGYIFANYGM

SEQ ID NO: 77 - protein, 10 aa, Artificial Sequence
  Description of Artificial Sequence Synthetic peptide
SGYNFTNYGM

SEQ ID NO: 78 - protein, 10 aa, Artificial Sequence
  Description of Artificial Sequence Synthetic peptide
SGYTFTNYGM

SEQ ID NO: 79 - protein, 10 aa, Artificial Sequence
  Description of Artificial Sequence Synthetic peptide
SGYTFTDYYI

SEQ ID NO: 80 - protein, 10 aa, Artificial Sequence
  Description of Artificial Sequence Synthetic peptide
SGYTFTDYYI

SEQ ID NO: 81 - protein, 10 aa, Artificial Sequence
  Description of Artificial Sequence Synthetic peptide
SGNTFTNFYL

SEQ ID NO: 82 - protein, 10 aa, Artificial Sequence
  Description of Artificial Sequence Synthetic peptide
SGYTFTNYGM

SEQ ID NO: 83 - protein, 10 aa, Artificial Sequence
  Description of Artificial Sequence Synthetic peptide
SEFTFSNYAM

SEQ ID NO: 84 - protein, 10 aa, Artificial Sequence
  Description of Artificial Sequence Synthetic peptide
SGYTFTSYRM

SEQ ID NO: 85 - protein, 10 aa, Artificial Sequence
  Description of Artificial Sequence Synthetic peptide
SGFNIKDTYM

SEQ ID NO: 86 - protein, 10 aa, Artificial Sequence
  Description of Artificial Sequence Synthetic peptide
SGYSFTDYTM

SEQ ID NO: 87 - protein, 10 aa, Artificial Sequence
  Description of Artificial Sequence Synthetic peptide
SGYMFTNHGM

SEQ ID NO: 88 - protein, 10 aa, Artificial Sequence
  Description of Artificial Sequence Synthetic peptide
SGYMFTNYGM

SEQ ID NO: 89 - protein, 10 aa, Artificial Sequence
  Description of Artificial Sequence Synthetic peptide
SGYIFTNYGM

SEQ ID NO: 90 - protein, 10 aa, Artificial Sequence
  Description of Artificial Sequence Synthetic peptide
SGFNIKDYYI

SEQ ID NO: 91 - protein, 10 aa, Artificial Sequence
  Description of Artificial Sequence Synthetic peptide
SGYTFINYGM

SEQ ID NO: 92 - protein, 10 aa, Artificial Sequence
  Description of Artificial Sequence Synthetic peptide
SGYTFTDYFI

SEQ ID NO: 93 - protein, 10 aa, Artificial Sequence
  Description of Artificial Sequence Synthetic peptide
SGYIFTGYNI

SEQ ID NO: 94 - protein, 10 aa, Artificial Sequence
  Description of Artificial Sequence Synthetic peptide
LINPYNGGTT

SEQ ID NO: 95 - protein, 10 aa, Artificial Sequence
  Description of Artificial Sequence Synthetic peptide
LINPYNGGTM

SEQ ID NO: 96 - protein, 10 aa, Artificial Sequence
  Description of Artificial Sequence Synthetic peptide
LINPYNGGTM

SEQ ID NO: 97 - protein, 10 aa, Artificial Sequence
  Description of Artificial Sequence Synthetic peptide
LINPYNGGTM

SEQ ID NO: 98 - protein, 10 aa, Artificial Sequence
  Description of Artificial Sequence Synthetic peptide
LINPYNGGTT

SEQ ID NO: 99 - protein, 9 aa, Artificial Sequence
  Description of Artificial Sequence Synthetic peptide
SISSGGNTF

SEQ ID NO: 100 - protein, 9 aa, Artificial Sequence
  Description of Artificial Sequence Synthetic peptide
SISSGGSTY

SEQ ID NO: 101 - protein, 10 aa, Artificial Sequence
  Description of Artificial Sequence Synthetic peptide
SIDPYYGDTK

SEQ ID NO: 102 - protein, 10 aa, Artificial Sequence
  Description of Artificial Sequence Synthetic peptide
SIDPYYGDTK

SEQ ID NO: 103 - protein, 10 aa, Artificial Sequence
  Description of Artificial Sequence Synthetic peptide
LISSNSGDVS

SEQ ID NO: 104 - protein, 10 aa, Artificial Sequence
  Description of Artificial Sequence Synthetic peptide
LISTSSGDVS

SEQ ID NO: 105 - protein, 10 aa, Artificial Sequence
  Description of Artificial Sequence Synthetic peptide
LISSNSGDVS

SEQ ID NO: 106 - protein, 10 aa, Artificial Sequence
  Description of Artificial Sequence Synthetic peptide
LISSNSGDVS

SEQ ID NO: 107 - protein, 10 aa, Artificial Sequence
  Description of Artificial Sequence Synthetic peptide
RIDPANGNTK

SEQ ID NO: 108 - protein, 10 aa, Artificial Sequence
  Description of Artificial Sequence Synthetic peptide
MIHPSDSETR

SEQ ID NO: 109 - protein, 9 aa, Artificial Sequence
  Description of Artificial Sequence Synthetic peptide
VIWSGGNTD

SEQ ID NO: 110 - protein, 9 aa, Artificial Sequence
  Description of Artificial Sequence Synthetic peptide
VIWSGGNTD

SEQ ID NO: 111 - protein, 10 aa, Artificial Sequence
  Description of Artificial Sequence Synthetic peptide
AINSNGDITY

SEQ ID NO: 112 - protein, 10 aa, Artificial Sequence
  Description of Artificial Sequence Synthetic peptide
LINPYNGGTR

SEQ ID NO: 113 - protein, 10 aa, Artificial Sequence
  Description of Artificial Sequence Synthetic peptide
WINTYTGEPT

SEQ ID NO: 114 - protein, 10 aa, Artificial Sequence
  Description of Artificial Sequence Synthetic peptide
WINTYTGEPT

SEQ ID NO: 115 - protein, 10 aa, Artificial Sequence
  Description of Artificial Sequence Synthetic peptide
WINTYTGEPT

SEQ ID NO: 116 - protein, 10 aa, Artificial Sequence
  Description of Artificial Sequence Synthetic peptide
WIYPGGGNTR

SEQ ID NO: 117 - protein, 10 aa, Artificial Sequence
  Description of Artificial Sequence Synthetic peptide
WIYPGGGNTR

SEQ ID NO: 118 - protein, 10 aa, Artificial Sequence
  Description of Artificial Sequence Synthetic peptide
CIYPGNVKTK

SEQ ID NO: 119 - protein, 10 aa, Artificial Sequence
  Description of Artificial Sequence Synthetic peptide
WINTYTGEPT

SEQ ID NO: 120 - protein, 10 aa, Artificial Sequence
  Description of Artificial Sequence Synthetic peptide
TISSGGSYTY

SEQ ID NO: 121 - protein, 10 aa, Artificial Sequence
  Description of Artificial Sequence Synthetic peptide
RIDPYDSGTH

SEQ ID NO: 122 - protein, 10 aa, Artificial Sequence
  Description of Artificial Sequence Synthetic peptide
RIDPANGNTK

SEQ ID NO: 123 - protein, 10 aa, Artificial Sequence
  Description of Artificial Sequence Synthetic peptide
LINPYNGGTR

SEQ ID NO: 124 - protein, 10 aa, Artificial Sequence
  Description of Artificial Sequence Synthetic peptide
WINTYTGEPT

SEQ ID NO: 125 - protein, 10 aa, Artificial Sequence
  Description of Artificial Sequence Synthetic peptide
WINTYTGEPT

SEQ ID NO: 126 - protein, 10 aa, Artificial Sequence
  Description of Artificial Sequence Synthetic peptide
WINTYTGEPT

SEQ ID NO: 127 - protein, 10 aa, Artificial Sequence
  Description of Artificial Sequence Synthetic peptide
WIDPENGRTE

SEQ ID NO: 128 - protein, 10 aa, Artificial Sequence
  Description of Artificial Sequence Synthetic peptide
WINTYTGEPT

SEQ ID NO: 129 - protein, 10 aa, Artificial Sequence
  Description of Artificial Sequence Synthetic peptide
EIYPGSSNTY

SEQ ID NO: 130 - protein, 10 aa, Artificial Sequence
  Description of Artificial Sequence Synthetic peptide
AVYPGNGDTS

SEQ ID NO: 131 - protein, 15 aa, Artificial Sequence
  Description of Artificial Sequence Synthetic peptide
CARDYYGSSPDFDYW

SEQ ID NO: 132 - protein, 15 aa, Artificial Sequence
  Description of Artificial Sequence Synthetic peptide
CARDNYGSSPDFDYW

SEQ ID NO: 133 - protein, 15 aa, Artificial Sequence
  Description of Artificial Sequence Synthetic peptide
CARDNYGSSPYFDYW

SEQ ID NO: 134 - protein, 15 aa, Artificial Sequence
  Description of Artificial Sequence Synthetic peptide
CARDNYGSSPYFDYW

SEQ ID NO: 135 - protein, 15 aa, Artificial Sequence
  Description of Artificial Sequence Synthetic peptide
CARDYYGSSPDFDYW

SEQ ID NO: 136 - protein, 16 aa, Artificial Sequence
  Description of Artificial Sequence Synthetic peptide
CVRYYYGVTYWYFDVW

SEQ ID NO: 137 - protein, 16 aa, Artificial Sequence
  Description of Artificial Sequence Synthetic peptide
CVRYYYGIRYWYFDVW

SEQ ID NO: 138 - protein, 16 aa, Artificial Sequence
  Description of Artificial Sequence Synthetic peptide
CARRMITMGDWYFDVW

SEQ ID NO: 139 - protein, 16 aa, Artificial Sequence
  Description of Artificial Sequence Synthetic peptide
CARRMITTGDWYFDVW

SEQ ID NO: 140 - protein, 14 aa, Artificial Sequence
  Description of Artificial Sequence Synthetic peptide
CARHYGAHNYFDYW

SEQ ID NO: 141 - protein, 14 aa, Artificial Sequence
  Description of Artificial Sequence Synthetic peptide
CARHYGANNYFDYW

SEQ ID NO: 142 - protein, 14 aa, Artificial Sequence
  Description of Artificial Sequence Synthetic peptide
CARHYGAHNYFDYW

SEQ ID NO: 143 - protein, 14 aa, Artificial Sequence
  Description of Artificial Sequence Synthetic peptide
CARHYGAHNYFDYW

SEQ ID NO: 144 - protein, 16 aa, Artificial Sequence
  Description of Artificial Sequence Synthetic peptide
CAREENYYGTYYFDYW

SEQ ID NO: 145 - protein, 14 aa, Artificial Sequence
  Description of Artificial Sequence Synthetic peptide
CARWGDHDDAMDFW

SEQ ID NO: 146 - protein, 17 aa, Artificial Sequence
  Description of Artificial Sequence Synthetic peptide
CARNHGDGYFNWYFDVW

SEQ ID NO: 147 - protein, 17 aa, Artificial Sequence
  Description of Artificial Sequence Synthetic peptide
CARNHGDGYYNWYFDVW

SEQ ID NO: 148 - protein, 11 aa, Artificial Sequence
  Description of Artificial Sequence Synthetic peptide
CARGTAWFTYW

SEQ ID NO: 149 - protein, 14 aa, Artificial Sequence
  Description of Artificial Sequence Synthetic peptide
CARDGDDGWDIDVW

SEQ ID NO: 150 - protein, 13 aa, Artificial Sequence
  Description of Artificial Sequence Synthetic peptide
CARRGTYWHFDVW

SEQ ID NO: 151 - protein, 13 aa, Artificial Sequence
  Description of Artificial Sequence Synthetic peptide
CARRGSYWHFDVW

SEQ ID NO: 152 - protein, 12 aa, Artificial Sequence
  Description of Artificial Sequence Synthetic peptide
CARRSTLVFDYW

SEQ ID NO: 153 - protein, 12 aa, Artificial Sequence
  Description of Artificial Sequence Synthetic peptide
CARNGYWYFDVW

SEQ ID NO: 154 - protein, 12 aa, Artificial Sequence
  Description of Artificial Sequence Synthetic peptide
CARNGYWYFDVW

SEQ ID NO: 155 - protein, 16 aa, Artificial Sequence
  Description of Artificial Sequence Synthetic peptide
CAKEGDYDGTAYFDYW

SEQ ID NO: 156 - protein, 12 aa, Artificial Sequence
  Description of Artificial Sequence Synthetic peptide
CARRRDGNFDYW

SEQ ID NO: 157 - protein, 10 aa, Artificial Sequence
  Description of Artificial Sequence Synthetic peptide
CVRHGYFDVW

SEQ ID NO: 158 - protein, 9 aa, Artificial Sequence
  Description of Artificial Sequence Synthetic peptide
CAFYDGAYW

SEQ ID NO: 159 - protein, 9 aa, Artificial Sequence
  Description of Artificial Sequence Synthetic peptide
CASYDPDYW

SEQ ID NO: 160 - protein, 14 aa, Artificial Sequence
  Description of Artificial Sequence Synthetic peptide
CARDTTATYYFDYW

SEQ ID NO: 161 - protein, 12 aa, Artificial Sequence
  Description of Artificial Sequence Synthetic peptide
CARRVATYFDVW

SEQ ID NO: 162 - protein, 12 aa, Artificial Sequence
  Description of Artificial Sequence Synthetic peptide
CTRRSHITLDYW

SEQ ID NO: 163 - protein, 12 aa, Artificial Sequence
  Description of Artificial Sequence Synthetic peptide
CARRRTTAFDYW

SEQ ID NO: 164 - protein, 12 aa, Artificial Sequence
  Description of Artificial Sequence Synthetic peptide
CTRRREITFDYW

SEQ ID NO: 165 - protein, 12 aa, Artificial Sequence
  Description of Artificial Sequence Synthetic peptide
CARSGISPFTYW

SEQ ID NO: 166 - protein, 11 aa, Artificial Sequence
  Description of Artificial Sequence Synthetic peptide
CAKYDRFFASW

SEQ ID NO: 167 - protein, 8 aa, Artificial Sequence
  Description of Artificial Sequence Synthetic peptide
SQGISNHL

SEQ ID NO: 168 - protein, 15 aa, Artificial Sequence
  Description of Artificial Sequence Synthetic peptide
CNNGNYVRHYYFDYW

SEQ ID NO: 169 - protein, 8 aa, Artificial Sequence
  Description of Artificial Sequence Synthetic peptide
SQGISNHL

SEQ ID NO: 170 - protein, 8 aa, Artificial Sequence
  Description of Artificial Sequence Synthetic peptide
SQGINNYL

SEQ ID NO: 171 - protein, 8 aa, Artificial Sequence
  Description of Artificial Sequence Synthetic peptide
SQGISNHL

SEQ ID NO: 172 - protein, 12 aa, Artificial Sequence
  Description of Artificial Sequence Synthetic peptide
SQSVDHDGDSYM

SEQ ID NO: 173 - protein, 12 aa, Artificial Sequence
  Description of Artificial Sequence Synthetic peptide
SQSVDYDGDSYM

SEQ ID NO: 174 - protein, 8 aa, Artificial Sequence
  Description of Artificial Sequence Synthetic peptide
SQDISNYL

SEQ ID NO: 175 - protein, 8 aa, Artificial Sequence
  Description of Artificial Sequence Synthetic peptide
SQDISTYL

SEQ ID NO: 176 - protein, 9 aa, Artificial Sequence
  Description of Artificial Sequence Synthetic peptide
TSSISSSYL

SEQ ID NO: 177 - protein, 9 aa, Artificial Sequence
  Description of Artificial Sequence Synthetic peptide
NSSVSSSYL

SEQ ID NO: 178 - protein, 9 aa, Artificial Sequence
  Description of Artificial Sequence Synthetic peptide
TSSISSSYL

SEQ ID NO: 179 - protein, 9 aa, Artificial Sequence
  Description of Artificial Sequence Synthetic peptide
TSSISSSYL

SEQ ID NO: 180 - protein, 8 aa, Artificial Sequence
  Description of Artificial Sequence Synthetic peptide
SENIYYNL

SEQ ID NO: 181 - protein, 8 aa, Artificial Sequence
  Description of Artificial Sequence Synthetic peptide
SENIYGYF

SEQ ID NO: 182 - protein, 8 aa, Artificial Sequence
  Description of Artificial Sequence Synthetic peptide
SQDINNYI

SEQ ID NO: 183 - protein, 8 aa, Artificial Sequence
  Description of Artificial Sequence Synthetic peptide
SQDINKYI

SEQ ID NO: 184 - protein, 8 aa, Artificial Sequence
  Description of Artificial Sequence Synthetic peptide
SENIYSYL

SEQ ID NO: 185 - protein, 8 aa, Artificial Sequence
  Description of Artificial Sequence Synthetic peptide
SQGIRNYL

SEQ ID NO: 186 - protein, 8 aa, Artificial Sequence
  Description of Artificial Sequence Synthetic peptide
SQDVRTDV

SEQ ID NO: 187 - protein, 8 aa, Artificial Sequence
  Description of Artificial Sequence Synthetic peptide
SQDVITAV

SEQ ID NO: 188 - protein, 8 aa, Artificial Sequence
  Description of Artificial Sequence Synthetic peptide
SQSIGTSI

SEQ ID NO: 189 - protein, 12 aa, Artificial Sequence
  Description of Artificial Sequence Synthetic peptide
SSQSLLNQKNYL

SEQ ID NO: 190 - protein, 9 aa, Artificial Sequence
  Description of Artificial Sequence Synthetic peptide
SSSVSSSYL

SEQ ID NO: 191 - protein, 8 aa, Artificial Sequence
  Description of Artificial Sequence Synthetic peptide
SENIYYNL

SEQ ID NO: 192 - protein, 8 aa, Artificial Sequence
  Description of Artificial Sequence Synthetic peptide
SQTIGTSI

SEQ ID NO: 193 - protein, 14 aa, Artificial Sequence
  Description of Artificial Sequence Synthetic peptide
SQSLLYSSDQKNYL

SEQ ID NO: 194 - protein, 7 aa, Artificial Sequence
  Description of Artificial Sequence Synthetic peptide
NSSVSYM

SEQ ID NO: 195 - protein, 8 aa, Artificial Sequence
  Description of Artificial Sequence Synthetic peptide
SENIYYNL

SEQ ID NO: 196 - protein, 7 aa, Artificial Sequence
  Description of Artificial Sequence Synthetic peptide
SSSLSYM

SEQ ID NO: 197 - protein, 8 aa, Artificial Sequence
  Description of Artificial Sequence Synthetic peptide
SQRIGTSM

SEQ ID NO: 198 - protein, 8 aa, Artificial Sequence
  Description of Artificial Sequence Synthetic peptide
SQSIGTSI

SEQ ID NO: 199 - protein, 8 aa, Artificial Sequence
  Description of Artificial Sequence Synthetic peptide
SQNIGTSI

SEQ ID NO: 200 - protein, 7 aa, Artificial Sequence
  Description of Artificial Sequence Synthetic peptide
ISSVSYM

SEQ ID NO: 201 - protein, 8 aa, Artificial Sequence
  Description of Artificial Sequence Synthetic peptide
SQTIATSI

SEQ ID NO: 202 - protein, 13 aa, Artificial Sequence
  Description of Artificial Sequence Synthetic peptide
SQSLVHSNGNTYL

SEQ ID NO: 203 - protein, 12 aa, Artificial Sequence
  Description of Artificial Sequence Synthetic peptide
NESVEYSGTSLM

SEQ ID NO: 204 - protein, 5 aa, Artificial Sequence
  Description of Artificial Sequence Synthetic peptide
YFTSS

SEQ ID NO: 205 - protein, 5 aa, Artificial Sequence
  Description of Artificial Sequence Synthetic peptide
YFTSS

SEQ ID NO: 206 - protein, 5 aa, Artificial Sequence
  Description of Artificial Sequence Synthetic peptide
YFTSS

SEQ ID NO: 207 - protein, 5 aa, Artificial Sequence
  Description of Artificial Sequence Synthetic peptide
YYTSS

SEQ ID NO: 208 - protein, 5 aa, Artificial Sequence
  Description of Artificial Sequence Synthetic peptide
YFTSS

SEQ ID NO: 209 - protein, 5 aa, Artificial Sequence
  Description of Artificial Sequence Synthetic peptide
YAASN

SEQ ID NO: 210 - protein, 5 aa, Artificial Sequence
  Description of Artificial Sequence Synthetic peptide
YAASN

SEQ ID NO: 211 - protein, 5 aa, Artificial Sequence
  Description of Artificial Sequence Synthetic peptide
YYTSR

SEQ ID NO: 212 - protein, 5 aa, Artificial Sequence
  Description of Artificial Sequence Synthetic peptide
FYTSR

SEQ ID NO: 213 - protein, 5 aa, Artificial Sequence
  Description of Artificial Sequence Synthetic peptide
YGTSN

SEQ ID NO: 214 - protein, 5 aa, Artificial Sequence
  Description of Artificial Sequence Synthetic peptide
YGTSN

SEQ ID NO: 215 - protein, 5 aa, Artificial Sequence
  Description of Artificial Sequence Synthetic peptide
YGTSN

SEQ ID NO: 216 - protein, 5 aa, Artificial Sequence
  Description of Artificial Sequence Synthetic peptide
YGTSN

SEQ ID NO: 217 - protein, 5 aa, Artificial Sequence
  Description of Artificial Sequence Synthetic peptide
YNANS

SEQ ID NO: 218 - protein, 5 aa, Artificial Sequence
  Description of Artificial Sequence Synthetic peptide
YNAKT

SEQ ID NO: 219 - protein, 5 aa, Artificial Sequence
  Description of Artificial Sequence Synthetic peptide
HYTST

SEQ ID NO: 220 - protein, 5 aa, Artificial Sequence
  Description of Artificial Sequence Synthetic peptide
HYTST

SEQ ID NO: 221 - protein, 5 aa, Artificial Sequence
  Description of Artificial Sequence Synthetic peptide
YNAKT

SEQ ID NO: 222 - protein, 5 aa, Artificial Sequence
  Description of Artificial Sequence Synthetic peptide
YHTST

SEQ ID NO: 223 - protein, 5 aa, Artificial Sequence
  Description of Artificial Sequence Synthetic peptide
YSASF

SEQ ID NO: 224 - protein, 5 aa, Artificial Sequence
  Description of Artificial Sequence Synthetic peptide
YSASY

SEQ ID NO: 225 - protein, 5 aa, Artificial Sequence
  Description of Artificial Sequence Synthetic peptide
KSASE

SEQ ID NO: 226 - protein, 5 aa, Artificial Sequence
  Description of Artificial Sequence Synthetic peptide
YWAST

SEQ ID NO: 227 - protein, 5 aa, Artificial Sequence
  Description of Artificial Sequence Synthetic peptide
YSTSN

SEQ ID NO: 228 - protein, 5 aa, Artificial Sequence
  Description of Artificial Sequence Synthetic peptide
YNANS

SEQ ID NO: 229 - protein, 5 aa, Artificial Sequence
  Description of Artificial Sequence Synthetic peptide
KNASE

SEQ ID NO: 230 - protein, 5 aa, Artificial Sequence
  Description of Artificial Sequence Synthetic peptide
YWAST

SEQ ID NO: 231 - protein, 5 aa, Artificial Sequence
  Description of Artificial Sequence Synthetic peptide
YDTSK

SEQ ID NO: 232 - protein, 5 aa, Artificial Sequence
  Description of Artificial Sequence Synthetic peptide
YNANS

SEQ ID NO: 233 - protein, 5 aa, Artificial Sequence
  Description of Artificial Sequence Synthetic peptide
YDTSN

SEQ ID NO: 234 - protein, 5 aa, Artificial Sequence
  Description of Artificial Sequence Synthetic peptide
KSASE

SEQ ID NO: 235 - protein, 5 aa, Artificial Sequence
  Description of Artificial Sequence Synthetic peptide
KSASE

SEQ ID NO: 236 - protein, 5 aa, Artificial Sequence
  Description of Artificial Sequence Synthetic peptide
KDASE

SEQ ID NO: 237 - protein, 5 aa, Artificial Sequence
  Description of Artificial Sequence Synthetic peptide
YATSN

SEQ ID NO: 238 - protein, 5 aa, Artificial Sequence
  Description of Artificial Sequence Synthetic peptide
KNASE

SEQ ID NO: 239 - protein, 5 aa, Artificial Sequence
  Description of Artificial Sequence Synthetic peptide
YKVSN

SEQ ID NO: 240 - protein, 5 aa, Artificial Sequence
  Description of Artificial Sequence Synthetic peptide
SAASN

SEQ ID NO: 241 - protein, 11 aa, Artificial Sequence
  Description of Artificial Sequence Synthetic peptide
CQQYSNLPYTF

SEQ ID NO: 242 - protein, 11 aa, Artificial Sequence
  Description of Artificial Sequence Synthetic peptide
CQQYSNLPYTF

SEQ ID NO: 243 - protein, 11 aa, Artificial Sequence
  Description of Artificial Sequence Synthetic peptide
CQQYSNLPYTF

SEQ ID NO: 244 - protein, 11 aa, Artificial Sequence
  Description of Artificial Sequence Synthetic peptide
CQQYSKIPYTC

SEQ ID NO: 245 - protein, 11 aa, Artificial Sequence
  Description of Artificial Sequence Synthetic peptide
CQQYSNLPYTF

SEQ ID NO: 246 - protein, 10 aa, Artificial Sequence
  Description of Artificial Sequence Synthetic peptide
CQQNYEDPTF

SEQ ID NO: 247 - protein, 10 aa, Artificial Sequence
  Description of Artificial Sequence Synthetic peptide
CQQSNEDPTF

SEQ ID NO: 248 - protein, 11 aa, Artificial Sequence
  Description of Artificial Sequence Synthetic peptide
CQQGDALPWTF

SEQ ID NO: 249 - protein, 11 aa, Artificial Sequence [sequence omitted]

SEQ ID NO: 250 - protein, 11 aa, Artificial Sequence
  Description of Artificial Sequence Synthetic peptide
CQQWSSRPPTF

SEQ ID NO: 251 - protein, 11 aa, Artificial Sequence [sequence omitted]

SEQ ID NO: 252 - protein, 11 aa, Artificial Sequence [sequence omitted]

SEQ ID NO: 253 - protein, 11 aa, Artificial Sequence
  Description of Artificial Sequence Synthetic peptide
CQQRSYFPFTF

SEQ ID NO: 254 - protein, 11 aa, Artificial Sequence
  Description of Artificial Sequence Synthetic peptide
CKQVYDVPFTF

SEQ ID NO: 255 - protein, 11 aa, Artificial Sequence
  Description of Artificial Sequence Synthetic peptide
CQHHYGTPFTF

SEQ ID NO: 256 - protein, 10 aa, Artificial Sequence
  Description of Artificial Sequence Synthetic peptide
CLQYDNLWTF

SEQ ID NO: 257 - protein, 10 aa, Artificial Sequence
  Description of Artificial Sequence Synthetic peptide
CLQYDNLWTF

SEQ ID NO: 258 - protein, 11 aa, Artificial Sequence
  Description of Artificial Sequence Synthetic peptide
CQHHYGYPYTF

SEQ ID NO: 259 - protein, 11 aa, Artificial Sequence [sequence omitted]

SEQ ID NO: 260 - protein, 11 aa, Artificial Sequence
  Description of Artificial Sequence Synthetic peptide
CQQHYTSPWTF

SEQ ID NO: 261 - protein, 11 aa, Artificial Sequence [sequence omitted]

SEQ ID NO: 262 - protein, 11 aa, Artificial Sequence
  Description of Artificial Sequence Synthetic peptide
CQQSNRWPLTF

SEQ ID NO: 263 - protein, 11 aa, Artificial Sequence
  Description of Artificial Sequence Synthetic peptide
CQNDYDYPYTF

SEQ ID NO: 264 - protein, 11 aa, Artificial Sequence
  Description of Artificial Sequence Synthetic peptide
CHQYHRSPLTF

SEQ ID NO: 265 - protein, 11 aa, Artificial Sequence
  Description of Artificial Sequence Synthetic peptide
CQQTFDVPWTF

SEQ ID NO: 266 - protein, 11 aa, Artificial Sequence
  Description of Artificial Sequence Synthetic peptide
CQQSNSWPLTY

SEQ ID NO: 267 - protein, 11 aa, Artificial Sequence [sequence omitted]

SEQ ID NO: 268 - protein, 11 aa, Artificial Sequence
  Description of Artificial Sequence Synthetic peptide
CQQWSSNPFTF

SEQ ID NO: 269 - protein, 11 aa, Artificial Sequence
  Description of Artificial Sequence Synthetic peptide
CKQAYDVPWTF

SEQ ID NO: 270 - protein, 11 aa, Artificial Sequence
  Description of Artificial Sequence Synthetic peptide
CQQWSSFPPTF

SEQ ID NO: 271 - protein, 11 aa, Artificial Sequence
  Description of Artificial Sequence Synthetic peptide
CQQSNSWPLTF

SEQ ID NO: 272 - protein, 11 aa, Artificial Sequence
  Description of Artificial Sequence Synthetic peptide
CQQSNSWPLTF

SEQ ID NO: 273 - protein, 11 aa, Artificial Sequence
  Description of Artificial Sequence Synthetic peptide
CQQSDSWPLTF

SEQ ID NO: 274 - protein, 11 aa, Artificial Sequence
  Description of Artificial Sequence Synthetic peptide
CQQWSSNPRTF

SEQ ID NO: 275 - protein, 11 aa, Artificial Sequence
  Description of Artificial Sequence Synthetic peptide
CQQSNSWPLTF

SEQ ID NO: 276 - protein, 11 aa, Artificial Sequence
  Description of Artificial Sequence Synthetic peptide
CWQNTHFPQTF

SEQ ID NO: 277 - protein, 11 aa, Artificial Sequence
  Description of Artificial Sequence Synthetic peptide
CQQSRQVPLTF

SEQ ID NO: 278 - protein, 6 aa, Artificial Sequence
  Description of Artificial Sequence Synthetic peptide
STYWIS

SEQ ID NO: 279 - protein, 17 aa, Artificial Sequence
  Description of Artificial Sequence Synthetic peptide
KIYPGDSYTNYSPSFQG

SEQ ID NO: 280 - protein, 8 aa, Artificial Sequence
  Description of Artificial Sequence Synthetic peptide
RGYGIFDY

SEQ ID NO: 281 - protein, 11 aa, Artificial Sequence
  Description of Artificial Sequence Synthetic peptide
SGDNIGDQYAH

SEQ ID NO: 282 - protein, 7 aa, Artificial Sequence
  Description of Artificial Sequence Synthetic peptide
QDKNRPS

SEQ ID NO: 283 - protein, 11 aa, Artificial Sequence
  Description of Artificial Sequence Synthetic peptide
ATYTGFGSLAV

SEQ ID NO: 284 - protein, 11 aa, Artificial Sequence
  Description of Artificial Sequence Synthetic peptide
STYTFVGFTTV

SEQ ID NO: 285 - protein, 6 aa, Artificial Sequence
  Description of Artificial Sequence Synthetic peptide
NSYAIS

SEQ ID NO: 286 - protein, 17 aa, Artificial Sequence
  Description of Artificial Sequence Synthetic peptide
GIIPGFGTANYAQKFQG

SEQ ID NO: 287 - protein, 11 aa, Artificial Sequence
  Description of Artificial Sequence Synthetic peptide
RKNEEDGGFDH

SEQ ID NO: 288 - protein, 11 aa, Artificial Sequence
  Description of Artificial Sequence Synthetic peptide
SGDNLGDYYAS

SEQ ID NO: 289 - protein, 7 aa, Artificial Sequence
  Description of Artificial Sequence Synthetic peptide
DDSNRPS

SEQ ID NO: 290 - protein, 10 aa, Artificial Sequence
  Description of Artificial Sequence Synthetic peptide
QTWDGTLHFV

SEQ ID NO: 291 - protein, 6 aa, Artificial Sequence
  Description of Artificial Sequence Synthetic peptide
SDYYMH

SEQ ID NO: 292 - protein, 17 aa, Artificial Sequence
  Description of Artificial Sequence Synthetic peptide
VISGSGSNTYYADSVKG

SEQ ID NO: 293 - protein, 10 aa, Artificial Sequence
  Description of Artificial Sequence Synthetic peptide
RLYAQFEGDF

SEQ ID NO: 294 - protein, 11 aa, Artificial Sequence
  Description of Artificial Sequence Synthetic peptide
SGDNIGSKYVS

SEQ ID NO: 295 - protein, 7 aa, Artificial Sequence
  Description of Artificial Sequence Synthetic peptide
SDSERPS

SEQ ID NO: 296 - protein, 10 aa, Artificial Sequence
  Description of Artificial Sequence Synthetic peptide
QSWDGSISRV

SEQ ID NO: 297 - protein, 453 aa, Artificial Sequence
  Description of Artificial Sequence Synthetic polypeptide
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 214 aa, Artificial Sequence
  Description of Artificial Sequence Synthetic polypeptide
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 9 aa, Artificial Sequence
  Description of Artificial Sequence Synthetic peptide
SGYTFTSYW

SEQ ID NO: 300 - protein, 9 aa, Artificial Sequence
  Description of Artificial Sequence Synthetic peptide
NIYPSDSYT

SEQ ID NO: 301 - protein, 15 aa, Artificial Sequence
  Description of Artificial Sequence Synthetic peptide
TRNGVEGYPHYYAME

SEQ ID NO: 302 - protein, 7 aa, Artificial Sequence
  Description of Artificial Sequence Synthetic peptide
SQDLSNH

SEQ ID NO: 303 - protein, 4 aa, Artificial Sequence
  Description of Artificial Sequence Synthetic peptide
YYTS

SEQ ID NO: 304 - protein, 9 aa, Artificial Sequence
  Description of Artificial Sequence Synthetic peptide
CQQGYTLPY

SEQ ID NO: 305 - protein, 5 aa, Artificial Sequence
  Description of Artificial Sequence Synthetic peptide
SYWIN

SEQ ID NO: 306 - protein, 17 aa, Artificial Sequence
  Description of Artificial Sequence Synthetic peptide
NIYPSDSYTNYNQKFKD

SEQ ID NO: 307 - protein, 14 aa, Artificial Sequence
  Description of Artificial Sequence Synthetic peptide
NGVEGYPHYYAMEY

SEQ ID NO: 308 - protein, 11 aa, Artificial Sequence
  Description of Artificial Sequence Synthetic peptide
RASQDLSNHLY

SEQ ID NO: 309 - protein, 7 aa, Artificial Sequence
  Description of Artificial Sequence Synthetic peptide
YTSRLHS

SEQ ID NO: 310 - protein, 9 aa, Artificial Sequence
  Description of Artificial Sequence Synthetic peptide
QQGYTLPYT

SEQ ID NO: 311 - protein, 123 aa, Artificial Sequence
  Description of Artificial Sequence Synthetic polypeptide
QVQLQQPGAELVRPGASVKLSCKASGYTFTSYWINWVKQRPGQGLEWIGNIYPSDSYTNYNQKFKDKATLTVDKSSNTVYMQLNSPTSEDSAVYYCTRNGVEGYPHYYAMEYWGQGTSVTVSS

SEQ ID NO: 312 - protein, 107 aa, Artificial Sequence
  Description of Artificial Sequence Synthetic polypeptide
DIQMTQTTSALSASLGDRVTIGCRASQDLSNHLYWYQQKPDGTVKLLIYYTSRLHSGVPSRFSGSGSGTDYSLTIRNLEQEDVATYFCQQGYTLPYTFGGGTKLEIK

SEQ ID NO: 313 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 314 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 315 - protein, 127 aa, Artificial Sequence
  Description of Artificial Sequence Synthetic polypeptide
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 316 - protein, 107 aa, Artificial Sequence
  Description of Artificial Sequence Synthetic polypeptide
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 317 - protein, 8 aa, Artificial Sequence
  Description of Artificial Sequence Synthetic peptide
GFTFSNYA

SEQ ID NO: 318 - protein, 8 aa, Artificial Sequence
  Description of Artificial Sequence Synthetic peptide
ISGSGGAT

SEQ ID NO: 319 - protein, 20 aa, Artificial Sequence
  Description of Artificial Sequence Synthetic peptide
TKDRLIMATVRGPYYYGMDV

SEQ ID NO: 320 - protein, 6 aa, Artificial Sequence
  Description of Artificial Sequence Synthetic peptide
QSISSY

SEQ ID NO: 321 - protein, 3 aa, Artificial Sequence
  Description of Artificial Sequence Synthetic peptide
AAS

SEQ ID NO: 322 - protein, 9 aa, Artificial Sequence
  Description of Artificial Sequence Synthetic peptide
QQSHSVSFT

SEQ ID NO: 323 - protein, 449 aa, Artificial Sequence
  Description of Artificial Sequence Synthetic polypeptide
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 324 - protein, 214 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 8 aa, Artificial Sequence
  Description of Artificial Sequence Synthetic peptide
GFTFNSYA

SEQ ID NO: 326 - protein, 8 aa, Artificial Sequence
  Description of Artificial Sequence Synthetic peptide
ISGSGGFT

SEQ ID NO: 327 - protein, 13 aa, Artificial Sequence
  Description of Artificial Sequence Synthetic peptide
AKDRLVAPGTFDY

SEQ ID NO: 328 - protein, 6 aa, Artificial Sequence
  Description of Artificial Sequence Synthetic peptide
QGISSW

SEQ ID NO: 329 - protein, 3 aa, Artificial Sequence
  Description of Artificial Sequence Synthetic peptide
AAS

SEQ ID NO: 330 - protein, 9 aa, Artificial Sequence
  Description of Artificial Sequence Synthetic peptide
QQYNSYPYT

SEQ ID NO: 331 - protein, 120 aa, Artificial Sequence
  Description of Artificial Sequence Synthetic polypeptide
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 332 - protein, 107 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 333 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 334 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 335 - protein, 1193 aa, Homo sapiens
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 336 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 337 - protein, 122 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSA

SEQ ID NO: 338 - protein, 112 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKR

SEQ ID NO: 339 - protein, 438 aa, Homo sapiens
MVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 340 - protein, 5 aa, Artificial Sequence
  Description of Artificial Sequence Synthetic peptide
TSWIG

SEQ ID NO: 341 - protein, 17 aa, Artificial Sequence
  Description of Artificial Sequence Synthetic peptide
IIYPGDSDTRYSPSFQG

SEQ ID NO: 342 - protein, 14 aa, Artificial Sequence
  Description of Artificial Sequence Synthetic peptide
HGLGYNGYEGAFDI

SEQ ID NO: 343 - protein, 11 aa, Artificial Sequence
  Description of Artificial Sequence Synthetic peptide
RASQGIGSALA

SEQ ID NO: 344 - protein, 7 aa, Artificial Sequence
  Description of Artificial Sequence Synthetic peptide
DASNLET

SEQ ID NO: 345 - protein, 9 aa, Artificial Sequence
  Description of Artificial Sequence Synthetic peptide
QQLNGYPLT

SEQ ID NO: 346 - protein, 123 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 347 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 348 - protein, 9 aa, Artificial Sequence
  Description of Artificial Sequence Synthetic peptide
FTFSDADMD

SEQ ID NO: 349 - protein, 19 aa, Artificial Sequence
  Description of Artificial Sequence Synthetic peptide
RTRNKAGSYTTEYAASVKG

SEQ ID NO: 350 - protein, 12 aa, Artificial Sequence
  Description of Artificial Sequence Synthetic peptide
AREPKYWIDFDL

SEQ ID NO: 351 - protein, 11 aa, Artificial Sequence
  Description of Artificial Sequence Synthetic peptide
RASQSISSYLN

SEQ ID NO: 352 - protein, 7 aa, Artificial Sequence
  Description of Artificial Sequence Synthetic peptide
AASSLQS

SEQ ID NO: 353 - protein, 9 aa, Artificial Sequence
  Description of Artificial Sequence Synthetic peptide
QQSYIAPYT

SEQ ID NO: 354 - protein, 121 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 355 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK